(19) United States
(12) Patent Application Publication
(10) Pub. No.: US 2023/0201183 A1
(43) Pub. Date: Jun. 29, 2023

(54) METHODS TO SYNERGISTICALLY ENHANCE MULTIPLE CELLULAR PROTEOSTASIS PATHWAYS TO TREAT NEURODEGENERATION AND STORAGE DISEASES

(71) Applicant: Northwestern University, Evanston, IL (US)

(72) Inventors: Joseph Robert Mazzulli, Chicago, IL (US); Iva Stojkovska, Evanston, IL (US); Willayat Yousuf Wani, Evanston, IL (US)

(21) Appl. No.: 18/056,634

(22) Filed: Nov. 17, 2022

Related U.S. Application Data
(60) Provisional application No. 63/264,224, filed on Nov. 17, 2021.

(51) Int. Cl. A61K 31/454; A61K 31/4709

(52) U.S. Cl. CPC A61K 31/454; A61K 31/4709; G01N 33/6896

(57) ABSTRACT

Disclosed are methods for treating and/or preventing a disease, disorder, or condition that is associated with accumulation of α-synuclein in a subject in need thereof. The method comprises administering to the subject a therapeutically effective amount of (a) at least one ER-Golgi trafficking enhancer in combination with at least one ER protein folding enhancer, or (b) at least one N-glycosylation enhancer, or a pharmaceutical composition comprising a therapeutically effective amount of (a) at least one ER-Golgi trafficking enhancer and at least one ER protein folding enhancer, or (b) at least one N-glycosylation enhancer.

### CROSS-REFERENCE TO RELATED APPLICATIONS

This application claims the benefit of priority of U.S. Provisional patent application Ser. No. 63/264,224, filed Nov. 17, 2021, the contents of which is incorporated by reference in its entirety.

### STATEMENT REGARDING FEDERALLY SPONSORED RESEARCH OR DEVELOPMENT

This invention was made with government support under NS092823 awarded by the National Institutes of Health, and National Institute of Neurological Disorders and Stroke. The government has certain rights in the invention.

### REFERENCE TO AN ELECTRONIC SEQUENCE LISTING

The contents of the electronic sequence listing (702581.02259.xml; Size: 13,372 bytes; and Date of Creation: Nov. 16, 2022) is herein incorporated by reference in its entirety.

### BACKGROUND

Proteinopathies and other storage disorders result from the inability to degrade waste material from the cell, leading to pathological accumulation of inclusion bodies and toxicity. Storage material also builds up in a chronic manner during the normal aging process and is associated with cognitive decline. The toxicity of protein accumulation is best exemplified by age-related neurodegenerative disorders, including Parkinson's disease (PD), Lewy body Dementia (LBD), and Alzheimer's disease (AD), which are all characterized by the accumulation of insoluble protein and lipid aggregates within the nervous system.

Parkinson's disease (PD) is a neurodegenerative movement disorder that is primarily characterized by the loss of dopaminergic neurons in the substantia nigra pars compacta region of the midbrain. Pathologically, PD is characterized by the presence of protein inclusions called Lewy bodies and Lewy neurites that are largely comprised of the synaptic protein α-synuclein (α-syn) (Spillantini et al., 1997). Critical to the pathogenic mechanism of α-syn, A53T and other familial-linked point mutations in SNCA, the gene that codes for α-syn, result in the accelerated oligomerization or fibrillization of the protein (Conway et al., 1998). Aside from point mutations, multiplications of wild-type SNCA also lead to familial PD. Patients with SNCA multiplications accumulate wild-type α-syn protein, and the severity of their clinical phenotype is dependent on α-syn dosage. For example, the clinical presentation of SNCA duplication patients occurs much later and is not as severe as in SNCA triplications (Fuchs et al., 2007; Singleton et al., 2003). Moreover, patients with SNCA multiplications display the classical Lewy pathology, indicating that the mere overabundance of the wild-type protein leads to neurodegeneration. However, the molecular mechanism by which the overabundance of wild-type α-syn leads to cellular toxicity and contributes to PD pathogenesis is not completely understood.

The accumulation of α-syn into insoluble inclusions suggests that proteostasis pathways, including the lysosomal clearance pathway, are disrupted in the PD brain. Genome-wide association studies in PD patients have identified several risk genes, most of which have key roles in autophagy and lysosomal function (Chang et al., 2017; Nalls et al., 2014; Robak et al., 2017; Simon-Sanchez et al., 2009). Of these, loss-of-function mutations in GBA1 represent one of the strongest genetic risk factors for the development of PD and Dementia with Lewy bodies (DLB) (Chia et al., 2021; Sidransky et al., 2009). Homozygous mutations in the GBA1 gene, which encodes lysosomal β-glucocerebrosidase (GCase), cause the lysosomal storage disorder Gaucher's disease (GD) that is characterized by glycosphingolipid accumulation and neurodegeneration (Roshan Lal and Sidransky, 2017).

Aside from mutations in lysosomal components, genetic analyses indicate that defects in another critical component of the proteostasis network, vesicular trafficking, also contribute to PD pathogenesis (Abeliovich and Gitler, 2016; Hunn et al., 2015; Klein and Mazzulli, 2018; Martin et al., 2014; Singh and Muqit, 2020). Proper GCase maturation requires its trafficking from the endoplasmic reticulum (ER) to the Golgi portion of the secretory pathway. Previous work has shown that α-syn accumulation can impair ER-to-Golgi protein trafficking and disrupt protein maturation (Cooper et al., 2006; Gitler et al., 2008; Gosavi et al., 2002; Thayanidhi et al., 2010). Our recent studies showed that trafficking disruption occurs by α-syn-mediated inhibition of the SNARE protein ykt6, which prevents the fusion of ER-derived vesicles on the cis-Golgi causing downstream lysosomal depletion (Cuddy et al., 2019).

Perhaps the most critical function of the proteostasis network takes place in the ER compartment, where approximately one-third of the cell's proteome is synthesized, folded, and processed. Calcium-dependent molecular chaperones play a pivotal role in maintaining ER proteostasis. The calcium-dependent ER lectin-containing chaperone calnexin (CANX) is particularly important for maintaining proper protein folding and quality control of monomeric N-linked glycosylated proteins, including GCase and other lysosomal hydrolases (Ou et al., 1993; Tan et al., 2014). Disrupted protein trafficking and accumulation of immature proteins in the ER can overwhelm the folding machinery, invariably leading to ER stress and initiation of the unfolded protein response (UPR). The UPR constitutes a series of pathways that transduce ER stress signals to the nucleus for transcriptional upregulation of quality control machinery and expansion of the ER to accommodate excess protein load (Walter and Ron, 2011). The three main stress sensors that initiate the UPR include inositol-requiring enzyme (IRE1), double-stranded RNA-activated protein kinase (PKR)-like ER kinase (PERK), and activating transcription factor 6 (ATF6). These three branches can be stimulated separately or simultaneously, and attempt to restore ER proteostasis by reducing protein synthesis and stimulating gene expression of folding machinery including chaperones GRP78 and GRP94 (Kozutsumi et al., 1988). GBA1 mutations destabilize GCase structure, resulting in UPR induction, expansion of the ER compartment, and elimination of the protein through ER associated degradation (ERAD) (Fernandes et al., 2016; Garcia-Sanz et al., 2017; Ron and Horowitz, 2005). General markers of UPR activation have also been documented in various synucleinopathy models including α-syn overexpressing yeast (Cooper et al., 2006), human mutant SNCA A53 T transgenic mice (Colla et al., 2012a; Colla et al., 2018), as well as iPSC-derived cortical neuron models (Chung et al., 2013; Heman-Ackah et al., 2017). Importantly, evidence of UPR activation is present in the substantia nigra of post-mortem PD brains (Credle et al., 2015; Heman-Ackah et al., 2017; Hoozemans et al., 2007). Overexpression of the ER chaperone GRP78 can also reduce neurodegeneration in α-syn expressing animal models (Gorbatyuk et al., 2012), further emphasizing the importance of maintaining ER proteostasis in neuronal health. While these studies collectively suggest that ER dysfunction is associated with PD, the mechanistic link between α-syn accumulation, protein misfolding in the ER, and downstream lysosomal dysfunction has not been established. Furthermore, it is unknown whether enhancing ER proteostasis alone is sufficient to rescue lysosomal dysfunction and reduce pathological α-syn in PD models.

Glucose flux through the hexosamine biosynthetic pathway (HBP) is essential for generating the high-energy sugar-nucleotide donor uridine-diphosphate-N-acetylglucosamine (UDP-GlcNAc), which provides the main glycan source for protein N-glycosylation in the ER (Denzel and Antebi, 2015). The HBP begins with the conversion of obligatory intermediate fructose-6-phosphate (F-6-P) from the initial steps of glycolysis, and glutamine to form glucosamine-6-phosphate by the rate-limiting enzyme, glutamine:F-6-P transaminase (GFPT), ultimately forming UDP-GlcNAc. Two isozymes of GFPT exist (GFPT1 and GFPT2) and are expressed from distinct genes with variable expression patterns. In the central nervous system, GFPT2 (GFAT2, EC 2.6.1.16) is the predominant isoform (Oki et al., 1999). UDP-GlcNAc is utilized in the committed step of N-glycosylation by dolichyl-phosphate-N-acetyl-glucosamine-phosphotransferase-1 (DPAGT1), which is the tunicamycin target (Dong et al., 2018; Heifetz et al., 1979). The addition of N-glycans is required for the proper folding and trafficking of proteins that mature through the ER-Golgi pathway including lysosomal hydrolases (Hebert et al., 2014). In vivo studies have shown that enhancing glycosylation through activation of the HBP can improve proteostasis and extend lifespan in worms (Denzel et al., 2014; Wang et al., 2014), however the mechanism has yet to be fully delineated. The importance of the HBP and N-glycosylation in proteostasis is also demonstrated by genetic diseases caused by mutations in key HBP enzymes. For example, a rare congenital disorder of glycosylation caused by loss-of-function mutations in DPATG1 primarily affects the nervous system and is clinically characterized by hypokinesia, cognitive impairment, and microcephaly (Carrera et al., 2012; Freeze et al., 2015; Wu et al., 2003).

### SUMMARY

Disclosed are methods for treating and/or preventing a disease, disorder, or condition that is associated with accumulation of α-synuclein. The disclosed methods can be utilized to treat a neurodegenerative disorder, a rapid eye movement (REM) behavior sleep disorder, a pediatric lysosomal storage disorder, and/or a protein misfolding or amyloidosis disorder.

The disclosed methods may be directed to treatment and/or prevention of a disease, disorder, or condition that is associated with accumulation of α-synuclein in a subject in need thereof. The methods comprise administering to the subject a therapeutically effective amount of (a) at least one protein trafficking enhancer (including a farnesyltransferase inhibitor (FTI), ykt6 activator, Rab1A activator, or other methods to enhance trafficking between the endoplasmic reticulum (ER) and the Golgi) in combination with at least one ER protein folding enhancer (including a ryanodine receptor (RyR) inhibitor, an inositol triphosphate (IP3) receptor inhibitor, an activator of the sarco/endoplasmic reticulum Ca²⁺-ATPase (SERCA), or (b) at least one enhancer of N-glycosylation pathways in the ER, or other methods that activate the unfolded protein response (UPR)), or a pharmaceutical composition comprising a therapeutically effective amount of (a) at least one ER-Golgi trafficking enhancer and at least one ER protein folding enhancer, or (b) at least one N-glycosylation enhancer.

In some embodiments, the disclosed methods are directed to a disease, disorder, or condition that is a neurodegenerative disease, disorder, or condition selected from the group consisting of Parkinson's disease, Lewy body dementia, Alzheimer's disease, amyotrophic lateral sclerosis (ALS), multiple system atrophy, Huntington's disease, Prion disease, frontotemporal dementia, Picks disease, progressive supranuclear palsy, progeria, and any combinations thereof.

In other embodiments, the disclosed methods are directed to a rapid eye movement (REM) behavior sleep disorder linked to Parkinson's disease and/or GBA1 mutation carriers.

In certain embodiments, the disclosed methods are directed to a pediatric lysosomal storage disorder selected from the group consisting of glycogen storage disorder, neuronal ceroid lipofuscinosis disorder, sphingolipid storage disorder, cholesterol storage disorder, fatty acid storage disorder, and any combinations thereof.

In certain embodiments, the disclosed methods are directed to a protein misfolding and/or amyloidosis disease, disorder, or condition selected from the group consisting of cataract caused by α-crystallin aggregation, systemic amyloidosis, type 2 diabetes characterized by amylin aggregation, alpha-1-antitrypsin deficiency liver disease, and any combinations thereof.

In certain embodiments, the disclosed ER-Golgi trafficking enhancer and ER protein folding enhancer may be protonated, for example to form an ammonium moiety, optionally where the compound is present as a salt (e.g., an ammonium salt). The disclosed ER-Golgi trafficking enhancer and ER protein folding enhancer also may be non-protonated and/or dissociated, for example, where the carboxylic acid moiety is dissociated to from a carboxylate moiety, optionally where the compound is present as a salt (e.g., a carboxylate salt). The disclosed ER-Golgi trafficking enhancer and ER protein folding enhancer may be in zwitterionic form where the ER-Golgi trafficking enhancer and ER protein folding enhancer comprise a protonated ammonium moiety and a dissociated carboxylate moiety, optionally where the ER-Golgi trafficking enhancer and ER protein folding enhancer is present as a salt.

The ER-Golgi trafficking enhancer and ER protein folding enhancer disclosed herein are without stereochemical or configurational limitation and encompass all stereochemical or configurational isomers, unless stereochemical or configurational limitations are indicated. As illustrated and discussed below, such ER-Golgi trafficking enhancer and ER protein folding enhancer and/or their intermediates are available as single enantiomers, racemic mixtures from which isomers can be resolved, or diastereomers from which the corresponding enantiomers can be separated. Accordingly, any stereocenter can be (S) or (R) with respect to any other stereocenter(s). As another separate consideration, various ER-Golgi trafficking enhancer and ER protein folding enhancer can be present as an acid or base salt, either partially or fully protonated, for example at the amino group to form an ammonium moiety, and/or either partially or fully dissociated, for example at the carboxyl group to form a carboxylate substituent or moiety. In certain such embodiments, with respect to an ammonium substituent or moiety, the counter ion can be a conjugate base of a protic acid. In certain such or other embodiments, with respect to a carboxylate substituent or moiety, the counter ion can be an alkaline, alkaline-earth or ammonium cation. Further, it will be understood by those skilled in the art that any one or more the ER-Golgi trafficking enhancer and ER protein folding enhancer disclosed herein can be provided as part of a pharmaceutical composition comprising a pharmaceutically acceptable carrier component for use in conjunction with a treatment method or medicament.

In certain embodiments, β-glucocerebrosidase (GCase) maturation level is increased in the subject after administration of a therapeutically effective amount of (a) at least one ER-Golgi trafficking enhancer in combination with at least one ER protein folding enhancer, or (b) at least one N-glycosylation enhancer, or a pharmaceutical composition comprising a therapeutically effective amount of (a) at least one ER-Golgi trafficking enhancer and at least one ER protein folding enhancer, or (b) at least one N-glycosylation enhancer.

In certain embodiments, GCase solubility is increased in the subject after administration of a therapeutically effective amount of (a) at least one ER-Golgi trafficking enhancer in combination with at least one ER protein folding enhancer, or (b) at least one N-glycosylation enhancer, or a pharmaceutical composition comprising a therapeutically effective amount of (a) at least one ER-Golgi trafficking enhancer and at least one ER protein folding enhancer, or (b) at least one N-glycosylation enhancer.

In certain embodiments, the amount of α-synuclein is reduced in the subject after administration of a therapeutically effective amount of (a) at least one ER-Golgi trafficking enhancer in combination with at least one ER protein folding enhancer, or at least one N-glycosylation enhancer, or a pharmaceutical composition comprising a therapeutically effective amount of (a) at least one ER-Golgi trafficking enhancer and at least one ER protein folding enhancer, or (b) at least one N-glycosylation enhancer.

In certain embodiments, endoplasmic reticulum (ER) morphology is improved in the subject after administration of a therapeutically effective amount of (a) at least one ER-Golgi trafficking enhancer in combination with at least one ER protein folding enhancer, or (b) at least one N-glycosylation enhancer, or a pharmaceutical composition comprising a therapeutically effective amount of (a) at least one ER-Golgi trafficking enhancer and at least one ER protein folding enhancer, or (b) at least one N-glycosylation enhancer.

In certain embodiments, the administration of the at least one ER-Golgi trafficking enhancer and the at least one ER protein folding enhancer produces a synergistic effect.

In certain embodiments, the administration of the at least one ER-Golgi trafficking enhancer, the at least one ER protein folding enhancer, and the N-glycosylation enhancer produces a synergistic effect.

In certain embodiments, the (a) at least one ER-Golgi trafficking enhancer and the at least one ER protein folding enhancer, or (b) at least one N-glycosylation enhancer can be provided as part of a pharmaceutical composition.

More generally, the disclosed methods may be directed to treating and/or preventing a disease, disorder, or condition that is associated with accumulation of α-synuclein. Such a method can comprise providing a therapeutically effective amount of (a) at least one ER-Golgi trafficking enhancer in combination with at least one ER protein folding enhancer, or (b) at least one N-glycosylation enhancer, as described below, whether or not part of a pharmaceutical composition. Such ER-Golgi trafficking enhancer, ER protein folding enhancer, and N-glycosylation enhancer can include but are not limited to those illustrated by the following examples, referenced figures, incorporated references and/or accompanying synthetic schemes. In certain such embodiments, such ER-Golgi trafficking enhancer, ER protein folding enhancer, and N-glycosylation enhancer, and/or combination thereof can be present in an amount at least partially sufficient to increase GCase maturation, increase GCase solubility, reduce the amount of α-synuclein and/or improve endoplasmic reticulum morphology.

### BRIEF DESCRIPTION OF THE FIGURES

FIGS. 1A-1H. Defects in GCase maturation and in lysosomal function occur specifically through α-syn accumulation in SNCA-3X midbrain DA neurons. (A) Western blot analysis of immature (˜55-62 kDa) and mature (>62 kDa) GCase forms in day 90 healthy control and SNCA-3X DA neurons. Previously established lines, Est. 3× and Est. Ctrl are shown as a relative comparison. α-Syn (antibody C20) expression to confirm overexpression is included. Right, immature GCase levels were normalized to the Coomassie stained gel and GCase maturation was calculated by measuring the post-ER (mature) to ER (immature) GCase ratio. (B,C) Live-cell lysosomal GCase activity and neurofilament analysis of day 90 SNCA-3X DA neurons (3x−1, clones C2 and C3, are shown as a representation). Quantification is shown as a fold change to two healthy controls: Ctrl (C1) and Est. Ctrl (n=3 per line). (D) Quantitative RT-PCR analysis of SNCA mRNA expression relative to GAPDH in day 90 DA neurons of each SNCA-3X and isogenic control (n=5-9 per line). (E) Representative western blot analysis of soluble and insoluble α-syn (antibody C20) levels in SNCA-3X and isogenic control DA neurons. The healthy control (Ctrl) is included for a relative comparison. The dashed line indicates irrelevant lanes that were cropped out. Right, quantification of soluble α-syn levels in the isogenic control lines normalized to Coomassie, shown as a fold change to each parental SNCA-3X line (n=2). (F) Representative western blot of GCase maturation in day 90 SNCA-3X and isogenic control DA neurons. Right, quantification of immature GCase levels normalized to Coomassie and GCase maturation combined from lines 3x−1, 3x−2, and 3x−4 and their corresponding isogenic controls (n=2 from each line). (G) Representative western blot of GCase in endoglycosidase H (Endo H) digested lysates of day 120 SNCA-3X and isogenic control neurons. Right, levels of Endo H-resistant GCase were measured by quantifying mature/post-ER GCase from the Endo H lanes and normalizing to Coomassie (n=3 per line). (H) Live-cell lysosomal GCase activity in day 60 and day 120 3x−1 and isogenic control DA neurons (n=2-3). Activity is shown as a fold change to the isogenic control at both time points. For all quantifications, values are mean±SEM, *p<0.05; **p<0.01; ****p<0.0001; ns=not significant, using student's unpaired t-test.

FIGS. 2A-2D. Accumulation of insoluble immature GCase in SNCA-3X midbrain DA neurons and synucleinopathy brains. (A) Day 90 SNCA-3X DA neurons were sequentially extracted and the Triton X-100-insoluble fractions were analyzed by western blot for GCase. Three replicates of line 3x−4 are shown as a representative example. Right, quantification of insoluble GCase normalized to Coomassie combined from lines 3x−1, 3x−2, and 3x−4 (n=3-4 from each line, n=10 total). (B) PD patient line expressing A53T α-syn and isogenic control were analyzed as in A. (C) GCase western blot analysis of 1% sarkosyl insoluble extracts from the frontal cortex in controls or synucleinopathy patient brains (DLB, dementia with Lewy bodies; DLB+AD, DLB with Alzheimer's disease (AD) pathology). Right, quantification of insoluble GCase normalized to Coomassie and grouped by similar post mortem intervals (PMI) (blue, PMI=<10 hrs; black, PMI<20 hrs; red, PMI<30 hrs). (D) ER microsome fractions from the cingulate cortex of 3 controls and 3 PD brains were sequentially extracted and Triton X-100-insoluble lysates were analyzed by western blot for GCase. Right, quantification of insoluble GCase from microsome fractions, normalized to Coomassie. For all quantifications, values are the mean±SEM, *p<0.05; **p<0.01, using student's unpaired t-test (panels A, B, D) or ANOVA with Dunnetts T3 test (panel C). In B and C, the dashed line represents cropped out lanes from the same blot. See Tables 1-3 for clinical and pathological data and raw quantifications of patient brain.

FIGS. 3A-3E. Alterations in ER morphology and characterization of the ER stress response in SNCA-3X midbrain DA neurons. (A) Representative ER ultrastructure of day 65 SNCA-3X (3x−2), mutant GBA1 (N370S/84GG), and control DA neurons. Examples of ER segments are highlighted in red. The white boxes indicate the zoomed-in regions of the images underneath. N=nucleus, *=ER (endoplasmic reticulum). Scale bar, 500 nm. Right, quantifications of length and area of each clearly identifiable ER segment in each line (n=3-9 cells per line; each data point on the scatter plot indicates a measured ER segment). (B) Baseline expression of ER stress markers in day 90 SNCA-3X (lines 3x−1, 3x−2, 3x−4), mutant GBA1 (N370S/84GG and L444P/L444P), and corresponding isogenic and healthy control DA neurons (n=2-5 for each line). Levels of SNCA-3X and mutant GBA1 neuron ER stress markers were normalized to Coomassie, and are shown as a fold change to the isogenic or healthy controls, respectively. Each data point in the scatter plot indicates a biological replicate taken from several experiments. (C) GRP78 and CANX mRNA expression in day 90 SNCA-3X and isogenic controls was measured via quantitative RT-PCR, and was quantified relative to b-actin. Top, GRP78 mRNA was combined from 3x−1 and 3x−2 and corresponding isogenic controls (n=2-3 per line). Bottom, CANX mRNA was combined from 3x−1, 3x−2, and 3x−4 and corresponding isogenic controls (n=3-7 per line). (D) Healthy control (wild-type) or SNCA-3X neurons were (3x−2) were treated with lentiviral particles to express L444P GCase at a multiplicity of infection (MOI) of 1, followed by incubation for 2 weeks, and harvested at day 90. mRNA was quantified to confirm L444P GCase expression (left) and ER stress response was determined by GRP78 (middle) and GRP94 (right) (n=3). (E) Neurons were treated with vehicle (veh) or 30 nM thapsigargin (Tg) for 24 hours to induce ER stress and activate the UPR, and analyzed for induction of ER stress markers at day 70 and 90. Irrelevant lanes were cropped out from the blot and are indicated with a dashed line. Right, GRP78 and GRP94 levels were normalized to Coomassie, and are shown as a fold change to the vehicle for each set of lines. Analysis is of several lines of SNCA-3X: 3x−1, 3x−2, 3x−4, Est. 3×; isogenic controls: 3x−1 iso ctrl, 3x−2 iso ctrl, Est. 3×iso ctrl; healthy controls: Ctrl, Est. Ctrl. Each data point in the scatter plot indicates a biological replicate taken from several experiments. For all quantifications, values are mean±SEM, *p<0.05; **p<0.01; ***p<0.001; ****p<0.0001; ns=not significant, using student's unpaired (panels B,C), paired t-test (panel E), or ANOVA with Tukey's post-hoc test (panels A,B,D).

FIGS. 4A-4F. Analysis of EDEM expression and ERAD in SNCA-3X midbrain DA neurons. (A) EDEM1 mRNA expression relative to b-actin in day 90 SNCA-3X and isogenic control DA neurons. Analysis is combined from lines 3x−1, 3x−2, and 3x−4 and the corresponding isogenic (n=4 per line). (B) Representative western blot of EDEM1 in day 90 SNCA-3X and GBA1 mutant DA neurons that accumulate α-syn. (C) Quantification of EDEM1 expression in SNCA-3X and GBA1 mutant DA neurons shown as a fold change to the isogenic and healthy controls, respectively. Analysis is combined from 3x−1, 3x−2, 3x−4, and Est. 3×(n=5-13 per line) and the corresponding isogenic controls (n=5-12 per line), as well Ctrl and Est. Ctrl for the healthy controls (n=2-6 per line). (D) Correlation between EDEM1 and α-syn levels from SNCA-3X patient DA neurons indicated in panel C. Each data point in the scatter plot indicates a biological replicate of a patient sample. (E) Day 90 SNCA-3X and control DA neurons were treated with 50 nM epoxomicin (Epox) for 24 hours to inhibit proteasomal degradation, and GCase and ubiquitin levels were analyzed via western blot. Right, quantification of GCase and ubiquitin levels normalized to Coomassie. Analysis is combined from 3x−1, 3x−2, and Est. 3×, and isogenic controls are from 3x−2 and Est. 3×(n=3 per line). (F) GCase expression in GBA1 mutant DA neurons treated with 1 uM Epox. Right, quantification of GCase levels normalized to Coomassie (n=5-6). For all quantifications, values are mean±SEM, *p<0.05; **p<0.01; ***p<0.001; ****p<0.0001; ns=not significant, using student's unpaired (panels A,C,E,F) or paired t-test (panel E) or ANOVA with Tukey's post-hoc test (panel C).

FIGS. 5A-5B. Accumulation of α-syn leads to its increased association with ER chaperones. (A) H4 WT α-syn overexpressing cells were treated with 1 ug/ul DOX for 3 days to turn off α-syn expression. Increased association of α-syn with CANX was determined by a proximity ligation assay (PLA). Cell nuclei are stained with DAPI. Right, PLA signal was calculated as the number of red particles normalized to the number of nuclei within an acquired field of view (n=5), and is shown as a fold change to the +DOX condition. Scale bar, 25 um. (B) PLA analysis of Day 70 SNCA-3X and control DA neurons shows increased association of α-syn with CANX (top) and GRP94 (bottom). Cell nuclei are stained with DAPI. Right, PLA signal was quantified as in A, and each data point on the scatter plot represents the PLA signal from an acquired field of view from n=3-4 biological replicates per line. Scale bar, 25 um. For all quantifications, values are mean±SEM, *p<0.05; **p<0.01; ****p<0.0001, using student's unpaired t-test (panel A) or ANOVA with Tukey's post-hoc test (panel B).

FIGS. 6A-6F. Enhancement of ER proteostasis improves GCase solubility and function in SNCA-3X DA neurons but fails to reduce α-synuclein. SNCA-3X DA neurons were treated with vehicle (veh) or 25 uM diltiazem (DILT), a ryanodine receptor (RyR) inhibitor. (A) Neurons were treated for 2 or 8 weeks, sequentially extracted, and analyzed for levels of soluble and insoluble GCase. Western blot of day 90, 3x−1 DA neurons treated for 8 weeks is shown as a representation. Right, the percentage of insoluble GCase was calculated by dividing insoluble GCase intensity by total GCase taken from both soluble and insoluble fractions. Quantification for the 2-week treatment is combined from day 90 3x−1 (n=6) and 3x−2 (n=3) neurons; 8-week treatment is of day 120 3x−1 neurons only (n=3). (B) GCase maturation of day 90 SNCA-3X DA neurons treated with DILT for 2 weeks was analyzed by western blot. Coomassie is shown as a loading control. Right, quantification of GCase maturation is combined from lines 3x−1 (clones C2 and C3), 3x−2, and 3x−4 (n=3 per line). (C) Left, whole cell GCase activity of SNCA-3X lysates from panel B. Right, live-cell lysosomal GCase activity in day 90 SNCA-3X DA neurons treated with DILT for 2 weeks. (D) Western blot analysis was used to determine whether enhancing GCase proteostasis by improving ER function has an effect on α-syn levels. The blot from panel B was reprobed for α-syn and is shown here. GAPDH is shown as a loading control. Right, quantification of α-syn levels normalized to Coomassie at 2 and 8 weeks of DILT treatment. For the 2-week treatment, data is combined from lines 3x−1 (clones C2 and C3), 3x−2, and 3x−4 (n=3 per line); 8-week treatment is of day 120 3x−1 neurons only (n=3). (E) H4 wild-type α-syn overexpressing cells were treated for 5 days with veh or DILT and N-glycosylated proteins were pulled down using concanavalin A (Con-A). Binding of CANX to Con-A was assessed by western blot and used as a measure of CANX function. GCase and GAPDH serve as positive and negative controls for the pulldown, respectively. Right, quantification of CANX levels in the Con-A pulldown in DILT treated cells, normalized to GAPDH from the input (n=3-4). (F) SNCA-3X DA neurons were infected with scrambled (scrb) or RyR3 shRNA to knock-down (KD) RyR3 (MOI=5, 2 weeks), sequentially extracted, and analyzed for soluble and insoluble GCase. Western blot of day 90 3x−1 neurons is shown as a representation. Right, percentage of insoluble GCase and GCase maturation in RyR3 KD neurons (n=3). For all quantifications, values are mean±SEM, *p<0.05; ***p<0.001; ****p<0.0001; ns=not significant, using student's unpaired t-test.

FIGS. 7A-7E. Rescue of ER fragmentation and lysosomal GCase by synergistic enhancement of ER proteostasis and trafficking. SNCA-3X DA neurons were treated with vehicles, 5 nM farnesyl transferase inhibitor (FTI), 25 uM diltiazem (DILT), or combined FTI+DILT. (A) GCase maturation of day 90 3x−1 DA neurons treated with FTI+DILT for 2 weeks was analyzed by western blot. Coomassie is shown as a loading control. Right, quantification of GCase maturation for the 2-week treatment is combined from day 90 3x−1 (n=2) and 3x−2 (n=3) neurons; quantification for the 4-week treatment is from day 120 3x−1 neurons only (n=3). (B) Day 90 3x−2 DA neurons were treated with FTI+DILT for 2 weeks and ER morphology was assessed by EM imaging. Examples of ER segments are highlighted in red. Scale bar, 1 mm. Right, quantification of the length and area of each clearly identifiable ER segment (n=3 cells for vehicle condition, n=9 cells for FTI+DILT condition; each data point on the scatter plot indicates a measured ER segment). (C) Left, whole cell GCase activity of treated 3x−1 and 3x−2 lysates from panel A (n=5). Right, live cell GCase activity of veh or FTI+DILT treated 3x−2 DA neurons (n=4-5). GCase activity in healthy control neurons (Est. Ctrl) is shown as a comparison (n=7). (D) The blot from panel A was sequentially probed with 211, then 303 anti-α-syn antibodies. GAPDH is shown as a loading control. Right, quantification of total and pathological α-syn levels normalized to Coomassie in day 90 3x−1 (n=2) and 3x−2 (n=3) neurons treated with FTI+DILT for 2 weeks. (E) Day 90, 3x−2 DA neurons treated with FTI+DILT for 2 weeks were sequentially extracted and the Triton X-100-insoluble fractions were analyzed by western blot for α-syn. Membranes were sequentially probed with syn211 followed by syn303 anti-α-syn antibodies. Irrelevant lanes were cropped out from the blot and are indicated with a dashed line. Right, quantification of total and pathological α-syn levels normalized to Coomassie. For all quantifications, values are mean±SEM, *p<0.05; **p<0.01; ***p<0.001; ****p<0.0001, using student's unpaired t-test (panels B, C right) or ANOVA with Tukey's post-hoc test (panels A, D, C (left), E).

FIG. 8. Schematic overview of proteostasis dysfunction and rescue in PD patient neurons that accumulate wild-type α-synuclein. A pathogenic cascade is triggered upon α-synuclein (α-syn) accumulation at the ER, leading to ER fragmentation, failure of UPR activation, and accumulation and aggregation of immature β-glucocerebrosidase (GCase) (1, 2). Enhancing ER folding capacity promotes the formation of soluble, properly folded GCase, while combined treatment with protein trafficking enhancers rescues lysosomal function and reduces pathological α-syn (3).

FIGS. 9A-9F. Characterization of SNCA-3X iPSCs and midbrain dopaminergic neurons. FIGS. 9A-9F linked to FIGS. 1A-1H. (A) Quantitative RT-PCR analysis of Nanog mRNA expression in SNCA-3X and healthy control iPSC clones (n=3 technical replicates per line). Lymphoblast cDNA and a no-template sample (qRT-PCR neg) serve as negative controls. GAPDH serves as a reference gene. Data is shown as mean±SD. (B) Immunofluorescence staining of several pluripotency and embryonic stem cell markers in SNCA-3× and healthy control (Ctrl) iPSCs. Cell nuclei are stained with DAPI. (C) PCR analysis of Oct3/4 (O), Sox2 (S), Klf4 (K), and L-Myc (M) plasmid transgenes in SNCA-3X and Ctrl iPSCs. A previously established healthy control iPSC line (Est. Ctrl) and a no-template control (NTC) sample serve as negative controls. The plasmids serve as positive controls for each transgene. (D) SNCA copy number analysis of SNCA-3X and Ctrl iPSCs (n=3-6 replicates per line from 3 separate experiments). Previously established SNCA-3X (Est. 3×) and healthy control (Est. Ctrl) iPSCs are shown as a relative comparison. RNaseP serves as a reference gene. Data is shown as mean±SEM, ****p<0.0001, using student's unpaired t-test or ANOVA with Tukey's post-hoc test. (E) Representative fluorescence in-situ hybridization (FISH) analysis of SNCA-3X and Ctrl iPSCs. The red signal indicates a probe that spans SNCA located in 4q22.1 and the green signal indicates a control probe targeting the 4p16.3 chromosomal region. The result confirms a total of four SNCA copies in the PD patient iPSC (4 red, 2 green; 4R2G) indicative of a triplication, while the healthy control contains two copies (2 red, 2 green; 2R2G). (F) Representative immunofluorescence staining of FoxA2 (red) and tyrosine hydroxylase (TH; green) (top) or the neuronal marker β3-tubulin (red) and TH (green) (bottom) in day 90 SNCA-3X iPSC-derived DA neurons. Cell nuclei are stained with DAPI. Right, quantification of FoxA2+TH (top) or β3-tubulin+TH (bottom) positive neurons as a percent of total cells for each patient line. Each data point in the scatter plot represents a field of view from different batches of differentiation. Data is shown as mean±SD. Scale bar, 20 um.

FIGS. 10A-10F. Analysis of α-Syn accumulation and generation of SNCA-3X isogenic control midbrain DA neurons. FIGS. 10A-10F linked to FIGS. 1A-1H. (A) Immunofluorescence (IF) staining of α-syn (antibody LB509) and β3-tubulin in day 90 Ctrl and SNCA-3X DA neurons. Cell nuclei are stained with DAPI. Scale bar, 20 mm. (B) Representative co-staining of α-syn (antibody LB509) with thioflavin S (ThioS) in Ctrl and 3x−1 DA neurons. Cell nuclei are stained with DAPI. Scale bar, 20 mm. (C) Sequential protein extraction and western blot analysis of soluble and insoluble α-syn in day 90 Ctrl and SNCA-3X DA neurons. Membranes were sequentially probed with C20 and LB509 anti-α-syn antibodies. Irrelevant lanes were cropped out from the blot and are indicated with a dashed line. Right, quantification of soluble and insoluble α-syn levels normalized to Coomassie. Each data point on the scatter plot indicates a biological replicate taken from several culture batches. Data is shown as mean±SEM, *p<0.05; **p<0.01; ***p<0.001; ****p<0.0001, using ANOVA with Tukey's post-hoc test. (D) Successful insertion of the puromycin cassette in the isogenic control (iso ctrl) iPSCs for each SNCA-3X patient was evaluated by copy number analysis (n=3). RNAseP serves as the reference gene. Quantification is shown as a fold change to a previously established control α-syn knockout (Est. Ctrl KO) iPSC line (Zunke et al., 2018), and statistical analysis was performed between each SNCA-3X line and its corresponding isogenic control. Data is shown as mean±SEM, **p<0.01; ***p<0.001, using student's unpaired t-test. (E) Predicted off-target effects for the selected guide RNAs were tested for mismatches via the T7 Endonuclease I (T7EI) assay, and the PCR products were analyzed by an agarose gel. Representative analysis of 3x−1 and its corresponding isogenic control are shown. Right, T7EI assay positive control. NTC, no template control. (F) Representative TH and FoxA2 immunofluorescence staining of day 90 DA neurons (n=3) indicates that midbrain neuron development is unaffected due to CRISPR editing or α-syn depletion. Cell nuclei are stained with DAPI. Right, quantification of FoxA2+TH positive neurons as a percent of total cells. Each data point on the scatter plot indicates a field of view. Data is shown as mean SD. Scale bar, 20 mm.

FIGS. 11A-11D. Specificity analysis of insoluble lysosomal hydrolases in models and in DLB patient brain. FIGS. 11A-11D linked to FIGS. 2A-2D. (A) Isogenic controls from line 3x−2 were treated with 30 nM thapsigargin (Tg) for 24 hours, followed by sequential extraction/western blot to assess the levels of insoluble GCase. GRP78 was analyzed to confirm the induction of ER stress. Quantification is shown to the right (n=3) (B) Brain extracts from 10 month old LIP2−/− mice were analyzed by sequential extraction/western blot for GCase as in panel A. LIMP2−/− blocked GCase maturation and resulted in an overall reduction of GCase. The asterisk indicates a putative non-specific band. Quantification is shown to the right (n=4). (C) Sequential extraction and western blot analysis for cathepsin D was performed as in FIG. 2C. Quantification of insoluble immature forms of cathepsin D, normalized to Coomassie stained gels is shown to the right (n=4-5). (D) Sequential extraction and western blot analysis of hexosaminidase B (Hex B). Only soluble forms of the hydrolase were detected. See Table 3 for clinical and pathological patient data and raw cathepsin D quantifications of the brain samples. For all quantifications, data represents mean±SEM, *p<0.05; ***p<0.001; ns=not significant, using student's unpaired t-test.

FIGS. 12A-12F. SNCA-3X midbrain DA neurons and cell lines that accumulate α-syn do not show signs of UPR activation, but respond to ER stressors. FIGS. 12A-12F linked to FIGS. 3A-3E and 4A-4F. (A) Semi-quantitative PCR agarose gel analysis of unspliced (U) and spliced (S) XBP1 mRNA in day 90 SNCA-3X and control DA neurons (top) and H4 α-syn overexpressing cells (bottom). Treatment with 50 ng/ml brefeldin A (BFA) was done as a positive control. The dashed line indicates irrelevant lanes that were cropped out. (B) XBP1-S mRNA expression in SNCA-3X and control DA neurons was measured by quantitative RT-PCR analysis, and is shown relative to b-actin. Left, baseline XBP1-S mRNA is combined from day 90 3x−1, 3x−2, and 3x−4 and the isogenic controls of 3x−1 and 3x−4 (n=3 per line). Right, XBP1-S mRNA from day 110 3x−2 and isogenic controls treated with 50 ng/ml BFA for 24 hours (n=3). (C,D) Day 90 SNCA-3X and control DA neurons (n=2 per line) and H4 α-syn overexpressing cells (n=4) were treated for 24 hours with ER stress inducers (30 nM Tg or 50 ng/ml BFA). mRNA expression of GRP78 and CANX was quantified relative to b-actin. In panel C, analysis is combined from lines 3x−2 and 3x−4, and the isogenic control is from 3x−4. (E) H4 WT α-syn overexpressing cells were first treated with 1 ug/ul doxycycline (DOX) for 3 days to turn off α-syn expression, followed by a 24-hour treatment with Tg to induce ER stress. Right, GRP78 and GRP94 quantification was normalized to Coomassie (n=3). (F) Representative western blot showing UPR induction in day 90 SNCA-3X and isogenic control DA neurons treated with 30 nM Tg. Right, quantification of the ratio of phosphorylated to total eIF2α and GRP78 expression normalized to Coomassie. Analysis is combined from 3x−1 and Est. 3×, and the isogenic control is from Est. 3×(n=3 per line). For all quantifications, values are mean±SEM, *p<0.05; **p<0.01; ***p<0.001; ns=not significant, using student's unpaired (panels B, C, E, F) or paired t-test (panels C, E, F) or ANOVA with Tukey's post-hoc test (panels B, D).

FIGS. 13A-C. Analysis of PLA negative and positive controls and the aberrant localization of α-syn to the ER in SNCA-3X DA neurons. FIGS. 13A-13C linked to FIGS. 5A-5B. (A) Super-resolution SIM analysis of protein disulfide isomerase (PDI) and colocalization with α-syn (antibody 211). Quantification of the colocalization in 3x−2 neurons compared to healthy control neurons that express normal levels of α-syn is shown on the right (n=3). (B) Subcellular fractionation of day 120 DA neurons and western blot analysis of α-syn (antibodies C20 and LB509) demonstrates its accumulation within ER microsome fractions in SNCA-3X neurons compared to controls. ER (GRP78, GRP94, CANX) and cytosolic (NSE) markers were analyzed to assess enrichment of the ER. Right, quantification of C20 and LB509 α-syn levels in ER microsome fractions of SNCA-3X and isogenic and healthy control neurons (n=3, except n=1 for Ctrl). (C) The PLA background signal was determined by staining with a single primary antibody (α-syn (211), α-syn (C20), or CANX). α-Syn aggregate formation under high α-syn overexpression was used as a positive control, as indicated by a positive α-syn (211)-α-syn (C20) PLA signal. PLA signal was calculated as the number of red particles normalized to the number of nuclei within an acquired field of view (n=3-7). Scale bar, 25 mm. For all quantifications, data is shown as mean±SEM, *p<0.05; ****p<0.0001, using ANOVA with Tukey's post-hoc test (panel A) or student's unpaired t-test (panels B, C).

FIGS. 14A-14F. Enhancing ER chaperone function chemically or genetically improves GCase proteostasis in α-syn overexpressing cell lines and SNCA-3X DA neurons. FIGS. 14A-14F linked to FIGS. 6A-6F. (A) Day 120 healthy control (Ctrl) DA neurons were treated with 25 mM DILT for 2 weeks and GCase was analyzed by western blot. Right, quantifications of total GCase, post-ER, and ER forms of GCase were normalized to coomassie (n=3). The post-ER to ER GCase ratio (maturation) is also shown (n=3) (B, C) To validate that enhancing ER function can improve GCase proteostasis in α-syn overexpressing models, cells were treated with 40 mM dantrolene (DANT), another RyR inhibitor. H4 α-syn overexpressing cells (panel B) were treated for 5 days and day 90 SNCA-3X DA neurons (panel C) were treated for 2 weeks. GCase maturation after DANT treatment was quantified by western blot, and coomassie gel staining is shown as a loading control. In panel B, quantification of GCase maturation is from the indicated blot (n=3). In panel C, analysis of GCase maturation is combined from 3x−1 (clones C2 and C3), 3x−2, and 3x−4 (n=3 per line). (D) α-Syn overexpressing cells were treated with a RyR inhibitor, 1 mm DHBP, for 5 days. Analysis of soluble and insoluble GCase was performed by sequential extraction and western blot. Right, quantification of percent insoluble GCase normalized to coomassie in DHBP treated cells (n=3). (E, F) To genetically validate that RyR inhibition is sufficient in improving GCase proteostasis, H4 α-syn overexpressing cells were infected with scrambled (scrb) or RyR3 shRNA containing lentivirus for 5 days. (E) RyR3 knock-down (KD) efficiency was measured by quantitative RT-PCR analysis of RyR3 mRNA (n=3). (F) Western blot analysis of soluble and insoluble GCase after RyR3 KD in H4 cells. Addition of DILT following scrb or RyR3 KD was used to determine the specificity of the compound. Coomassie is shown as a loading control. Irrelevant lanes were cropped out from the blots and are indicated with a dashed line. Right, quantifications of percent insoluble GCase normalized to coomassie and GCase maturation in H4 cells after DILT treatment in scrb or RyR3 KD cells (n=9-13). For all quantifications, values are mean±SEM, *p<0.05; **p<0.01; ****p<0.0001; ns=not significant, using student's unpaired t-test (panels A-F) or ANOVA with Tukey's post-hoc test (panel F).

FIGS. 15A-15H. Combining ER proteostasis and trafficking enhancers synergistically improves GCase functionality and reduces α-syn. FIGS. 15A-15H linked to FIGS. 7A-7E. (A) SNCA-3X DA neurons were treated with vehicle, 5 nM FTI, 25 mM DILT or an FTI+DILT combination for 2 or 4 weeks, and the effect on GCase maturation due to each individual compound was added and compared to the combination treatment. (B) H4 α-syn overexpressing cells were treated for 5 days with vehicle, 5 nM FTI, 25 mM DILT or an FTI+DILT combination and analyzed for GCase maturation and α-syn levels. GAPDH and Coomassie are shown as loading controls. (C) Quantifications of GCase maturation and α-syn (antibody 211) levels normalized to Coomassie (n=3 per condition, combined from 3 individual experiments for n=9 total), as well as whole cell GCase activity (n=5 per condition), in H4 treated cells. (D) The effect on soluble α-syn of each individual compound was added and compared to the combination treatment (antibodies syn211 and syn303) in SNCA-3X DA neurons as in panel A. (E) GCase maturation in RyR3 KD 3x−1 DA neurons in combination with 5 nM FTI treatment was analyzed by western blot (MOI=3, 2 week treatment). Coomassie is shown as a loading control. Right, quantification of GCase maturation (n=3). (F) H4 α-syn overexpressing cells were lentivirally infected with vector (vect) or ykt6-CS and treated with DILT in combination (MOI=3, 5 day treatment). Sequential extraction and western blot analysis of GCase and soluble and insoluble α-syn was performed. Blotting for ykt6 was done to confirm successful overexpression. Coomassie is shown as a loading control. (G) Analysis of GCase maturation and α-syn levels from panel F (n=3). (H) Western blot analysis of GCase and soluble and insoluble α-syn in 3x−1 DA neurons overexpressing ykt6-CS in combination with DILT treatment (MOI=3, 2 week treatment). Blotting for ykt6 was done to confirm successful overexpression. Coomassie is shown as a loading control. Right, quantification of GCase maturation and α-syn levels. For all quantifications, values are mean±SEM, *p<0.05; **p<0.01; ***p<0.001; ****p<0.0001, using student's unpaired t-test (panels A, D, E, H) or ANOVA with Tukey's post-hoc test (panel C, G).

FIGS. 16A-16D. Rescue of GCase maturation and reduction of α-syn in PD patient lines with GBA1 mutations by synergistic enhancement of ER proteostasis and trafficking. FIGS. 16A-16D linked to FIGS. 7A-7E. A) Cultures from a GD patient expression N370S/84GG GCase were treated for two weeks with FTI, DILT, or in combination as described in FIG. 7, followed by analysis of GCase maturation by western blot (n=3). B) GD cultures were treated as in A, followed by analysis of soluble α-syn by western blot using syn211 (n=3). C) iPSC midbrain neurons from a PD patient expressing N370S/wt GCase were treated and analyzed as in panel A (n=3-4). D) Soluble α-syn levels were determined in GBA-PD neurons as in panel B (n=3). For all quantifications, values are mean±SEM, *p<0.05; **p<0.01; ***p<0.001; an ANOVA with Tukey's post-hoc test.

FIGS. 17A-17E. Cellular and in vivo models of PD exhibit decreased N-glycosylated proteins. Western blot analysis of N-glycosylated proteins in A) A53T or isogenic corrected (Corr) iPSn at day 90 in the presence or absence of Tunicamycin (Tunica) for 24 hours B) Cerebellar brain lysate from symptomatic A53T α-syn transgenic and age-matched control (Con) mice in the age range of 10-14 mon, using biotinylated Con-A, detected by IRDye labeled streptavidin antibody. GAPDH is a loading control. Quantification is shown on the right. n=3, +/−SEM, *p<0.05, **p<0.01. C) N-glycan synthesis was measured by metabolic labeling of Corr and A53T-patient neurons with Ac4 ManNAz, followed by cell lysis at 18, 36, 54, and 72 h. Labeled glycans were conjugated to biotin using biotin-phosphine. Biotinylated proteins were analyzed by Western blot of analysis using IRDye labeled streptavidin antibody. GAPDH is a loading control. n=3, +/−SEM, *p<0.05. D) Western blot analysis of Hex B in A53T or Corr iPSn at day 90 in the presence or absence of Tunicamycin (Tunica). GAPDH was used as a loading control. Quantification of glycosylated and mature forms of Hex-B is shown on the right of blot. n=3, +/−SEM, *p<0.05. E) Western blot of analysis of N-glycosylated GCase and Hex-B from Corr and A53T iPSn. N-glycosylated proteins were precipitated using Con-A conjugated to biotin and streptavidin agarose beads were used to collect precipitated N-glycosylated proteins. Quantifications are shown on the right and normalized to input. n=3, +/−SEM, **p<0.01*p<0.05.

FIGS. 18A-18L. Hexosamine-biosynthetic pathway (HBP) is disrupted in PD models and synucleinopathy patient brain. A) Schematic showing HBP and N-glycosylation pathway. UDP-GlcNAc, Uridine-diphosphate-N-acetylglucosamine; F-6-P, Fructose-6-phosphate; HK-1, Hexokinase-1; G-6-P, glucose-6-phosphate; GFPT2, glutamine: F-6-P transaminase-2; GNA1, GlcN-6P acetyltransferase; acetyl-CoA, acetyl coenzyme A; PGM3, GlcNAc phosphomutase; UAP1, UDP-GlcNAc pyrophosphorylase; NAGK, N-acetylglucosamine kinase; OST, oligosaccharyltransferase; B-F) Metabolomic analysis of A53T or Corr iPSn at day 90 by Liquid Chromatography-Mass Spectrometry LC/MS. (n=3-4, +/−SEM, *p<0.05) B) Glucose C) Glucose-6-Phosphate D) Uridine triphosphate (UTP) E) Glutamine F) Glutamate. G) Quantitative RT-PCR analysis of DPAGT1 (n=6, +/−SEM, NS, not significant. H) Quantitative RT-PCR analysis of GFPT2 mRNA in A53T or Corr iPSn at day 90, n=3, +/−SEM, **p<0.01. I-L) Western blot analysis of GFPT2 in I) SH-SY5Y vector (vec) and stable line expressing WT-α-Syn (Syn), J) iPSn (Corr and A53T) K) Non-transgenic control (Con) and A53T transgenic mouse brain (cerebellum) and L) post-mortem cortical human brain samples from Control (Con) and Dementia with Lewy body patients (DLB). GAPDH was used as loading control. Quantifications are shown on the right. n=3-6, +/−SEM, **p<0.01, *p<0.05.

FIGS. 19A-19E. GFPT2 is upregulated in response to tunicamycin and disrupted in PD patient neurons. A) Quantitative RT-PCR analysis of GFPT2 mRNA in Corr and A53T iPSn at day 90, after treatment with vehicle (DMSO) and tunicamycin (5 ug/ml) for 24 h n=6, +/−SEM, ***p<0.001. B) Western blot analysis of GFPT2 protein in iPSn (SNCA-3X−1 and Corr) after treatment with vehicle (DMSO) and tunicamycin (5 ug/ml) for 24 h with GAPDH as loading control. Quantifications are shown on the right. n=3, +/−SEM, *p<0.05. C) Quantitative RT-PCR analysis of XBP1 mRNA in Corr and A53T iPSn at day 90, after treatment with vehicle (DMSO) and tunicamycin (5 ug/ml) for 24 h n=3, +/−SEM, **p<0.01. ****p<0.0001. D) Quantitative RT-PCR analysis of GRP78 mRNA in Corr and A53T iPSn at day 90, after treatment with vehicle (DMSO) and tunicamycin (5 ug/ml) for 24 h n=3, +/−SEM, *p<0.05. ****p<0.0001. E) Quantitative RT-PCR analysis of XBP1, GFPT2, GRP78 and GRP94 in A53T iPSn, at day 120 infected with lentivirus expressing GFP (Control) and XBP1^{s} at MOI-1, 16 days after infection. n=3-4, +/−SEM, *p<0.05 & **p<0.01.

FIGS. 20A-20H. GFPT2 restores ER-Golgi trafficking of lysosomal hydrolases, GCase activity and reduces α-Synuclein accumulation in PD midbrain neurons A) Western blot analysis of GFPT2 and GFP in iPSn (A53T) infected with lentivirus expressing GFP (Control) and GFPT2 at MOI-3, 15 days after infection. GAPDH is a loading control. Quantification is shown on the right. n=3-4, +/−SEM, ***p<0.001. B) Western blot of N-glycosylated proteins using biotinylated Con-A, in iPSn (A53T) infected with lentivirus expressing GFP (Control) and GFPT2 GAPDH is a loading control. Quantification is shown on the right. n=3+/−SEM, *p<0.05. C) GCase maturation was measured by western blot analysis using endoglycosidase H (endo H) digestion of lysates from GFP and GFPT2 infected iPSn (A53T). GAPDH is a loading control. n=3, +/−SEM, *p<0.05. Total GCase levels were quantified from the non-endo H digested lanes. D) Western blot analysis of Hex B in A53T iPSn infected with GFP and GFPT2, using GAPDH as a loading control, Quantifications of mature Hex-B is shown on the right. n=3-4, +/−SEM, *p<0.05. E) Quantitative RT-PCR analysis of GBA, Hex-B and GFPT2 in A53T iPSn at day 90, infected with control GFPT2 lentivirus at MOI-3, 15 days after infection. n=3, +/−SEM, ****p<0.0001. F) Analysis of lysosomal GCase activity in live A53T patient neurons infected with vector and GFPT2 lentivirus at MOI-3. Fluorescent substrate degradation was evaluated in a microplate reader for 3 h. Activity within acidic cellular compartments was determined by quantifying the response to bafilomycin A1 (Baf A1), shown on right. (n=4), +/−SEM, **p<0.01. G) Western blot analysis of α-syn from triton X-100-Soluble (T-sol) and triton-insoluble (T-insol) fractions using C20 antibody, in A53T iPSn infected with vector and GFPT2 lentivirus, using Coomassie brilliant blue (CBB) as a loading control. Quantification is shown on the right. n=4, +/−SEM*p<0.05. H) Neuron viability was assessed in iPSn A53T infected with vector and GFPT2 lentivirus, by quantification of neurofilament (NFM) content normalized to a general cell volume stain (cell-tag). Quantification is shown on the right. n=19-20, +/−SEM, **p<0.01.

FIGS. 21A-21I. GlcNAc supplementation rescues N-glycosylation and downstream pathogenic phenotypes in PD patient midbrain neurons. A) Western blot of N-glycosylated proteins using biotinylated Con-A, in iPSn (Corr and A53T at day 122) treated with 10 mM GlcNAc for 7 days. GAPDH is a loading control. Quantification is shown on the right. n=4, +/−SEM, **p<0.01 & *p<0.05. B) Western blot analysis of GCase from triton X-100-Soluble (T-sol) and triton-insoluble (T-insol) fractions in Corr and A53T iPSn in the presence or absence of GlcNAc, using coomassie brilliant blue (CBB) as a loading control. Quantification is shown on the right. n=3, +/−SEM, *p<0.05. (C) GCase maturation was measured by western blot using endoglycosidase H (endo H) digestion of lysates from Veh and GlcNAc supplemented A53T iPSn. GAPDH is a loading control. Quantification is shown on the right. n=3, +/−SEM, **p<0.01. (D) Analysis of lysosomal GCase activity in live A53T patient neurons after supplementation of GlcNAc. Fluorescent substrate degradation was evaluated in a microplate reader for 3 h. Activity in acidic compartments was determined by measuring the response to a lysosomal inhibitor-bafilomycin A1. For all quantifications, values are the mean+/−SEM, *p<0.05, n=4. E and F) Western blot analysis of Hex B in Corr and A53T iPSn at day 122 in the presence or absence of GlcNAc, using Coomassie brilliant blue (CBB) as loading control, Quantifications of mature and immature Hex-B are shown in (F) n=3, +/−SEM, *p<0.05. G and H) Western blot analysis of α-syn from triton X-100-Soluble (T-sol) and triton-insoluble (T-insol) fractions using C20 or syn303 antibodies, in Corr and A53T iPSn in the presence or absence of GlcNAc, using coomassie brilliant blue (CBB) as loading control. α-syn knock-out iPSn was used as a negative control. * Indicates non-specific bands. Quantification are shown in (H) n=3, +/−SEM, *p<0.05. I) Neuron viability was assessed in iPSn A53T at day 122 supplemented with GlcNAc (10 mM) for 1 week, by quantification of neurofilament (NFM) content normalized to cell volume using cell-tag. Quantification is shown below. n=9-10 culture wells, +/−SEM, ***p<0.001.

FIGS. 22A-22C. Con-A specifically detects N-glycosylated Proteins and ManNAz is incorporated into N-glycosylated Proteins A) H4 cell lysates were subjected to PNGase-F digestion and run on a 10% SDS-PAGE along with control lysate and subjected to western blotting against biotinylated Con-A, followed by detection with IRDye labeled streptavidin antibody. GAPDH is used as a loading control. B) Metabolic labeling of Ac4 ManNAz differentiated SHSY5Y cells (Retinoic acid 10 μM for 5 days), followed by cell lysis at 96 h. Labeled glycans were conjugated to biotin using biotin-phosphine. Lysates were subjected to PNGase digestion; undigested and digested lysates were subjected to western blot analysis. B) Biotinylated proteins were detected by IRDye labeled streptavidin antibody. GAPDH is a loading control. n=3, +/−SEM, ****p<0.0001& ***p<0.001 C) protein synthesis remains unaltered in A53T patient neurons Western blot image showing newly synthesized proteins labeled with puromycin using the SUnSET technique. Corr and A53T neurons were treated with puromycin (5 μg/mL) for 1 h. Quantifications are shown on right, (n=4). Lysate from corrected neurons not treated with puromycin was used as a negative control for puromycin incorporation.

FIGS. 23A-23E. A) Western blot analysis of HK-1 in iPSn (Corr and A53T at d90), using GAPDH loading controls. Quantification is shown on the right. n=4, +/−SEM. GAPDH levels are also quantified and normalized to CBB, which remain unchanged between Corr and A53T neurons. B) Western blot analysis of GFPT2 in iPSn (SNCA-3X-2 and Corr) with GAPDH as a loading control. Quantifications are shown on the right. n=4-6, +/−SEM, *p<0.05.C, D and E) Western blot analysis of GNA1, NAGK and OST in iPSn (Corr and A53T at d90), using GAPDH loading control. Quantification is shown on the right. n=4, +/−SEM.

FIGS. 24A-24B. GFPT2 Overexpression in SNCA triplication (SNCA-3X) enhances GCase activity and reduces pathological α-Synuclein. A) Analysis of lysosomal and non-lysosomal GCase activity in live SNCA-3X patient neurons (Est. 3X, and line 1A) infected with vector and GFPT2 lentivirus at MOI-3. Fluorescent substrate degradation was evaluated in a microplate reader for 3 h. Activity within acidic cellular compartments was determined by quantifying the response to bafilomycin A1 (Baf A1). Quantifications for the total GCase—black solid lines and lysosomal GCase—The area between the DMSO and BafA1 curves shown by dotted lines. The area under purple dotted lines represents the non-lysosomal GCase activity (n=3-4), +/−SEM, SEM, *p<0.05 & **p<0.01. B) Western blot analysis of GFPT2 and α-synuclein in PD-triplication neurons (SNCA-3X, Est 3X and line 1A) infected with lentivirus expressing vector and GFPT2 at MOI-3, 15 days after infection. GAPDH is a loading control. α-syn was detected with LB509 antibody and quantification is shown on the right. n=4, +/−SEM, *p<0.05 & **p<0.01.

### DETAILED DESCRIPTION

The disclosed subject matter may be further described using definitions and terminology as follows. The definitions and terminology used herein are for the purpose of describing particular embodiments only, and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the claimed subject matter and does not pose a limitation on the scope of the claimed subject matter.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

As used herein, a “subject in need thereof” may include a human and/or non-human animal. A “subject in need thereof” may include a subject having a disease, disorder, or condition associated with accumulation of α-synuclein. A “subject in need thereof” may include a subject having a neurodegenerative disease, disorder, or condition, which may include, but is not limited to Parkinson's disease, Lewy body dementia, Alzheimer's disease, amyotrophic lateral sclerosis (ALS), multiple system atrophy, Huntington's disease, Prion disease, frontotemporal dementia, Picks disease, progressive supranuclear palsy, and progeria. A “subject in need thereof” may also include a subject having a rapid eye movement (REM) behavior sleep disorder linked to Parkinson's disease and/or GBA1 mutation carriers. A “subject in need thereof” may additionally include a subject having a pediatric lysosomal storage disorder, which may include, but is not limited to glycogen storage disorder, neuronal ceroid lipofuscinosis disorder, sphingolipid storage disorder, cholesterol storage disorder, and fatty acid storage disorder. A “subject in need thereof” may also include a subject having a protein misfolding and/or amyloidosis disease, disorder, or condition, which may include, but is not limited to cataract caused by α-crystallin aggregation, systemic amyloidosis, type 2 diabetes characterized by amylin aggregation, and alpha-1-antitrypsin deficiency liver disease.

As used herein the term “therapeutically effective amount” refers to the amount or dose of the compound or composition, upon single or multiple dose administration to the subject, which provides the desired effect in the subject under diagnosis or treatment. The disclosed methods may include administering a therapeutically effective amount of (a) at least one ER-Golgi trafficking enhancer in combination with at least one ER protein folding enhancer, or (b) at least one N-glycosylation enhancer, or a pharmaceutical composition comprising a therapeutically effective amount of (a) at least one ER-Golgi trafficking enhancer and at least one ER protein folding enhancer, or (b) at least one N-glycosylation enhancer, for treating and/or preventing a disease, disorder, or condition associated with accumulation of α-synuclein in a patient. Administration of the therapeutically effective amount may inhibit accumulation of α-synuclein in the patient, increase β-glucocerebrosidase (GCase) maturation level, increase GCase solubility, improve endoplasmic reticulum (ER) morphology, or any combination thereof.

A therapeutically effective amount can be readily determined by the attending diagnostician, as one skilled in the art, by the use of known techniques and by observing results obtained under analogous circumstances. In determining the therapeutically effective amount or dose of compound administered, a number of factors can be considered by the attending diagnostician, such as: the species of the subject; its size, age, and general health; the degree of involvement or the severity of the disease or disorder involved; the response of the individual patient; the particular compound administered; the mode of administration; the bioavailability characteristics of the preparation administered; the dose regimen selected; the use of concomitant medication; and other relevant circumstances.

In some embodiments, a daily dose of the disclosed farnesyltransferase inhibitor or ryanodine receptor inhibitor may contain from about 0.01 mg/kg to about 100 mg/kg (such as from about 0.05 mg/kg to about 50 mg/kg and/or from about 0.1 mg/kg to about 25 mg/kg) of each compound used in the present method of treatment. The dose may be administered under any suitable regimen (e.g., weekly, daily, twice daily).

The pharmaceutical compositions for use according to the methods as disclosed herein may include a combination of compounds as active ingredients. For example, the methods disclosed herein may be practiced using a composition containing one or more compounds that are ER-Golgi trafficking enhancer and one or more compounds that are ER protein folding enhancer.

In some embodiments, the disclosed methods may be practiced by administering a first pharmaceutical composition (e.g., a pharmaceutical composition comprising a ER-Golgi trafficking enhancer, a ER protein folding enhancer, or a pharmaceutical composition comprising an N-glycosylation enhancer) and administering a second pharmaceutical composition (e.g., a pharmaceutical composition comprising a ER-Golgi trafficking enhancer, a ER protein folding enhancer, or a pharmaceutical composition comprising an N-glycosylation enhancer), where the first composition may be administered before, concurrently with, or after the second composition. As such, the first pharmaceutical composition and the second pharmaceutical composition may be administered concurrently or in any order, irrespective of their names.

As one skilled in the art will also appreciate, the disclosed pharmaceutical compositions can be prepared with materials (e.g., actives excipients, carriers, and diluents etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

The at least one ER-Golgi trafficking enhancer, the at least one ER protein folding enhancer, and the at least one N-glycosylation enhancer utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof. Alternatively, the compounds utilized in the methods disclosed herein may be formulated as a pharmaceutical composition in liquid form (e.g., an injectable liquid or gel).

The at least one ER-Golgi trafficking enhancer, the at least one ER protein folding enhancer, and the at least one N-glycosylation enhancer utilized in the methods disclosed herein may be formulated as a pharmaceutical composition that includes an excipient, carrier, or diluent. For example, the excipient, carrier, or diluent may be selected from the group consisting of proteins, carbohydrates, sugar, talc, magnesium stearate, cellulose, calcium carbonate, and starch-gelatin paste.

The at least one ER-Golgi trafficking enhancer, the at least one ER protein folding enhancer, and the at least one N-glycosylation enhancer utilized in the methods disclosed herein also may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil®200, talc, stearic acid, magnesium stearate, calcium stearate, and silica gel. Examples of sweeteners may include any natural or artificial sweetener, such as sucrose, xylitol, sodium saccharin, cyclamate, aspartame, and acesulfame. Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

The disclosed pharmaceutical compositions also may include disintegrants. Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

The disclosed pharmaceutical compositions also may include effervescent agents. Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

Pharmaceutical compositions comprising (a) the at least one ER-Golgi trafficking enhancer and the at least one ER protein folding enhancer, or (b) the at least one N-glycosylation enhancer may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams.

For applications to the eye or other external tissues, for example the mouth and skin, the pharmaceutical compositions are preferably applied as a topical ointment or cream. When formulated in an ointment, the compound may be employed with either a paraffinic or a water-miscible ointment base. Alternatively, the compound may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

As used herein, “α-synuclein” refers to a protein that is encoded by the SNCA gene in humans. Alpha-synuclein is a neuronal protein that is found to be concentrated within presynaptic terminals and regulates synaptic vesicle fusion, clustering, and subsequent neurotransmitter release.

As used herein, “ER-Golgi trafficking enhancer” refers to a farnesyltransferase inhibitor (FTI), ykt6 activator, Rab1A activator, or other compounds that enhance trafficking between the endoplasmic reticulum (ER) and the Golgi.

As used herein, “ER protein folding enhancer” refers to a Ryanodine receptor (RyR) inhibitor, an inositol triphosphate (IP3) receptor inhibitor, an activator of the sarco/endoplasmic reticulum Ca²⁺-ATPase (SERCA), small molecule compounds that directly bind and promote the folding of beta-glucocerebrosidase, or other compounds that activate the unfolded protein response (UPR) and result in increased chaperone expression.

As used herein, “N-glycosylation enhancer” refers to N-acetylglucosamine, N-acetylglucosamine-6-acetate, L-glutamine, fructose-6-phosphate, an allosteric activator of glutamine:F-6-P transaminase-1, an allosteric activator of glutamine:F-6-P transaminase-2, or other compounds that enhances the N-glycosylation pathways in endoplasmic reticulum.

As used herein, a “neurodegenerative disease, disorder, or condition” refers to a disease, disorder, or condition that causes progressive loss of structure or function of neurons and ultimately may cause cell death.

As used herein, a “rapid eye movement (REM) behavior sleep disorder” refers to a parasomnia characterized by dream-enactment behaviors that emerge during a loss of REM sleep atonia.

As used herein, “GBA1” refers to the glucocerebrosidase gene located on chromosome 1q21 and encodes for the lysosomal enzyme beta-glucocerebrosidase.

As used herein, “lysosomal storage disorder” refers to inherited metabolic diseases that are characterized by an abnormal build-up of various toxic materials in the body's cells as a result of enzyme deficiencies. Lysosomal storage disorders may affect different parts of the body, including the skeleton, brain, skin, heart, and central nervous system.

As used herein, “β-glucocerebrosidase maturation” refers to the maturation of the enzyme β-glucocerebrosidase (GCase), which refers to a process leading to the attainment of the full functional capacity of GCase. Proper GCase maturation requires its trafficking from the endoplasmic reticulum (ER) to the Golgi portion of the secretory pathway.

As used herein, “pharmaceutically acceptable salt” refers to those salts which are, within the scope of sound medical judgement, suitable for use in contact with the tissues of humans and lower animals without undue toxicity, irritation, allergic response and the like and are commensurate with a reasonable benefit/risk ratio. Pharmaceutically acceptable salts have been described in S. M. Berge et al. J. Pharmaceutical Sciences, 1977, 66: 1-19.

The compounds disclosed herein may contain either a basic or an acidic functionality, or both, and can be converted to a pharmaceutically acceptable salt, when desired, by using a suitable acid or base. The salts may be prepared in situ during the final isolation and purification of the compounds of the invention.

Examples of acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethanesulfonate (isothionate), lactate, malate, maleate, methanesulfonate, nicotinate, 2-naphthalenesulfonate, oxalate, palmitate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as, but not limited to, methyl, ethyl, propyl, and butyl chlorides, bromides and iodides; dialkyl sulfates like dimethyl, diethyl, dibutyl and diamyl sulfates; long chain halides such as, but not limited to, decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid, and citric acid.

Basic addition salts may be prepared in situ during the final isolation and purification of compounds of this invention by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other examples of organic amines useful for the formation of base addition salts include ethylenediamine, ethanolamine, diethanolamine, piperidine, piperazine and the like.

Compounds described herein may exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

ER-Golgi Trafficking Enhancer

New uses for ER-Golgi trafficking enhancer are disclosed herein.

Farnesyltransferase Inhibitor

As used herein, “farnesyltransferase inhibitor” (FTI) refers to an inhibitor of farnesyltransferase (FTase). FTIs have been designed to inhibit the activity of Ras oncoproteins and have been used as anti-Ras and anti-cancer drugs. Farnesyltransferase inhibitors may include but is not limited to LNK-754, lonafarnib, gliotoxin, gingerol, tipifarnib, α-hydroxy farnesyl phosphonic acid, manumycin A, L-744832 dihydrochloride, FTI-277 trifluoroacetate salt, FTI-276 trifluoroacetate salt, FTase inhibitor II, and FTase inhibitor I.

As used herein, LNK-754 refers to the chemical 6-[(R)-(4-chlorophenyl)hydroxy(1-methyl-1H-imidazol-5-yl)methyl]-4-(3-ethynylphenyl)-1-methyl-2(1H)-Quinolinone and has a CAS No. of 1190094-64-4. LNK-754 has a chemical structure as follows.

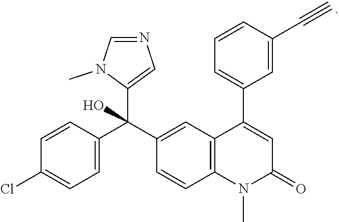 [SMILES: C#Cc1cccc(-c2cc(=O)n(C)c3ccc([C@](O)(c4ccc(Cl)cc4)c4cncn4C)cc23)c1]

As used herein, lonafarnib refers to the chemical 4-[2-[4-[(2R)-6,15-dibromo-13-chloro-4-azatricyclo[9.4.0.0^{3,8}]pentadeca-1(11),3(8),4,6,12,14-hexaen-2-yl]piperidin-1-yl]-2-oxoethyl]piperidine-1-carboxamide and has a CAS No. of 193275-84-2. Lonafarnib has a chemical structure as follows:

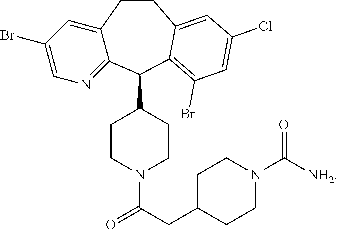 [SMILES: NC(=O)N1CCC(CC(=O)N2CCC([C@H]3c4ncc(Br)cc4CCc4cc(Cl)cc(Br)c43)CC2)CC1]

As used herein, gliotoxin refers to the chemical (1R,7S,8S,11R)-7-hydroxy-11-(hydroxymethyl)-15-methyl-12,13-dithia-9,15-diazatetracyclo[9.2.2.0^{1,9}.0^{3,8}]pentadeca-3,5-diene-10,14-dione and has a CAS No. of 67-99-2. Gliotoxin has a chemical structure as follows:

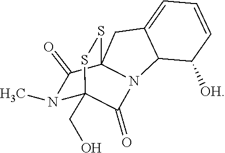 [SMILES: CN1C(=O)C23CC4=CC=C[C@H](O)C4N2C(=O)C1(CO)SS3]

As used herein, gingerol refers to the chemical (5S)-5-hydroxy-1-(4-hydroxy-3-methoxyphenyl)decan-3-one and has a CAS No. of 23513-14-6. Gingerol has a chemical structure as follows:

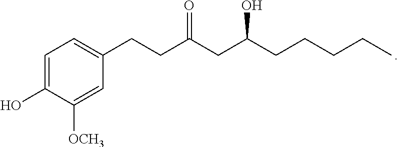 [SMILES: CCCCC[C@H](O)CC(=O)CCc1ccc(O)c(OC)c1]

As used herein, tipifarnib refers to the chemical 6-[(R)-amino-(4-chlorophenyl)-(3-methylimidazol-4-yl)methyl]-4-(3-chlorophenyl)-1-methylquinolin-2-one and has a CAS No. of 192185-72-1. Tipifarnib has a chemical structure as follows:

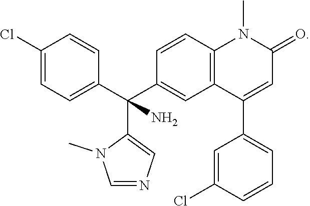 [SMILES: Cn1cncc1[C@](N)(c1ccc(Cl)cc1)c1ccc2c(c1)c(-c1cccc(Cl)c1)cc(=O)n2C]

As used herein, α-hydroxy farnesyl phosphonic acid refers to the chemical [(2E,6E)-1-hydroxy-3,7,11-trimethyldodeca-2,6,10-trienyl]phosphonic acid and has a CAS No. of 148796-53-6. α-hydroxy farnesyl phosphonic acid has a chemical structure as follows:

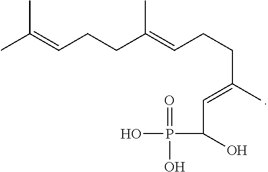 [SMILES: CC(C)=CCC/C(C)=C/CC/C(C)=C/C(O)P(=O)(O)O]

As used herein, manumycin A refers to the chemical (2E,4E,6R)—N-[(1S,5S,6R)-5-hydroxy-5-[(1E,3E,5E)-7-[(2-hydroxy-5-oxocyclopenten-1-yl)amino]-7-oxohepta-1,3,5-trienyl]-2-oxo-7-oxabicyclo[4.1.0]hept-3-en-3-yl]-2,4,6-trimethyldeca-2,4-dienamide and has a CAS No. of 52665-74-4. Manumycin A has a chemical structure as follows:

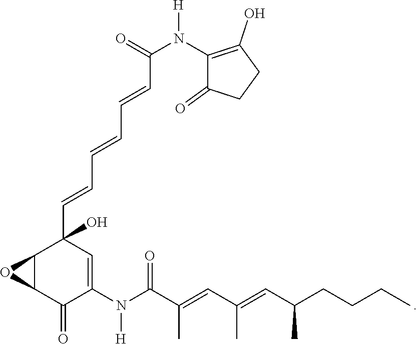 [SMILES: CCCC[C@@H](C)/C=C(C)/C=C(\C)C(=O)NC1=C[C@@](O)(/C=C/C=C/C=C/C(=O)NC2=C(O)CCC2=O)[C@@H]2O[C@@H]2C1=O]

As used herein, L-744832 dihydrochloride refers to the chemical propan-2-yl (2S)-2-[[(2S)-2-[(2S,3R)-2-[[(21)-2-amino-3-sulfanylpropyl]amino]-3-methylpentoxy]-3-phenylpropanoyl]amino]-4-methylsulfonylbutanoate; dihydrochloride and has a CAS No. of 1177806-11-9. L-744832 dihydrochloride has a chemical structure as follows:

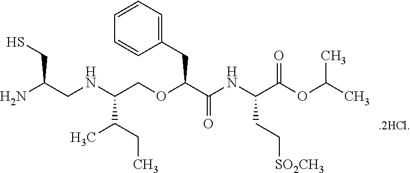 [SMILES: CCC(C)[C@@H](CO[C@@H](Cc1ccccc1)C(=O)N[C@@H](CCS(C)(=O)=O)C(=O)OC(C)C)NC[C@@H](N)CS]

As used herein, FTI-277 trifluoroacetate salt refers to the chemical methyl (2S)-2-[[4-[[(2R)-2-amino-3-sulfanylpropyl]amino]-2-phenylbenzoyl]amino]-4-methylsulfanylbutanoate; 2,2,2-trifluoroacetic acid and has a CAS No. of 170006-73-2. FTI-277 trifluoroacetate salt has a chemical structure as follows:

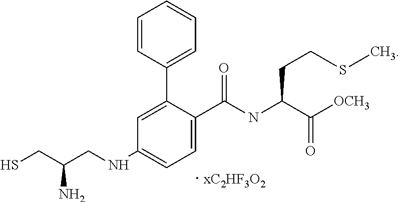 [SMILES: COC(=O)[C@H](CCSC)NC(=O)c1ccc(NC[C@@H](N)CS)cc1-c1ccccc1]

As used herein, FTase inhibitor II refers to the chemical (2S)-2-[[4-[[(2R)-2-azaniumyl-3-sulfanylpropanoyl]amino]benzoyl]amino]-4-methylsulfanylbutanoate and has a CAS No. of 156707-43-6. FTase inhibitor II has a chemical structure as follows:

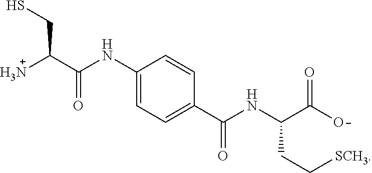 [SMILES: CSCC[C@H](NC(=O)c1ccc(NC(=O)[C@@H]([NH3+])CS)cc1)C(=O)[O-]]

As used herein, FTase inhibitor I refers to the chemical (2S)-2-[[(2S)-2-[[(2S)-[[(2R)-2-amino-3-sulfanylpropyl]amino]-3-methylbutyl]amino]-3-phenylpropanoyl]amino]-4-methylsulfanylbutanoic acid and has a CAS No. of 149759-96-6. FTase inhibitor I has a chemical structure as follows:

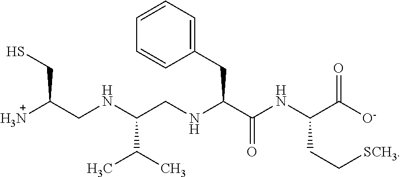 [SMILES: CSCC[C@H](NC(=O)[C@H](Cc1ccccc1)NC[C@@H](NC[C@@H]([NH3+])CS)C(C)C)C(=O)[O-]]

In some embodiments, FTI is selected from the following compounds or a pharmaceutically acceptable salt thereof:

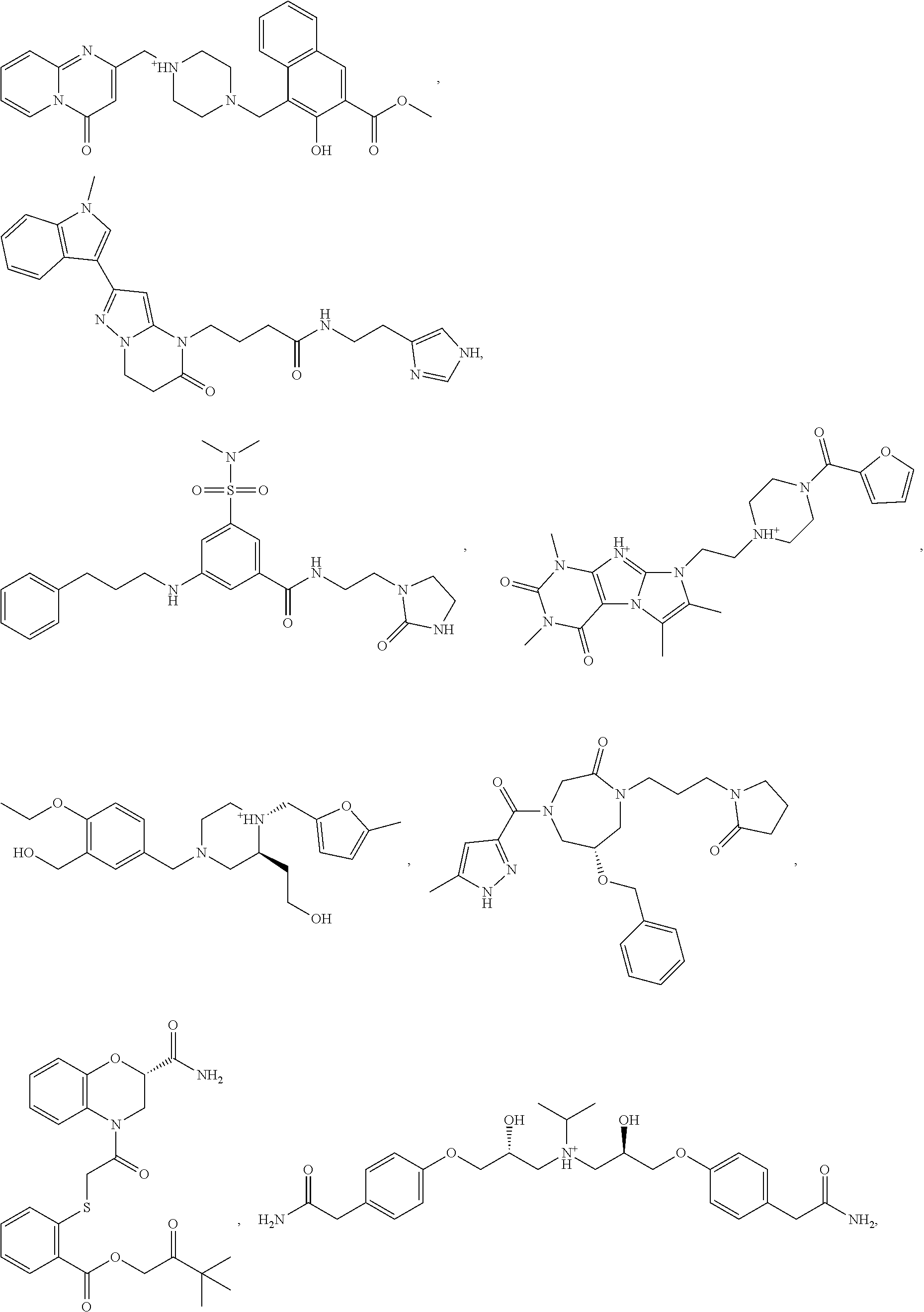

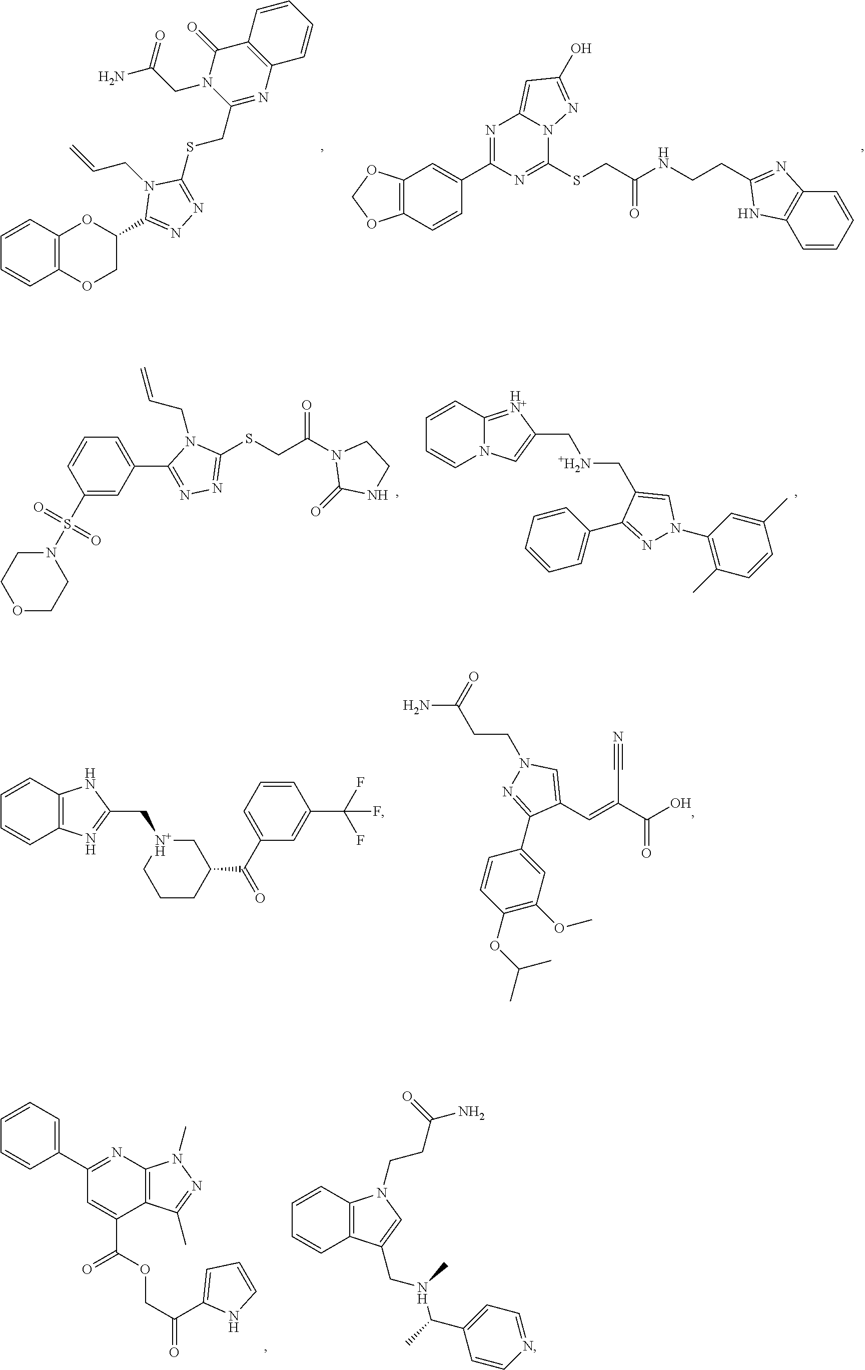

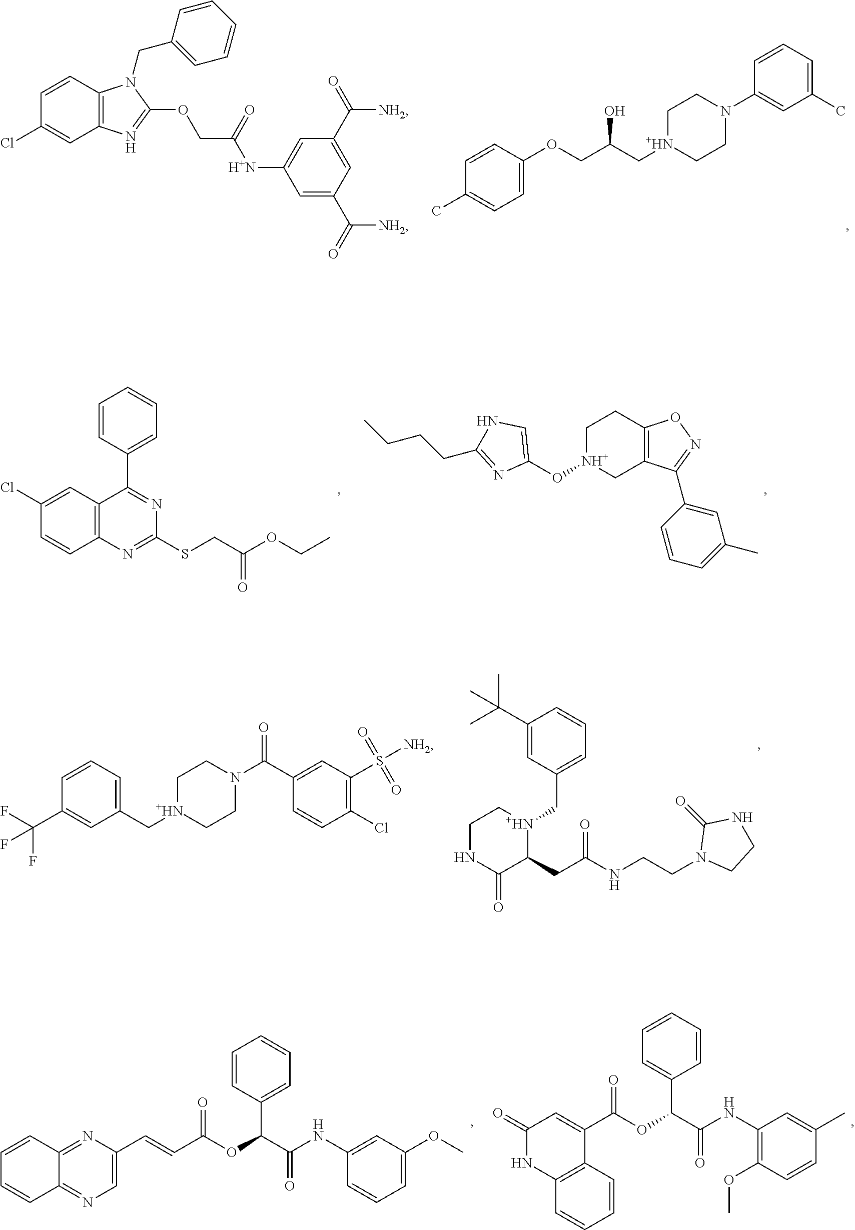

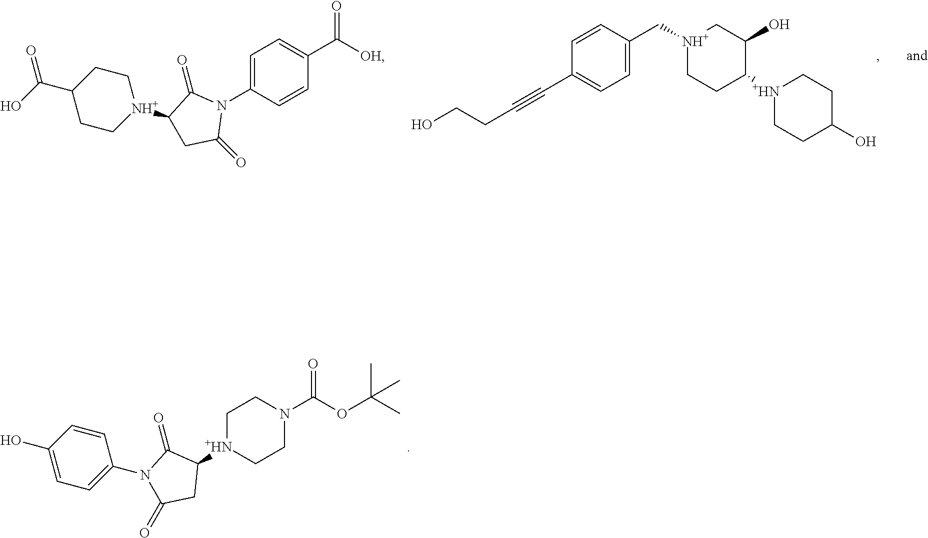 [SMILES: O=C(O)c1ccc(N2C(=O)C[C@@H]([NH+]3CCC(C(=O)O)CC3)C2=O)cc1 ; OCCC#Cc1ccc(C[N@H+]2CC[C@@H]([NH+]3CCC(O)CC3)[C@H](O)C2)cc1 ; CC(C)(C)OC(=O)N1CC[NH+]([C@H]2CC(=O)N(c3ccc(O)cc3)C2=O)CC1]

Ykt6 Activator

As used herein, “ykt6 activator” refers to a compound that activates the biological activity of cellular protein ykt6. As used herein, the term “activate” means increasing or augmenting activity. The ykt6 activators utilized in the disclosed methods may activate the biological activity of ykt6 directly and/or indirectly by interacting with ykt6 directly and/or indirectly. In some embodiments, the ykt6 activators disclosed herein activate ykt6 by inhibiting the biological activity of farnesyltransferase. In other embodiments, the ykt6 activators disclosed herein inhibit and/or disrupt the molecule interaction between farnesyl-ykt6 and the therapeutic agents may promote the open, active conformation of ykt6 versus the closed, inactive conformation of ykt6.

ykt6 activators that can be utilized in the methods disclosed herein are described in the U.S. patent application US2021/0113552, the content of which is incorporated herein.

In some embodiments, ykt6 activator is selected from the following compounds or a pharmaceutically acceptable salt thereof.

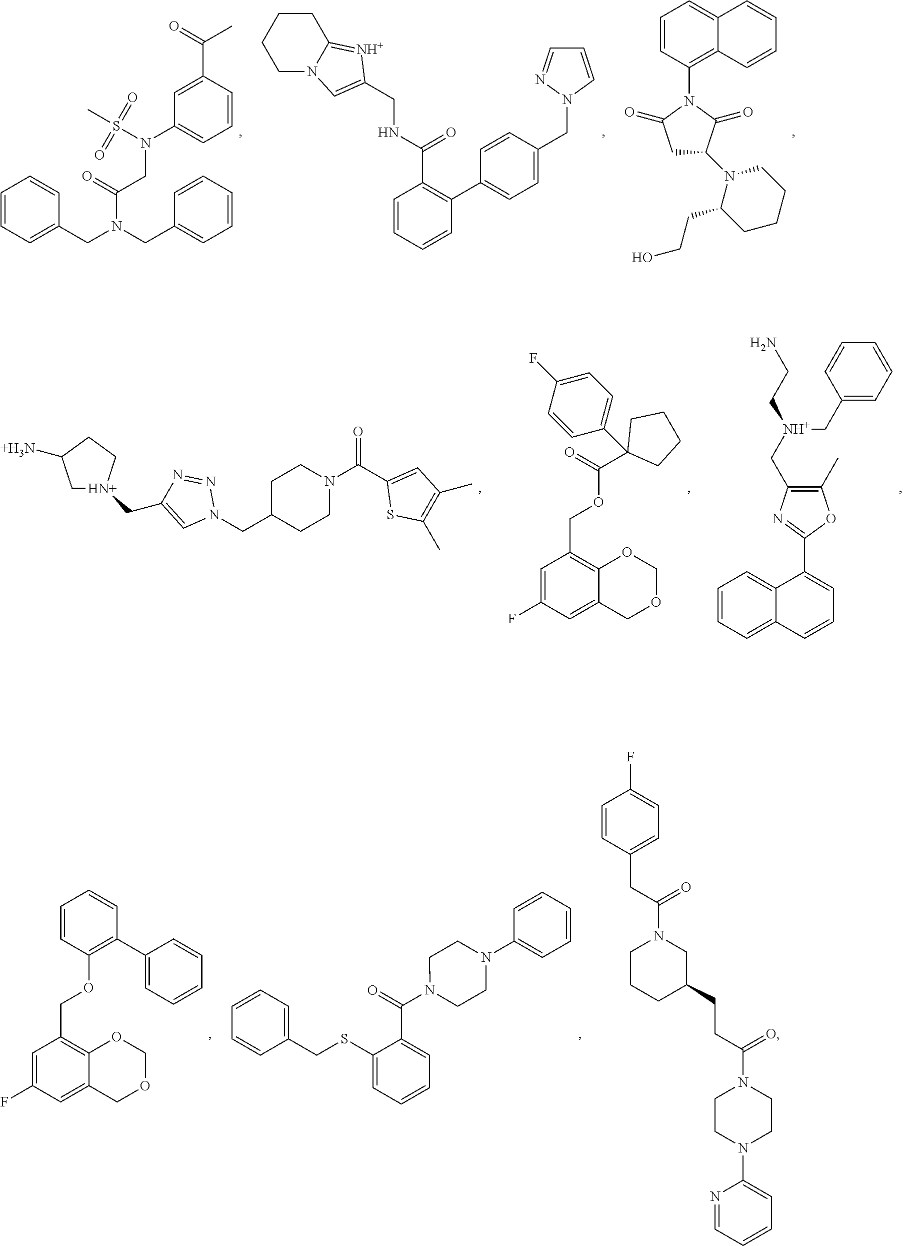

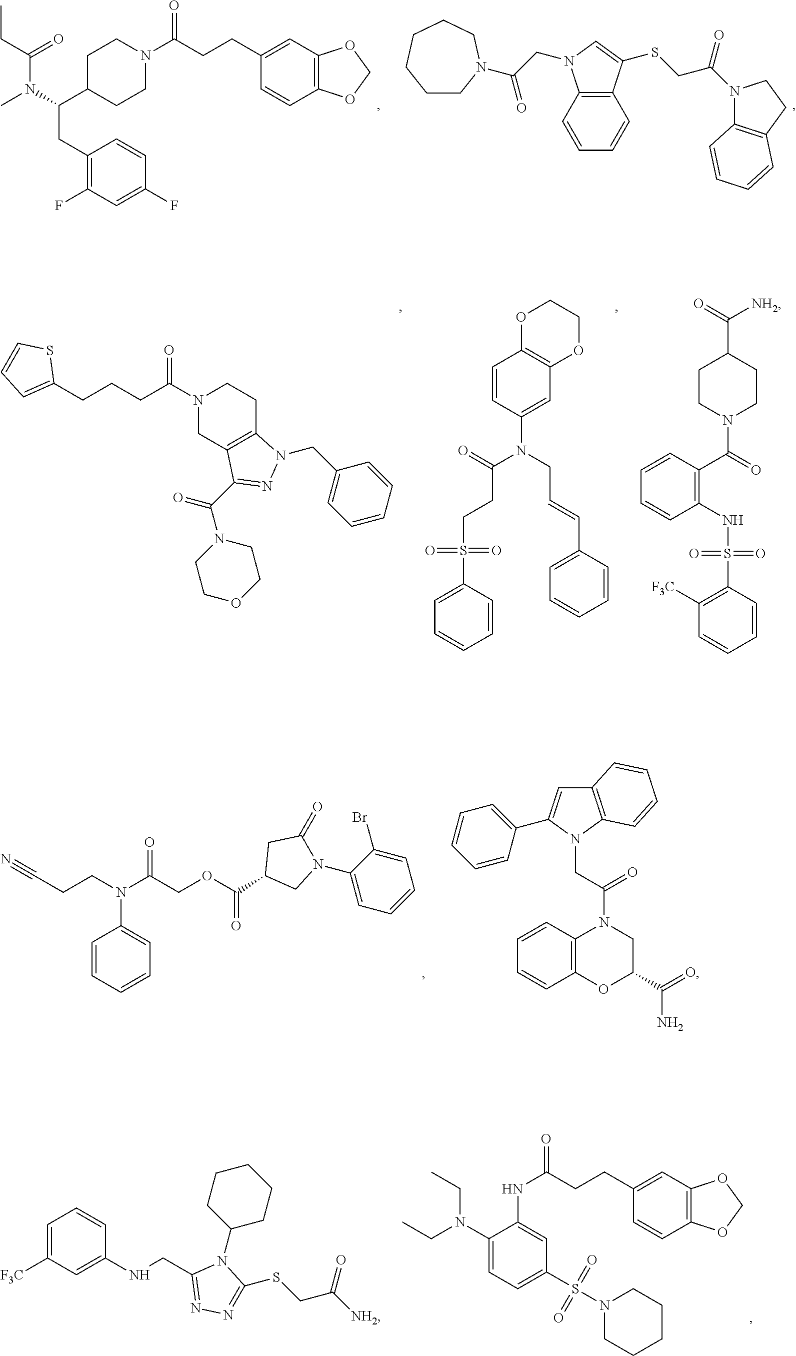

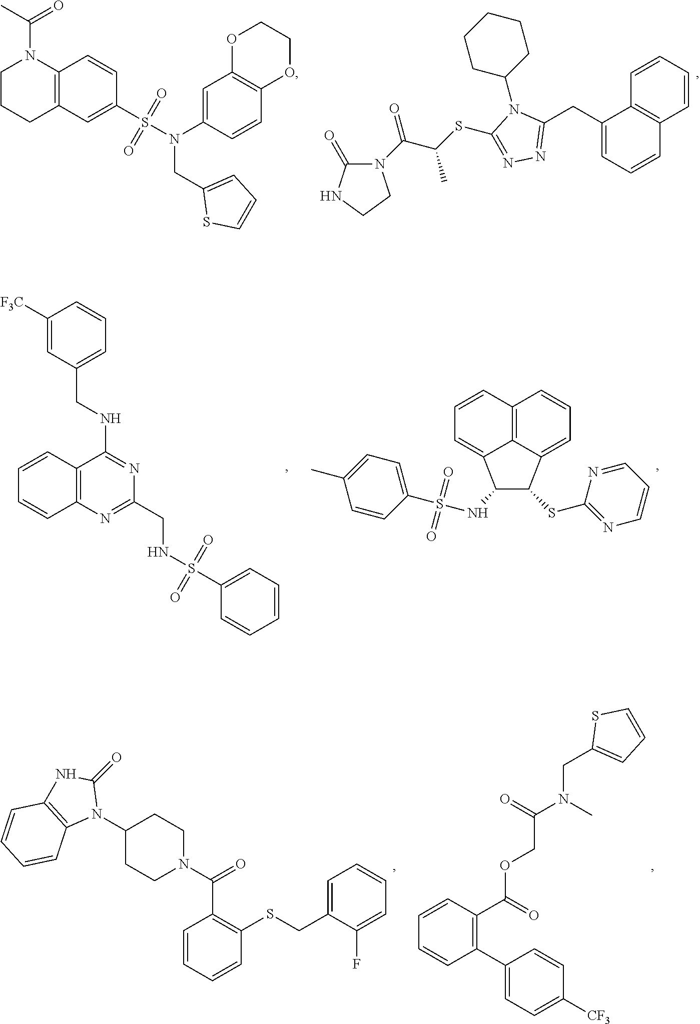

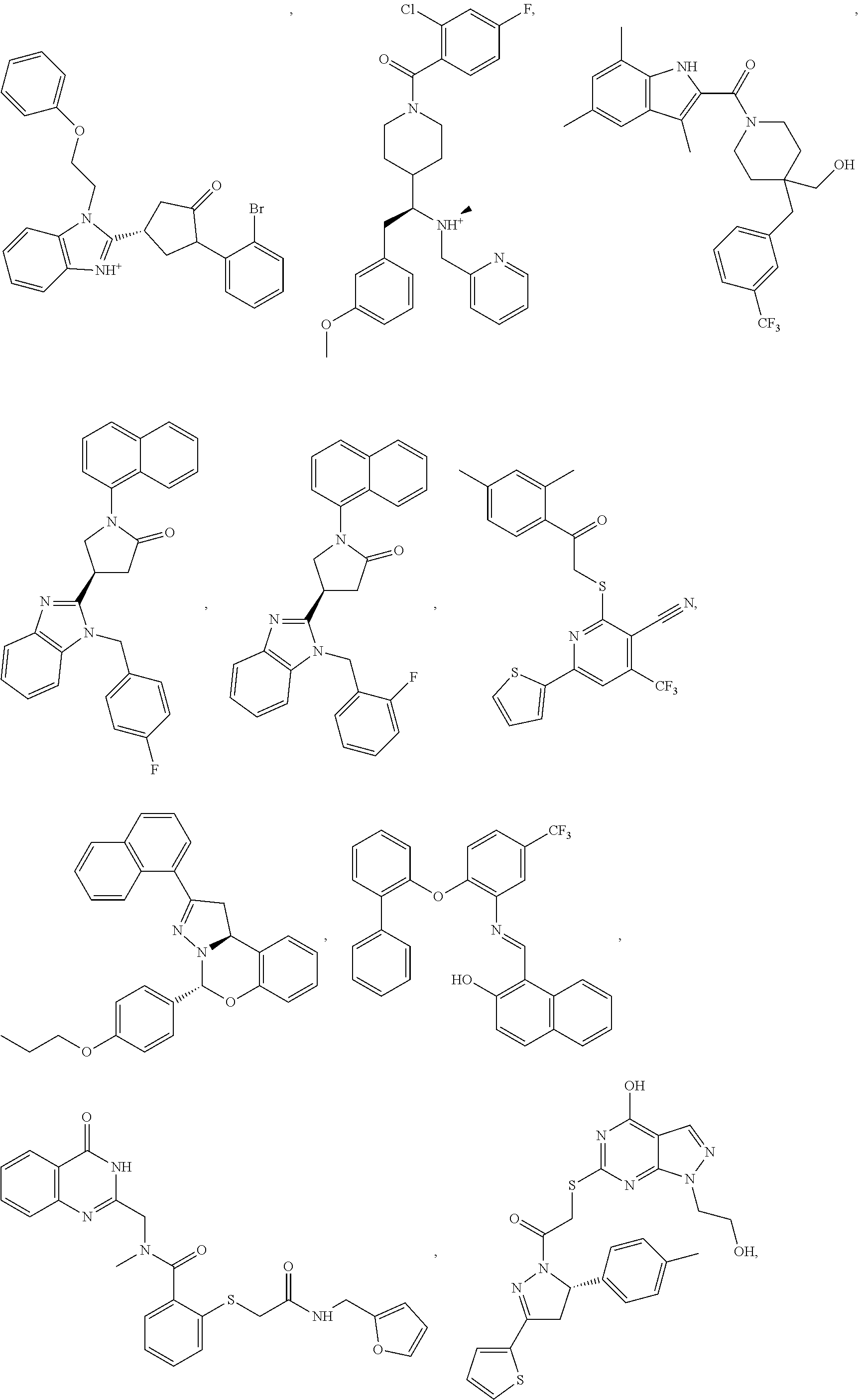

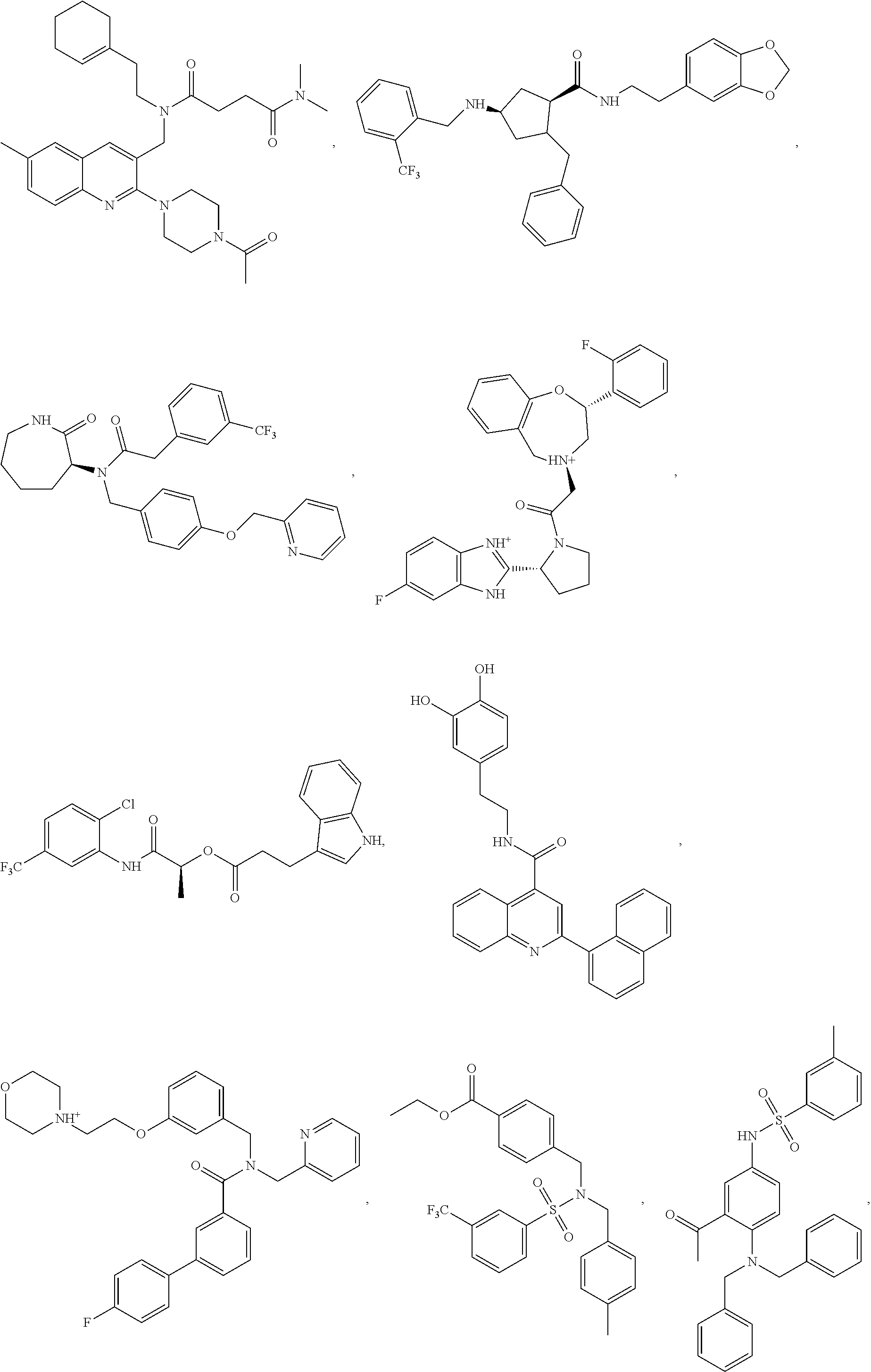

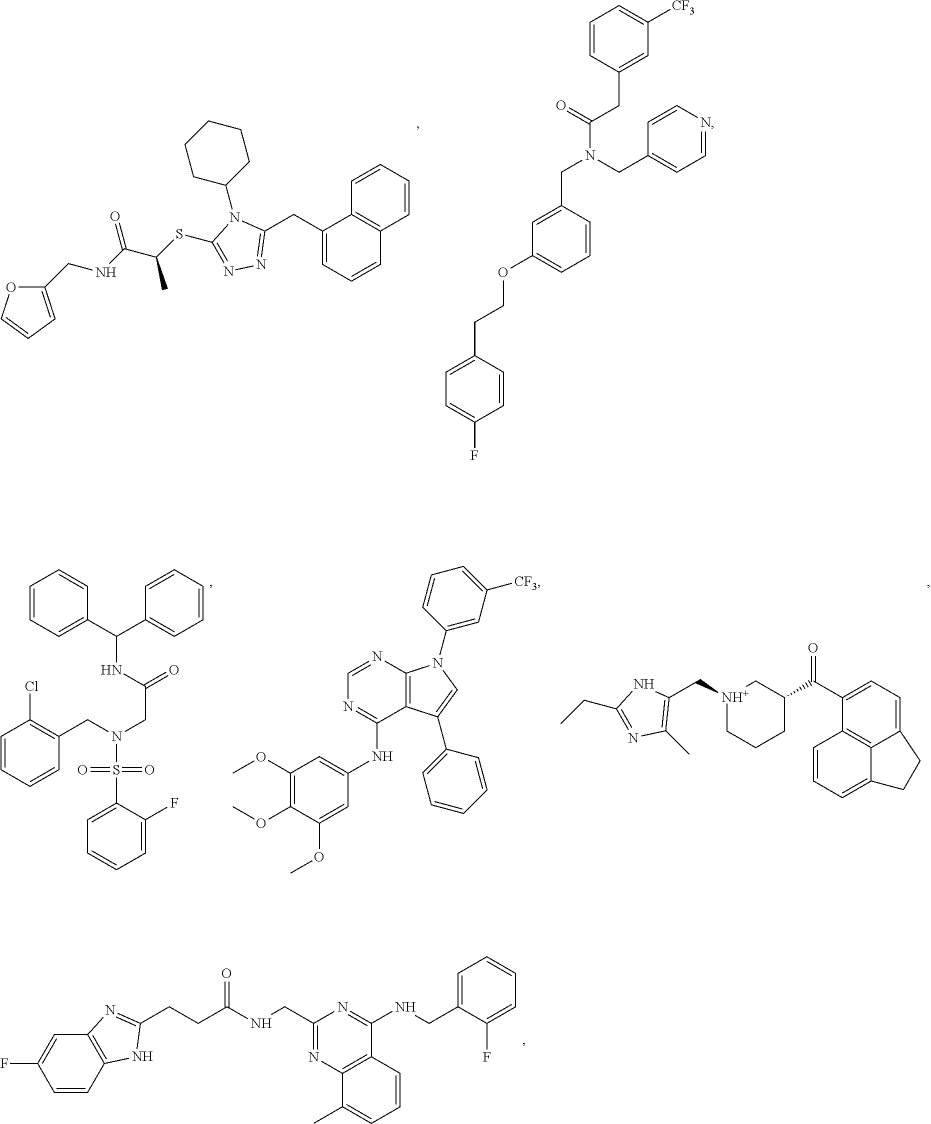 [SMILES: C[C@H](Sc1nnc(Cc2cccc3ccccc23)n1C1CCCCC1)C(=O)NCc1ccco1 ; O=C(Cc1cccc(C(F)(F)F)c1)N(Cc1ccncc1)Cc1cccc(OCCc2ccc(F)cc2)c1 ; O=C(CN(Cc1ccccc1Cl)S(=O)(=O)c1ccccc1F)NC(c1ccccc1)c1ccccc1 ; COc1cc(Nc2ncnc3c2c(-c2ccccc2)cn3-c2cccc(C(F)(F)F)c2)cc(OC)c1OC ; CCc1nc(C)c(C[N@@H+]2CCC[C@@H](C(=O)c3ccc4c5c(cccc35)CC4)C2)[nH]1 ; Cc1cccc2c(NCc3ccccc3F)nc(CNC(=O)CCc3nc4cc(F)ccc4[nH]3)nc12]

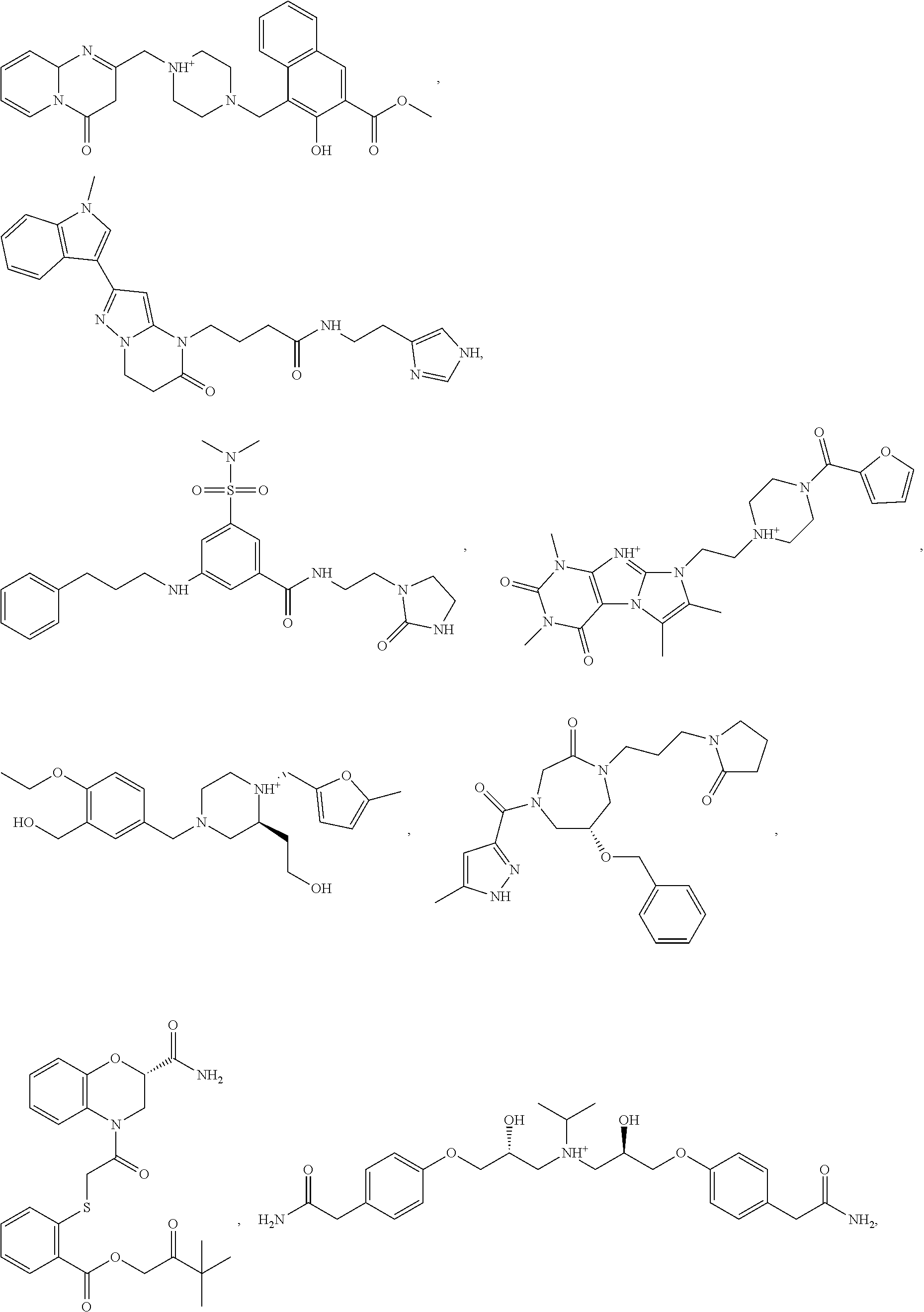

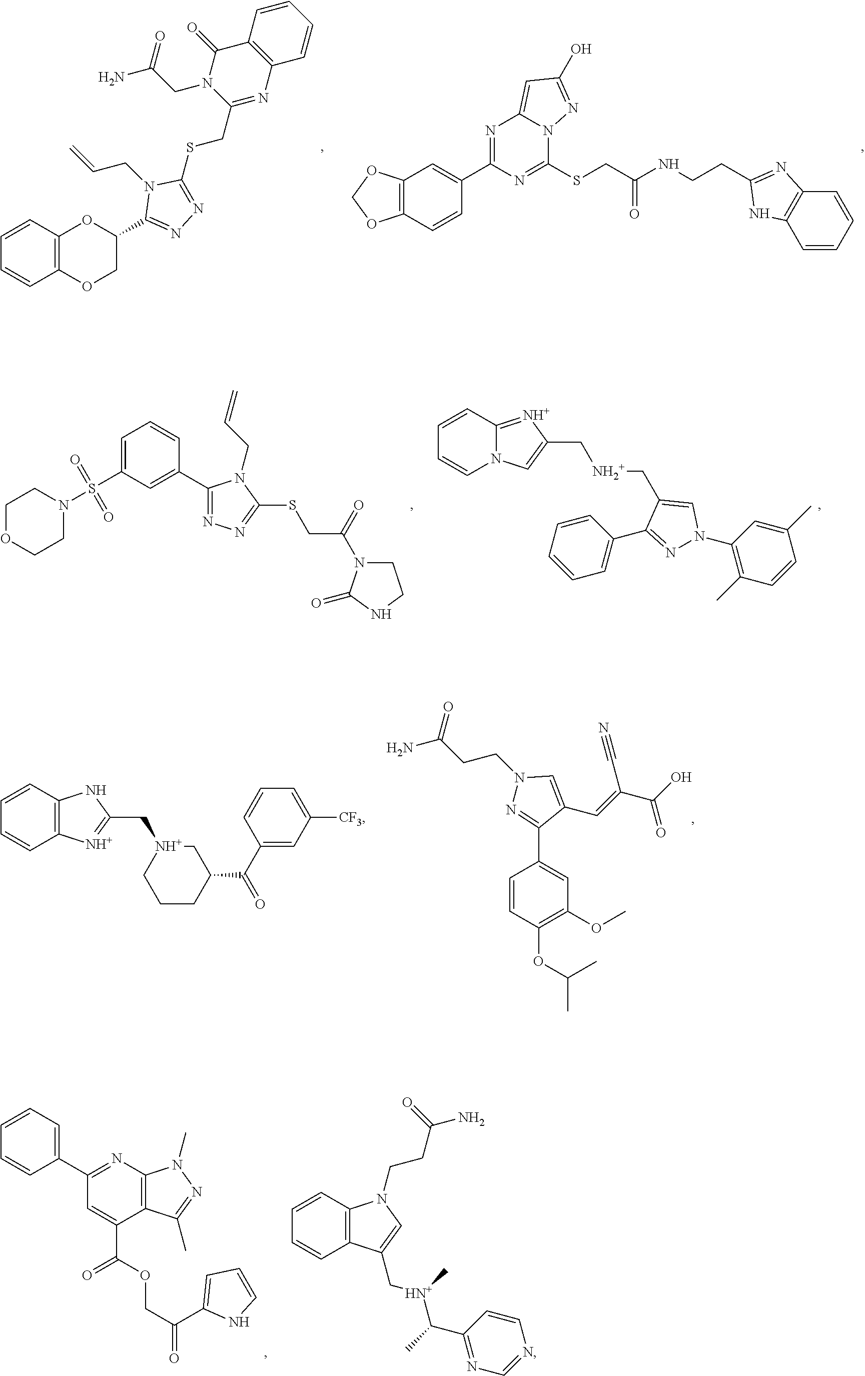

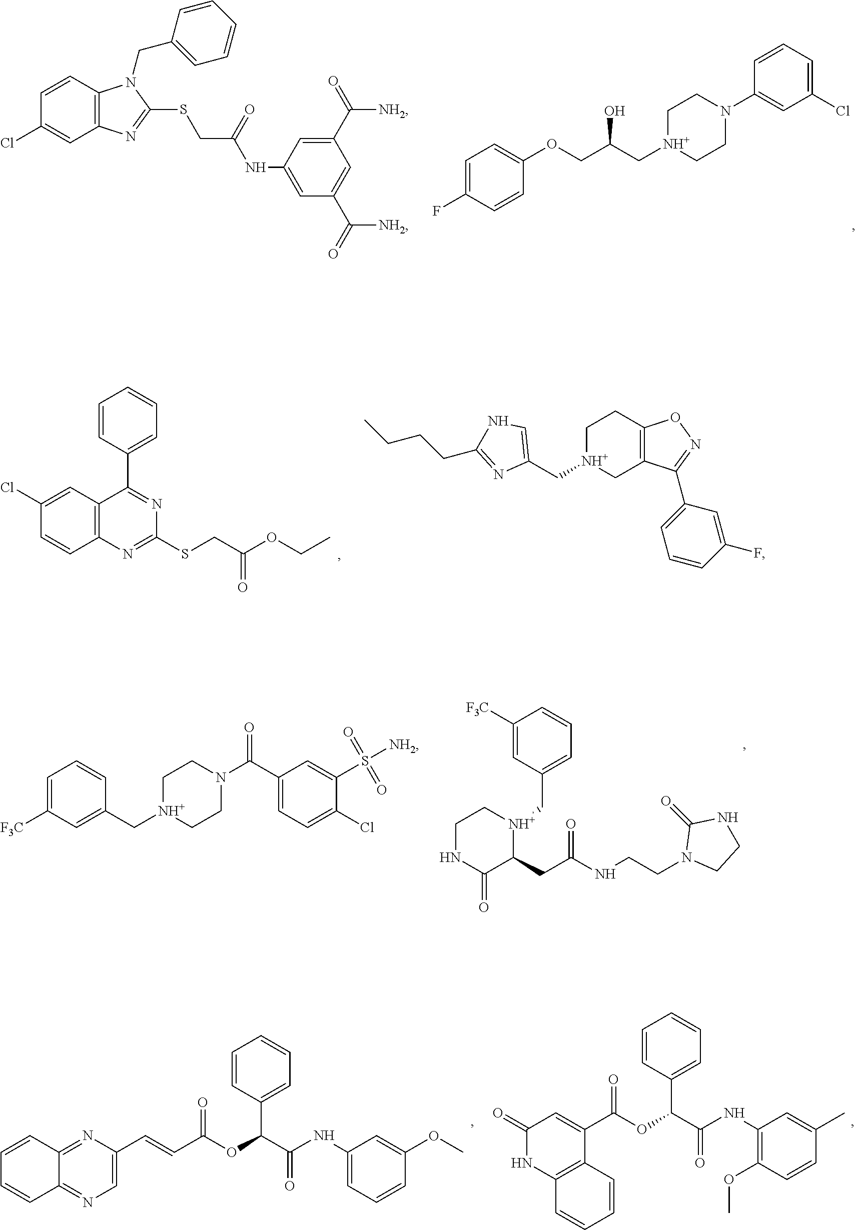

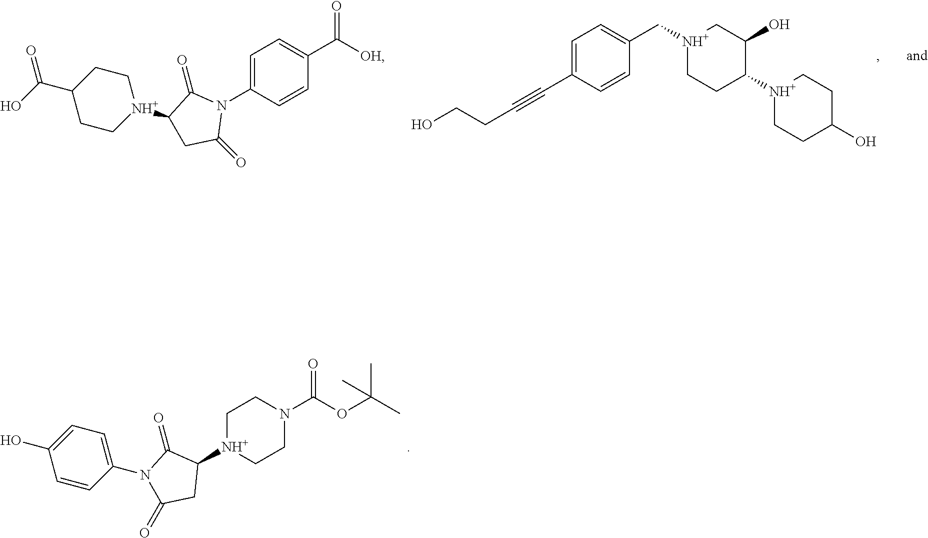

ER Protein Folding Enhancer

New uses for ER protein folding enhancer are disclosed herein.

Ryanodine Receptor (RyR) Inhibitor

As used herein, “ryanodine receptor inhibitor” (RyR inhibitor) refers to an inhibitor of ryanodine receptor. RyR inhibitors may include but is not limited to diltiazem (DILT), dantrolene (DANT), 1,1′-diheptyl-4,4′-bipyridinium (DHBP), JTV 519 fumarate, ruthenium red, and ryanodine.

As used herein, diltiazem (DILT) refers to the chemical [(2S,3S)-5-[2-(dimethylamino)ethyl]-2-(4-methoxyphenyl)-4-oxo-2,3-dihydro-1,5-benzothiazepin-3-yl]acetate and has a CAS No. of 56209-45-1. Diltiazem (DILT) has a chemical structure as follows:

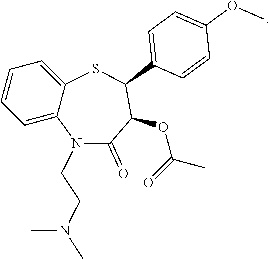 [SMILES: COc1ccc([C@@H]2Sc3ccccc3N(CCN(C)C)C(=O)[C@@H]2OC(C)=O)cc1]

As used herein, dantrolene (DANT) refers to the chemical 1-[(E)-[5-(4-nitrophenyl)furan-2-yl]methylideneamino]imidazolidine-2,4-dione and has a CAS No. of 7261-97-4. Dantrolene (DANT) has a chemical structure as follows:

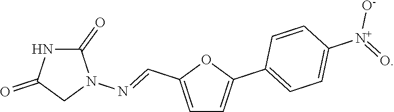 [SMILES: O=C1CN(/N=C/c2ccc(-c3ccc([N+](=O)[O-])cc3)o2)C(=O)N1]

As used herein, 1,1′-diheptyl-4,4′-bipyridinium (DHBP) refers to the dibromide of DHBP (i.e. 1,1′-diheptyl-4,4′-bipyridinium dibromide). 1,1′-diheptyl-4,4′-bipyridinium dibromide has a CAS No. of 6159-05-3 and has a chemical structure as follows:

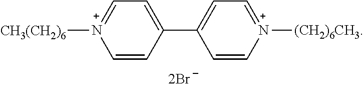

As used herein, JTV 519 fumarate refers to the chemical 3-(4-benzylpiperidin-1-yl)-1-(7-methoxy-3,5-dihydro-2H-1,4-benzothiazepin-4-yl)propan-1-one; (E)-but-2-enedioic acid and has a CAS No. of 1883549-36-7. JTV 519 fumarate has a chemical structure as follows:

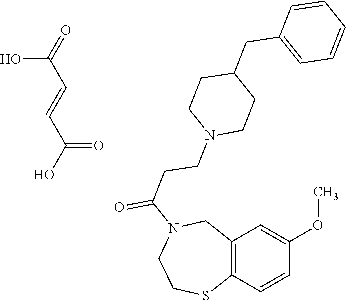 [SMILES: O=C(O)/C=C/C(=O)O ; COc1ccc2c(c1)CN(C(=O)CCN1CCC(Cc3ccccc3)CC1)CCS2]

As used herein, ruthenium red refers to the chemical ammoniated ruthenium oxychloride and has a CAS No. of 11103-72-3. Ruthenium red has a chemical structure as follows:

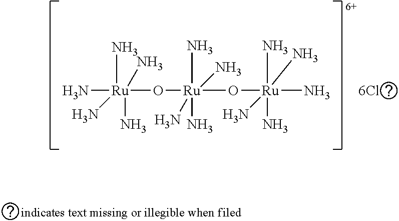

As used herein, ryanodine refers to the chemical [(1R,2R,3S,6S,7S,9S,10R,11S,12R,13S,14R)-2,6,9,11,13,14-hexahydroxy-3,7,10-trimethyl-11-propan-2 yl-15-oxapentacyclo[7.5.1.0^{1,6}.0^{7,13}.0^{10,14}]pentadecan-12-yl] 1H-pyrrole-2-carboxylate and has a CAS No. of 15662-33-6. Ryanodine has a chemical structure as follows:

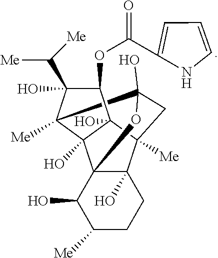

Other ER protein folding enhancer are described in U.S. patent documents U.S. Pat. Nos. 10,786,508, 10,751,341, 10,570,135, U.S. 2019/0092789, U.S. 2018/0185368, U.S. Pat. Nos. 9,920,061, 9,868,742, 9,840,510, and 9,732,089, the content of which are incorporated herein.

N-Glycosylation Enhancer

New uses for N-glycosylation enhancers are disclosed herein.

N-Acetylglucosamine

As used herein, “N-acetylglucosamine” refers to the compound β-D-(acetylamino)-2-deoxy-glucopyranose. N-acetylglucosamine has a chemical structure as follows:

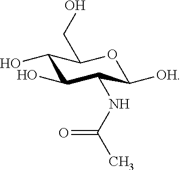

As used herein, “N-acetylglucosamine-6-acetate” refers to the compound 2-(acetylamino)-2-deoxy-D-glucose 6-Acetate and has a chemical structure as follows:

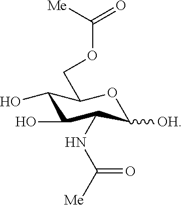

“L-glutamine” refers to the compound (2S)-2-amino-4-carbamoylbutanoic acid and has a chemical structure as follows:

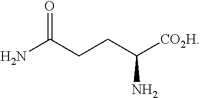

“Fructose-6-phosphate” refers to a derivative of fructose that has been phosphorylated at the 6-hydroxy group. Based on specific applications, a skilled artisan would be able to pick a suitable form of fructose-6-phosphate that contain suitable counter cations, including, but are not limited to, sodium, potassium, etc.

As used herein, the term “allosteric activator of glutamine:F-6-P transaminase-1” refers to a molecule that binds to locations on the glutamine-fructose-6-phosphate transaminase-1 enzyme away from the active site, inducing a conformational change that increases the affinity of the enzyme's active site(s) for its substrate(s).

As used herein, the term “allosteric activator of glutamine:F-6-P transaminase-2” refers to a molecule that binds to locations on the glutamine-fructose-6-phosphate transaminase-2 enzyme away from the active site, inducing a conformational change that increases the affinity of the enzyme's active site(s) for its substrate(s).

The compounds of the disclosure may be isomeric. In some embodiments, the disclosed compounds may be isomerically pure, wherein the compounds represent greater than about 99% of all compounds within an isomeric mixture of compounds. Also contemplated herein are compositions comprising, consisting essentially of, or consisting of an isomerically pure compound and/or compositions that are isomerically enriched, which compositions may comprise, consist essential of, or consist of at least about 50%, 60%, 70%, 80%, 90%, 95%, 96%, 97%, 98%, 99%, or 100% of a single isomer of a given compound.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” or “+” or “−” depending on the configuration of substituents around the chiral or stereogenic carbon atom and or the optical rotation observed. The disclosed compounds encompass various stereo isomers and mixtures thereof. Stereoisomers include enantiomers and diastereomers. Mixtures of enantiomers or diastereomers may be designated (±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise. Also contemplated herein are compositions comprising, consisting essentially of, or consisting of an enantiopure compound and/or compositions that are enantiomer enriched, which compositions may comprise, consist essential of, or consist of at least about 50%, 60%, 70%, 80%, 90%, 95%, 96%, 97%, 98%, 99%, or 100% of a single enantiomer of a given compound (e.g., at least about 95% of an R enantiomer of a given compound).

In some embodiments, the disclosed subject matter relates to one or more ER-Golgi trafficking enhancer and ER protein folding enhancer, as set forth above, formulated into compositions together with one or more physiologically tolerable or acceptable diluents, carriers, adjuvants or vehicles that are collectively referred to herein as carriers. Compositions suitable for such contact or administration can comprise physiologically acceptable aqueous or nonaqueous solutions, dispersions, suspensions or emulsions, whether or not sterile. Amounts of a compound effective to reduce α-synuclein may be determined empirically, and making such determinations is within the skill in the art.

It is understood by those skilled in the art that dosage amount will vary with the activity of a particular inhibitor or inactivator compound, disease state, route of administration, duration of treatment, and like factors well-known in the medical and pharmaceutical arts. In general, a suitable dose will be an amount which is the lowest dose effective to produce a therapeutic or prophylactic effect. If desired, an effective dose of such a compound, pharmaceutically acceptable salt thereof, or related composition may be administered in two or more sub-doses, administered separately over an appropriate period of time.

Methods of preparing pharmaceutical formulations or compositions include the step of bringing an inhibitor or inactivator compound into association with a carrier and, optionally, one or more additional adjuvants or ingredients. For example, standard pharmaceutical formulation techniques can be employed, such as those described in Remington's Pharmaceutical Sciences, Mack Publishing Company, Easton, Pa.

Regardless of composition or formulation, those skilled in the art will recognize various avenues for medicament administration, together with corresponding factors and parameters to be considered in rendering such a medicament suitable for administration. Accordingly, with respect to one or more non-limiting embodiments, the disclosed compounds may be utilized as inhibitor or inactivator compounds for the manufacture of a medicament for therapeutic use in the treatment or prevention of a disease or disorder associated with accumulation of α-synuclein. Suitable diseases, disorders, or conditions may include (i) a neurodegenerative disease, disorder, or condition, which may include, but is not limited to Parkinson's disease, Lewy body dementia, Alzheimer's disease, amyotrophic lateral sclerosis (ALS), multiple system atrophy, Huntington's disease, Prion disease, frontotemporal dementia, Picks disease, progressive supranuclear palsy, and progeria, (ii) a rapid eye movement (REM) behavior sleep disorder linked to Parkinson's disease and/or GBA1 mutation carriers, (iii) a pediatric lysosomal storage disorder, which may include, but is not limited to glycogen storage disorder, neuronal ceroid lipofuscinosis disorder, sphingolipid storage disorder, cholesterol storage disorder, and fatty acid storage disorder, and/or (iv) a protein misfolding or amyloidosis disease, disorder, or condition, which may include, but is not limited to cataract caused by α-crystallin aggregation, systemic amyloidosis, type 2 diabetes characterized by amylin aggregation, and alpha-1-antitrypsin deficiency liver disease.

Generally, with respect to various embodiments, the disclosed subject matter can be directed to method(s) for the treatment of a neurodegenerative disorder, a rapid eye movement (REM) behavior sleep disorder, a pediatric lysosomal storage disorder, and/or a protein misfolding or amyloidosis disorder. As used herein, the term “disorder” refers to a condition in which there is a disturbance of normal functioning. A “disease” is any abnormal condition of the body or mind that causes discomfort, dysfunction, or distress to the person affected or those in contact with the person. Sometimes the term is used broadly to include injuries, disabilities, syndromes, symptoms, deviant behaviors, and atypical variations of structure and function, while in other contexts these may be considered distinguishable categories. It should be noted that the terms “disease”, “disorder”, “condition” and “illness”, are equally used herein.

The compounds and compositions disclosed herein may be administered in methods of treatment as known in the art. Accordingly, various such compounds and compositions can be administered in conjunction with such a method in any suitable way. For example, administration may comprise oral, intravenous, intraarterial, intramuscular, subcutaneous, intraperitoneal, parenteral, transdermal, intravaginal, intranasal, mucosal, sublingual, topical, rectal or subcutaneous administration, or any combination thereof.

According to some embodiments, the treated subject may be a mammalian subject. Although the methods disclosed herein are particularly intended for the treatment of neurodegenerative disorders, rapid eye movement (REM) behavior sleep disorders, pediatric lysosomal storage disorders, and/or protein misfolding or amyloidosis disorders in humans, other mammals are included. By way of non-limiting examples, mammalian subjects include monkeys, equines, cattle, canines, felines, mice, rats and pigs.

The terms “treat, treating, treatment” as used herein and in the claims mean ameliorating one or more clinical indicia of disease activity in a subject having a pathologic disorder. “Treatment” refers to therapeutic treatment. Those in need of treatment are mammalian subjects suffering from any pathologic disorder. By “patient” or “subject in need” is meant any mammal for which administration of a compound or any pharmaceutical composition of the sort described herein is desired, in order to prevent, overcome, modulate or slow down such infliction. To provide a “preventive treatment” or “prophylactic treatment” is acting in a protective manner, to defend against or prevent something, especially a condition or disease.

More generally, the disclosed methods may be directed to affecting, modulate, reducing, inhibiting and/or preventing the initiation, and/or progression of a neurodegenerative disorder, a rapid eye movement (REM) behavior sleep disorder, a pediatric lysosomal storage disorder, and/or a protein misfolding or amyloidosis disorder associated with accumulation of α-synuclein.

### EXAMPLES

The following Examples are illustrative and should not be interpreted to limit the scope of the claimed subject matter. The following non-limiting Examples and data illustrate various aspects and features relating to the disclosed compounds, compositions, and methods including the treatment of diseases, disorders, and conditions associated with accumulation of α-synuclein. While the utility of this invention is illustrated through the use of several compounds and compositions which can be used therewith, it will be understood by those skilled in the art that comparable results are obtainable with various other compound(s), as are commensurate with the scope of this invention.

### Example 1—Rescue of α-Synuclein Aggregation in Parkinson's Patient Neurons by Synergistic Enhancement of ER Proteostasis and Protein Trafficking

Neurodegenerative disorders are characterized by a collapse in proteostasis, shown by the accumulation of insoluble protein aggregates in the brain. Proteostasis involves a balance of protein synthesis, folding, trafficking, and degradation, but how aggregates perturb these pathways is unknown. Using Parkinson's disease (PD) patient midbrain cultures, one finds that aggregated α-synuclein induces endoplasmic reticulum (ER) fragmentation and compromises ER protein folding capacity, leading to misfolding and aggregation of immature lysosomal b-glucocerebrosidase. Despite this, PD neurons fail to initiate the unfolded protein response, indicating perturbations in sensing or transducing protein misfolding signals in the ER. Small molecule enhancement of ER proteostasis machinery promotes b-glucocerebrosidase solubility, while simultaneous enhancement of trafficking improves ER morphology, lysosomal function, and reduces α-synuclein. The Examples demonstrate that aggregated α-synuclein perturbs the ability of neurons to respond to misfolded proteins in the ER, and that synergistic enhancement of multiple proteostasis branches may provide therapeutic benefit in PD.

### INTRODUCTION

Parkinson's disease (PD) is a neurodegenerative movement disorder that is primarily characterized by the loss of dopaminergic neurons in the substantia nigra pars compacta region of the midbrain. Pathologically, PD is characterized by the presence of protein inclusions called Lewy bodies and Lewy neurites that are largely comprised of the synaptic protein α-synuclein (α-syn) (Spillantini et al., 1997). Critical to the pathogenic mechanism of α-syn, A53T and other familial-linked point mutations in SNCA, the gene that codes for α-syn, result in the accelerated oligomerization or fibrillization of the protein (Conway et al., 1998). Aside from point mutations, multiplications of wild-type SNCA also lead to familial PD. Patients with SNCA multiplications accumulate wild-type α-syn protein, and the severity of their clinical phenotype is dependent on α-syn dosage. For example, the clinical presentation of SNCA duplication patients occurs much later and is not as severe as in SNCA triplications (Fuchs et al., 2007; Singleton et al., 2003). Moreover, patients with SNCA multiplications display the classical Lewy pathology, indicating that the mere overabundance of the wild-type protein leads to neurodegeneration. However, the molecular mechanism by which the overabundance of wild-type α-syn leads to cellular toxicity and contributes to PD pathogenesis is not completely understood.

The accumulation of α-syn into insoluble inclusions suggests that proteostasis pathways, including the lysosomal clearance pathway, are disrupted in the PD brain. Genome-wide association studies in PD patients have identified several risk genes, most of which have key roles in autophagy and lysosomal function (Chang et al., 2017; Nalls et al., 2014; Robak et al., 2017; Simon-Sanchez et al., 2009). Of these, loss-of-function mutations in GBA1 represent one of the strongest genetic risk factors for the development of PD and Dementia with Lewy bodies (DLB) (Chia et al., 2021; Sidransky et al., 2009). Homozygous mutations in the GBA1 gene, which encodes lysosomal β-glucocerebrosidase (GCase), cause the lysosomal storage disorder Gaucher's disease (GD) that is characterized by glycosphingolipid accumulation and neurodegeneration (Roshan Lal and Sidransky, 2017).

Aside from mutations in lysosomal components, genetic analyses indicate that defects in another critical component of the proteostasis network, vesicular trafficking, also contribute to PD pathogenesis (Abeliovich and Gitler, 2016; Hunn et al., 2015; Klein and Mazzulli, 2018; Martin et al., 2014; Singh and Muqit, 2020). Proper GCase maturation requires its trafficking from the endoplasmic reticulum (ER) to the Golgi portion of the secretory pathway. Previous work has shown that α-syn accumulation can impair ER-to-Golgi protein trafficking and disrupt protein maturation (Cooper et al., 2006; Gitler et al., 2008; Gosavi et al., 2002; Thayanidhi et al., 2010). Our recent studies showed that trafficking disruption occurs by α-syn-mediated inhibition of the SNARE protein ykt6, which prevents the fusion of ER-derived vesicles on the cis-Golgi causing downstream lysosomal depletion (Cuddy et al., 2019).

Perhaps the most critical function of the proteostasis network takes place in the ER compartment, where approximately one-third of the cell's proteome is synthesized, folded, and processed. Calcium-dependent molecular chaperones play a pivotal role in maintaining ER proteostasis. The calcium-dependent ER lectin-containing chaperone calnexin (CANX) is particularly important for maintaining proper protein folding and quality control of monomeric N-linked glycosylated proteins, including GCase and other lysosomal hydrolases (Ou et al., 1993; Tan et al., 2014). Disrupted protein trafficking and accumulation of immature proteins in the ER can overwhelm the folding machinery, invariably leading to ER stress and initiation of the unfolded protein response (UPR). The UPR constitutes a series of pathways that transduce ER stress signals to the nucleus for transcriptional upregulation of quality control machinery and expansion of the ER to accommodate excess protein load (Walter and Ron, 2011). The three main stress sensors that initiate the UPR include inositol-requiring enzyme (IRE1), double-stranded RNA-activated protein kinase (PKR)-like ER kinase (PERK), and activating transcription factor 6 (ATF6). These three branches can be stimulated separately or simultaneously, and attempt to restore ER proteostasis by reducing protein synthesis and stimulating gene expression of folding machinery including chaperones GRP78 and GRP94 (Kozutsumi et al., 1988). GBA1 mutations destabilize GCase structure, resulting in UPR induction, expansion of the ER compartment, and elimination of the protein through ER associated degradation (ERAD) (Fernandes et al., 2016; Garcia-Sanz et al., 2017; Ron and Horowitz, 2005). General markers of UPR activation have also been documented in various synucleinopathy models including α-syn overexpressing yeast (Cooper et al., 2006), human mutant SNCA A53T transgenic mice (Colla et al., 2012a; Colla et al., 2018), as well as iPSC-derived cortical neuron models (Chung et al., 2013; Heman-Ackah et al., 2017). Importantly, evidence of UPR activation is present in the substantia nigra of post-mortem PD brains (Credle et al., 2015; Heman-Ackah et al., 2017; Hoozemans et al., 2007). Overexpression of the ER chaperone GRP78 can also reduce neurodegeneration in α-syn expressing animal models (Gorbatyuk et al., 2012), further emphasizing the importance of maintaining ER proteostasis in neuronal health. While these studies collectively suggest that ER dysfunction is associated with PD, the mechanistic link between α-syn accumulation, protein misfolding in the ER, and downstream lysosomal dysfunction has not been established. Furthermore, it is unknown whether enhancing ER proteostasis alone is sufficient to rescue lysosomal dysfunction and reduce pathological α-syn in PD models.

To study the role of ER proteostasis and its mechanistic connection to lysosomal function in PD pathogenesis, a novel induced pluripotent stem cell (iPSC)-derived midbrain dopaminergic (DA) models from PD patients that carry a triplication (3X) in SNCA were developed and characterized. These models show accumulation of insoluble α-syn and GCase dysfunction that is associated with morphological and biochemical alterations in the ER. Lysosomal function in these models can be rescued by simultaneously enhancing protein folding in the ER and trafficking via small molecule modulators. The results provide evidence that comprehensive strategies that target multiple branches of the proteostasis pathway may be beneficial to patients with PD and other proteinopathies.

Results

Novel PD iPSC-Derived Midbrain Models Demonstrate α-Syn Accumulation and Lysosomal Dysfunction.

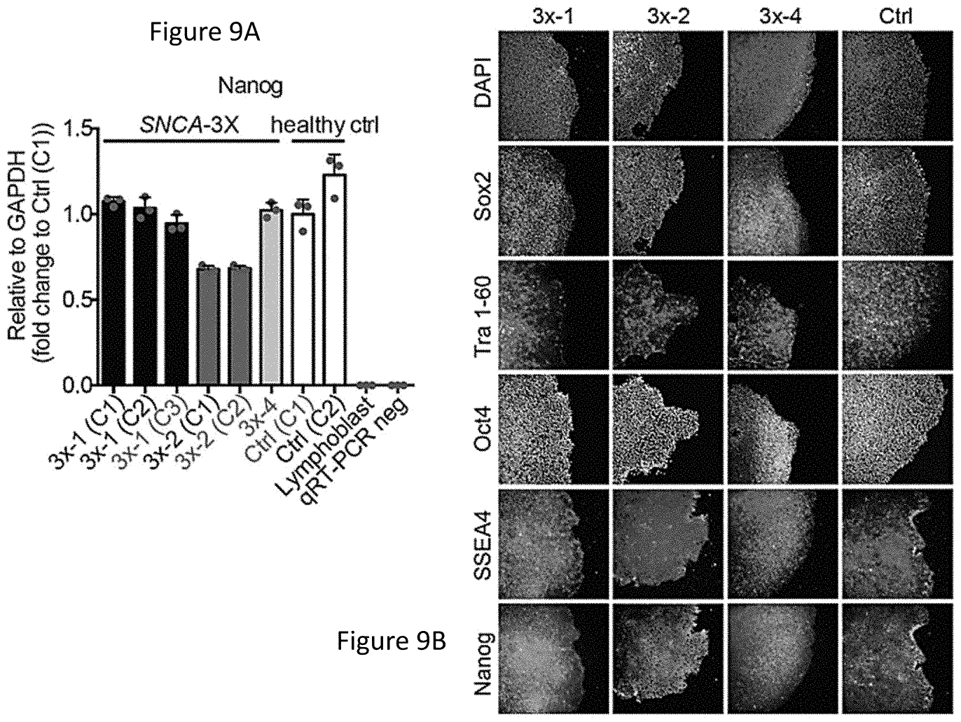

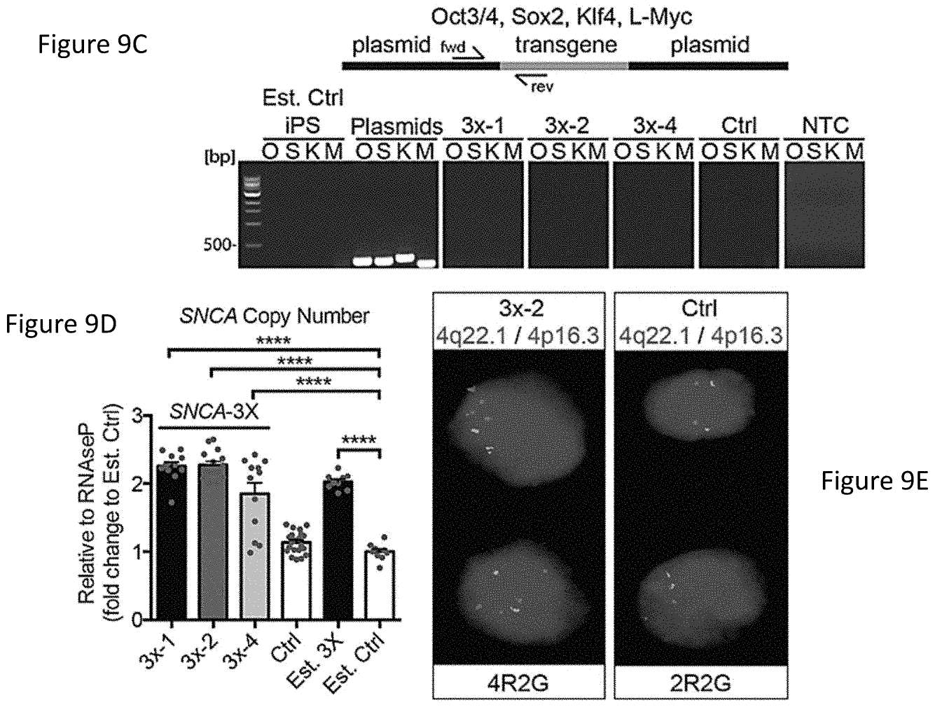

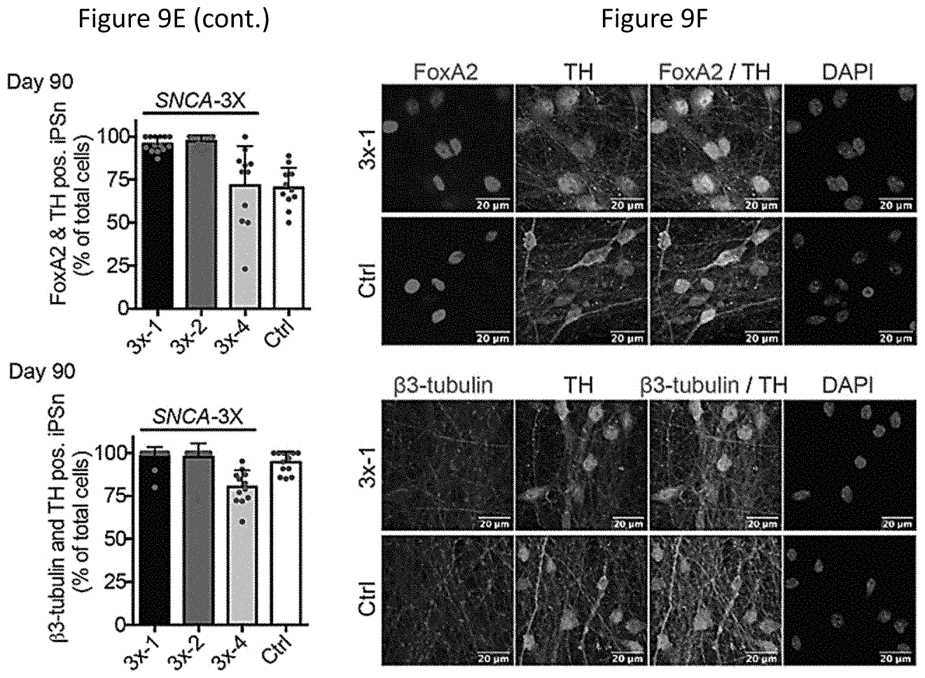

Previous work indicated that α-syn accumulation causes lysosomal dysfunction in PD patient midbrain neurons (Cuddy et al., 2019; Mazzulli et al., 2016a). To further examine the mechanism of this process, we generated several new iPSC lines from B-lymphocytes of a healthy control (Ctrl) and three distinct patients that carry a triplication (3X) in the SNCA genomic region that exhibit early onset parkinsonism and dementia (Singleton et al., 2003). Several clones were expanded and extensively characterized for pluripotency, residual transgene expression of reprogramming factors, SNCA-3X genotype retention, and neural differentiation efficiency (FIG. 9A-F). The most robust iPSC lines (highlighted in red in FIG. 9A) were chosen to carry out experiments, and termed 3x−1 (clone 3; C3), 3x−2 (clone 2; C2), 3x−4, and Ctrl (clone 1; C1).

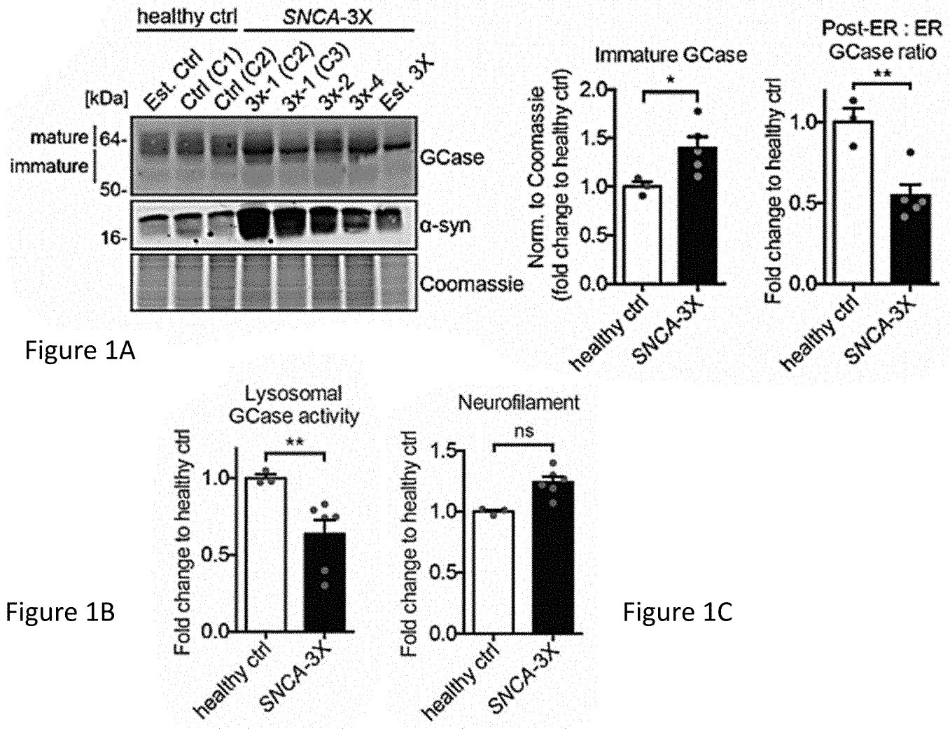

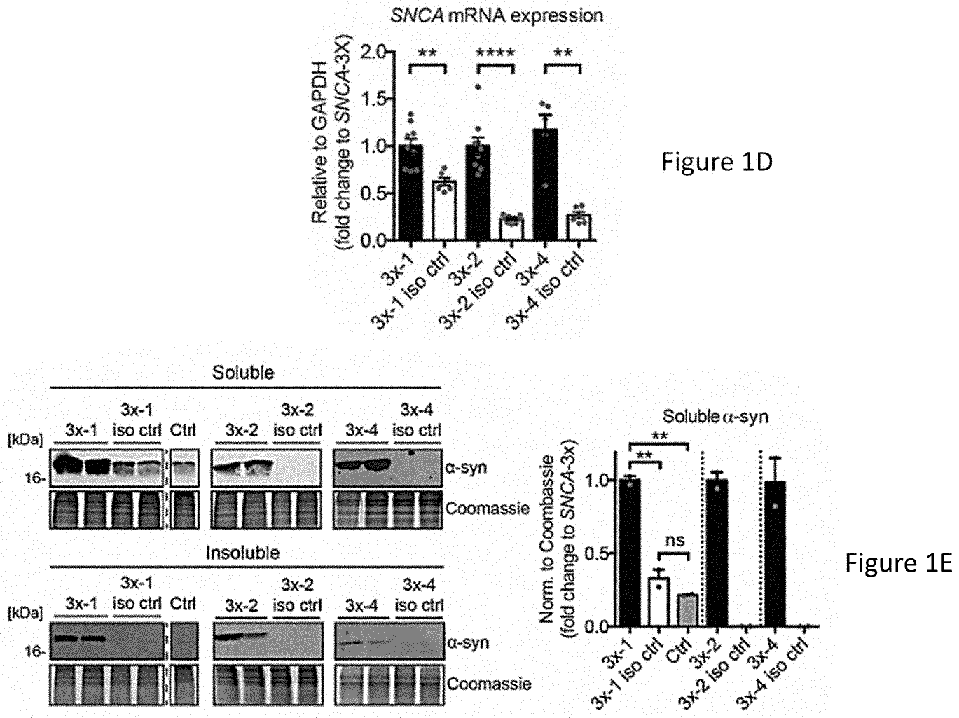

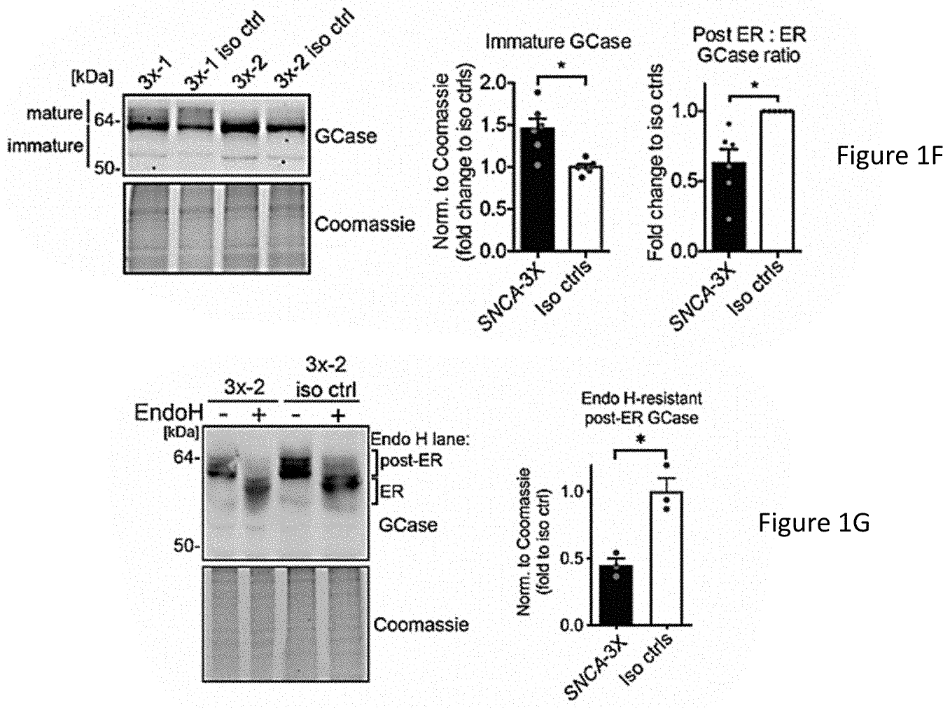

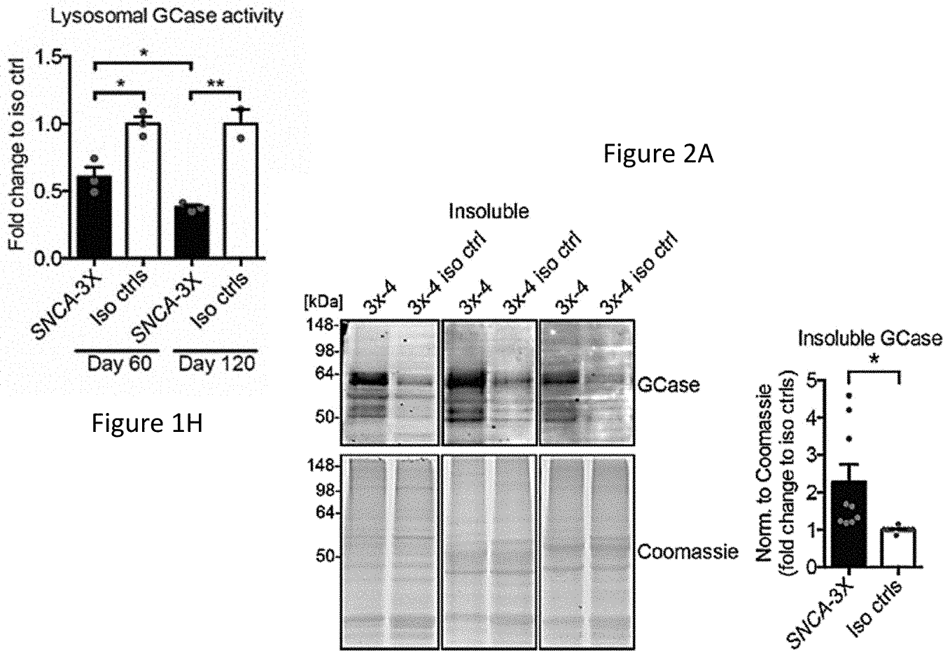

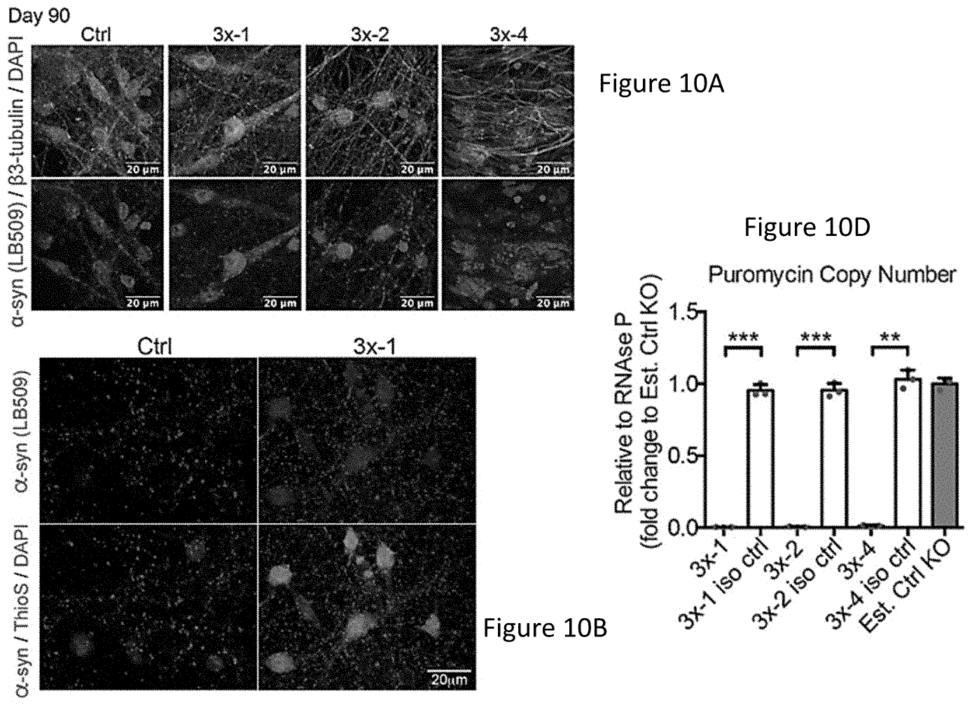

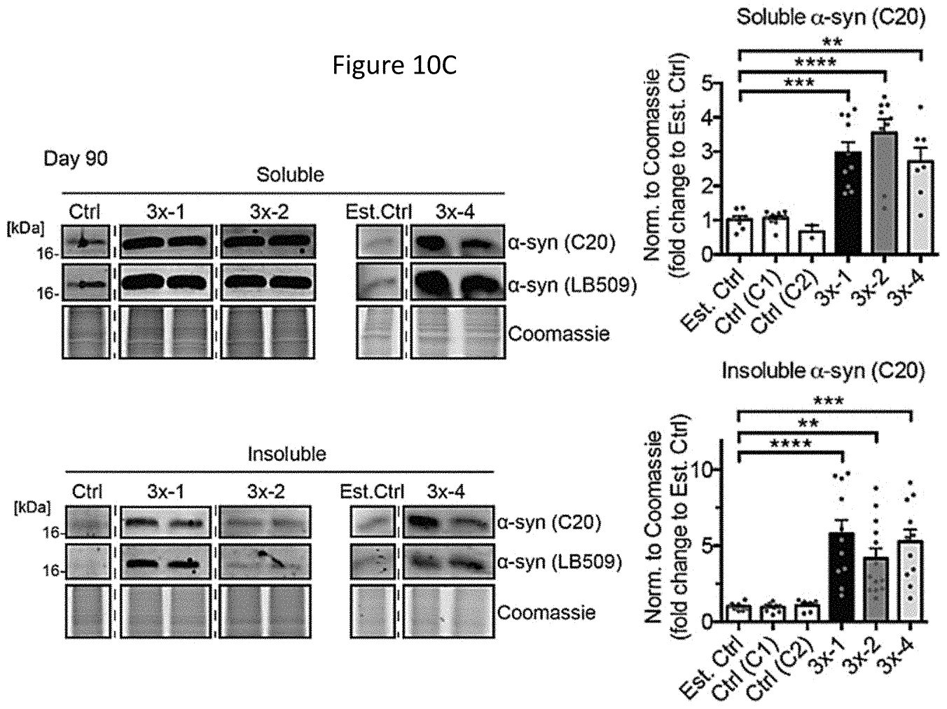

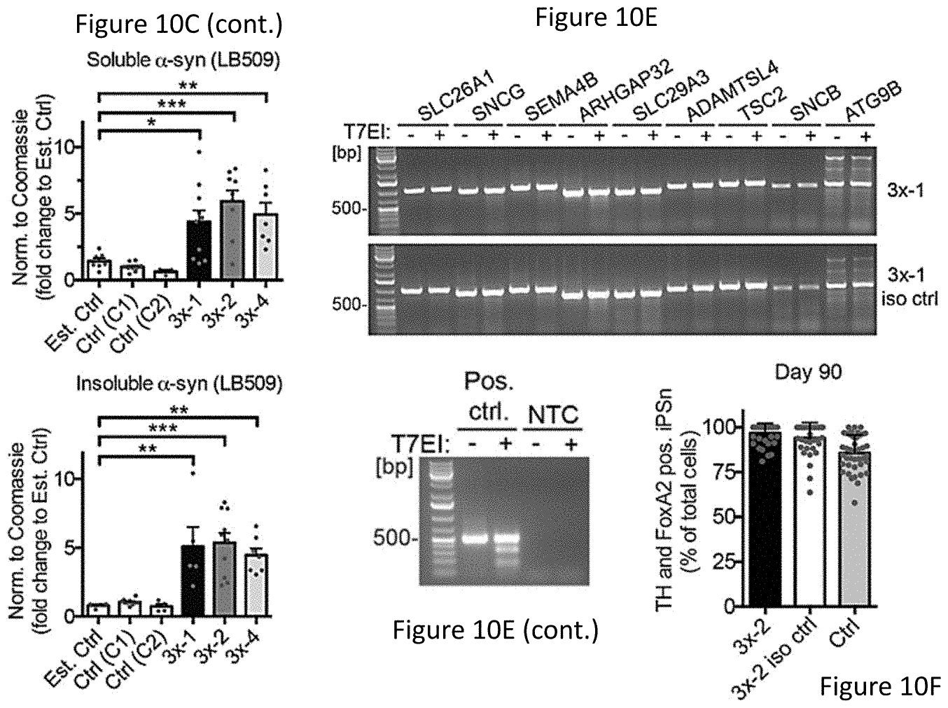

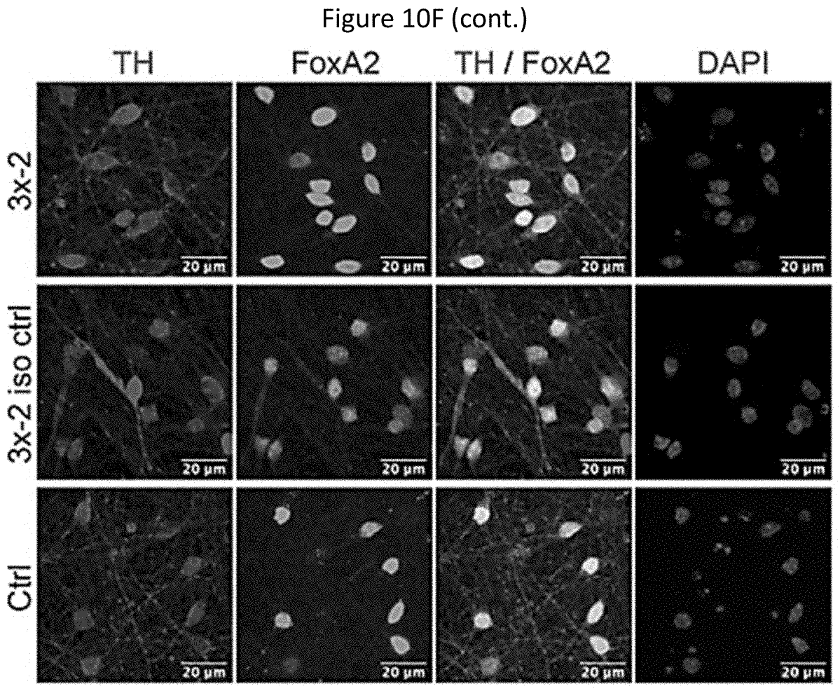

To determine if the novel lines could recapitulate key features of the PD brain, we differentiated iPSCs into midbrain dopamine (DA) neurons (Kriks et al., 2011; Mazzulli et al., 2016a), matured the cultures for 90 days, and analyzed for the presence of aggregated α-syn. In addition to the novel iPSC lines, we also incorporated previously established lines in our study (Est. 3×, and Est. Ctrl) (Mazzulli et al., 2016a). Immunofluorescence analysis indicated that patient lines accumulated α-syn inclusions within neurites and the cell body that were thioflavin positive, while healthy control neurons displayed lower α-syn levels and a punctate pattern that is expected for a synaptic protein (FIG. 10A, B). Biochemical sequential extraction into soluble and insoluble protein fractions showed significant accumulation of α-syn in both fractions of the SNCA-3X DA neurons relative to healthy controls (FIG. 10C). We next investigated GCase maturation and lysosomal function. As GCase matures from the ER to the Golgi, it acquires N-linked glycans resulting in an increase in its molecular weight (Bergmann and Grabowski, 1989). Thus, GCase maturity can be assessed by SDS-PAGE/western blot analysis. We found that GCase maturation was reduced in SNCA-3X DA neurons indicated by the accumulation of immature, low molecular weight forms of GCase (˜55-62 kDa) (FIG. 1A). We also observed a decline in GCase activity specifically within lysosomal compartments in living SNCA-3X DA neurons compared to the healthy control (FIG. 1B). Analysis of neurite degeneration by staining for neurofilament intensity indicated no change, suggesting that the decline in activity is not due to cell toxicity (FIG. 1C).

Since the triplication region in the SNCA-3X PD patients includes 16 other genes aside from SNCA, we aimed to determine if lysosomal dysfunction occurred specifically from α-syn accumulation. We created isogenic controls of all three PD patient iPSC lines by targeted disruption of the SNCA gene using previously established CRISPR/Cas9 constructs (Zunke et al., 2018) to reduce the expression of α-syn. We found that our constructs were efficiently integrated into the SNCA gene and chose clones that lacked off-target effects for further studies (FIG. 10D, E). At the mRNA level, we found that CRISPR/Cas9 editing caused an approximately 50% decrease in SNCA expression in the 3x−1 isogenic control (FIG. 1D), corresponding to a 70% decline in soluble α-syn protein that is comparable to α-syn levels in healthy controls (FIG. 1E). For the isogenic controls of patients 3x−2 and 3x−4, SNCA mRNA expression was reduced by approximately 75% (FIG. 1D), and soluble α-syn protein levels were undetectable (FIG. 1E). Moreover, no insoluble α-syn was detected in any of the isogenic control lines (FIG. 1E). Analysis of DA neuron markers showed that the isogenic controls could differentiate as efficiently as the parental lines, confirming that CRISPR/Cas9 editing or α-syn depletion/knockout has no adverse effects on neurodevelopment in vitro (FIG. 10F), which has similarly been previously described in vivo using α-syn−/− mice (Abeliovich et al., 2000). Analysis of GCase showed that α-syn reduction improved its maturation by reducing the accumulation of immature GCase, while promoting mature GCase (FIG. 1F). Digestion with endoglycosidase H (Endo H), an enzyme that only cleaves glycans from immature GCase forms, validated that ER forms of GCase accumulate in SNCA-3X neurons and that isogenic correction of SNCA increases mature forms (FIG. 1G). Consistent with this, lysosomal GCase activity was also increased in isogenic controls (FIG. 1H). Collectively, these data validate previous findings in novel, robust iPSC-derived synucleinopathy models, and indicate that wild-type GCase trafficking and activity is reduced by α-syn accumulation.

Defects in GCase Maturation Induce its Aggregation in the ER of Patient Midbrain Neurons and Synucleinopathy Brains.

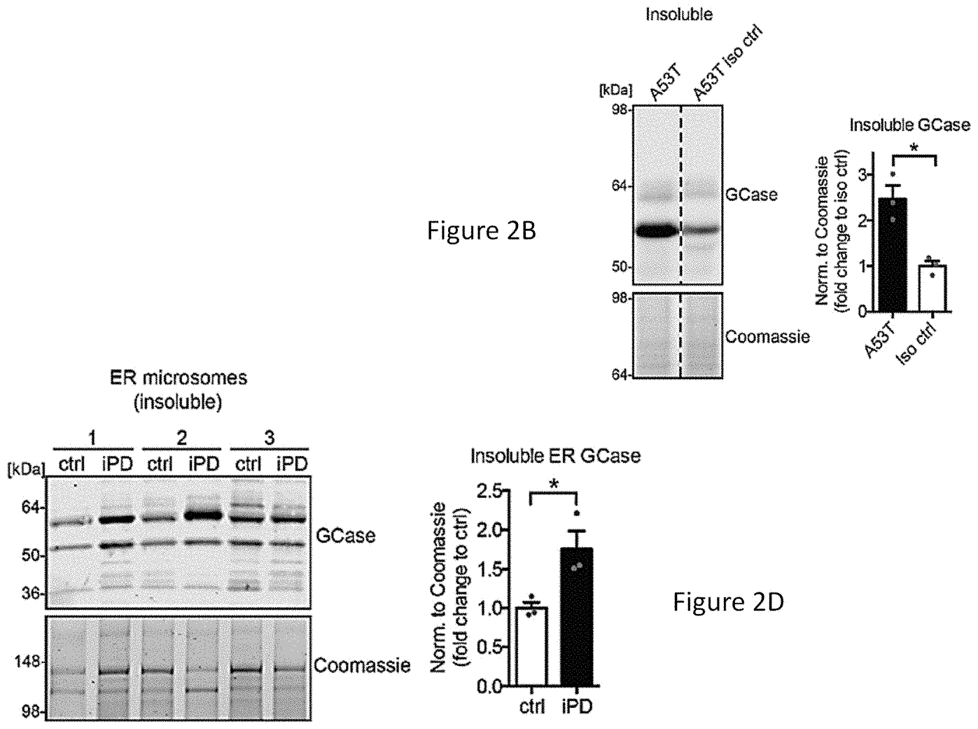

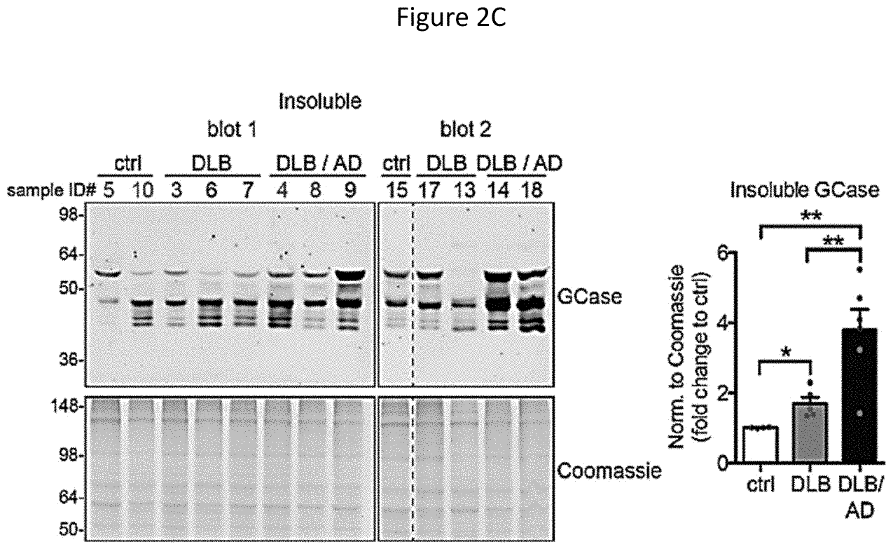

The accumulation of immature proteins in the ER can overwhelm the folding machinery, leading to protein misfolding (Marquardt and Helenius, 1992). Since immature forms of GCase accumulate in SNCA-3X DA neurons, we hypothesized that α-syn-induced trafficking disruptions may result in GCase instability, misfolding and aggregation. To test this, lysates from SNCA-3X DA neurons were sequentially extracted and analyzed by western blot. We found elevated levels of aggregated, immature GCase in Triton X-100-insoluble fractions compared to isogenic controls (FIG. 2A). This was confirmed in a distinct synucleinopathy patient model expressing A53T α-syn that previously demonstrated disrupted protein trafficking of GCase (Cuddy et al., 2019) (FIG. 2B).

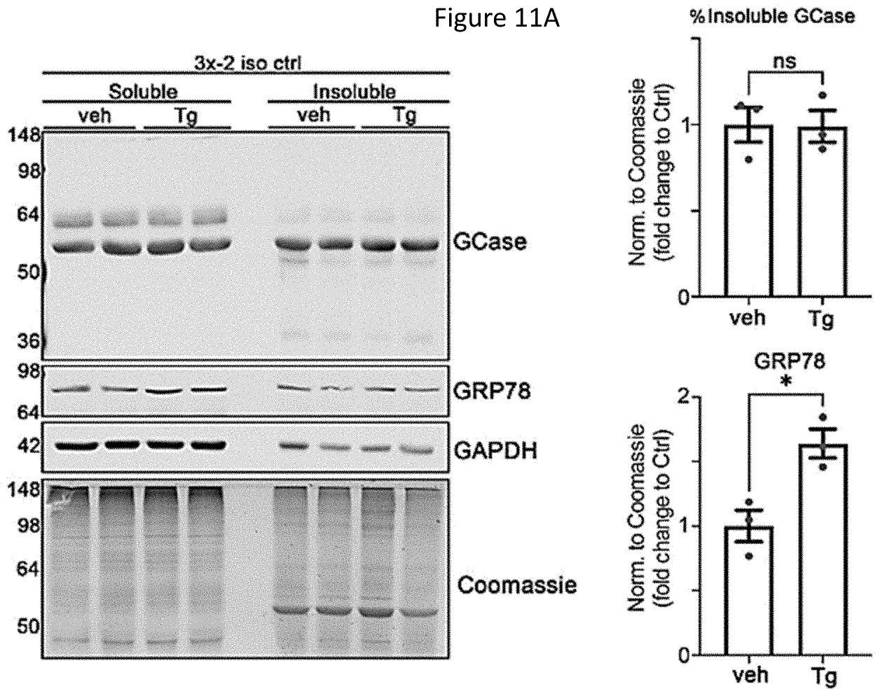

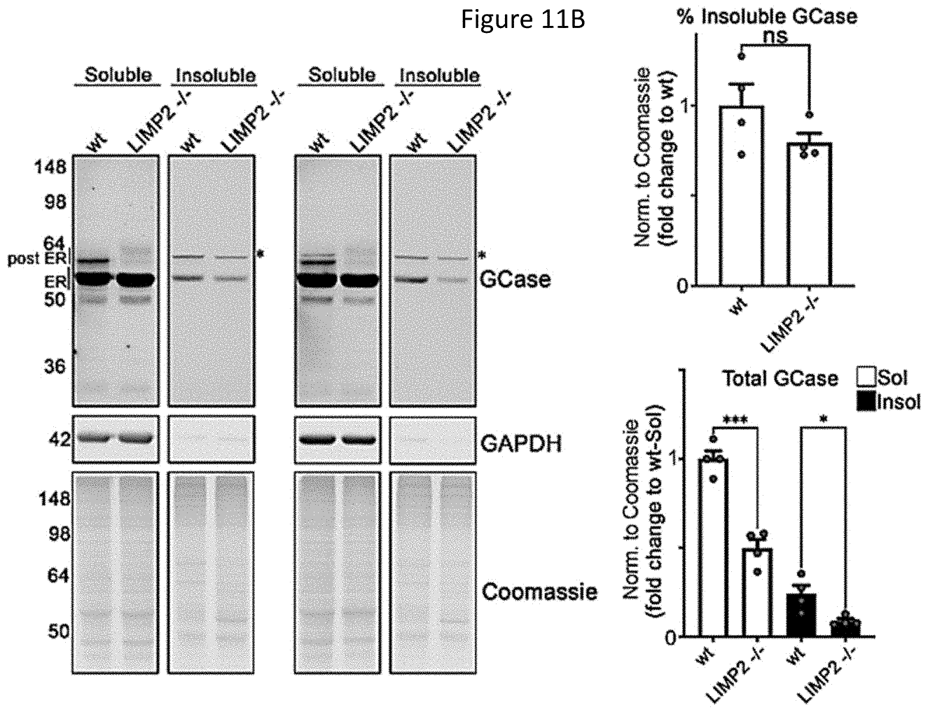

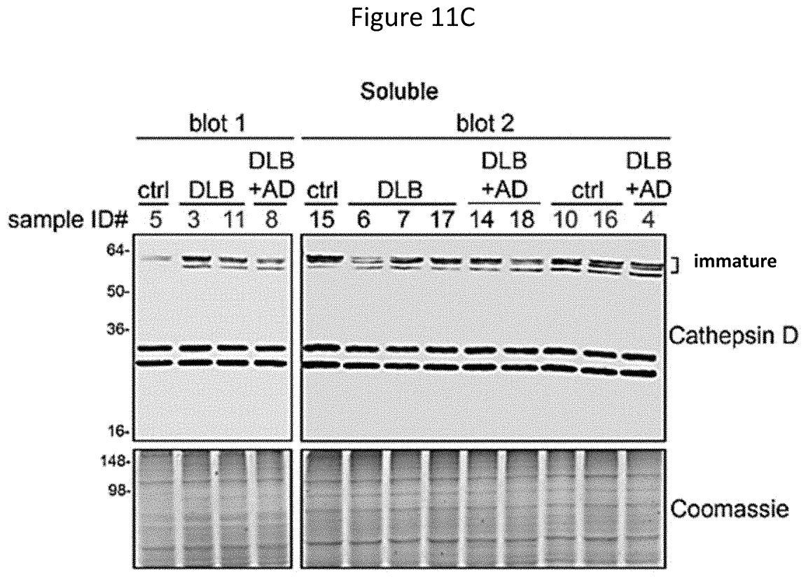

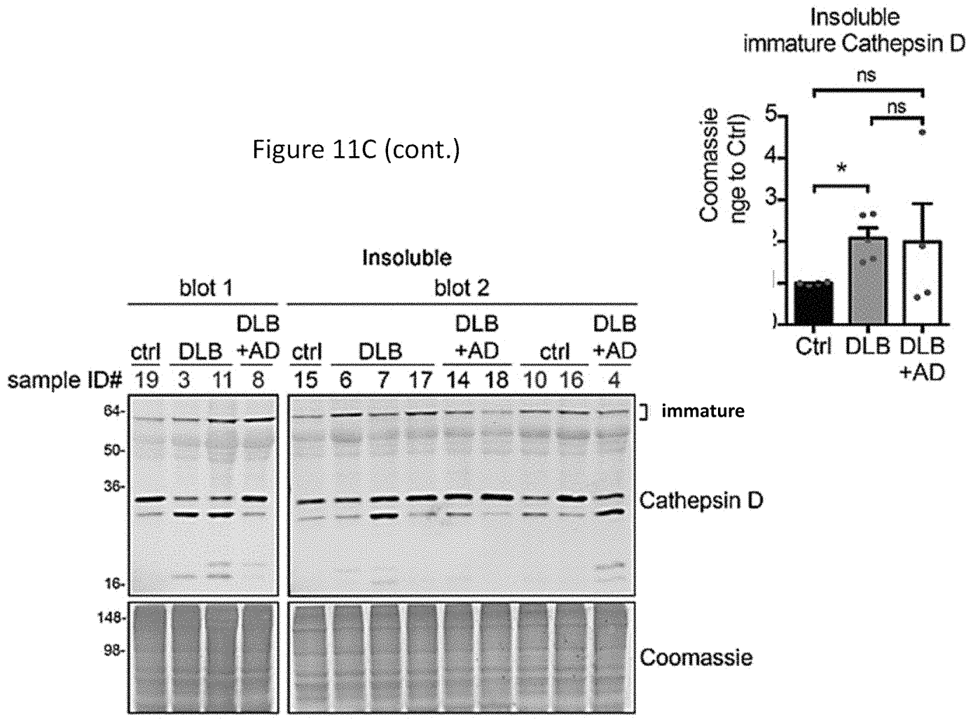

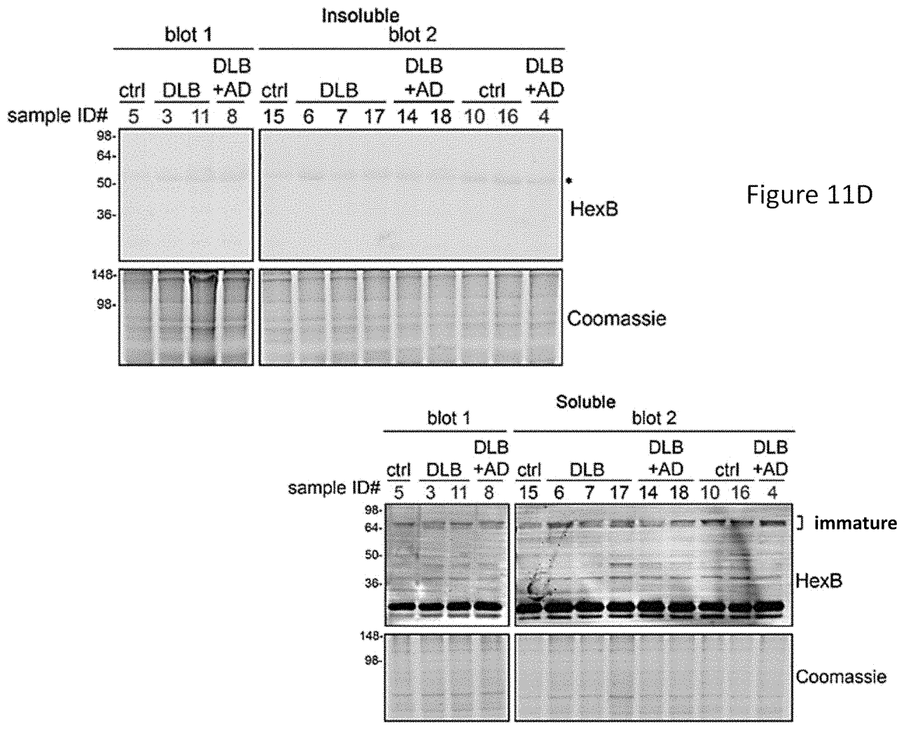

GCase insolubility may result from general perturbations in ER-Golgi trafficking, or may occur specifically through α-syn accumulation. To test these possibilities, we treated wild-type or isogenic control neurons with thapsigargin (Tg) to induce ER stress and perturb ER-Golgi trafficking. Although Tg induced ER stress as shown by increased GRP78 expression, we found no evidence of GCase accumulation in the insoluble fractions (FIG. 11A). Additionally, we assessed GCase aggregation in the brains of LIMP2 knock-out mice, since LIMP2 is required for GCase trafficking between the ER and Golgi (Reczek et al., 2007; Rothaug et al., 2014). While the post-ER forms of GCase were depleted as expected, GCase did not accumulate but was instead depleted in LIMP2−/− mice (FIG. 11B). This is consistent with previous studies showing that LIMP2 depletion reduces GCase levels through aberrant secretion. Collectively, our studies indicate that GCase accumulates into insoluble species selectively upon α-syn-induced inhibition of ER-Golgi trafficking, but not upon general ER stress induction or LIMP2 knock out.

To determine if GCase misfolds and aggregates in vivo, we compared the levels of GCase in 1% sarkosyl-insoluble fractions from brains of patients with either Dementia with Lewy bodies (DLB), or DLB with co-existing Alzheimer's disease (AD) pathology. In age-matched healthy control brains, we detected mild amounts of insoluble GCase that migrated at 55 kDa likely representing the non-glycosylated immature protein, as well as insoluble GCase fragments that migrated between 42 and 48 kDa (FIG. 2C). Even though we observed some variability between control brains, comparison with age and post-mortem interval (PMI)-matched synucleinopathy brain showed a 1.8-fold elevation of insoluble GCase in DLB brain, and a more dramatic increase of nearly 4-fold in DLB+AD brain (FIG. 2C, Table 1). To determine if aggregated GCase occurs in the ER in vivo, we isolated microsomes from idiopathic PD brain and performed a sequential extraction. We found a significant elevation of insoluble GCase in PD brain compared to controls (FIG. 2D, Table 3). We next assessed the specificity of hydrolase aggregation by analyzing the solubility of two additional hydrolases, including cathepsin D and hexosaminidase B. We found that insoluble immature forms of cathepsin D accumulated in DLB brain, but hexosaminidase B was only found in the soluble fraction with no changes in the total levels observed between control and disease (FIG. 11C, D, Table 2). Together, these data indicate that α-syn-induced perturbations in hydrolase maturation induce the accumulation of aggregated, insoluble hydrolases in the ER.

ER Fragmentation in SNCA-3X DA Neurons that Accumulate Immature Wild-Type GCase.

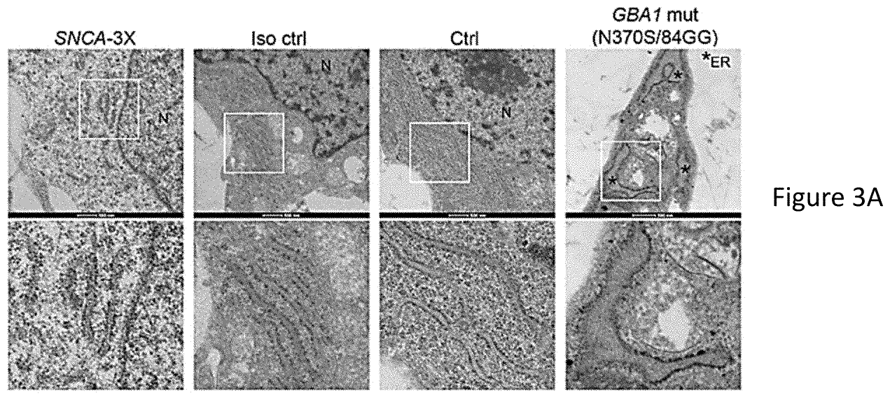

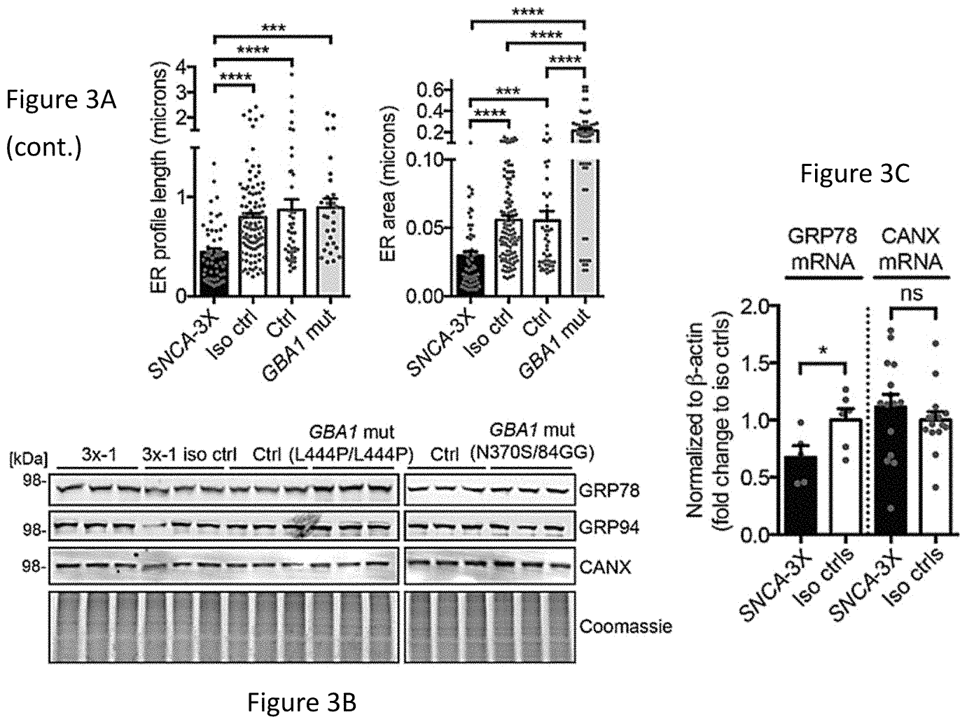

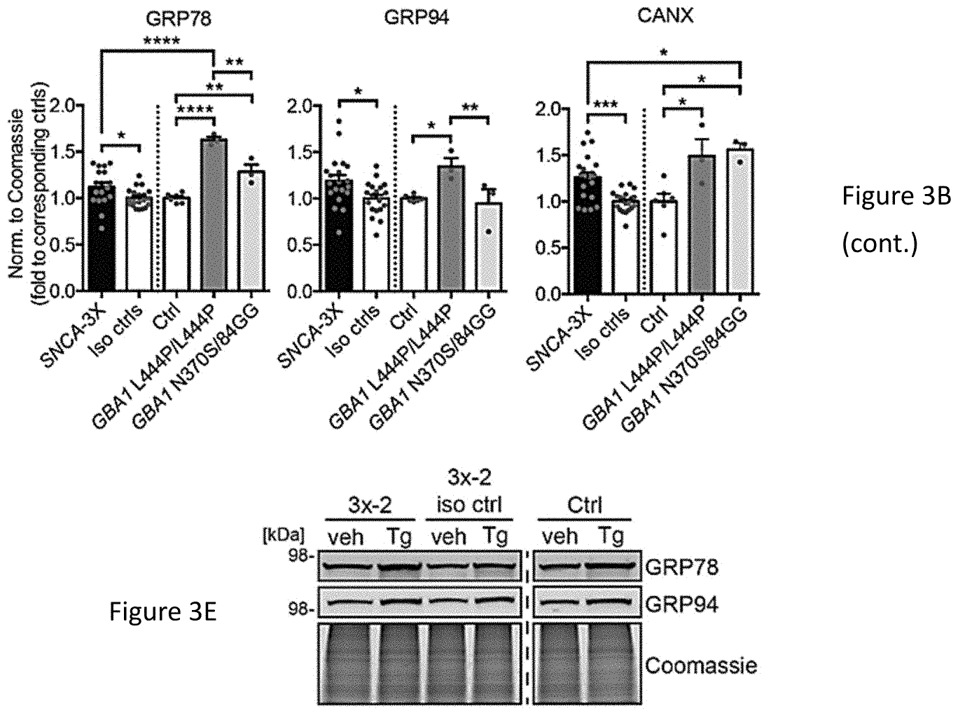

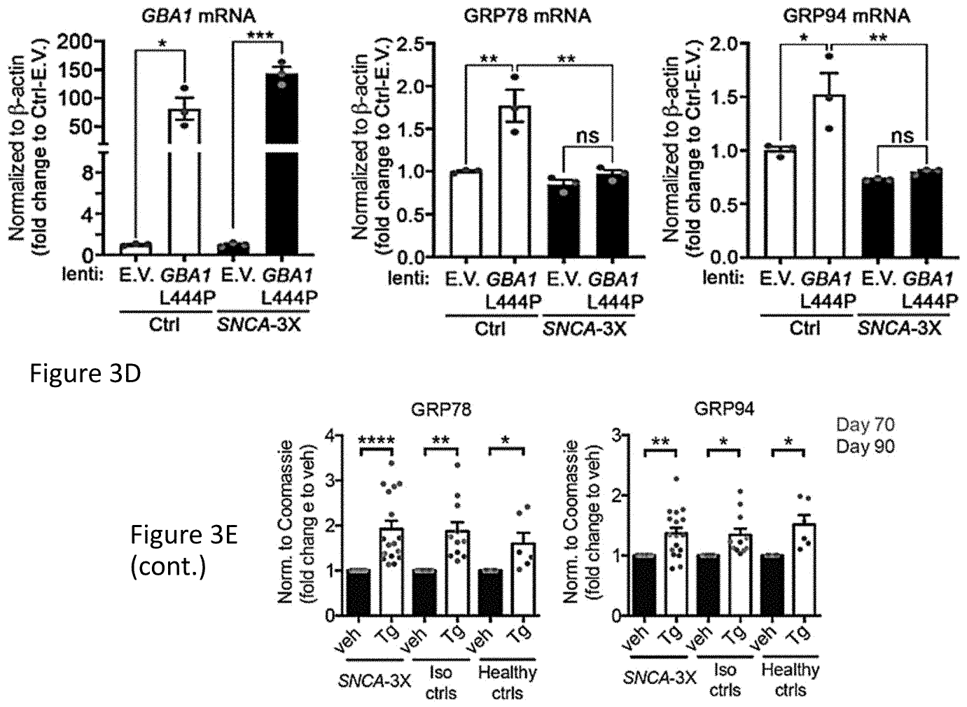

We hypothesized that reduced GCase maturation and accumulation of immature, aggregated GCase in the ER would induce ER stress. When misfolded proteins accumulate in the ER, the UPR normally acts to increase protein folding capacity by expansion of the ER compartment and upregulation of ER chaperones to accommodate the added protein load (Fujiwara et al., 1988; Schuck et al., 2009; Walter and Ron, 2011). To assess ER stress induction in SNCA-3X DA neurons, we first examined the ER morphology by electron microscopy. Despite the accumulation of immature GCase, SNCA-3X DA neurons did not exhibit ER expansion but instead showed a decrease in total ER area relative to isogenic controls (FIG. 3A), with shorter, fragmented ER tubules (FIG. 3A). In contrast, Gaucher's disease (GD) neurons that express and retain mutant GCase (GBA1 N370S/84GG) in the ER demonstrated a severely dilated ER, consistent with an activation of the UPR as previously noted (Ron and Horowitz, 2005) (FIG. 3A). This was confirmed by quantifying the area of each clearly identifiable ER segment in neurons. These data indicate that the ER fails to accommodate the accumulation of misfolded, aggregated GCase by ER expansion, suggesting that SNCA-3X DA neurons may lack the ability to initiate a UPR response.

SNCA-3X DA Neurons Exhibit Mild Elevation of ER Stress-Related Chaperones in the Absence of UPR Activation.

We next examined the levels of ER chaperones known to be involved in GCase folding or upregulated during ER stress including GRP78, GRP94, and calnexin (CANX) by western blot (Kozutsumi et al., 1988; Tan et al., 2014). GRP78 and GRP94 preferentially bind to misfolded or aggregated proteins with exposed hydrophobic patches (Marquardt and Helenius, 1992; Melnick et al., 1994), while CANX normally binds to monoglucosylated N-glycan branches of non-aggregated folding intermediates (Ou et al., 1993) and retains them in the ER until properly folded (Rajagopalan et al., 1994). Compared to isogenic control lines, we observed mild elevations in all three chaperones that ranged from 10-25% (FIG. 3B). In comparison, GD-derived DA neurons carrying either the N370S/84GG or L444P/L444P mutation in GCase showed a dramatic increase (˜25-60%) in GRP78 and CANX relative to the healthy control, while GRP94 levels were elevated by ˜25% only in the GBA1 L444P/L444P mutant (FIG. 3B). The increased levels of GRP78, GRP94, and CANX in GD neurons compared to SNCA-3X DA neurons are likely due to the destabilizing effect of the GCase mutations and are consistent with previous findings (Ron and Horowitz, 2005).

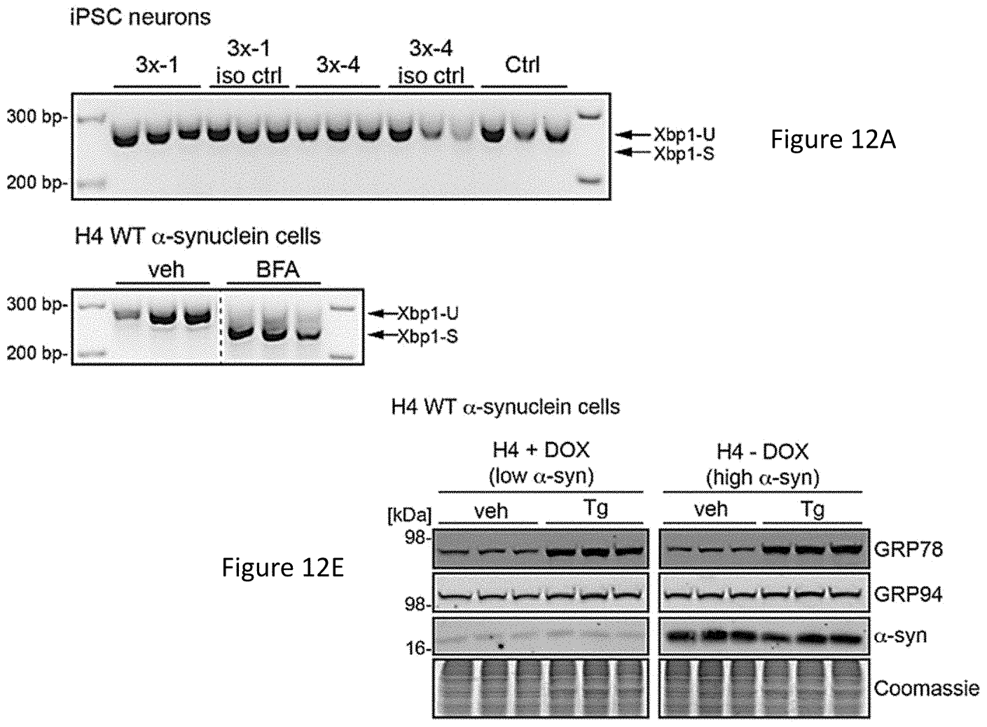

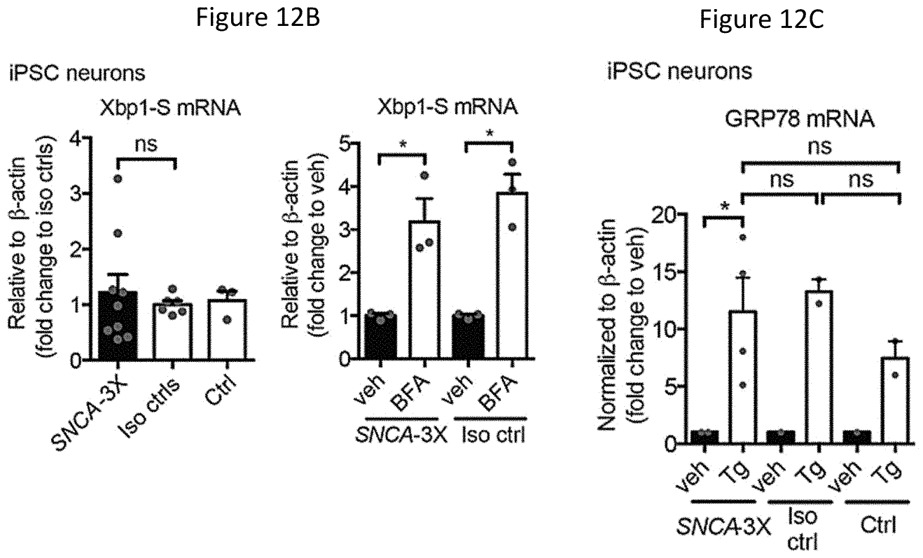

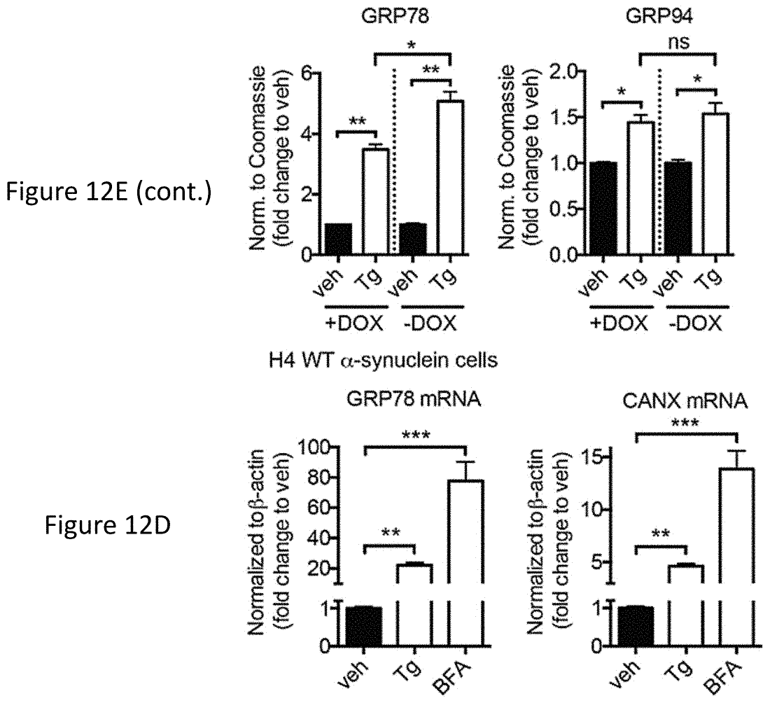

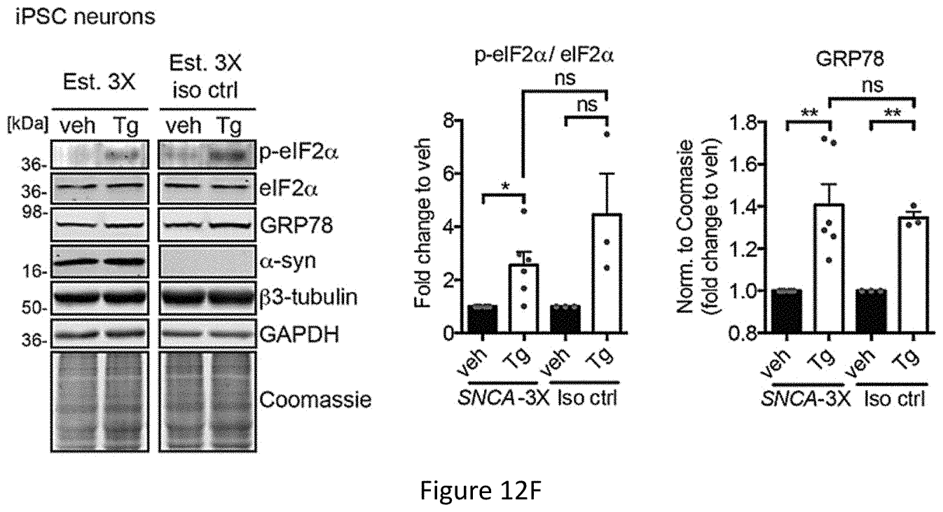

The dramatic ER fragmentation phenotype and accumulation of aggregated immature GCase prompted us to examine UPR signaling pathways in SNCA-3X DA neurons in more detail. During UPR, activation of the IRE1 pathway increases splicing of XBP1 mRNA, resulting in a functionally active transcription factor XBP1-S that upregulates expression of ER chaperones and ERAD machinery (Calfon et al., 2002; Yoshida et al., 2001). Therefore, we measured XBP1-S and the expression of downstream transcriptional targets in SNCA-3X DA neurons. Using two independent assays, we surprisingly did not observe an increase of XBP1-S in SNCA-3X DA neurons compared to controls (FIG. 12A; FIG. 12B, left). Moreover, the mRNA of GRP78 was reduced and CANX was not changed in SNCA-3X DA neurons, indicating the absence of UPR-induced transcriptional response (FIG. 3C). To determine if the UPR could be induced in SNCA-3X DA neurons by misfolded GCase, we expressed the L444P mutant GCase by lentiviral infection. We achieved a dramatic expression of L444P GCase in both healthy control and SNCA-3x neurons (FIG. 3D, left). However, while L444P-infected control neurons responded by robust upregulation of GRP78 and GRP94 mRNA, SNCA-3X DA neurons showed no response (FIG. 3D, middle and right). This indicates that PD patient neurons fail to sense or transmit misfolded protein stress signals in the ER to initiate the UPR.

We next determined if UPR could be activated in SNCA-3x DA neurons by established chemical ER stressors that induce the UPR through pleotropic effects. Tg and brefeldin A (BFA) activate the UPR through either disturbing calcium homeostasis, or directly block ER-Golgi trafficking machinery, respectively (Booth and Koch, 1989; Helms and Rothman, 1992; Price et al., 1992). We found that Tg and BFA successfully induced a dramatic upregulation of XBP1-S (FIG. 12B, right), and significantly increased mRNA/protein levels of ER chaperones including GRP78, GRP94, and CANX in SNCA-3X DA neurons and α-syn overexpressing cell lines (FIG. 3E; FIG. 12C-F). In ER stress-induced SNCA-3X and control DA neurons, XBP1-S mRNA and ER chaperone mRNA/protein levels were increased to a similar degree (FIG. 3E; FIG. 12B, C, F). This indicates that while the UPR is capable of activation by chemical stressors that broadly activate the UPR, PD neurons are specifically deficient in handling misfolded proteins in the ER.

We next assessed the PERK pathway of the UPR in SNCA-3X DA neurons by measuring eIF2α, a eukaryotic initiation factor which upon phosphorylation by PERK leads to global translational attenuation (Harding et al., 1999). We did not observe baseline elevation of phospho-eIF2α in patient neurons (FIG. 12F), indicating that the PERK pathway is not active in PD neurons. However, treatment with Tg increased phospho-eIF2α to a similar degree in both isogenic controls and SNCA-3X DA neurons (FIG. 12F). Collectively these results are consistent with the notion that PD neurons fail to recognize or transduce perturbations in ER proteostasis induced by α-syn, despite dramatic ER fragmentation and accumulation of aggregated GCase.

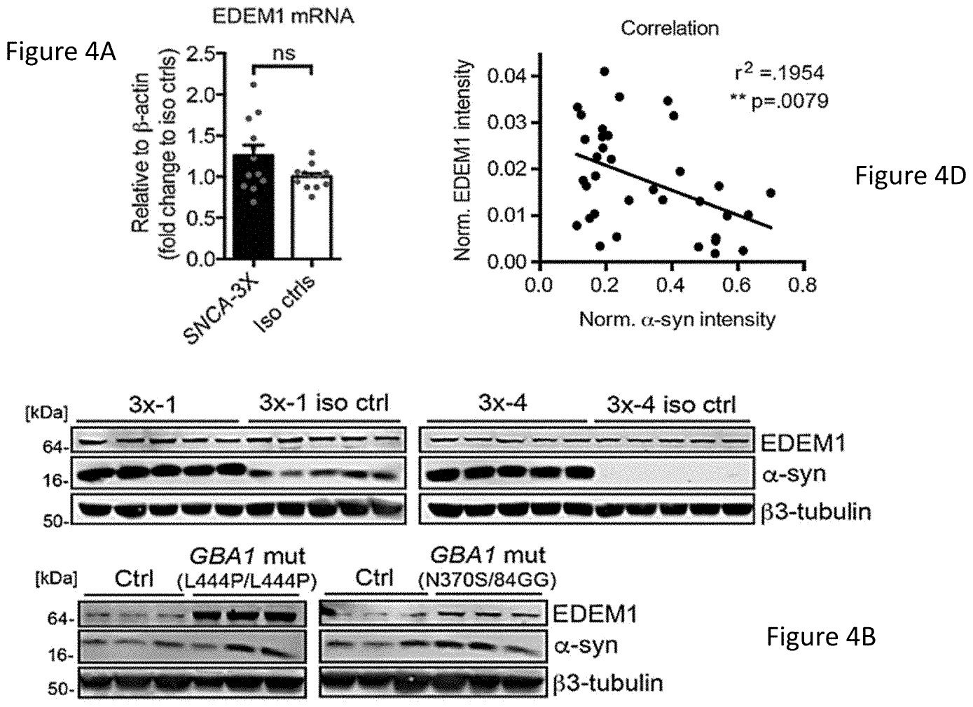

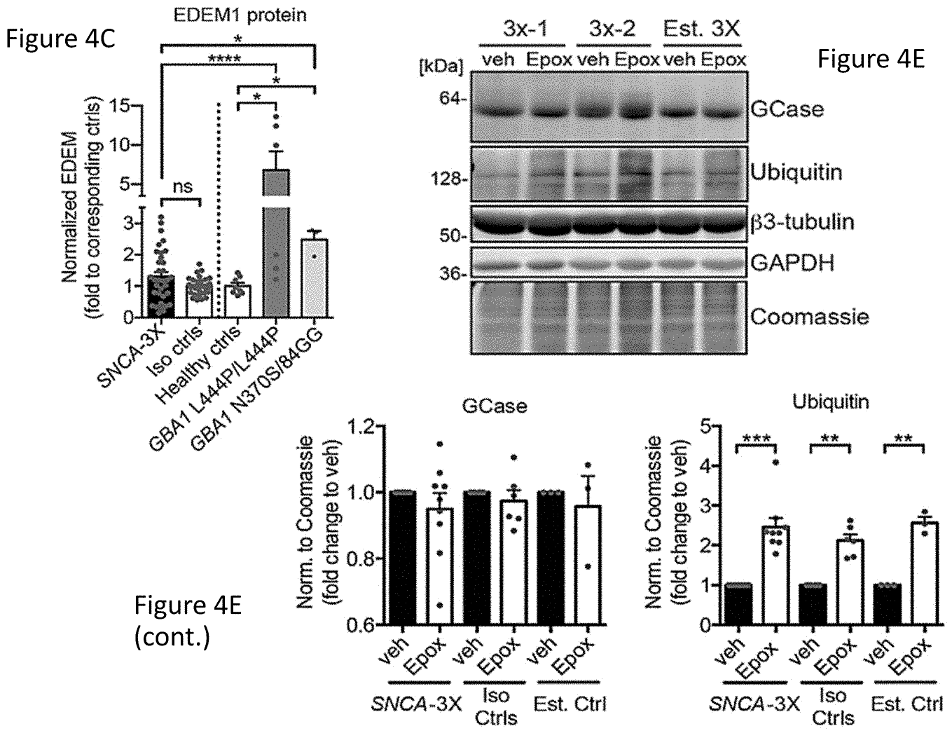

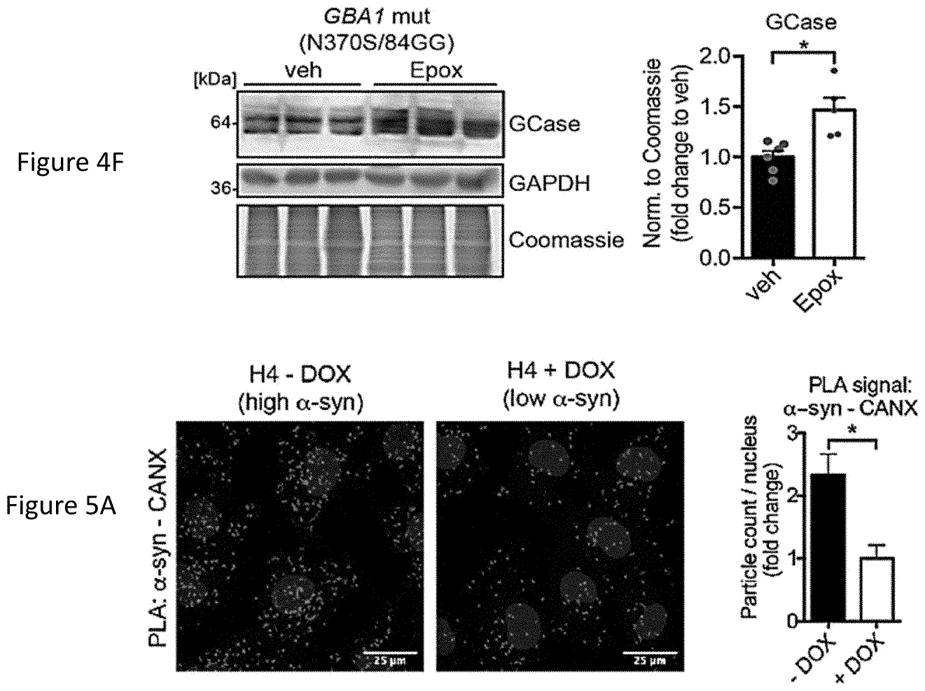

Misfolded proteins that are retained and accumulate in the ER are normally recognized by quality control machinery and eliminated by ERAD. For retained glycoproteins including lysosomal hydrolases, this process is mediated by EDEM1 (ER degradation-enhancing α-mannosidase-like protein 1). EDEM1 is a lectin-containing adapter protein that removes misfolded glycoproteins from the CANX folding cycle and delivers them to the cytosol for degradation through the ubiquitin-proteosome system (Smith et al., 2011). EDEM1 expression is upregulated by ER stress through the IRE1 pathway of the UPR, helping to rebalance ER proteostasis (Lee et al., 2003). To determine if ERAD is altered in patient-derived SNCA-3X DA neurons, we measured the expression levels of EDEM1. Compared to the isogenic controls, we found no change in EDEM1 mRNA or protein levels (FIG. 4A-C). Since variability was observed in the levels of EDEM1 between culture samples of SNCA-3X lines, we correlated EDEM1 and α-syn protein levels in patient neurons and found a significant negative relationship (FIG. 4D). This negative correlation suggests that samples with abundant α-syn pathology have reduced EDEM1 levels and therefore compromised ability to remove retained proteins from the CANX folding cycle. Consistent with a lack of ERAD enhancement, we found no change in the levels of immature wild-type GCase upon proteosomal inhibition of SNCA-3X DA neurons indicating the protein is not cleared through ERAD (FIG. 4E). In contrast, GD neurons showed a dramatic upregulation of EDEM1 compared to both healthy controls and SNCA-3X lines (FIG. 4B, C), and significant elevation of GCase protein upon proteosomal inhibition (FIG. 4F). Overall, these data indicate that despite retaining immature GCase in the ER, the EDEM1/ERAD pathway is not altered in SNCA-3X DA neurons. In contrast, ERAD is elevated in GD patient DA neurons and leads to elimination of mutant GCase, consistent with previous findings in other cell models (Ron and Horowitz, 2005).

α-Synuclein Accumulates at the ER and is Proximal to ER Chaperones in SNCA-3X Da Neurons.

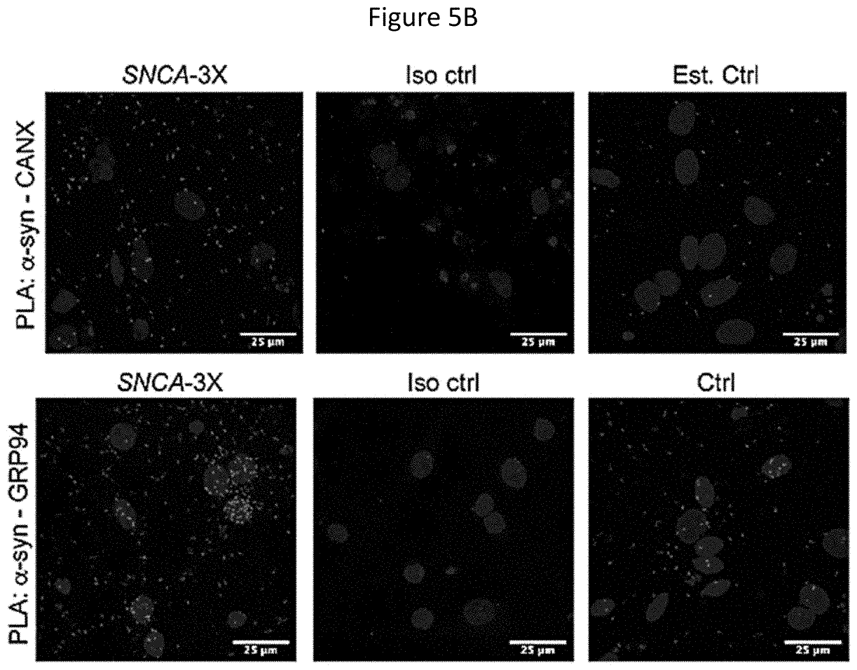

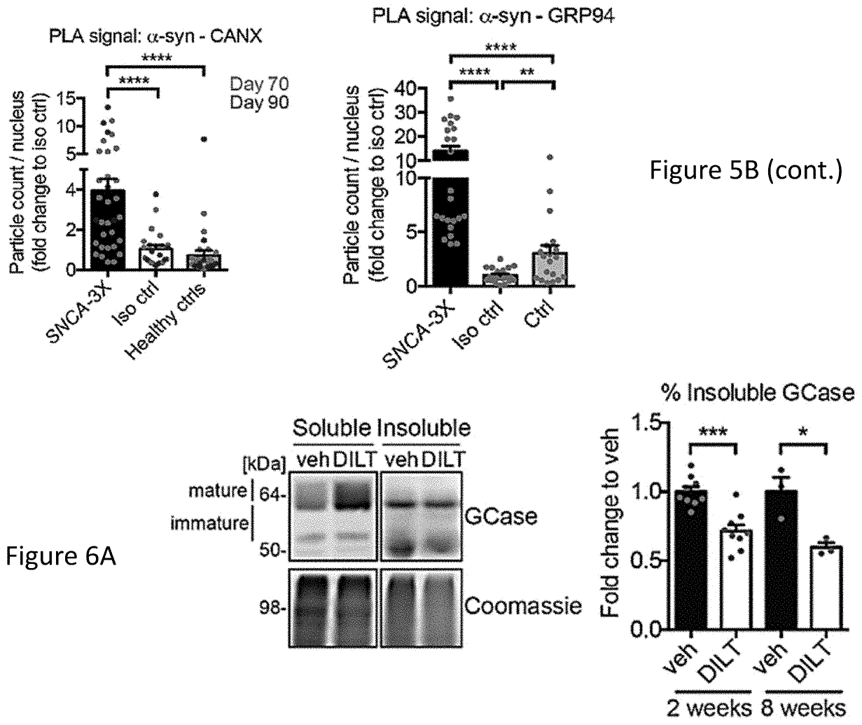

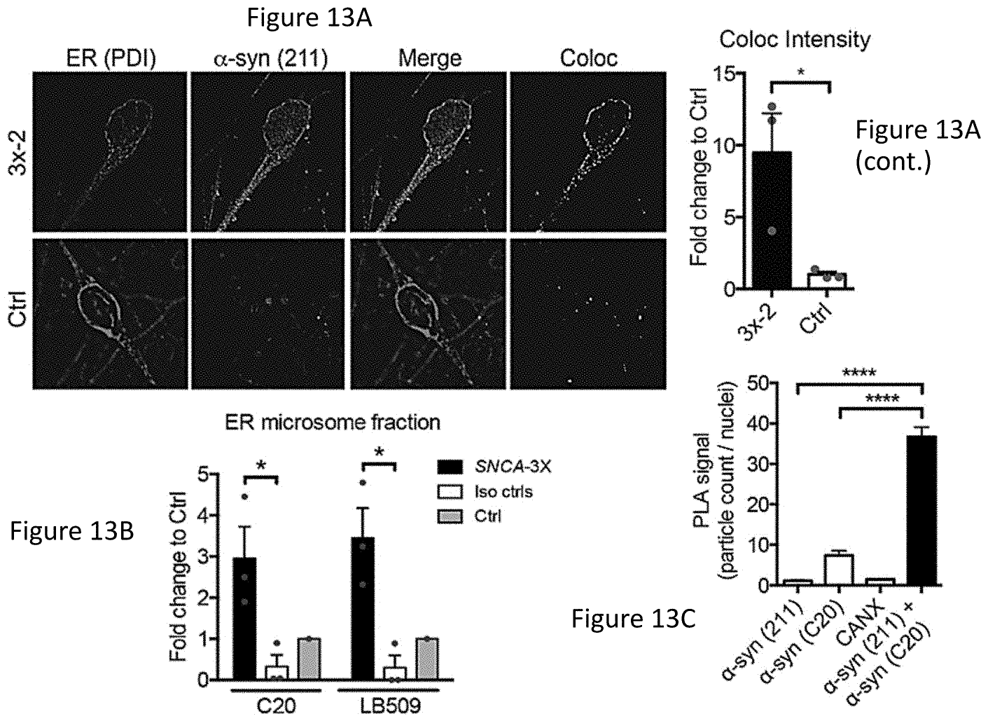

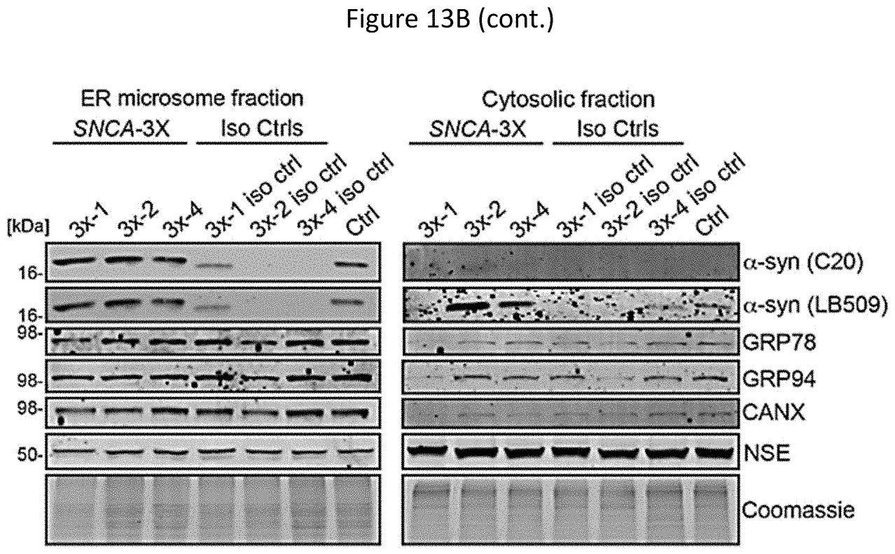

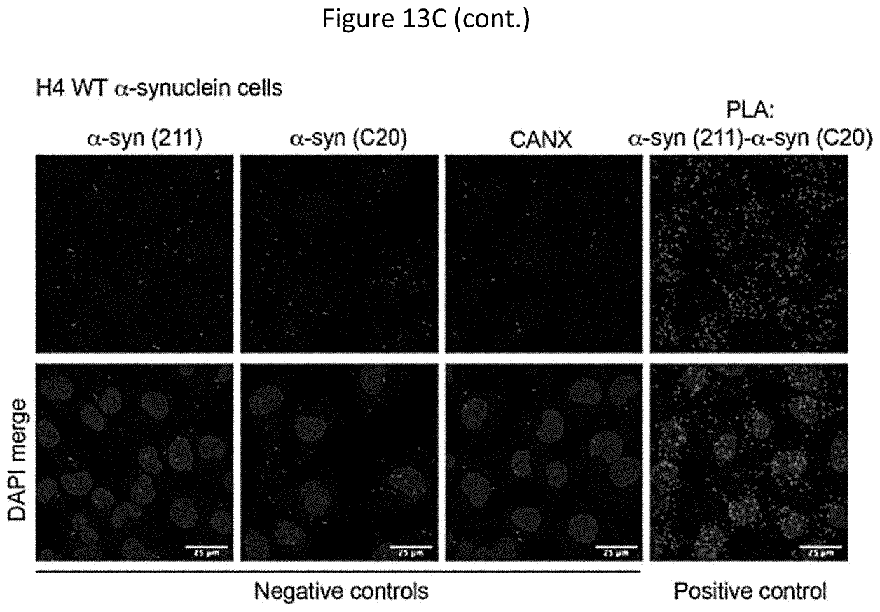

Although α-syn is known to be a synaptic protein under physiological conditions, our immunofluorescence analysis indicated its accumulation at the cell body in SNCA-3X DA neurons (FIG. 10A, B), consistent with previously characterized PD patient lines (Cuddy et al., 2019; Mazzulli et al., 2016a). Further, studies using transgenic α-syn overexpression models indicated that pathological α-syn can abnormally localize to the ER compartment (Bellucci et al., 2011; Colla et al., 2012a; Colla et al., 2012b; Colla et al., 2018; Guardia-Laguarta et al., 2014; Masliah et al., 2000; Paillusson et al., 2017). To determine if a-syn associates with the ER in SNCA-3×DA neurons, we used super-resolution imaging to examine the colocalization of a-syn with the established ER marker PDI, as well as enrichment of ER microsomes in neurons. We found that α-syn aberrantly associates with the ER in SNCA-3X patient neurons and is enriched within microsomal fractions (FIG. 13A, B). To examine the potential mechanism by which α-syn could disrupt ER proteostasis, we analyzed its proximity to ER chaperones including CANX and GRP94 by in-situ proximity ligation assays (PLA). PLA negative controls indicated a minimal background signal, suggesting specificity of the antibodies under these conditions (FIG. 13C). Analysis in α-syn overexpressing cell lines and SNCA-3X patient DA neurons showed that α-syn localizes to and potentially interacts with both CANX and GRP94 (FIG. 5). Together, these results suggest that α-syn may disrupt ER proteostasis and GCase trafficking through aberrant association and sequestration of ER chaperones.

Synergistic Enhancement of ER Proteostasis and Trafficking Rescues Lysosomal Function and Reduces α-Syn in SNCA-3X DA Neurons.

The aberrant association of α-syn with ER chaperones and aggregation of immature GCase suggested that ER chaperone function may be overwhelmed in SNCA-3X patient neurons. Furthermore, previous studies have shown that accumulation of the GCase substrate glucosylceramide, which occurs in both SNCA-3X and A53T α-syn iPSC-midbrain cultures (Cuddy et al., 2019; Mazzulli et al., 2016a), reduces ER calcium in vitro and in vivo (Korkotian et al., 1999; Liou et al., 2016; Pelled et al., 2005). This, in turn, compromises the function of calcium-dependent chaperones such as CANX. Disrupted calcium homeostasis has also been independently observed in several PD models (Apicco et al., 2021; Caraveo et al., 2014), and is consistent with fragmentation of the ER in SNCA-3X DA neurons (FIG. 3A) (Ha et al., 2020; Koch et al., 1988). Therefore, we next determined if enhancing ER chaperone function by increasing ER calcium can rescue wild-type GCase proteostasis and its downstream lysosomal activity.

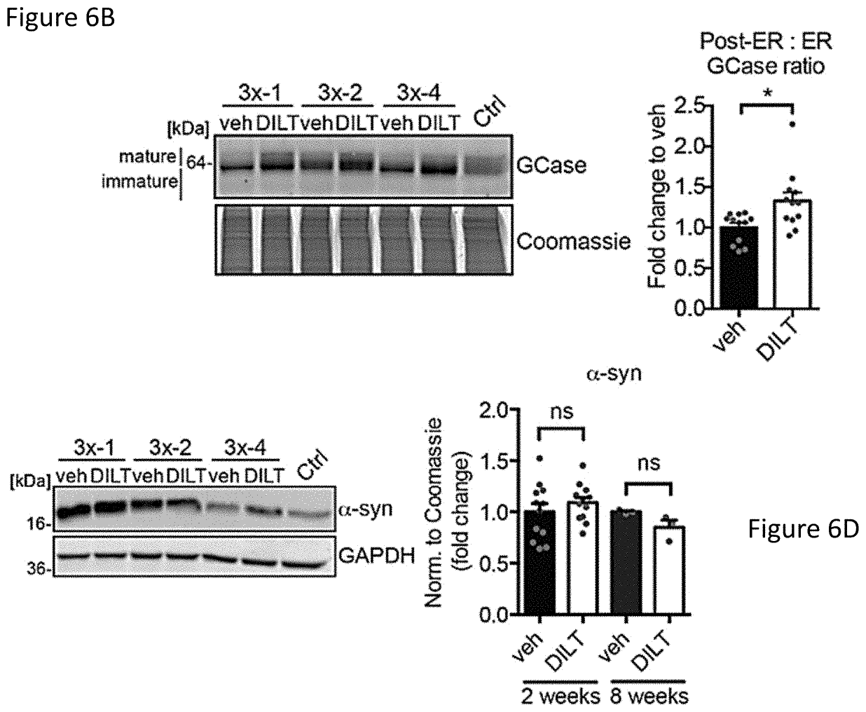

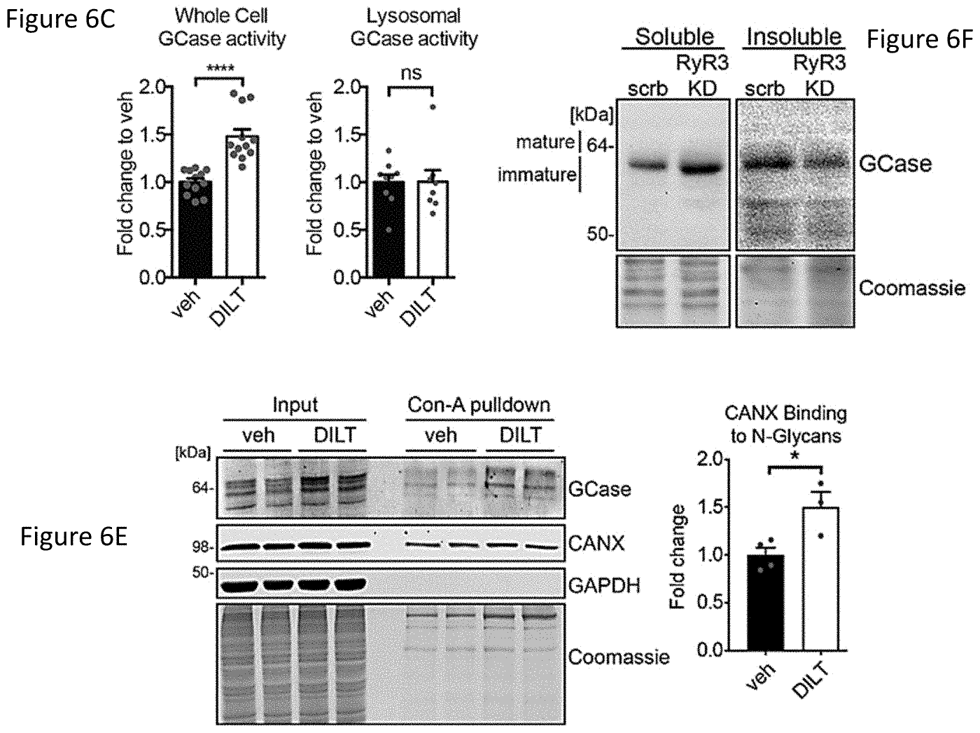

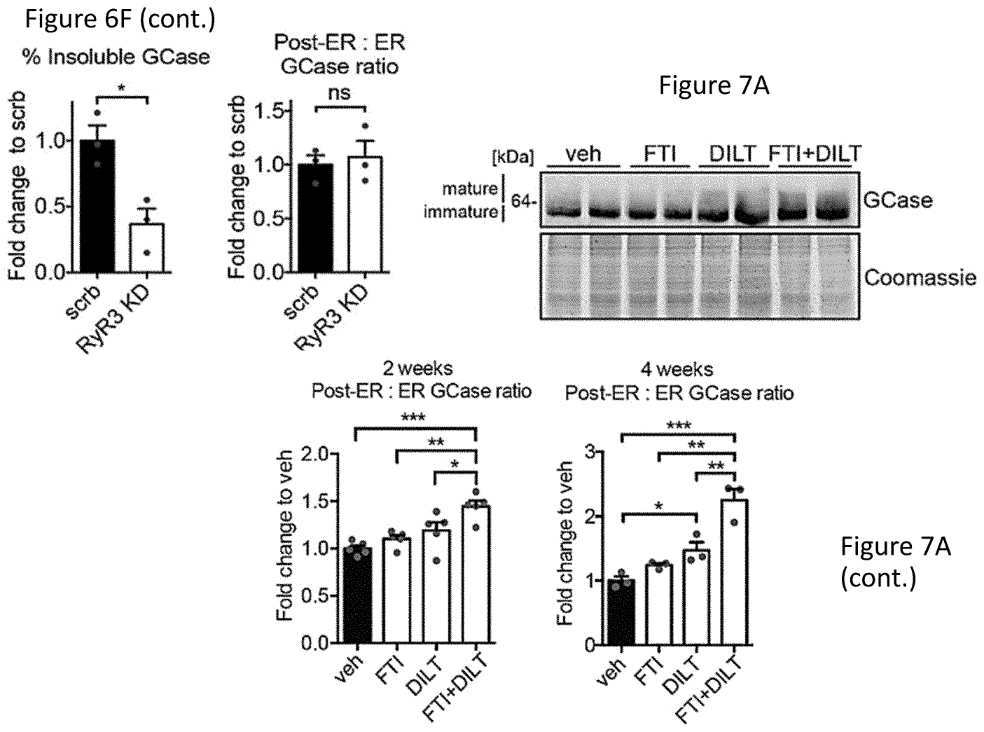

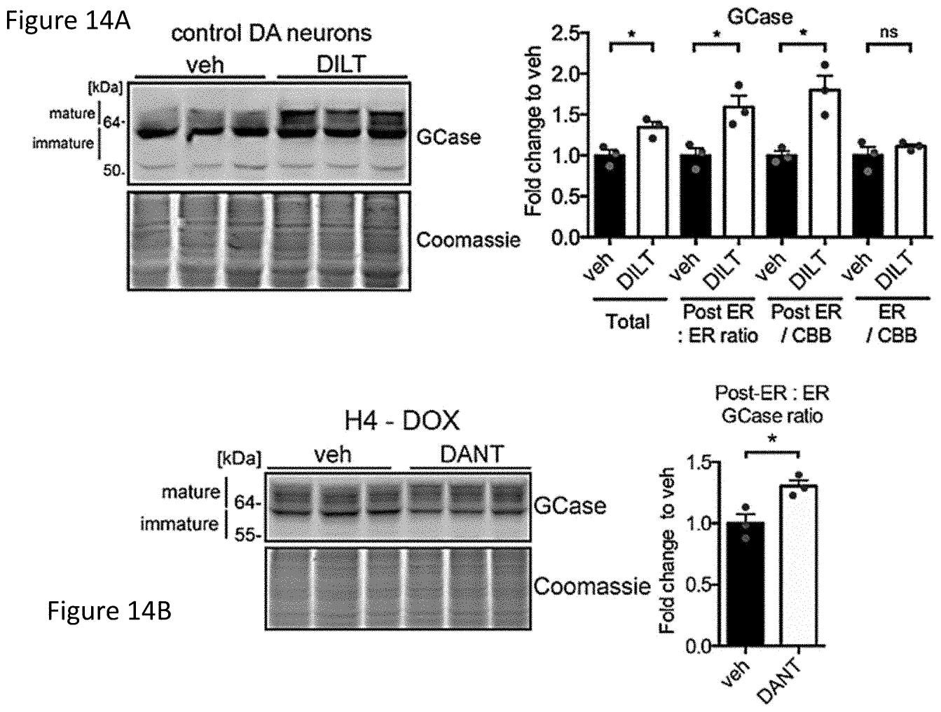

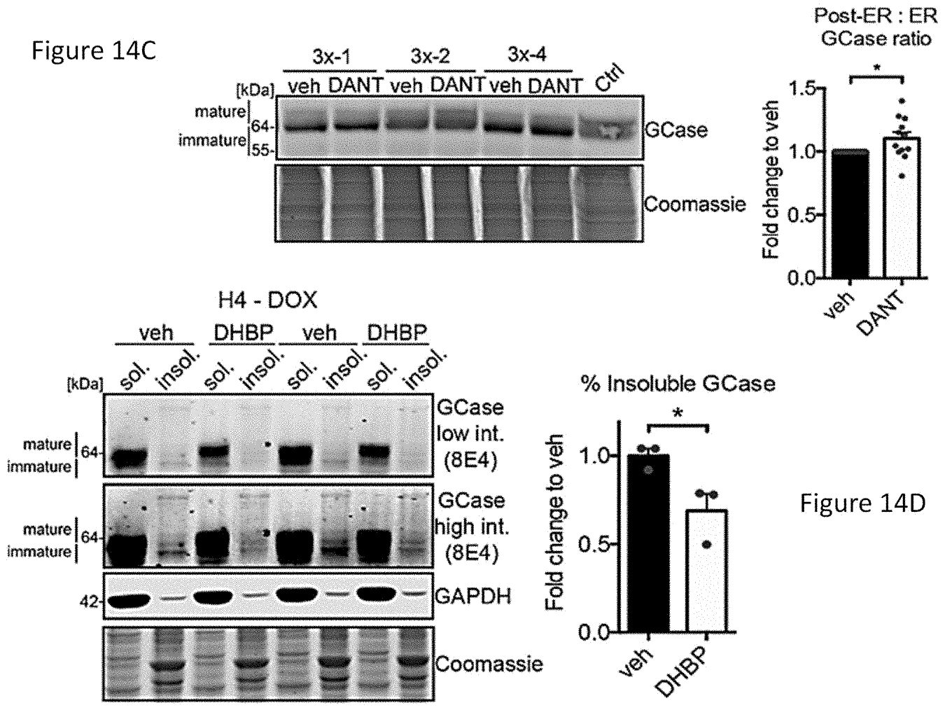

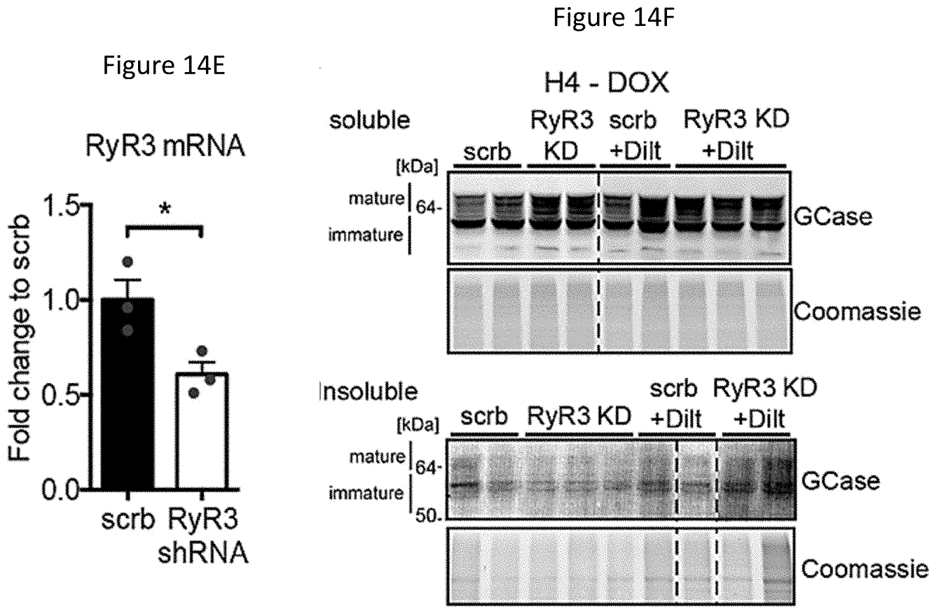

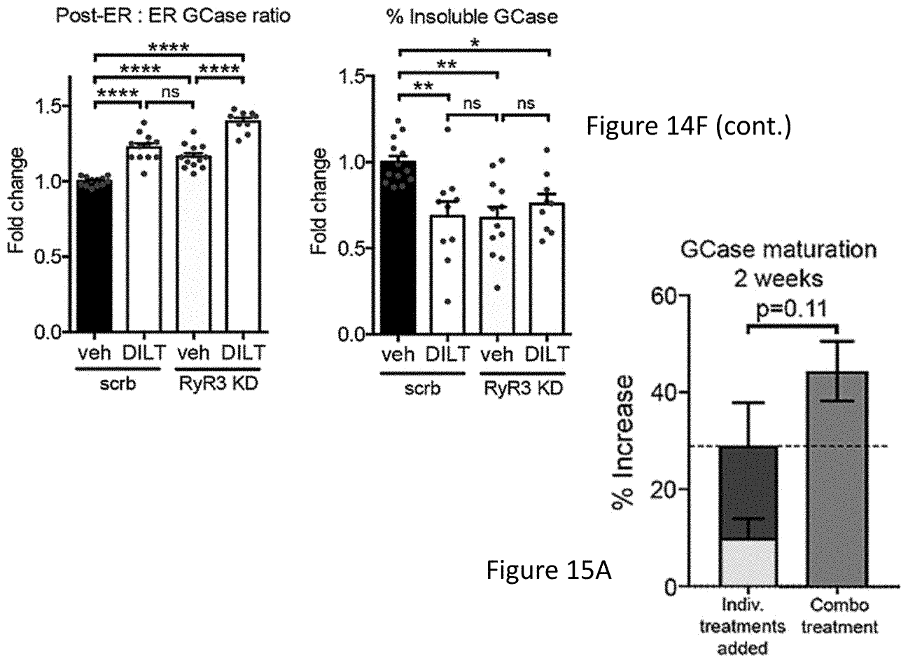

Previous culture studies showed that elevating ER proteostasis by enhancing ER calcium levels can be achieved by blocking ryanodine receptors (RyRs) that mediate calcium efflux from the ER, thereby increasing CANX function (Mu et al., 2008; Ong et al., 2010). We selected the RyR inhibitor diltiazem (DILT), since it is non-toxic and is an FDA-approved drug for the treatment of high blood pressure and angina. DILT treatment is an established method to enhance CANX function and can improve the folding, trafficking and function of mutant GCase in GD patient fibroblasts (Ong et al., 2010; Sun et al., 2009). Initial dose response studies of DILT indicated that iPSC-midbrain cultures could tolerate a maximum dose of 25 mM without toxicity (not shown). Treatment of SNCA-3X patient DA neurons with 25 mM DILT abrogated the build-up of insoluble GCase while concomitantly elevating soluble forms of the enzyme starting at 2 weeks and continuing to 8 weeks of treatment (FIG. 6A). Although DILT mainly increased immature forms of GCase, we observed a slight elevation in post-ER forms, indicating a mild improvement in maturation (FIG. 6B). DILT also elevated GCase protein levels and maturation in healthy control DA neurons, suggesting that enhancement of the folding pathway can be achieved in neurons without α-syn pathology or ER perturbations (FIG. 14A). DILT enhanced properly folded, functional GCase, as demonstrated by increased GCase activity in whole cell lysates that include both ER and post-ER forms (FIG. 6C, left). Despite this, the in situ assay that measures GCase activity within lysosomes of living neurons indicated no change in activity (FIG. 6C, right). Consistently, we found that α-syn levels were also not changed, indicating that DILT treatment alone could not sufficiently induce lysosomal α-syn degradation, even after a prolonged incubation of 8 weeks (FIG. 6D). To validate that DILT could enhance chaperone function, we measured the binding activity of CANX to N-glycosylated proteins using the lectin concanavalin A (Con-A). Compared to vehicle treatment, CANX binding activity was significantly increased in DILT treated cells, demonstrated by increased CANX and GCase pulldown with Con-A (FIG. 6E). Taken together, these results suggest that enhancing ER proteostasis with DILT can promote functional, soluble forms of GCase, but cannot improve lysosomal function in a sufficient manner to reduce α-syn.

To confirm that enhancing ER proteostasis and wild-type GCase can be improved by RyR inhibition, we treated α-syn overexpressing cell lines and SNCA-3X DA neurons with two additional, distinct and specific RyR inhibitors, dantrolene (DANT) and 1,1′-diheptyl-4,4′-bipyridinium (DHBP) (Fruen et al., 1997; Kang et al., 1994). DANT and DHBP treatment elevated soluble GCase levels in cell models, although not as robustly as DILT (FIG. 14B-D). When higher concentrations or longer incubation periods were attempted, we observed cell toxicity, consistent with previous findings (Ong et al., 2010; Wang et al., 2011). Finally, we directly tested the effect of RyR inhibition on GCase solubility by knocking down RyR with shRNA constructs. We specifically knocked down RyR3 since it is expressed widely throughout the brain (Giannini et al., 1995) and previous studies indicated that RyR3 depletion could rescue the severe mutant (L444P) form of GCase in patient cells (Ong et al., 2010). RT-PCR analysis showed a 50% knock-down (KD) of RyR3 (FIG. 14E), resulting in increased solubility of GCase in both cell lines and SNCA-3X DA neurons (FIG. 6F; FIG. 14F). The rescue effect of RyR3 KD was identical to that of DILT treatment (FIG. 14F), suggesting that the solubilizing activity of DILT can be attributed to inhibition of RyR3 rather than off-target effects. Further, DILT had no effect on GCase solubility in RyR3 KD cells (FIG. 14F), once again indicating that DILT acts through RyR3 receptors on the ER. Analysis of GCase maturation in RyR3 KD cells showed a mild improvement in cell lines similar to DILT treatment, and no change in SNCA-3X DA neurons (FIG. 6F; FIG. 14F). DILT caused a mild elevation of GCase maturation in RyR3 KD cell lines, a result that likely occurred from the inhibition of other RyR isoforms (FIG. 14F). Together, these data show that RyR inhibition with three distinct compounds and shRNA can improve GCase proteostasis in the ER, but has little effect on increasing GCase trafficking in cells that accumulate α-syn.

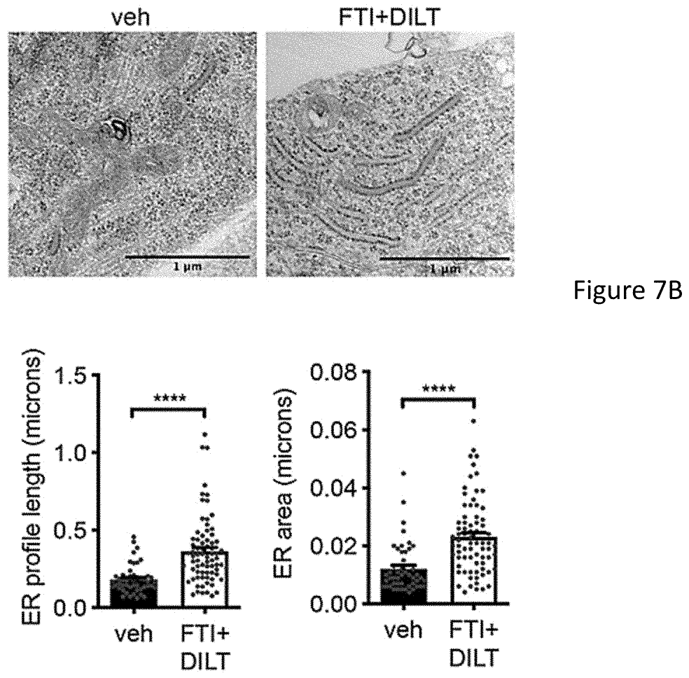

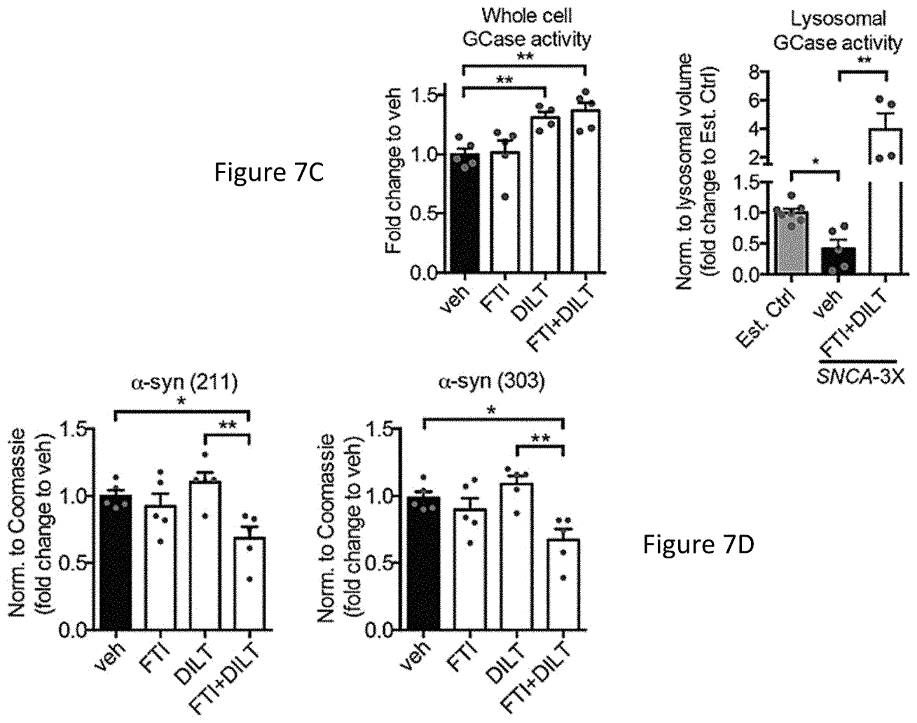

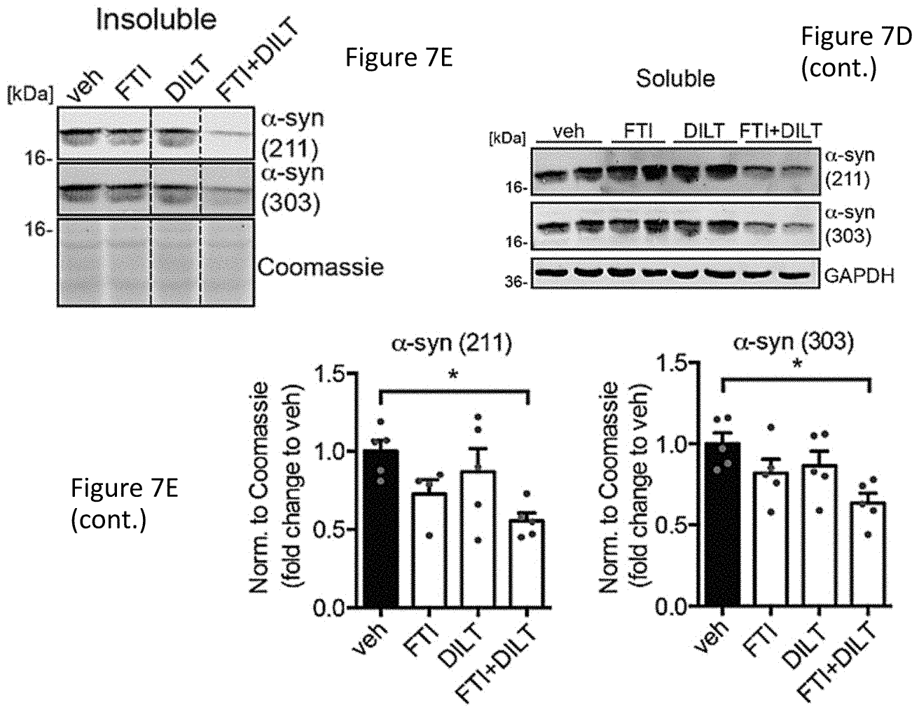

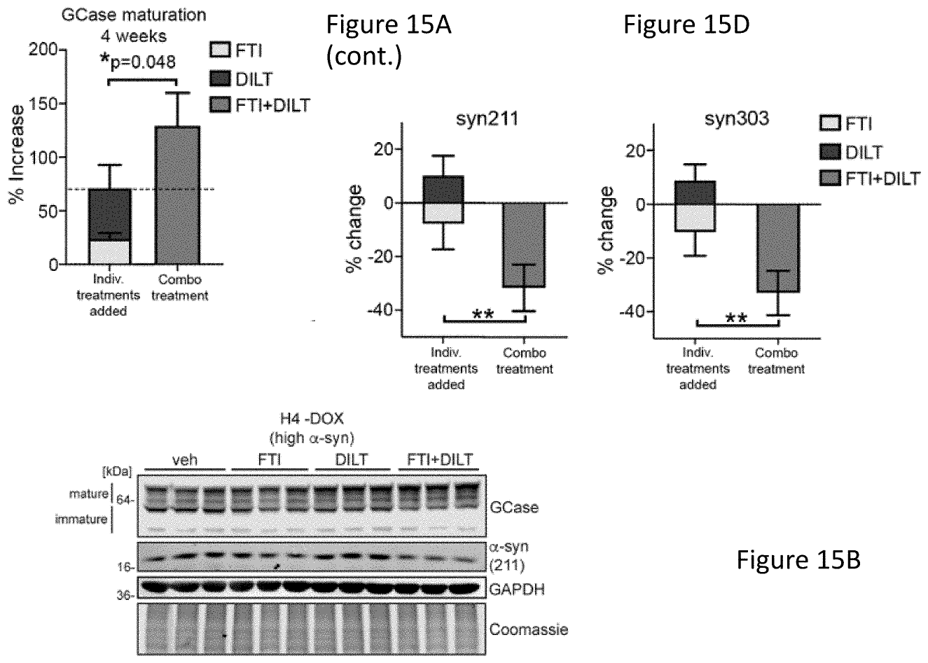

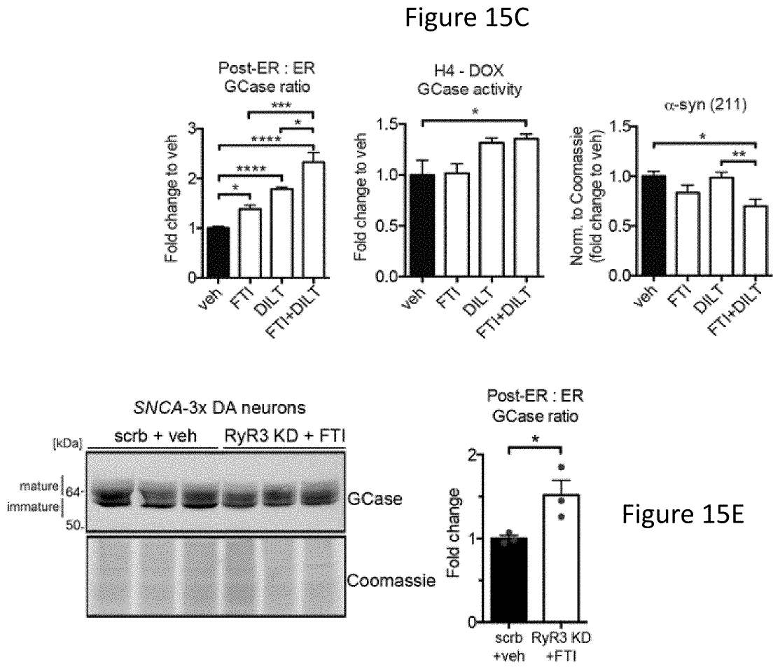

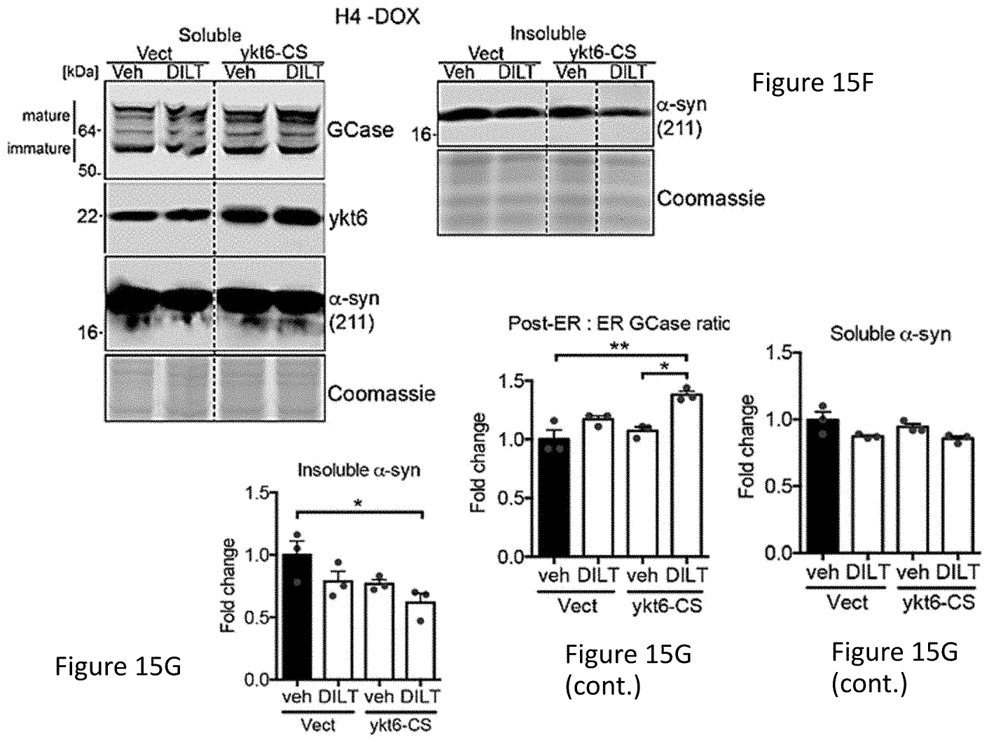

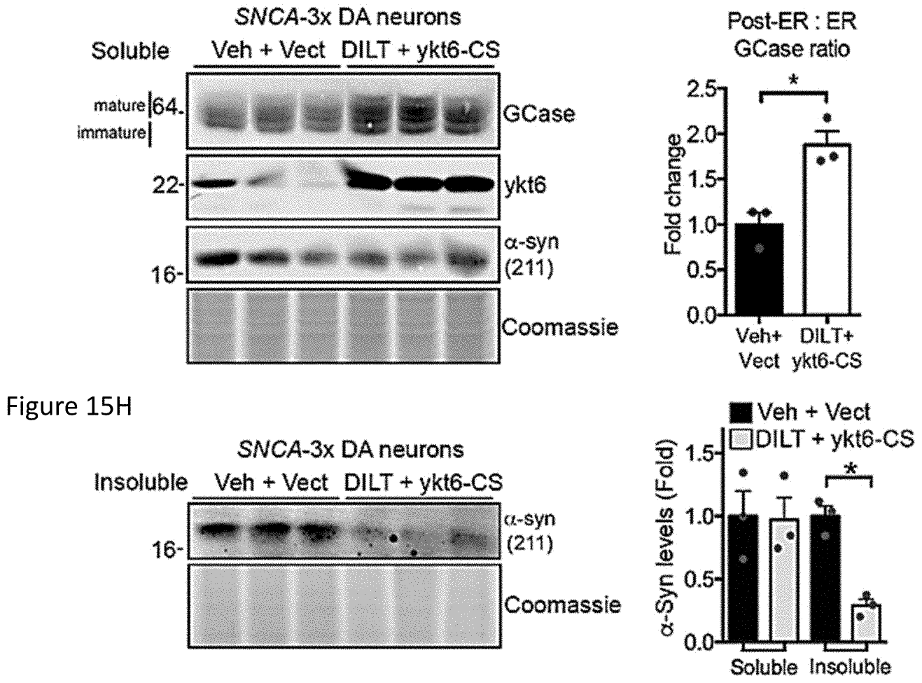

The failure to rescue lysosomal GCase activity by RyR inhibition, despite the elevation of properly folded, soluble ER GCase, suggests that factors downstream of the ER may inhibit hydrolase trafficking. Our previous work showed that α-syn inhibits GCase trafficking by preventing ER-Golgi vesicle fusion through impeding the function of the SNARE protein ykt6 (Cuddy et al., 2019). Further, farnesyltransferase inhibitors (FTIs) can restore ykt6 activity, thereby improving GCase trafficking and lysosomal activity in PD neurons (Cuddy et al., 2019). Therefore, we next determined whether enhancing ER-to-Golgi vesicle fusion, together with ER proteostasis, could cooperate to rescue GCase activity and reduce pathological α-syn levels. We found that treatment with the FTI (LNK-754) and DILT resulted in a significant increase of GCase maturation compared to each compound alone (FIG. 7A, FIG. 15A-C). This effect was not additive but synergistic, since the percent increase caused by FTI+DILT was greater than the sum of the enhancement by each individual compound alone after 4 weeks of treatment (FIG. 15A). This is consistent with the notion that each compound targets a distinct portion of the proteostasis pathway. To determine if improved folding and trafficking of GCase out of the ER could restore ER morphology, EM analysis was performed. We found that FTI+DILT treatment substantially increased ER length and area, consistent with increased GCase solubility and function (FIG. 7B). FTI+DILT treatment also elevated functional, soluble forms of GCase in both whole cell lysates and live-cell in situ lysosomal assays (FIG. 7C). The GCase activity enhancement was sufficient to synergistically reduce both soluble and insoluble α-syn in patient neurons and cell lines (FIG. 7D, E; FIG. 15B-D). Next, we confirmed our findings by genetic manipulations, either by combining RyR3 KD with FTI, or DILT with expression of ykt6-CS that cannot be farnesylated (Cuddy et al., 2019). These combinations could effectively enhance GCase trafficking and reduce α-syn better than either treatment alone (FIG. 15E-H).

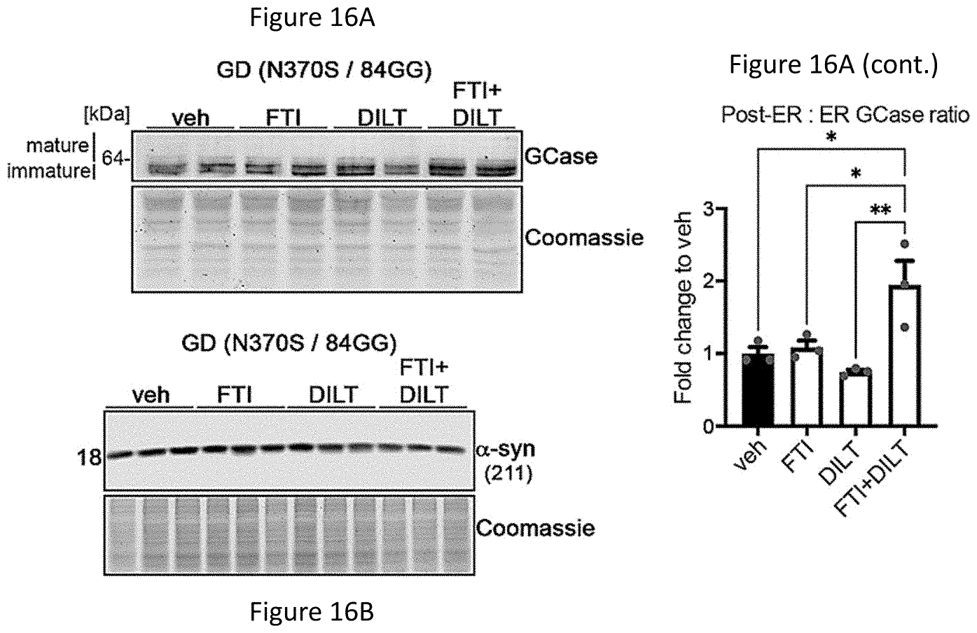

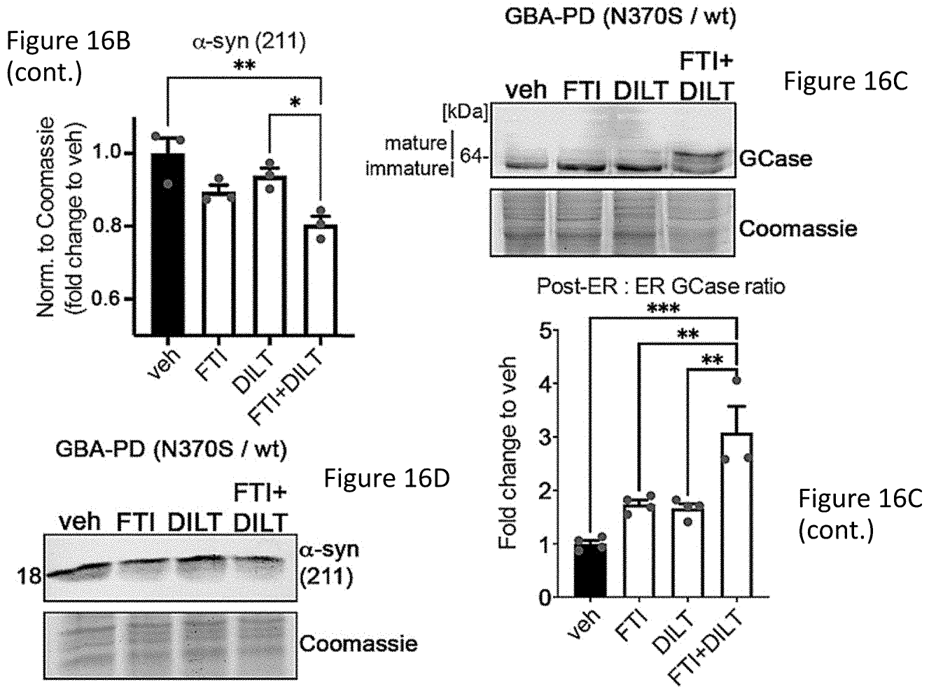

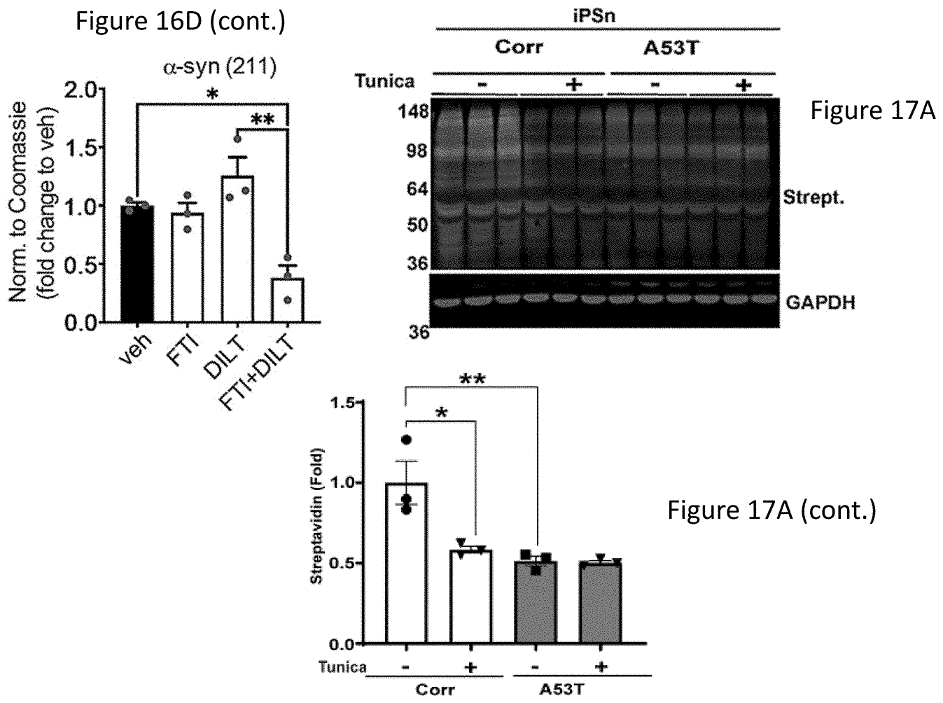

We sought to determine if the combination treatment could improve GCase proteostasis and reduce α-syn levels in patient iPSC neurons that express GBA1 mutations. We treated midbrain neurons from a GD (N370S/84GG) and a PD patient that harbors a GBA1 mutation (N370S/wt). We found that while individual treatments of FTI or DILT had minimal effects, combining FTI and DILT significantly increased GCase maturation and reduced α-syn levels in both lines (FIG. 16). Our data suggests that simultaneous enhancement of two critical proteostasis branches, ER proteostasis and downstream trafficking pathways, provides a more effective rescue of GCase function compared to either treatment alone, resulting in a reduction of pathological α-syn in patient neurons that harbor wild-type as well as mutant GBA1.

### DISCUSSION

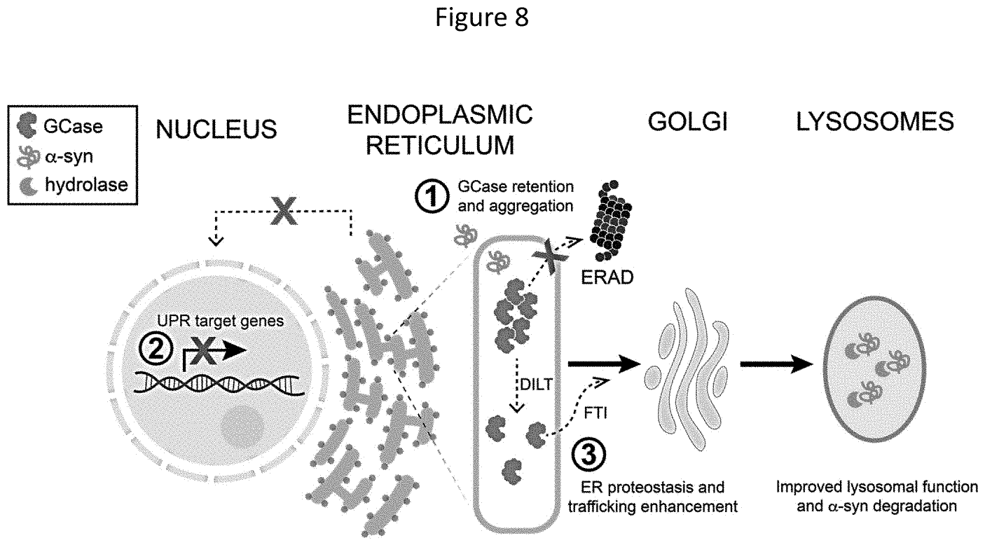

Recent advances in genetics and pathology have highlighted perturbations in the proteostasis network that center on lysosomal degradation and trafficking pathways in both PD and DLB. Using novel PD models that naturally accumulate α-syn through endogenous mutations, we examined changes in the proteostasis pathway that occur during the initial stages of α-syn pathology when insoluble aggregates start to accumulate, but prior to neurotoxicity. Our studies indicated that a critical pathogenic cascade is triggered upon α-syn accumulation at the ER, leading to its fragmentation, compromised folding capacity, and GCase dysfunction (FIG. 8). Other studies using transgenic overexpression models of α-syn have documented the association of α-syn with ER components (Colla et al., 2012a), and are consistent with our findings in PD patient neurons that naturally develop α-syn pathology (FIG. 5). Although normally a cytosolic protein enriched in synapses, pathogenic α-syn accumulates in both neurites and at the cell body in PD neurons. Our data indicates that the mislocalization of α-syn promotes its association and possible interaction with ER chaperones that are important for maintaining GCase folding (FIG. 5; FIG. 13). This association may overwhelm the proteostasis capacity of the ER, leading to ER dysfunction. In addition, α-syn can perturb protein trafficking at the Golgi, which likely slows the export of cargo from the ER, resulting in the accumulation of immature proteins. Overall, our data highlight the negative effects of α-syn on multiple branches of the proteostasis pathway.

A surprising consequence of α-syn-induced trafficking disruption was the aggregation of immature GCase into insoluble species (FIG. 2). While several destabilizing point mutations of lysosomal hydrolases can cause multiple lysosomal storage diseases (LSDs) (Zunke and Mazzulli, 2019), we document a unique example where lysosomal dysfunction occurs through the misfolding and aggregation of wild-type immature GCase. Other lysosomal diseases that are caused by specific genetic mutations in trafficking machinery including I-Cell disease, or LIMP2 depletion that occurs in acute myoclonus renal failure (AMRF), do not show accumulation of immature hydrolases but instead are characterized by aberrant secretion into the extracellular space (FIG. 11B) (Reczek et al., 2007; Wiesmann et al., 1971). Therefore, the aggregation of immature hydrolases induced by α-syn appears to be unique to synucleinopathies. The lack of ERAD activity in SNCA-3X DA cultures (FIG. 4) likely contributes to the aberrant accumulation and destabilization of GCase in the ER. GCase may also be particularly susceptible to aggregation as a membrane-associated enzyme, since aberrant exposure of hydrophobic patches during prolonged folding cycles in the ER may promote its self-association into insoluble aggregates. However, in addition to GCase, we also found the accumulation of immature, insoluble cathepsin D, but not hexosaminidase B (FIG. 11). This indicates that while not all hydrolases are susceptible to aggregation, the effect is not specific for GCase. One other study has shown that a rare point mutation in b-hexosaminidase that causes Tay-sachs disease results in the accumulation of an insoluble enzyme precursor, preventing its trafficking to the lysosome (Proia and Neufeld, 1982). It will be of interest in future studies to examine the factors that induce aggregation of GCase, cathepsin D, and other hydrolases in proteinopathies beyond PD and DLB, that are characterized by lysosomal dysfunction. This may illuminate novel therapeutic opportunities centered on enhancing lysosomal function by targeting protein folding in the ER, and promoting soluble enzymes that can be delivered to the lysosome.

Unexpectedly, we did not observe activation of the UPR, which normally acts to circumvent the accumulation of proteins by expanding ER volume and upregulating folding machinery. Other synucleinopathy models generated by transgenic α-syn overexpression or patient-derived iPSC cortical models exhibited signs of UPR activation (Colla et al., 2012a; Heman-Ackah et al., 2017). The pathological stage at which the models were examined is an important consideration. In some studies, ER stress markers are only elevated during the latest stages of the pathological cascade, indicated by elevated UPR markers in symptomatic transgenic mice or post-mortem brain (Colla et al., 2012a; Credle et al., 2015; Heman-Ackah et al., 2017; Hoozemans et al., 2007). However, UPR activation at this stage does not appear to be selective for synucleinopathies, as many other neurodegenerative diseases exhibit similar features, suggesting the phenomenon may be a general characteristic for late-stage, age-related diseases (Wang and Kaufman, 2016). Our studies were focused on the early stages of α-syn pathology, between day 60 and 90 in our midbrain models (Cuddy et al., 2019; Mazzulli et al., 2016a), in order to capture phenotypic events that occur prior to lysosomal dysfunction and neurodegeneration. Our findings indicate that PD neurons lack the ability to detect misfolded proteins and/or are deficient in their ability to transduce ER stress to activate the UPR (FIG. 8). The finding of insoluble GCase aggregation in the ER, along with severe ER fragmentation, indicates that ER perturbations have reached a critical disease threshold. Furthermore, the UPR is normally triggered by sensors that are highly sensitive, and can efficiently detect protein misfolding (Walter and Ron, 2011). Under periods of ER stress, one of the goals of the UPR is to decrease the overall ER protein load by halting global protein synthesis. However, our recent work showed that GCase mRNA is elevated in PD midbrain neurons that accumulate α-syn, further suggesting communication problems between the ER and the nucleus (FIG. 8) (Cuddy et al., 2019). Even though UPR signaling was not triggered by misfolded GCase or ER fragmentation (FIG. 2 and FIG. 3), we found that patient neurons were capable of responding to chemical ER stress inducers such as Tg and BFA that induce non-specific, pleiotropic effects on the UPR. This suggests that while PD neurons retain their ability to trigger the UPR upon severe stress, they are specifically deficient in handling misfolded proteins in the ER. The mechanism for how protein misfolding goes undetected in PD require further study, but may involve aberrant interactions of α-syn with ER stress triggers including GRP78 or IRE1. Misfolded GCase can trigger the UPR in GD patient neurons, and when expressed in wild-type neurons by lentivirus (FIG. 3), directly demonstrating that the UPR sensors are capable of detecting misfolded GCase. Therefore, it is likely that α-syn impedes the ability of the UPR sensors to detect misfolded GCase, or downstream signal transduction required to activate UPR genes.

Recent work has shown that the UPR can be harnessed to provide protection in neurodegenerative diseases (Grandjean et al., 2020; Vidal et al., 2021). Since our data indicates that the UPR is still capable of activation, it is possible that enhancers of the UPR will provide benefit in synucleinopathies. Such strategies could restore ER proteostasis by stimulating XBP1-S-mediated ER compartment expansion and elevation of chaperones, providing a more conducive environment for GCase folding while preventing the growth of GCase aggregates. These methods would have to avoid maladaptive UPR signaling pathways that promote apoptosis from prolonged stimulation (Wang and Kaufman, 2016).

The unique finding that insoluble GCase accumulates in PD patient neurons prompted us to examine the effect enhancing its solubility on lysosomal function. Our data indicates that enhancing ER proteostasis with diltiazem (DILT), dantrolene (DANT), or DHBP, three established methods of enhancing ER folding capacity (Ong et al., 2010), can promote GCase folding and solubility (FIG. 6; FIG. 14). However, the downstream therapeutic effects of enhancing this pathway alone were marginal. We observed only mild elevations in GCase maturation, and no change in lysosomal activity or α-syn levels in patient neurons. This marginal rescue was due to the previously established effects of α-syn inhibiting protein trafficking downstream of the ER (Cooper et al., 2006; Cuddy et al., 2019; Gitler et al., 2008). Our previous work showed that enhancement of protein trafficking can be achieved with a clinically validated FTI that activates lysosomes by enhancing the ER-Golgi SNARE protein ykt6 (Cuddy et al., 2019). Interestingly, FTIs have been shown to activate lysosomes and reduce pathology in other neurodegenerative disease models characterized by tau accumulation (Hernandez et al., 2019). We show that enhancing ER proteostasis and simultaneously targeting downstream trafficking pathways can effectively improve GCase maturation and reduce pathological α-syn (FIG. 7, FIG. 15). Once in the lysosome, GCase activity reduces α-syn by degrading glycosphingolipid substrates that interact and stabilize toxic α-syn (Zunke et al., 2018). Importantly, this strategy was also effective in patient neurons that harbor GBA1 mutations (FIG. 16), indicating a potential to treat neuronopathic GD and GBA-PD with these compounds. Enhancement of the lysosomal pathway, and proteostasis in general, holds promise as a disease-modifying therapeutic strategy for synucleinopathies as well as other protein aggregation diseases. Our study provides a novel method for simultaneously targeting multiple branches of the proteostasis pathway, including folding, trafficking and degradation, to restore balance in PD patient neurons. Therapeutic enhancement of multiple proteostasis pathways may provide optimal benefit in PD, given the pleiotropic deleterious effects of α-syn accumulation in multiple subcellular locations. Furthermore, combining two treatments that target distinct cellular pathways may enable administration of lower doses of each drug, which would limit compound toxicity if these treatments should progress to the clinic.

### REFERENCES

Abeliovich, A., and Gitler, A. D. (2016). Defects in trafficking bridge Parkinson's disease pathology and genetics. Nature 539, 207-216. 10.1038/nature20414.
Abeliovich, A., Schmitz, Y., Farinas, I., Choi-Lundberg, D., Ho, W. H., Castillo, P. E., Shinsky, N., Verdugo, J. M., Armanini, M., Ryan, A., et al. (2000). Mice lacking alpha-synuclein display functional deficits in the nigrostriatal dopamine system. Neuron 25, 239-252. 10.1016/s0896-6273(00)80886-7.
Apicco, D. J., Shlevkov, E., Nezich, C. L., Tran, D. T., Guilmette, E., Nicholatos, J. W., Bantle, C. M., Chen, Y., Glajch, K. E., Abraham, N. A., et al. (2021). The Parkinson's disease-associated gene ITPKB protects against alpha-synuclein aggregation by regulating ER-to-mitochondria calcium release. Proc Natl Acad Sci USA 118. 10.1073/pnas.2006476118.
Bellucci, A., Navarria, L., Zaltieri, M., Falarti, E., Bodei, S., Sigala, S., Battistin, L., Spillantini, M., Missale, C., and Spano, P. (2011). Induction of the unfolded protein response by alpha-synuclein in experimental models of Parkinson's disease. J Neurochem 116, 588-605. 10.1111/j.1471-4159.2010.07143.x.
Bergmann, J. E., and Grabowski, G. A. (1989). Posttranslational processing of human lysosomal acid beta-glucosidase: a continuum of defects in Gaucher disease type 1 and type 2 fibroblasts. Am J Hum Genet 44, 741-750.
Booth, C., and Koch, G. L. (1989). Perturbation of cellular calcium induces secretion of luminal ER proteins. Cell 59, 729-737. 10.1016/0092-8674(89)90019-6.
Calfon, M., Zeng, H., Urano, F., Till, J. H., Hubbard, S. R., Harding, H. P., Clark, S. G., and Ron, D. (2002). IRE1 couples endoplasmic reticulum load to secretory capacity by processing the XBP-1 mRNA. Nature 415, 92-96. 10.1038/415092a.
Caraveo, G., Auluck, P. K., Whitesell, L., Chung, C. Y., Baru, V., Mosharov, E. V., Yan, X., Ben-Johny, M., Soste, M., Picotti, P., et al. (2014). Calcineurin determines toxic versus beneficial responses to alpha-synuclein. Proc Natl Acad Sci USA 111, E3544-3552. 10.1073/pnas.1413201111.
Chang, D., Nalls, M. A., Hallgrimsdottir, I. B., Hunkapiller, J., van der Brug, M., Cai, F., International Parkinson's Disease Genomics, C., and Me Research, T., Kerchner, G. A., Ayalon, G., et al. (2017). A meta-analysis of genome-wide association studies identifies 17 new Parkinson's disease risk loci. Nat Genet 49, 1511-1516. 10.1038/ng.3955.
Chia, R., Sabir, M. S., Bandres-Ciga, S., Saez-Atienzar, S., Reynolds, R. H., Gustavsson, E., Walton, R. L., Ahmed, S., Viollet, C., Ding, J., et al. (2021). Genome sequencing analysis identifies new loci associated with Lewy body dementia and provides insights into its genetic architecture. Nat Genet. 10.1038/s41588-021-00785-3.
Chung, C. Y., Khurana, V., Auluck, P. K., Tardiff, D. F., Mazzulli, J. R., Soldner, F., Baru, V., Lou, Y., Freyzon, Y., Cho, S., et al. (2013). Identification and rescue of α-synuclein toxicity in Parkinson patient-derived neurons. Science (New York, N.Y.) 342, 983-987. 10.1126/science.1245296.
Colla, E., Coune, P., Liu, Y., Pletnikova, O., Troncoso, J. C., Iwatsubo, T., Schneider, B. L., and Lee, M. K. (2012a). Endoplasmic reticulum stress is important for the manifestations of α-synucleinopathy in vivo. The Journal of neuroscience: the official journal of the Society for Neuroscience 32, 3306-3320. 10.1523/JNEUROSCI.5367-11.2012.
Colla, E., Jensen, P. H., Pletnikova, O., Troncoso, J. C., Glabe, C., and Lee, M. K. (2012b). Accumulation of toxic alpha-synuclein oligomer within endoplasmic reticulum occurs in alpha-synucleinopathy in vivo. J Neurosci 32, 3301-3305. 10.1523/JNEUROSCI.5368-11.2012.
Colla, E., Panattoni, G., Ricci, A., Rizzi, C., Rota, L., Carucci, N., Valvano, V., Gobbo, F., Capsoni, S., Lee, M. K., and Cattaneo, A. (2018). Toxic properties of microsome-associated alpha-synuclein species in mouse primary neurons. Neurobiol Dis 111, 36-47. 10.1016/j.nbd.2017.12.004.
Conway, K. A., Harper, J. D., and Lansbury, P. T. (1998). Accelerated in vitro fibril formation by a mutant alpha-synuclein linked to early-onset Parkinson disease. Nat Med 4, 1318-1320. 10.1038/3311.
Cooper, A. A., Gitler, A. D., Cashikar, A., Haynes, C. M., Hill, K. J., Bhullar, B., Liu, K., Xu, K., Strathearn, K. E., Liu, F., et al. (2006). Alpha-synuclein blocks ER-Golgi traffic and Rab1 rescues neuron loss in Parkinson's models. Science 313, 324-328. 10.1126/science.1129462.
Credle, J. J., Forcelli, P. A., Delannoy, M., Oaks, A. W., Permaul, E., Berry, D. L., Duka, V., Wills, J., and Sidhu, A. (2015). alpha-Synuclein-mediated inhibition of ATF6 processing into COPII vesicles disrupts UPR signaling in Parkinson's disease. Neurobiol Dis 76, 112-125. 10.1016/j.nbd.2015.02.005.
Cuddy, L. K., and Mazzulli, J. R. (2021). Analysis of lysosomal hydrolase trafficking and activity in human iPSC-derived neuronal models. STAR Protoc 2, 100340. 10.1016/j.xpro.2021.100340.
Cuddy, L. K., Wani, W. Y., Morella, M. L., Pitcairn, C., Tsutsumi, K., Fredriksen, K., Justman, C. J., Grammatopoulos, T. N., Belur, N. R., Zunke, F., et al. (2019). Stress-Induced Cellular Clearance Is Mediated by the SNARE Protein ykt6 and Disrupted by alpha-Synuclein. Neuron 104, 869-884 e811. 10.1016/j.neuron.2019.09.001.
Fernandes, H. J. R., Hartfield, E. M., Christian, H. C., Emmanoulidou, E., Zheng, Y., Booth, H., Bogetofte, H., Lang, C., Ryan, B. J., Sardi, S. P., et al. (2016). ER Stress and Autophagic Perturbations Lead to Elevated Extracellular α-Synuclein in GBA-N370S Parkinson's iPSC-Derived Dopamine Neurons. Stem cell reports 6, 342-356. 10.1016/j.stemcr.2016.01.013.
Fruen, B. R., Mickelson, J. R., and Louis, C. F. (1997). Dantrolene inhibition of sarcoplasmic reticulum Ca2+ release by direct and specific action at skeletal muscle ryanodine receptors. J Biol Chem 272, 26965-26971. 10.1074/jbc.272.43.26965.
Fuchs, J., Nilsson, C., Kachergus, J., Munz, M., Larsson, E. M., Schule, B., Langston, J. W., Middleton, F. A., Ross, O. A., Hulihan, M., et al. (2007). Phenotypic variation in a large Swedish pedigree due to SNCA duplication and triplication. Neurology 68, 916-922. 10.1212/01.wnl.0000254458.17630.c5.
Fujiwara, T., Oda, K., Yokota, S., Takatsuki, A., and Ikehara, Y. (1988). Brefeldin A causes disassembly of the Golgi complex and accumulation of secretory proteins in the endoplasmic reticulum. J Biol Chem 263, 18545-18552.
Garcia-Sanz, P., Orgaz, L., Bueno-Gil, G., Espadas, I., Rodriguez-Traver, E., Kulisevsky, J., Gutierrez, A., Davila, J. C., Gonzalez-Polo, R. A., Fuentes, J. M., et al. (2017). N370S-GBA1 mutation causes lysosomal cholesterol accumulation in Parkinson's disease. Mov Disord 32, 1409-1422. 10.1002/mds.27119.
Giannini, G., Conti, A., Mammarella, S., Scrobogna, M., and Sorrentino, V. (1995). The ryanodine receptor/calcium channel genes are widely and differentially expressed in murine brain and peripheral tissues. J Cell Biol 128, 893-904. 10.1083/jcb.128.5.893.
Gitler, A. D., Bevis, B. J., Shorter, J., Strathearn, K. E., Hamamichi, S., Su, L. J., Caldwell, K. A., Caldwell, G. A., Rochet, J. C., McCaffery, J. M., et al. (2008). The Parkinson's disease protein alpha-synuclein disrupts cellular Rab homeostasis. Proc Natl Acad Sci USA 105, 145-150. 10.1073/pnas.0710685105.
Gorbatyuk, M. S., Shabashvili, A., Chen, W., Meyers, C., Sullivan, L. F., Salganik, M., Lin, J. H., Lewin, A. S., Muzyczka, N., and Gorbatyuk, O. S. (2012). Glucose regulated protein 78 diminishes alpha-synuclein neurotoxicity in a rat model of Parkinson disease. Mol Ther 20, 1327-1337. 10.1038/mt.2012.28.
Gosavi, N., Lee, H. J., Lee, J. S., Patel, S., and Lee, S. J. (2002). Golgi fragmentation occurs in the cells with prefibrillar alpha-synuclein aggregates and precedes the formation of fibrillar inclusion. J Biol Chem 277, 48984-48992. 10.1074/jbc.M208194200.
Grandjean, J. M. D., Madhavan, A., Cech, L., Seguinot, B. O., Paxman, R. J., Smith, E., Scampavia, L., Powers, E. T., Cooley, C. B., Plate, L., et al. (2020). Pharmacologic IRE1/XBP1s activation confers targeted ER proteostasis reprogramming. Nat Chem Biol 16, 1052-1061. 10.1038/s41589-020-0584-z.
Guardia-Laguarta, C., Area-Gomez, E., Rub, C., Liu, Y., Magrane, J., Becker, D., Voos, W., Schon, E. A., and Przedborski, S. (2014). alpha-Synuclein is localized to mitochondria-associated ER membranes. J Neurosci 34, 249-259. 10.1523/JNEUROSCI.2507-13.2014.
Ha, T. W., Jeong, J. H., Shin, H., Kim, H. K., Im, J. S., Song, B. H., Hanna, J., Oh, J. S., Woo, D. H., Han, J., and Lee, M. R. (2020). Characterization of Endoplasmic Reticulum (ER) in Human Pluripotent Stem Cells Revealed Increased Susceptibility to Cell Death upon ER Stress. Cells 9. 10.3390/cells9051078.
Harding, H. P., Zhang, Y., and Ron, D. (1999). Protein translation and folding are coupled by an endoplasmic-reticulum-resident kinase. Nature 397, 271-274. 10.1038/16729.
Helms, J. B., and Rothman, J. E. (1992). Inhibition by brefeldin A of a Golgi membrane enzyme that catalyses exchange of guanine nucleotide bound to ARE. Nature 360, 352-354. 10.1038/360352a0.
Heman-Ackah, S. M., Manzano, R., Hoozemans, J. J. M., Scheper, W., Flynn, R., Haerty, W., Cowley, S. A., Bassett, A. R., and Wood, M. J. A. (2017). Alpha-synuclein induces the unfolded protein response in Parkinson's disease SNCA triplication iPSC-derived neurons. Hum Mol Genet 26, 4441-4450. 10.1093/hmg/ddx331.
Hernandez, I., Luna, G., Rauch, J. N., Reis, S. A., Giroux, M., Karch, C. M., Boctor, D., Sibih, Y. E., Storm, N. J., Diaz, A., et al. (2019). A farnesyltransferase inhibitor activates lysosomes and reduces tau pathology in mice with tauopathy. Sci Transl Med 11. 10.1126/scitranslmed.aat3005.
Hoozemans, J. J., van Haastert, E. S., Eikelenboom, P., de Vos, R. A., Rozemuller, J. M., and Scheper, W. (2007). Activation of the unfolded protein response in Parkinson's disease. Biochem Biophys Res Commun 354, 707-711. 10.1016/j.bbrc.2007.01.043.
Hunn, B. H., Cragg, S. J., Bolam, J. P., Spillantini, M. G., and Wade-Martins, R. (2015). Impaired intracellular trafficking defines early Parkinson's disease. Trends Neurosci 38, 178-188. 10.1016/j.tins.2014.12.009.
Kang, J. J., Hsu, K. S., and Lin-Shiau, S. Y. (1994). Effects of bipyridylium compounds on calcium release from triadic vesicles isolated from rabbit skeletal muscle. Br J Pharmacol 112, 1216-1222. 10.1111/j.1476-5381.1994.tb13213.x.
Klein, A. D., and Mazzulli, J. R. (2018). Is Parkinson's disease a lysosomal disorder?Brain 141, 2255-2262. 10.1093/brain/awy147.
Koch, G. L., Booth, C., and Wooding, F. B. (1988). Dissociation and re-assembly of the endoplasmic reticulum in live cells. J Cell Sci 91 (Pt 4), 511-522.
Korkotian, E., Schwarz, A., Pelled, D., Schwarzmann, G., Segal, M., and Futerman, A. H. (1999). Elevation of intracellular glucosylceramide levels results in an increase in endoplasmic reticulum density and in functional calcium stores in cultured neurons. J Biol Chem 274, 21673-21678.
Kozutsumi, Y., Segal, M., Normington, K., Gething, M. J., and Sambrook, J. (1988). The presence of malfolded proteins in the endoplasmic reticulum signals the induction of glucose-regulated proteins. Nature 332, 462-464. 10.1038/332462a0.
Kriks, S., Shim, J. W., Piao, J., Ganat, Y. M., Wakeman, D. R., Xie, Z., Carrillo-Reid, L., Auyeung, G., Antonacci, C., Buch, A., et al. (2011). Dopamine neurons derived from human ES cells efficiently engraft in animal models of Parkinson's disease. Nature 480, 547-551. 10.1038/nature10648.
Lee, A. H., Iwakoshi, N. N., and Glimcher, L. H. (2003). XBP-1 regulates a subset of endoplasmic reticulum resident chaperone genes in the unfolded protein response. Mol Cell Biol 23, 7448-7459. 10.1128/mcb.23.21.7448-7459.2003.
Liou, B., Peng, Y., Li, R., Inskeep, V., Zhang, W., Quinn, B., Dasgupta, N., Blackwood, R., Setchell, K. D., Fleming, S., et al. (2016). Modulating ryanodine receptors with dantrolene attenuates neuronopathic phenotype in Gaucher disease mice. Hum Mol Genet 25, 5126-5141. 10.1093/hmg/ddw322.
Marquardt, T., and Helenius, A. (1992). Misfolding and aggregation of newly synthesized proteins in the endoplasmic reticulum. J Cell Biol 117, 505-513. 10.1083/jcb.117.3.505.
Martin, I., Kim, J. W., Dawson, V. L., and Dawson, T. M. (2014). LRRK2 pathobiology in Parkinson's disease. J Neurochem 131, 554-565. 10.1111/jnc.12949.
Masliah, E., Rockenstein, E., Veinbergs, I., Mallory, M., Hashimoto, M., Takeda, A., Sagara, Y., Sisk, A., and Mucke, L. (2000). Dopaminergic loss and inclusion body formation in alpha-synuclein mice: implications for neurodegenerative disorders. Science 287, 1265-1269. 10.1126/science.287.5456.1265.
Mazzulli, J. R., Xu, Y. H., Sun, Y., Knight, A. L., McLean, P. J., Caldwell, G. A., Sidransky, E., Grabowski, G. A., and Krainc, D. (2011). Gaucher disease glucocerebrosidase and alpha-synuclein form a bidirectional pathogenic loop in synucleinopathies. Cell 146, 37-52. 10.1016/j.cell.2011.06.001.
Mazzulli, J. R., Zunke, F., Isacson, O., Studer, L., and Krainc, D. (2016a). alpha-Synuclein-induced lysosomal dysfunction occurs through disruptions in protein trafficking in human midbrain synucleinopathy models. Proc Natl Acad Sci USA 113, 1931-1936. 10.1073/pnas.1520335113.
Mazzulli, J. R., Zunke, F., Isacson, O., Studer, L., and Krainc, D. (2016b). alpha-Synuclein-induced lysosomal dysfunction occurs through disruptions in protein trafficking in human midbrain synucleinopathy models. Proc Natl Acad Sci USA 113, 1931-1936. 10.1073/pnas.1520335113.
Melnick, J., Dul, J. L., and Argon, Y. (1994). Sequential interaction of the chaperones BiP and GRP94 with immunoglobulin chains in the endoplasmic reticulum. Nature 370, 373-375. 10.1038/370373a0.
Mu, T. W., Ong, D. S., Wang, Y. J., Balch, W. E., Yates, J. R., 3rd, Segatori, L., and Kelly, J. W. (2008). Chemical and biological approaches synergize to ameliorate protein-folding diseases. Cell 134, 769-781. 10.1016/j.cell.2008.06.037.
Nalls, M. A., Pankratz, N., Lill, C. M., Do, C. B., Hernandez, D. G., Saad, M., DeStefano, A. L., Kara, E., Bras, J., Sharma, M., et al. (2014). Large-scale meta-analysis of genome-wide association data identifies six new risk loci for Parkinson's disease. Nat Genet 46, 989-993. 10.1038/ng.3043.
Ong, D. S. T., Mu, T. W., Palmer, A. E., and Kelly, J. W. (2010). Endoplasmic reticulum Ca2+ increases enhance mutant glucocerebrosidase proteostasis. Nature chemical biology 6, 424-432. 10.1038/nchembio.368.
Ou, W. J., Cameron, P. H., Thomas, D. Y., and Bergeron, J. J. (1993). Association of folding intermediates of glycoproteins with calnexin during protein maturation. Nature 364, 771-776. 10.1038/364771a0.
Paillusson, S., Gomez-Suaga, P., Stoica, R., Little, D., Gissen, P., Devine, M. J., Noble, W., Hanger, D. P., and Miller, C. C. J. (2017). alpha-Synuclein binds to the ER-mitochondria tethering protein VAPB to disrupt Ca(2+) homeostasis and mitochondrial ATP production. Acta Neuropathol 134, 129-149. 10.1007/s00401-017-1704-z.
Pelled, D., Trajkovic-Bodennec, S., Lloyd-Evans, E., Sidransky, E., Schiffmann, R., and Futerman, A. H. (2005). Enhanced calcium release in the acute neuronopathic form of Gaucher disease. Neurobiol Dis 18, 83-88. 10.1016/j.nbd.2004.09.004.
Price, B. D., Mannheim-Rodman, L. A., and Calderwood, S. K. (1992). Brefeldin A, thapsigargin, and AIF4—stimulate the accumulation of GRP78 mRNA in a cycloheximide dependent manner, whilst induction by hypoxia is independent of protein synthesis. J Cell Physiol 152, 545-552. 10.1002/jcp.1041520314.
Proia, R. L., and Neufeld, E. F. (1982). Synthesis of beta-hexosaminidase in cell-free translation and in intact fibroblasts: an insoluble precursor alpha chain in a rare form of Tay-Sachs disease. Proc Natl Acad Sci USA 79, 6360-6364. 10.1073/pnas.79.20.6360.
Rajagopalan, S., Xu, Y., and Brenner, M. B. (1994). Retention of unassembled components of integral membrane proteins by calnexin. Science 263, 387-390. 10.1126/science.8278814.
Reczek, D., Schwake, M., Schroder, J., Hughes, H., Blanz, J., Jin, X. Y., Brondyk, W., Van Patten, S., Edmunds, T., and Saftig, P. (2007). LIMP-2 is a receptor for lysosomal mannose-6-phosphate-independent targeting of beta-Glucocerebrosidase. Cell 131, 770-783. 10.1016/j.cell.2007.10.018.
Robak, L. A., Jansen, I. E., van Rooij, J., Uitterlinden, A. G., Kraaij, R., Jankovic, J., International Parkinson's Disease Genomics, C., Heutink, P., and Shulman, J. M. (2017). Excessive burden of lysosomal storage disorder gene variants in Parkinson's disease. Brain 140, 3191-3203. 10.1093/brain/awx285.
Ron, I., and Horowitz, M. (2005). ER retention and degradation as the molecular basis underlying Gaucher disease heterogeneity. Hum Mol Genet 14, 2387-2398. 10.1093/hmg/ddi240.
Roshan Lal, T., and Sidransky, E. (2017). The Spectrum of Neurological Manifestations Associated with Gaucher Disease. Diseases 5. 10.3390/diseases5010010.
Rothaug, M., Zunke, F., Mazzulli, J. R., Schweizer, M., Altmeppen, H., Lullmann-Rauch, R., Kallemeijn, W. W., Gaspar, P., Aerts, J. M., Glatzel, M., et al. (2014). LIMP-2 expression is critical for beta-glucocerebrosidase activity and alpha-synuclein clearance. Proc Natl Acad Sci USA 111, 15573-15578. 10.1073/pnas.1405700111.
Schuck, S., Prinz, W. A., Thorn, K. S., Voss, C., and Walter, P. (2009). Membrane expansion alleviates endoplasmic reticulum stress independently of the unfolded protein response. J Cell Biol 187, 525-536. 10.1083/jcb.200907074.
Sidransky, E., Nalls, M. A., Aasly, J. O., Aharon-Peretz, J., Annesi, G., Barbosa, E. R., Bar-Shira, A., Berg, D., Bras, J., Brice, A., et al. (2009). Multicenter analysis of glucocerebrosidase mutations in Parkinson's disease. N Engl J Med 361, 1651-1661. 10.1056/NEJMoa0901281.
Simon-Sanchez, J., Schulte, C., Bras, J. M., Sharma, M., Gibbs, J. R., Berg, D., Paisan-Ruiz, C., Lichtner, P., Scholz, S. W., Hernandez, D. G., et al. (2009). Genome-wide association study reveals genetic risk underlying Parkinson's disease. Nat Genet 41, 1308-1312. 10.1038/ng.487.
Singh, P. K., and Muqit, M. M. K. (2020). Parkinson's: A Disease of Aberrant Vesicle Trafficking. Annu Rev Cell Dev Biol 36, 237-264. 10.1146/annurev-cellbio-100818-125512.
Singleton, A. B., Farrer, M., Johnson, J., Singleton, A., Hague, S., Kachergus, J., Hulihan, M., Peuralinna, T., Dutra, A., Nussbaum, R., et al. (2003). alpha-Synuclein locus triplication causes Parkinson's disease. Science 302, 841. 10.1126/science.1090278.
Smith, M. H., Ploegh, H. L., and Weissman, J. S. (2011). Road to ruin: targeting proteins for degradation in the endoplasmic reticulum. Science 334, 1086-1090. 10.1126/science.1209235.
Soldner, F., Laganiere, J., Cheng, A. W., Hockemeyer, D., Gao, Q., Alagappan, R., Khurana, V., Golbe, L. I., Myers, R. H., Lindquist, S., et al. (2011). Generation of isogenic pluripotent stem cells differing exclusively at two early onset Parkinson point mutations. Cell 146, 318-331. 10.1016/j.cell.2011.06.019.
Spillantini, M. G., Schmidt, M. L., Lee, V. M., Trojanowski, J. Q., Jakes, R., and Goedert, M. (1997). Alpha-synuclein in Lewy bodies. Nature 388, 839-840. 10.1038/42166.
Stojkovska, I., and Mazzulli, J. R. (2021). Detection of pathological alpha-synuclein aggregates in human iPSC-derived neurons and tissue. STAR Protoc 2, 100372. 10.1016/j.xpro.2021.100372.
Stojkovska, Iva et al. “Rescue of α-Synuclein Aggregation in Parkinson's Patient Neurons by Synergistic Enhancement of ER Proteostasis and Protein Trafficking.” Neuron. 2022 Feb. 2; 110(3):436-451.e11.
Sun, Y., Liou, B., Quinn, B., Ran, H., Xu, Y. H., and Grabowski, G. A. (2009). In vivo and ex vivo evaluation of L-type calcium channel blockers on acid beta-glucosidase in Gaucher disease mouse models. PLoS One 4, e7320. 10.1371/journal.pone.0007320.
Tan, Y. L., Genereux, J. C., Pankow, S., Aerts, J. M., Yates, J. R., 3rd, and Kelly, J. W. (2014). ERdj3 is an endoplasmic reticulum degradation factor for mutant glucocerebrosidase variants linked to Gaucher's disease. Chem Biol 21, 967-976. 10.1016/j.chembiol.2014.06.008.
Thayanidhi, N., Helm, J. R., Nycz, D. C., Bentley, M., Liang, Y., and Hay, J. C. (2010). Alpha-synuclein delays endoplasmic reticulum (ER)-to-Golgi transport in mammalian cells by antagonizing ER/Golgi SNAREs. Mol Biol Cell 21, 1850-1863. 10.1091/mbc.E09-09-0801.
Vidal, R. L., Sepulveda, D., Troncoso-Escudero, P., Garcia-Huerta, P., Gonzalez, C., Plate, L., Jerez, C., Canovas, J., Rivera, C. A., Castillo, V., et al. (2021). Enforced dimerization between XBP1s and ATF6f enhances the protective effects of the UPR in models of neurodegeneration. Mol Ther. 10.1016/j.ymthe.2021.01.033.
Walter, P., and Ron, D. (2011). The unfolded protein response: from stress pathway to homeostatic regulation. Science 334, 1081-1086. 10.1126/science.1209038.
Wang, F., Agnello, G., Sotolongo, N., and Segatori, L. (2011). Ca2+ homeostasis modulation enhances the amenability of L444P glucosylcerebrosidase to proteostasis regulation in patient-derived fibroblasts. ACS Chem Biol 6, 158-168. 10.1021/cb100321m.
Wang, M., and Kaufman, R. J. (2016). Protein misfolding in the endoplasmic reticulum as a conduit to human disease. Nature 529, 326-335. 10.1038/nature17041.
Wiesmann, U. N., Lightbody, J., Vassella, F., and Herschkowitz, N. N. (1971). Multiple lysosomal enzyme deficiency due to enzyme leakage? N Engl J Med 284, 109-110. 10.1056/NEJM197101142840220.
Yoshida, H., Matsui, T., Yamamoto, A., Okada, T., and Mori, K. (2001). XBP1 mRNA is induced by ATF6 and spliced by IRE1 in response to ER stress to produce a highly active transcription factor. Cell 107, 881-891. 10.1016/s0092-8674(01)00611-0.
Zunke, F., and Mazzulli, J. R. (2019). Modeling neuronopathic storage diseases with patient-derived culture systems. Neurobiol Dis 127, 147-162. 10.1016/j.nbd.2019.01.018.
Zunke, F., Moise, A. C., Belur, N. R., Gelyana, E., Stojkovska, I., Dzaferbegovic, H., Toker, N. J., Jeon, S., Fredriksen, K., and Mazzulli, J. R. (2018). Reversible Conformational Conversion of alpha-Synuclein into Toxic Assemblies by Glucosylceramide. Neuron 97, 92-107 e110. 10.1016/j.neuron.2017.12.012.

### Example 2—Supplemental Material for Example 1

Materials and Methods

Human H4 Neuroglioma Cell Culture

Human H4 neuroglioma cells were stably transfected to overexpress wild-type α-syn under the control of a tetracycline-inducible promoter via a Tet-off system and described previously (Mazzulli et al., 2011). α-Syn expression was turned off by the addition of 1 ug/ml doxycycline (DOX) (Sigma), a tetracycline analog, for a minimum of 3 days. Cells were cultured in Optimem media with 5% heat-inactivated FBS, 0.2 mg/ml geneticin, 0.2 mg/ml hygromycin B, and 1% penicillin/streptomycin (Thermo Fisher Scientific).

Reprogramming and Culturing of Human Induced Pluripotent Stem Cells (iPSCs)

B-lymphocytes from healthy controls and PD patients that carry a triplication in the SNCA genomic region were obtained from the Coriell NINDS and NIGMS Human Genetic Cell Repositories: GM15845 (Ctrl), GM15010 (3x−1), ND00196 (3x−2), ND00139 (3x−4), ND34391 (Est. 3X). Phenotypic and genotypic data of these subjects is available on https://www.coriell.org. See Supplementary Table for more details, including information on Est. Ctrl, SNCA A53T mutant, and GBA1 mutant iPSC lines (N370S/84GG and L444P/L444P). The B-lymphocytes were reprogrammed into iPSCs by transfection with non-integrating episomal plasmids containing Oct3/4 (Addgene: pCXLE-hOCT3/4-shp53-F), L-Myc (Addgene: pCXLE-hUL), and Sox2 and Klf4 (Addgene: pCXLE-hSK). All iPSCs were maintained in mTeSR1 media on matrigel-coated plates.

Pluripotency Analysis of Reprogrammed iPSC Cells

Immunofluorescence Analysis of Pluripotency Markers

Cells plated on glass coverslips were fixed in 4% paraformaldehyde (Polysciences, Inc.) for 15 minutes, permeabilized with 0.3% Triton X-100 (Sigma) in PBS for 30 minutes, and blocked with 2% bovine serum albumin (BSA) (Roche) in Triton-PBS for 30 minutes to prevent non-specific antibody binding. Primary antibodies (Sox2, Tra-1-60, Oct4, SSEA4, Nanog) were added overnight, followed by incubation with secondary antibodies (Alexa Fluor 488 Goat anti-rabbit IgG and Alexa Fluor 568 Goat anti-mouse IgG) for 1 hour. The cells were then washed three times with Triton-PBS and mounted onto microscope slides with DAPI mounting media.

PCR Analysis of Reprogramming Factor Transgenes

Forward and reverse PCR primers for each of the reprogramming factor transgenes (Oct3/4, Sox2, Klf4, L-Myc) were designed so that the PCR product will span both the transgene and the plasmid backbone, as indicated in the schematic of FIG. 9C. See Key Resource Table for list of primers. The PCR was performed with Taq polymerase (NEB #M0273L) and 20 ng of genomic DNA using the following cycling conditions: Initial denaturation 95° C. 3 min; 40 cycles: Denaturation 95° C. 30s, Annealing 60° C. 30s, Extension 68° C. 30s; Final extension 68° C. 5 min; 4° C. hold. The PCR products were run on 1.5% agarose gel for 45 min at 120V and imaged on a Chemicdoc imaging system (Biorad).

Quantitative RT-PCR

Total RNA was isolated from cells in a 24 or 12 well format using an RNeasy Mini Prep kit (QIAGEN). cDNA was synthesized by reverse transcriptase PCR (RT-PCR) using the RevertAid First Strand cDNA synthesis kit (Thermo Fisher Scientific). Quantitative PCR was performed on the Applied Biosystems 7500 Fast system using the cDNA and pre-designed TaqMan-primer probes for the target genes. The target mRNA expression was quantified relative to GAPDH or 3-actin using the delta-delta-Ct method, and represented as a fold change.

Copy Number Analysis of SNCA and Puromycin

Genomic DNA was extracted from a 12 well plate of iPSCs using the DNeasy Blood and Tissue Kit (69504, Qiagen). Quantitative PCR was performed using default cycling conditions on the Applied Biosystems 7500 Fast system with 100 ng genomic DNA and pre-designed TaqMan probe for SNCA (Hs04791950_cn) or custom probe for puromycin (gi763524_CCN1F1Y). The copy number of each gene was quantified relative to reference RPPH1 copy number assay (4401631, Applied Biosystems). The analysis was performed using ddCt method and expressed as fold change.

Fluorescence In-Situ Hybridization (FISH) Analysis

To confirm the SNCA copy number in the reprogrammed iPSCs, fluorescent probes targeting SNCA (4q22.1; R: red) and a control region (4p16.3; G: green) were used for FISH analysis. The assay was performed as a service provided by Cell Line Genetics, Inc (www.clgenetics.com).

Differentiation of iPSCs into Midbrain Dopaminergic Neurons

The iPSCs were differentiated into midbrain dopaminergic neurons using a well-established dual SMAD inhibition protocol (Kriks et al., 2011), and have been previously described in detail (Mazzulli et al., 2016b). Neurons were cultured in neurobasal SM1 media (Thermo Fisher Scientific) containing NeuroCult SM1 supplement (StemCell Technologies), 1% penicillin/streptomycin, and 1% L-glutamine (Gibco). Neurons were aged to 60-90 days for each experiment as indicated in the text or figure legends.

Immunofluorescence Analysis of Midbrain Neuron Differentiation Efficiency, α-Synuclein Accumulation, and Thioflavin Staining

Neurons were fixed in 4% paraformaldehyde for 15 minutes, permeabilized with 0.1% Triton X-100 in PBS for 30 minutes, and blocked with 2% BSA and 5% normal goat serum (NGS) (Jackson Immuno Research) in Triton-PBS for 30 minutes to prevent non-specific antibody binding. Primary antibodies (anti-α-synuclein LB509, anti-tyrosine hydroxylase (TH), anti-FoxA2, anti-β3-tubulin) were added overnight, followed by incubation with secondary antibodies (Alexa Fluor 488 Goat anti-mouse IgG and Alexa Fluor 568 Goat anti-rabbit IgG) for 1 hour. The cells were then washed three times with Triton-PBS and mounted onto microscope slides with DAPI mounting media. For thioflavin S (Thio S) co-staining, following primary incubation with a-syn, 0.05% Thio S was directly added to cells and incubated for 15 min at RT. Next, cells were washed with a sequence of ethanol steps (twice with 50% ethanol for 20 min each, then once with 80% ethanol for 20 min) and then with Triton-PBS prior to mounting. The Thio S and a-synuclein staining has been described in detail (Stojkovska and Mazzulli, 2021). All images were obtained on a Leica confocal microscope, and image analysis was performed using ImageJ.

Dual Nickase CRISPR/Cas9 Strategy and Selection of iPSC Clones

A pair of guide RNAs (guide RNA 1: 5′-AGCAGCCACAACTCCCTCCTTGG-3′ SEQ ID NO: 1; guide RNA 2: 5′-TGAGAAAACCAAACAGGGTGTGG-3′ SEQ ID NO: 2) were designed using the Optimized CRISPR design tool (http://crispr.mit.edu/), and used to direct D10A mutant Cas9 to produce nicks within Exon 2 of the SNCA gene. A PITX3-2A-eGFP-PGK-Puro plasmid (Addgene) encoding a puromycin resistance cassette driven by a phosphoglycerate kinase (PGK) promoter was used as a template for homologous recombination (HR) and as a positive selection marker. The gRNAs were cloned into a Cas9-nickase plasmid PX335 (Addgene) and transfected into iPSCs using Lipofectamine 3000 (Thermo Fisher Scientific) along with a puromycin-containing HR plasmid. Two days following the transfection, iPSCs were cultured in 1 ug/ml puromycin containing media for several weeks. To confirm that the puromycin cassette was appropriately inserted in the targeted SNCA Exon 2 region, puromycin resistant clones were selected and genomic DNA was extracted and analyzed via PCR using the following primers: 5′ F: CATAAAATCTGTCTGCCCGCTCTC SEQ ID NO: 3, 5′ R: GTGGGCTTGTACTCGGTC SEQ ID NO: 4; 3′ F: CTTCTACGAGCGGCTCGGCTT SEQ ID NO: 5, 3′ R: TGTGGTCATCCTCCACCTGACT SEQ ID NO: 6. Puromycin copy number analysis and sequencing were also performed on selected clones.

Analysis of Off-Target Effects Using the T7EI Cleavage Assay

Genomic DNA was amplified using primers for each off-target gene (see Key Resource Table for list of primers). The PCR products were then denatured and allowed to re-anneal using a thermal cycler with the following settings: 95° C. for 10 minutes, 95-85° C. (ramp rate 2° C./sec), and 85-25° C. (ramp rate 0.2° C./sec). The hybridized product was then digested with T7 Endonuclease I for 1 hour at 37° C., and analyzed on an agarose gel along with a positive control (Genecopoeia).

Sequential Protein Extraction and Western Blotting Analysis

Cells were harvested in 1×PBS and pelleted by centrifugation at 400×g for 5 minutes. The cell pellets were extracted via homogenization in 1% Triton lysis buffer containing protease inhibitor cocktail (PIC) (Roche), phenylmethylsulfonyl fluoride (PMSF) (Sigma), sodium orthovanadate (Na3VO4) (Sigma) and sodium fluoride (NaF) (Sigma). The Triton extracted lysates were freeze-thawed three times and ultracentrifuged at 100,000×g for 30 minutes at 4° C. The Triton-insoluble pellets were further extracted in 2% SDS lysis buffer containing PIC via boiling for 10 minutes, followed by sonication and then ultracentrifugation at 100,000×g for 30 minutes at 22° C. The protein concentrations of the Triton and SDS fractions were measured via a BCA protein assay kit (Thermo Fisher Scientific) on a plate reader. Extracted protein lysates were loaded on an SDS-PAGE gel, transferred onto a PVDF membrane (Millipore), and post-fixed in 0.4% paraformaldehyde. Membranes were blocked in a 1:1 mixture of 1×TBS and Intercept blocking buffer (Li-Cor Biosciences), followed by overnight incubation with primary antibodies diluted in a 1:1 mixture of 1×TBS-Tween and blocking buffer. The following day, secondary antibodies were added for 1 hour, and the membranes were scanned using a Li-Cor Biosciences infrared imaging system. Quantification of band intensity was done using the ImageStudio software and analysis was performed on Excel and GraphPad Prism. A detailed protocol of this procedure has been published (Stojkovska and Mazzulli, 2021).

To quantify insoluble GCase, the intensity from the soluble and insoluble fractions (using Sigma antibody G4171) was normalized to total protein obtained from the Coomassie blue stained gel of the corresponding membrane. Normalized intensities of soluble and insoluble fractions were added to obtain the total GCase signal. The % insoluble GCase was calculated by dividing the insoluble intensity by the total ×100, then expressed as fold change compared to the control lines or vehicle treated samples. The proportion of insoluble GCase in healthy wild-type cells ranged between 10-20%.

Analysis of LIMP2 Knock-Out Mice

LIMP2 knock-out mice have been previously described and characterized (Rothaug et al., 2014). Brain tissue was sequentially extracted as described for cell cultures in “Sequential protein extraction and western blotting analysis”. An additional extraction step was added for both Triton and SDS steps to avoid carry over between the fractions. Protein assay was performed by BCA, and 40 ug of total protein was loaded per well. GCase solubility was assessed using the anti-GCase antibody from Sigma (G4171), and normalized to total protein obtained from Coomassie blue stained gels of the corresponding membranes.

Live-Cell Lysosomal GCase Activity Assay

The procedure and analysis method for the activity assay has been previously described in detail (Cuddy and Mazzulli, 2021) Briefly, cells were plated in 96-well plates. One day prior to the assay, cells were treated with 1 mg/ml cascade dextran blue (Life Technologies) for 24 hours. The next day, the cells were first treated with DMSO or 200 nM bafilomycin A1 (Santa Cruz) for 1 hour at 37° C., followed by a 1 hour pulse-chase with 100 ug/ml artificial fluorescent GCase substrate, 5-(pentafluoro-benzoylamino) fluorescein di-ß-D-glucopyranoside (PFB-FDGluc) (Life Technologies), at 37° C. The fluorescence signal was measured every 30 minutes for the span of 3-4 hours on a plate reader (Ex=485 nm, Em=530 nm, for the GCase substrates; Ex=400 nm, Em=430 nm for cascade dextran blue). For the analysis, the GCase fluorescence signal was normalized to either lysosomal mass by using cascade dextran blue signal or total cell volume by quantifying CellTag 700 staining signal.

Measurement of Neuron Viability Through Neurofilament Quantification

For this assay, the same cultures used in the live cell activity were used from a 96 well plate. Following the live-cell lysosomal GCase activity assay, the cells were fixed in 4% paraformaldehyde in PBS for 15 minutes, and stained with an anti-neurofilament antibody overnight at 4° C. (refer to (Mazzulli et al., 2016b) for details). The next day, IRdye 800CW goat anti-mouse IgG secondary antibody and CellTag 700 stain were added to the wells and incubated for 1 hour, and the plate was scanned on a Li-Cor infrared imaging system.

In Vitro Whole-Cell Lysate GCase Activity Assay

The procedure and analysis method for the activity assay has been previously described in detail (Mazzulli et al., 2011). Briefly, 1% BSA and 5 ug of Triton-soluble protein lysate treated with or without conduritol-β-epoxide (CBE, an inhibitor specific for lysosomal GCase) (Millipore) were added to GCase activity assay buffer (0.25% w/v sodium Taurocholate, 0.25% TritonX-100, 1 mM EDTA, into a citrate/phosphate buffer pH 5.4) to a final volume of 100 ul in a 96-well black bottom plate. The samples were incubated with 5 mM fluorescent GCase substrate 4-methylumbelliferyl β-glucopyranoside (4-MU-Gluc) (Chem-Impex) for 30 minutes at 37° C., and the reaction was stopped using equi-volume of 1 M glycine, pH 12.5. The fluorescence signal was measured on a plate reader (Ex=365 nm, Em=445). Relative fluorescence units from CBE treated lysates were subtracted from non-CBE treated lysates to obtain the activity of GCase.

Monitoring Protein Trafficking with Endoglycosidase H (Endo H)

These methods have been described in detail previously (Cuddy and Mazzulli, 2021). To study the subcellular localization and trafficking of GCase between the ER and Golgi, we digested protein lysates with Endoglycosidase H (Endo H) (New England Biolabs). The experimental procedure was performed according to the manufacturer's instructions. Briefly, 10× Glycoprotein Denaturing buffer was added to 40 μg of protein and the reaction was boiled at 100° C. for 10 minutes. Following the denaturation, 10× GlycoBuffer 3 and Endo H enzyme were added, and the reaction was incubated at 37° C. for 2 hours. Finally, the samples were boiled at 100° C. for 10 minutes after the addition of 5×Laemmli buffer and loaded on a 10% SD S-PAGE gel, followed by western blot analysis. A positive digestion results in a downward shift in the molecular size of GCase after it is subjected to SDS-PAGE. Post-ER (70-74 kDa) and ER (55 kDa) forms of GCase were analyzed using the Endo H digested lane, and used as a measure of GCase trafficking.

Insoluble Hydrolase Analysis of Synucleinopathy Brain Tissues

Sequential protein extraction was performed on post-mortem frontal cortex brain tissues (obtained from the Northwestern University Alzheimer's disease pathology core) obtained from controls, DLB, and DLB+AD patients. We employed a 5-step extraction protocol using high salt buffer, 1% Triton X-100, 1% Triton+30% sucrose (Sigma), 1% sarkosyl (Sigma), and sarkosyl-insoluble extracts. Brain tissues were homogenized in high-salt buffer (HSB) (50 mM Tris-HCl pH 7.4, 750 mM NaCl, 10 mM NaF, 5 mM EDTA) with protease and protein phosphatase inhibitors, incubated on ice for 20 minutes and centrifuged at 100,000×g for 30 minutes at 4° C. The pellets were then re-extracted with HSB, followed by sequential extractions with 1% Triton X-100-containing HSB and 1% Triton X-100-containing HSB with 30% sucrose. The pellets were then resuspended and homogenized in 1% sarkosyl-containing HSB, rotated at 4° C. overnight and centrifuged at 100,000×g for 30 min. The resulting sarkosyl-insoluble pellets were washed once with PBS and resuspended in PBS by brief sonication. This suspension was termed the ‘sarkosyl-insoluble fraction’, which was analyzed by western blot.

Insoluble GCase Analysis of ER Microsome-Enriched Idiopathic PD Brain Tissues

ER microsomes were enriched using subcellular fractionation and the purity of the fractions have been assessed previously (Mazzulli et al., 2011). Post-mortem cingulate cortex brain tissues obtained from idiopathic PD patients were lysed and homogenized in 0.25 M sucrose buffer containing 10 mM HEPES (pH 7.4) and 0.01 M EDTA, and centrifuged at 6,800×g for 5 minutes at 4° C. to remove nuclei and unbroken cells. The extraction was repeated to wash the pellet. The final supernatants were combined and further centrifuged at 17,000×g for 10 minutes at 4° C. to remove mitochondria. Further centrifugation of the resulting supernatant at 100,000×g for 1 hour was done to pellet the ER microsome components. Sequential extraction of soluble and insoluble protein from this final pellet was performed using 1% Triton and 2% SDS lysis buffer, respectively, as described above. Insoluble fractions were analyzed via western blot.

GBA1 Mutation Genotyping of Human Brain Samples

Genomic DNA was extracted from 50 mg human brain tissue (frontal/temporal cortex) using the PureLink genomic DNA kit (Invitrogen). To amplify the GBA1 gene, 25 ng genomic DNA was used as a template for PCR using the following forward and reverse primers, respectively: 5′-TGTGTGCAAGGTCCAGGATCAG-3′ SEQ ID NO: 7 and 5′-ACCACCTAGAGGGGAAAGTG-3′ SEQ ID NO: 8. The PCR products were run on a 1% agarose gel to confirm amplification of the GBA1 gene and to rule out accidental amplification of the GBA1 pseudogene (GBAP). Sequencing of the most common GBA1 mutations (L444P, N370S, E326K) was performed using primers listed in the Key Resource Table, and analysis was done using the Snapgene software.

Electron Microscopy (EM) Analysis

Neurons were fixed in 2.5% glutaraldehyde (Electron Microscopy Sciences) in PBS for 30 minutes, and then washed six times with PBS for 5 minutes. Cells were post-fixed with 1% osmium tetroxide (OsO4) (Electron Microscopy Sciences) in PBS for 1 hour, and then washed three times with H2O. Next, cells were dehydrated with ethanol (twice with 50% ethanol for 5 minutes, then twice with 70% ethanol for 10 minutes) and stained with 1% uranyl acetate (Electron Microscopy Sciences) in 70% ethanol for 45 minutes. Cells were further dehydrated with ethanol (once with 70% ethanol, then twice with 90% ethanol for 10 minutes, then three times with 100% ethanol for 10 minutes). To evaporate the ethanol, 100% ethanol was mixed at a 1:1 ratio with an LX112 resin mix containing LX112 (Ladd Research Industries), DDSA (Electron Microscopy Sciences), and NMA (Electron Microscopy Sciences), and added to the cells for 1 hour with the lid off. Next, LX112 resin mix alone was added to the cells for 1 hour. Finally, cells were embedded by combining LX112 resin mix with DMP-30 (Electron Microscopy Sciences) and allowing the resin to solidify overnight at 60° C. Samples were then thin sectioned (˜70 nm width) on a UC7 ultramicrotome, as a service provided by the Northwestern University Center for Advanced Microscopy, and viewed on a FEI Tecnai Spirit G2 TEM. For each cell that was imaged via EM, all clearly defined ER regions were analyzed for both length and area using the ‘Measure’ function in ImageJ. The length and ER area (in micrometers) of each individual ER profile were plotted on a graph using GraphPad Prism.

Proximity Ligation Assay (PLA)

H4 cells or iPSC neurons plated on coverglass were fixed with 4% paraformaldehyde for 20 minutes at RT. The cells were then washed three times with PBS, permeabilized with 0.3% Triton X-100 in PBS for 1 hour at 4° C., and then blocked with 2% BSA (Roche) and 5% NGS (Jackson Immuno Research) in Triton-PBS for 30 minutes at RT. Interaction between α-syn and ER chaperones was determined via the Duolink In Situ Red Starter Kit Mouse/Rabbit (Sigma). Cells were incubated with primary antibodies (anti-α-synuclein syn211, anti-α-synuclein C20, anti-CANX, anti-GRP94) overnight followed by a 1 hour, 37° C. incubation with the PLA probes (secondary antibodies labeled with distinct oligonucleotides) provided in the kit. If the PLA probes are in proximity, the addition of ligase and DNA polymerase results in rolling circle amplification. For the ligation step, cells were washed twice with 1×wash buffer A (provided in the PLA kit) for 5 minutes each, and incubated with ligase (1:40 dilution) for 30 minutes at 37° C. For the amplification step, cells were washed twice with 1×wash buffer A for 2 minutes each, and incubated with polymerase diluted (1:80) in an amplification buffer containing fluorescently labeled complementary nucleotide probes for 100 minutes at 37° C. After the incubation, the cells were washed twice with 1× wash buffer B (provided in the PLA kit) for 10 minutes each followed by a quick wash with 0.01× wash buffer B. Finally, the cover glass was mounted onto microscope slides with DAPI mounting media. All images were obtained on a Leica confocal microscope (PLA excitation: 488 nm). Counting of PLA particles was automated using ImageJ using the ‘Measure’ function. To determine the level of interaction, the number of PLA particles were normalized to the number of nuclei within an acquired field of view.

Super-Resolution Structured Illumination Microscopy (SIM)

iPSC neurons were plated on coverglass, fixed in 4% paraformaldehyde for 15 minutes, permeabilized with 0.1% Triton X-100 in PBS for 30 minutes, and blocked with 2% BSA and 5% NGS in Triton-PBS for 30 minutes. Primary antibodies (anti-α-synuclein syn211, anti-PDIA6) were added overnight, followed by incubation with secondary antibodies (Alexa Fluor 488 Goat anti-mouse IgG and Alexa Fluor 568 Goat anti-rabbit IgG) for 2 hours. The cells were then washed three times with Triton-PBS and mounted onto microscope slides with DAPI mounting media. Imaging was performed using an oil immersion 100× objective lens on a Nikon structured illumination microscope (N-SIM) at the Northwestern University Center for Advanced Microscopy. Images were captured and slices were reconstructed using the Nikon NIS Elements program.

ER Microsome-Enrichment of iPSC-Derived Neurons

ER microsomes were enriched using subcellular fractionation. SNCA-3X and healthy and isogenic control iPSC-derived neurons were gently homogenized in sucrose HEPES buffer (SHB). The homogenate was centrifuged at 6,800×g for 5 minutes at 4° C. to remove nuclei and unbroken cells. Following removal of the supernatant (S1), the extraction was repeated using SHB buffer and the second supernatant (S2) was combined with S1. The combined supernatants (S1+S2) were further centrifuged at 17,000×g for 10 minutes at 4° C. to remove mitochondria. Further centrifugation of the resulting supernatant (S3) at 100,000×g for 1 hour at 4° C. removes the cytosolic components (supernatant S4), leaving the ER microsomes in the third and final pellet, termed P3. The P3 pellet was extracted in 1% Triton lysis buffer and analyzed by western blot.

Semi-Quantitative RT-PCR Analysis of XBP1 mRNA

Using cDNA as the template, human XBP1 mRNA was detected using PCR primers (forward: TTACGAGAGAAAACTCATGGCC SEQ ID NO: 9; reverse: GGGTCCAAGTTGTCCAGAATGC SEQ ID NO: 10) specific for both spliced (S; product size 263 bp) and unspliced (U; product size 289) isoforms. The PCR product was analyzed on an agarose gel along with a brefeldin A positive control.

ER Stress Induction of H4 Cells or iPSC Neurons

To induce ER stress and activate the UPR, H4 cells or iPSC neurons were treated with 30 nM thapsigargin (Tg) (Sigma) or 50 ng/ml brefeldin A (BFA) for 24 hours prior to harvesting, and analysis of mRNA and/or protein expression of known ER stress markers was performed.

Proteasomal Inhibition of iPSC Neurons

iPSC neurons were treated with epoxomicin (Fisher) for 24 hours to inhibit the proteasome. Analysis of GCase levels following treatment was performed via western blot analysis. Successful proteasomal inhibition was confirmed by blotting for ubiquitin.

Assessment of Calnexin Activity by Concanavalin-A Pulldown

H4 neuroglioma cells were treated with vehicle or 25 uM Diltiazem (Sigma) for 4 days, harvested, and extracted in 0.3% CHAPS lysis buffer (0.3% CHAPS, 40 mM HEPES pH 7.4, 120 mM NaCl, 1 mM EDTA, 10% v/v glycerol). For pulldown of total N-linked glycosylated proteins, 1500 μg lysate was mixed with 20 μg/ml biotinylated Concanavalin A (CON-A) (Vector Laboratories) and the reaction mixture was incubated overnight at 4° C. under gentle rotation. To recover CON-A bound proteins, 25 μl neutrAvidin agarose beads (Thermo Fisher Scientific) were added to the reaction mix and samples were incubated at 4° C. for 1 hour. The beads were collected by centrifugation at 2500×g for 2 min, followed by three washes with lysis buffer. N-glycosylated proteins were eluted by boiling the samples at 95° C. for 10 min in 2×Laemmli sample buffer. Samples were analyzed by western blot for calnexin (CANX), GCase, and total N-glycosylated proteins by Coomassie brilliant blue staining. Calnexin activity was indirectly assessed by quantifying CANX levels in CON-A pulled down samples.

Ryanodine Receptor RyR3 Knockdown Using shRNA Constructs

MISSION shRNA sequences targeting human RyR3 were obtained from Sigma and tested for efficiency in HEK293T cells by quantitative RT-PCR analysis using RyR3 TaqMan probes. Clone ID #TRCN0000053349 was found to achieve the most efficient knock-down and was therefore used in subsequent experiments. This lentiviral plasmid was used to create lentiviral particles, as described under “Lentiviral preparation and transduction of H4 cells and iPSC neurons”.

Generation of the GBA1 L444P Plasmid for Overexpression in iPSC Neurons

GBA1 L444P was generated by site-directed mutagenesis (SDM) of the pER4-wild-type GBA1 lentiviral plasmid. Mutagenesis primers (5′-GTGCCACTGCGTCCGGGTCGTTCTTCTGA-3′ SEQ ID NO: 11 and 5′-TCAGAAGAACGACCCGGACGCAGTGGCAC-3′ SEQ ID NO: 12) were created using the Agilent tool. SDM was performed using the materials and procedures from the QuikChange XL Site-Directed Mutagenesis Kit (Agilent). The L444P mutation was confirmed by sequencing. The pER4 GBA1 L444P plasmid was then packaged into lentiviral particles, as described under “Lentiviral preparation and transduction of H4 cells and iPSC neurons”.

Lentiviral Preparation and Transduction of H4 Cells and iPSC Neurons

In combination with a packaging vector (psPAX2) and an envelope vector (VSV-G), lentiviral plasmids were used to create lentiviral particles by transfecting HEK293FT cells using X-tremeGENE transfection reagent (Roche). The lentiviral particles were collected and concentrated using Lenti-X concentrator (Clontech) and titered with a HIV1-p24 antigen ELISA kit (Zeptometrix). For RyR3 shRNA knock-down and ykt6-CS overexpression, H4 cells and iPSC neurons were infected at a multiplicity of infection (MOI) of 3-5 and were harvested 5 days or 2 weeks post-infection, respectively. For GBA1 L444P overexpression, neurons were infected at MOI of 1 and were harvested 2 weeks post-infection.

Treatment of H4 Cells or iPSC Neurons with Small Molecules

H4 cells or iPSC neurons were treated with vehicle or either 25 mM diltiazem (DILT) (Sigma), 40 mM dantrolene (DANT) (Sigma) or 1 mm DHBP (Sigma), and media was changed every other day for the duration of the experiment. For the combination compound treatments, H4 cells or iPSC neurons were treated with vehicle, 25 uM DILT, 5 nM farnesyl transferase inhibitor (FTI) (gift of Peter T. Lansbury, Jr.), or FTI+DILT combination, and media was changed every other day for the duration of the experiment. For the combination of genetic manipulation and compound treatment (e.g. RyR3 KD+FTI, DILT+ykt6-CS), cells were infected and treatment was begun at the same time, with media change every other day for the duration of the experiment.

Statistical Analysis

Analyzed data was plotted and tested for statistical significance using the GraphPad Prism software. Statistical significance between two samples was determined using a paired or unpaired t-test with Welch's correction. For more than two samples, significance was determined using a one-way ANOVA with Tukey's post-hoc test or Dunnett's T3 multiple comparisons test, as indicated in the figure legend. A p-value of <0.05 was considered to be significant (*p<0.05, **p<0.01, ***p<0.001, ****p<0.0001). For each quantification, the type of error bar used is specified in the figure legends.

### Example 3. Impaired Glucose Flux Through the Hexosamine Biosynthetic Pathway Induces Lysosomal Dysfunction and Proteostasis Failure in Parkinson's Disease

Disrupted glucose metabolism and misfolded protein are characteristics of age-related neurodegenerative disorders including Parkinson's disease (PD), however their mechanistic linkage is largely unexplored. The hexosamine biosynthetic pathway (HBP) utilizes glucose and uridine-5′-triphosphate (UTP) to generate N-glycan precursors required for folding and maturation of proteins in the endoplasmic reticulum. Targeted metabolomic analysis revealed that PD patient neurons accumulate HBP metabolites including glucose and UTP, while N-glycan synthesis rates are concomitantly decreased. Reduced glucose flux occurred by selective reduction of the rate-limiting HBP enzyme GFPT2, mediated by disrupted signaling between the unfolded protein response (UPR) and the HBP. Reduced N-glycosylation caused immature lysosomal hydrolases to misfold and accumulate, while accelerating glucose flux through the HBP rescued lysosomes and reduced pathological α-synuclein. Thus, the HBP integrates glucose metabolism with lysosomal function, and its failure occurs by uncoupling of the UPR-HBP axis. Our findings offer novel methods to restore proteostasis by HBP enhancement.

### INTRODUCTION

Deficits in protein quality control machinery occur during the aging process and are thought to play a major role in the etiology of chronic neurodegenerative diseases (Hipp et al., 2019). The accumulation of insoluble protein aggregates is a cardinal feature of all age-related neurodegenerative diseases and directly indicates a disruption in protein homeostasis (or proteostasis) (Labbadia and Morimoto, 2015). Parkinson's disease (PD) is characterized by aberrant aggregation of α-synuclein (α-syn) protein in the form of Lewy bodies and Lewy neurites that histopathologically define the disease (Spillantini et al., 1997). Genetic studies also implicate lysosomal components as key contributors to PD pathogenesis (Chang et al., 2017; Klein and Mazzulli, 2018; Robak et al., 2017). For example, loss of function mutations in GBA1 that encodes β-glucocerebrosidase (GCase) are among the strongest risk factors for PD (Sidransky and Lopez, 2012). Previous work from our group and others showed that reduced GCase activity results in lysosomal dysfunction and initiates pathogenic α-syn aggregation (Fredriksen et al., 2021; Mazzulli et al., 2011; Zunke et al., 2018). Multiple studies have shown that GCase activity is also reduced in PD patients that do not carry GBA1 mutations (reviewed in (Stojkovska et al., 2017)), suggesting that loss of GCase function plays a common role in sporadic synucleinopathies. We previously showed that α-syn accumulation disrupts the trafficking and activity of wild-type GCase and other lysosomal hydrolases, creating a pathogenic self-propagating cycle of a-syn accumulation (Chung et al., 2013; Cuddy et al., 2019; Mazzulli et al., 2011; Mazzulli et al., 2016). Multiple groups have shown that α-syn disrupts ER-Golgi trafficking in different PD models (Cooper et al., 2006; Gitler et al., 2008; Oaks et al., 2013; Thayanidhi et al., 2010), and we recently found that a-syn directly interferes with ER function and the unfolded protein response (UPR) causing the misfolding and aggregation of GCase (Stojkovska et al., 2022). Collectively, these studies indicate that α-syn interferes with multiple branches of the proteostasis network, however our understanding of the mechanisms that contribute to the chronic accumulation of protein aggregates is incomplete.

Parallel to an age-related decline in proteostasis, glucose utilization in the brain dramatically declines during normal aging (Goyal et al., 2017) and may confer susceptibility to age-related neurodegeneration. It is well established that dramatic changes in glucose utilization occur in the brains of living PD patients and correlate with the severity of clinical symptoms (Firbank et al., 2017; Hu et al., 2000; Pappata et al., 2011; Peppard et al., 1992). Cognitive dysfunction often occurs late in the clinical course of PD, and altered glucose metabolism can be observed in cortical regions of non-demented PD patients (Peppard et al., 1992), suggesting it may play an early causal role in neurodegeneration. Consistent with this, highly reproducible metabolic changes have been documented within specific neural networks of symptomatic PD brain (termed PD-related patterns or PDRP) (Niethammer and Eidelberg, 2012). PDRP signals are elevated in prodromal stages of PD, including REM behavior sleep disorder, and have a predicative value in phenoconversion (Holtbernd et al., 2014). Metabolic imaging studies of hemiparkinsonsism, a condition where newly diagnosed PD patients exhibit unilateral symptoms, showed increased uptake and accumulation of the glucose tracer (fluorodeoxyglucose, ¹⁸F-FDG) in the pre-symptomatic hemisphere suggesting that metabolic perturbations precede dysfunction (Tang et al., 2010). In addition to imaging studies in living patients, gene expression microarray studies of PD postmortem tissue have identified reduced expression of gene networks involving glucose metabolism and other bioenergetic pathways in the earliest stages of disease, suggesting that reduced glucose utilization precedes neuronal death (Zheng et al., 2010). Studies in animal models have shown that enhancing glucose flux protects against dopaminergic cell death and reduces α-syn pathology, although the mechanisms are not completely understood (Cai et al., 2019). These data suggest a close association between perturbed glucose metabolism and PD pathogenesis. However, it is unclear if impaired glucose flux plays an active role in driving disease pathogenesis, or occurs non-specifically as an indirect consequence of other primary pathological processes.

Glucose flux through the hexosamine biosynthetic pathway (HBP) is essential for generating the high-energy sugar-nucleotide donor uridine-diphosphate-N-acetylglucosamine (UDP-GlcNAc), which provides the main glycan source for protein N-glycosylation (Denzel and Antebi, 2015). The HBP begins with the conversion of obligatory intermediate fructose-6-phosphate (F-6-P) from the initial steps of glycolysis, and glutamine to form glucosamine-6-phosphate by the rate-limiting enzyme, glutamine:F-6-P transaminase (GFPT), ultimately forming UDP-GlcNAc. Two isozymes of GFPT exist (GFPT1 and GFPT2) and are expressed from distinct genes with variable expression patterns. In the central nervous system, GFPT2 (GFAT2, EC 2.6.1.16) is the predominant isoform (Oki et al., 1999). UDP-GlcNAc is utilized in the committed step of N-glycosylation by dolichyl-phosphate-N-acetyl-glucosamine-phosphotransferase-1 (DPAGT1), which is the tunicamycin target (Dong et al., 2018; Heifetz et al., 1979). The addition of N-glycans is required for the proper folding and trafficking of proteins that mature through the ER-Golgi pathway including lysosomal hydrolases (Hebert et al., 2014). In vivo studies have shown that enhancing glycosylation through activation of the HBP can improve proteostasis and extend lifespan in worms (Denzel et al., 2014; Wang et al., 2014), however the mechanism has yet to be fully delineated. The importance of the HBP and N-glycosylation in proteostasis is also demonstrated by genetic diseases caused by mutations in key HBP enzymes. For example, a rare congenital disorder of glycosylation caused by loss-of-function mutations in DPATG1 primarily affects the nervous system and is clinically characterized by hypokinesia, cognitive impairment, and microcephaly (Carrera et al., 2012; Freeze et al., 2015; Wu et al., 2003). Here, we explored the mechanistic link between glucose utilization by the HBP and proteostasis failure in PD. We found that the HBP integrates glucose metabolism with lysosomal function, and that impaired glucose flux through the HBP is a causative factor in driving proteostasis failure in PD.

Results

Reduced Levels of N-Glycosylated Proteins in PD Models Occurs Through Deficits in N-Glycan Synthesis

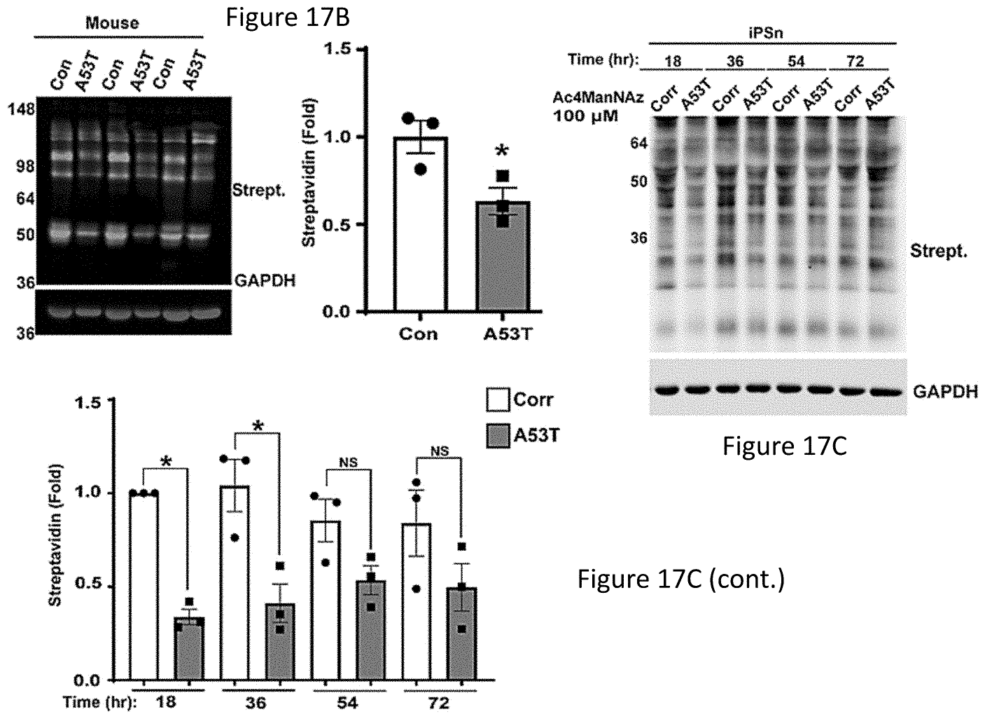

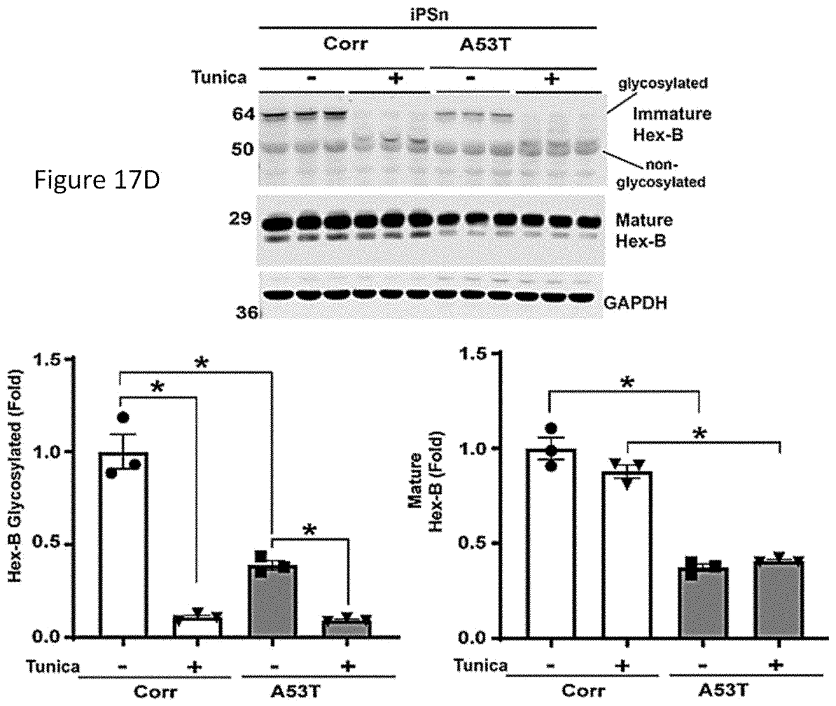

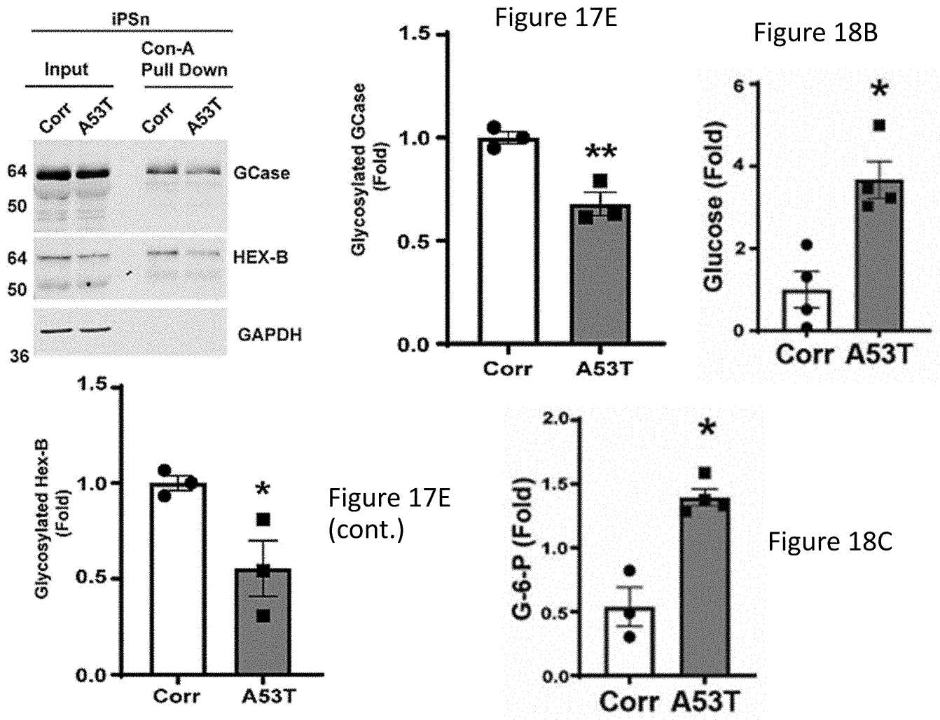

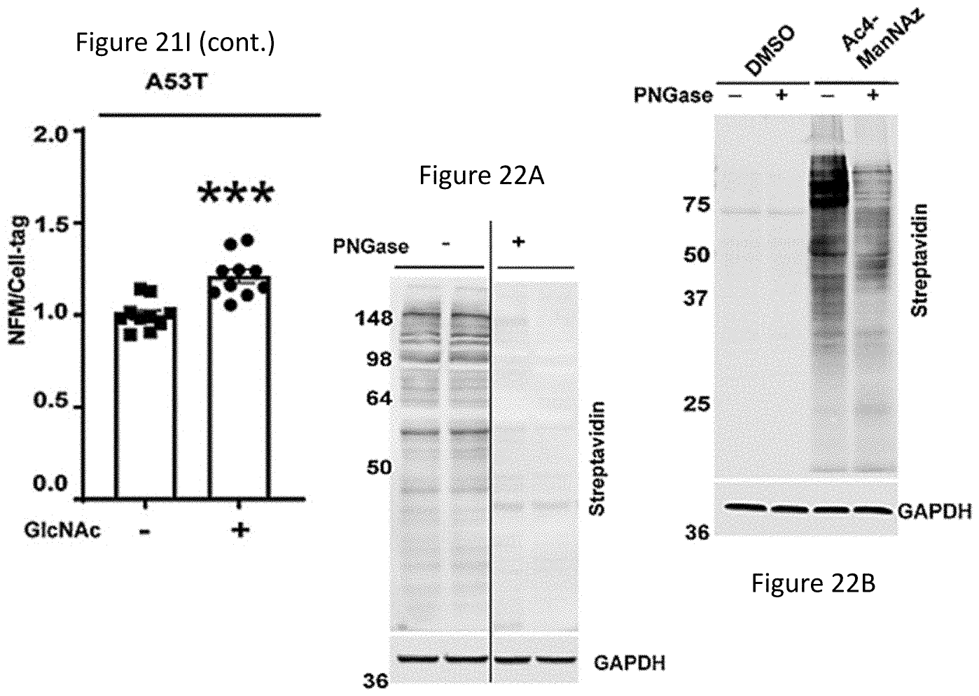

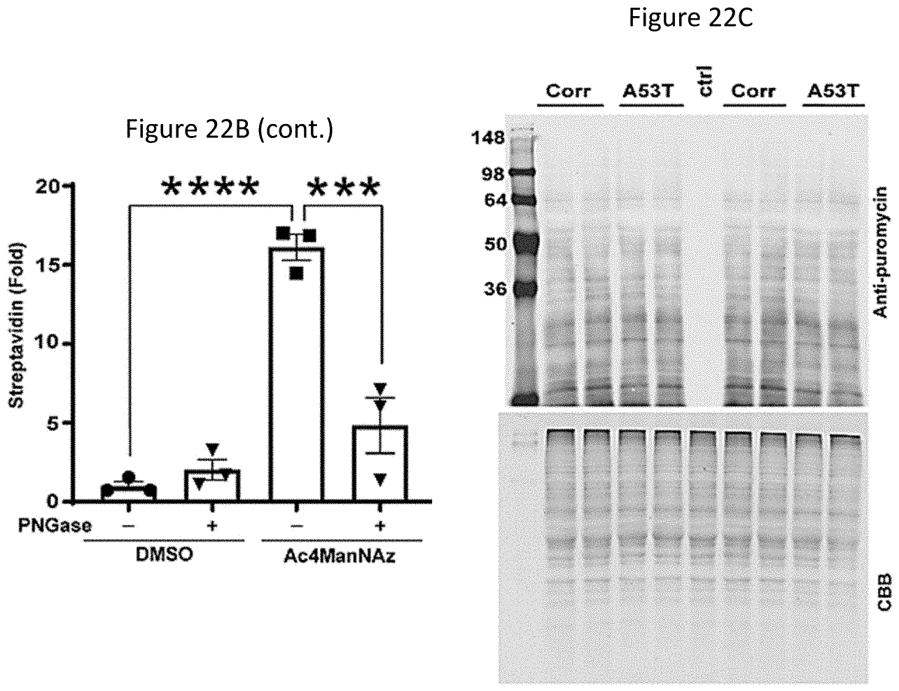

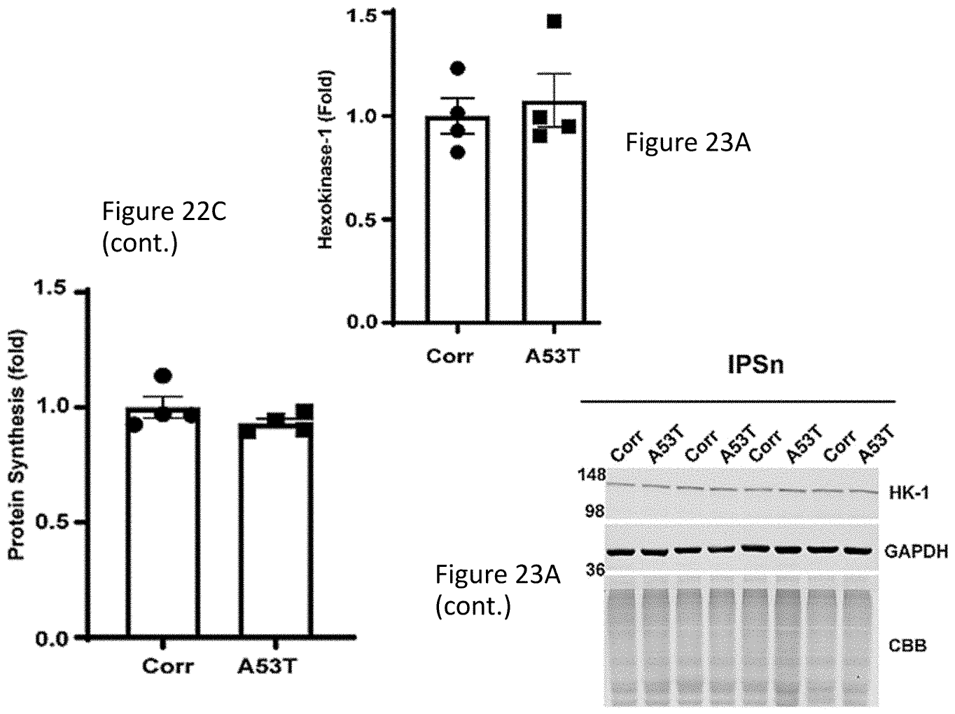

To assess changes in N-glycosylation, we measured the total levels of protein N-glycans in PD midbrain cultures derived from iPSCs (iPSn) that express the disease-causing A53T mutation in a-syn (Soldner et al., 2011). Previous analysis of this model showed that pathological a-syn aggregates develop by 60 days in culture, followed by the accumulation of immature GCase and lysosomal dysfunction at day 75, then neurodegeneration after day 100 (Cuddy et al., 2019). We directly measured N-glycosylated proteins using concanavalin A (Con A) conjugated to biotin, a lectin that is specific for N-glycans. The specificity of Con-A for binding N-glycans was confirmed by digestion of cell line extracts (H4 neuroglioma) with peptide-N-glycosidase F (PNGase) that removes N-linked oligosaccharides from glycoproteins. Western blot analysis using Con A-biotin indicated a reduction in the N-glycosylated proteins in PNGase-treated lysates indicating specific detection of N-glycans (FIG. 22A). Analysis of protein lysates from A53T iPSn with Con A-biotin indicated a dramatic decline in N-glycosylated proteins at day 90 compared to isogenic corrected controls (FIG. 17A). Although treatment of control iPSn with tunicamycin reduced N-glycans by ˜50% as expected, tunicamycin had no effect on A53T iPSn (FIG. 17A). This suggests that defects in N-glycosylation occur within the ER at the level of the tunicamycin target, DPAGT1, or on upstream HBP enzymes involved in the synthesis of N-glycan precursors.

We next determined if N-glycosylation was perturbed in vivo by analyzing brain lysates from A53T α-syn transgenic mice (Giasson et al., 2002). A53T mice develop insoluble a-syn inclusions throughout the neuraxis and neurological dysfunction that occurs in an age-dependent manner (Giasson et al., 2002; Tsika et al., 2010). We found that N-glycosylated proteins were dramatically reduced in brains from symptomatic A53T mice compared to age-matched non-transgenic controls (FIG. 17B). Taken together these results indicate that reduced N-glycosylation occurs in synucleinopathy models in vitro and in vivo.

To gain insight into the mechanism of N-glycan reduction, we measured the rate of N-glycan synthesis in PD culture models. The incorporation of oligosaccharides on proteins was examined by pulse-labeling living cultures with an azide-tagged mannose, N-azidoacetylmannosamine-tetraacylated (Ac4-ManNAz). Specific detection of proteins that incorporate Ac4-ManNAz is achieved through a Staudinger reaction with phosphine-biotin, followed by detection with streptavidin (Saxon and Bertozzi, 2000). We first established the specificity of Ac4-ManNAz labeling in differentiated human neuroblastoma (SH-SY5Y) cells by digesting protein lysates with PNGase, which resulted in a ˜50% reduction in Ac4-ManNAz signal (FIG. 22B). We next determined the levels of incorporated N-glycans in midbrain cultures and found a reduction at 18 and 36 hours post-labeling in A53T iPSn (FIG. 17C). Quantification of global protein translation showed that A53T iPSn were not different than controls, indicating that the reduction of incorporated N-glycans was not a reflection of changes in protein synthesis (FIG. 22C). This indicates that reduced levels of N-glycosylated protein occurs through a decline in the rate of N-glycan synthesis in the ER of A53T patient neurons.

Since we previously found that immature, non-glycosylated lysosomal hydrolases accumulate in PD iPSn (Mazzulli et al., 2011; Mazzulli et al., 2016; Stojkovska et al., 2022), we next measured the amount of N-glycosylation that occurs specifically on lysosomal hydrolases. We first measured the beta subunit of hexosaminidase that comprises the B isozyme (Hex B), since its glycosylation and maturation can be easily followed by molecular weight changes on SDS-PAGE gels (Hasilik and Neufeld, 1980). Treatment of control cells with tunicamycin blocked Hex B glycosylation, indicated by a reduction in the ˜63 kDa form and a concomitant increase in the immature, 55 kDa non-glycosylated form in isogenic control iPSn (FIG. 17D). Analysis of untreated A53T iPSn showed a 60% decrease in the levels of the 63 kDa glycosylated Hex B compared to controls, and a reduced response to tunicamycin (FIG. 17D). We confirmed this result by precipitating N-glycosylated proteins from iPSn lysates with Con A-biotin, followed by immunoblotting for GCase and Hex-B. This revealed a reduction in the amount of glycosylated GCase and Hex-B in A53T iPSn compared to isogenic controls (FIG. 17E). These data indicate that reduced N-glycosylation of GCase and Hex B occurs in A53T iPSn, compromising hydrolase maturation in PD.

Metabolomic Analysis Indicates Disruptions in the HBP in PD iPSn

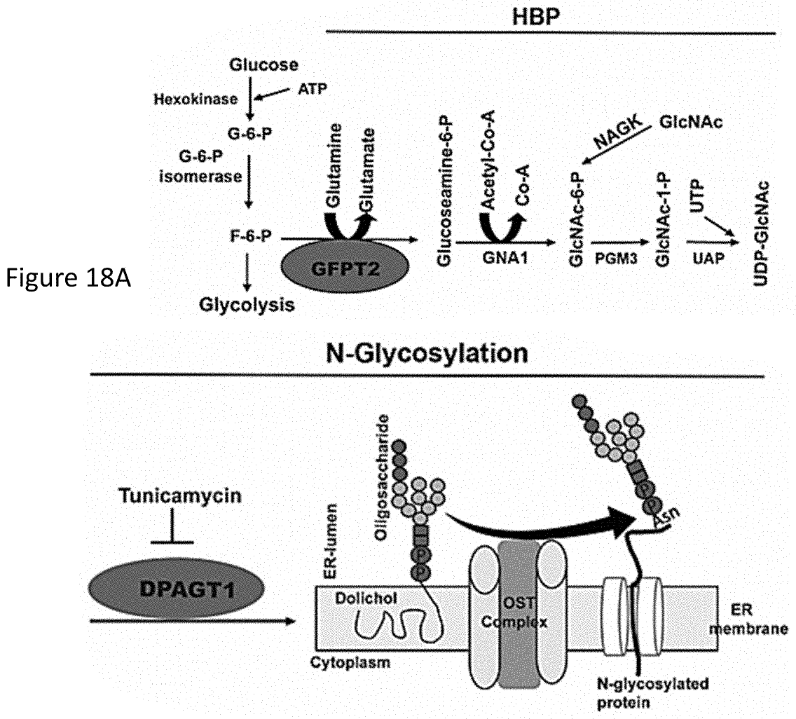

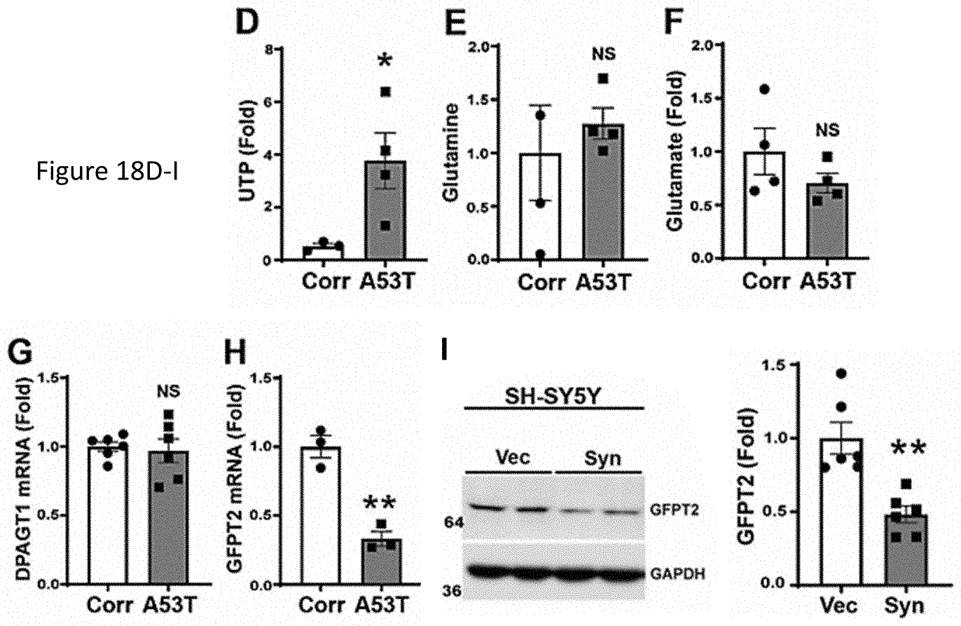

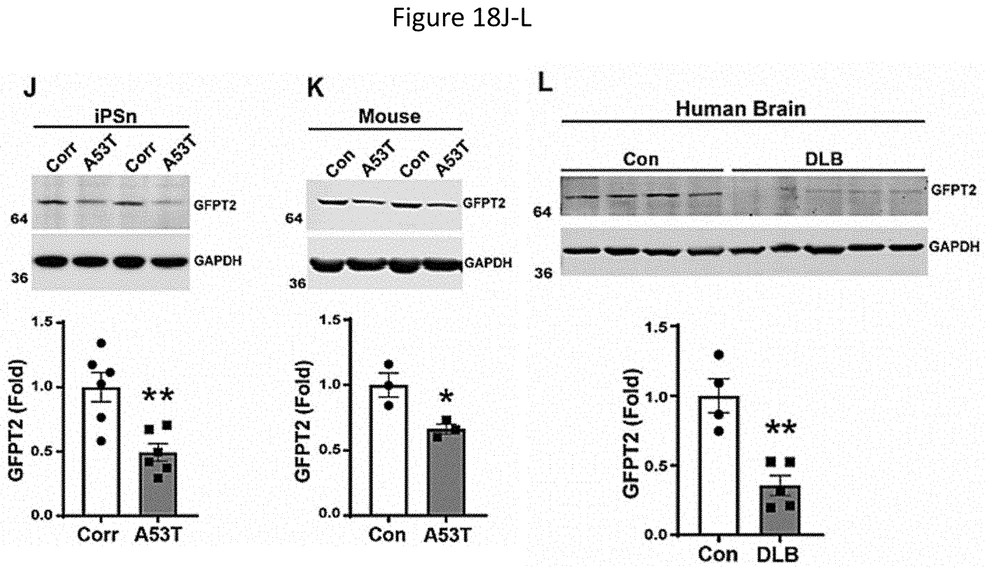

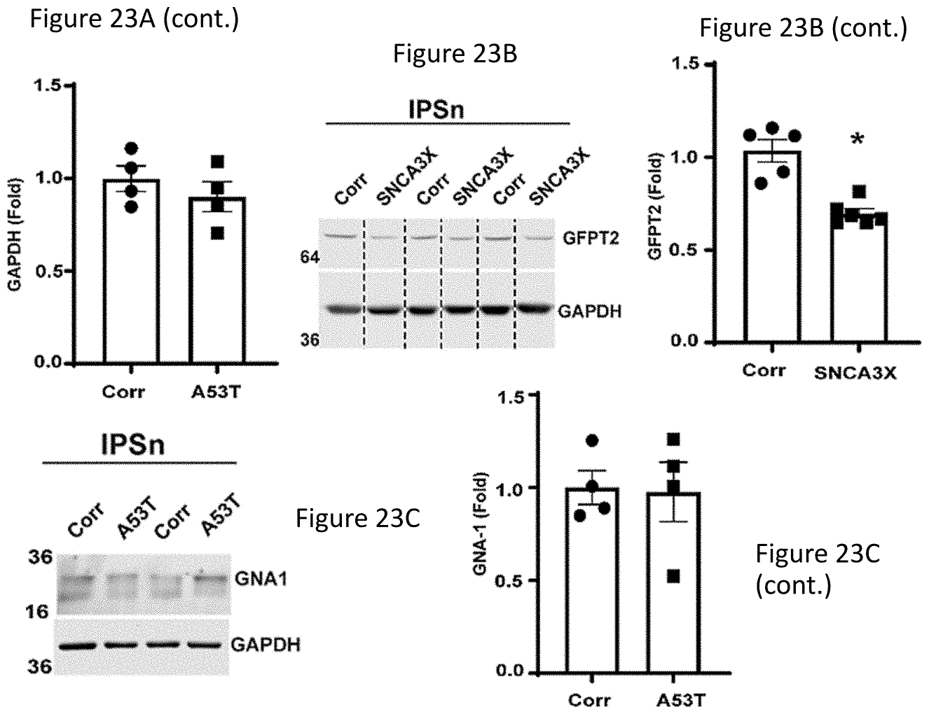

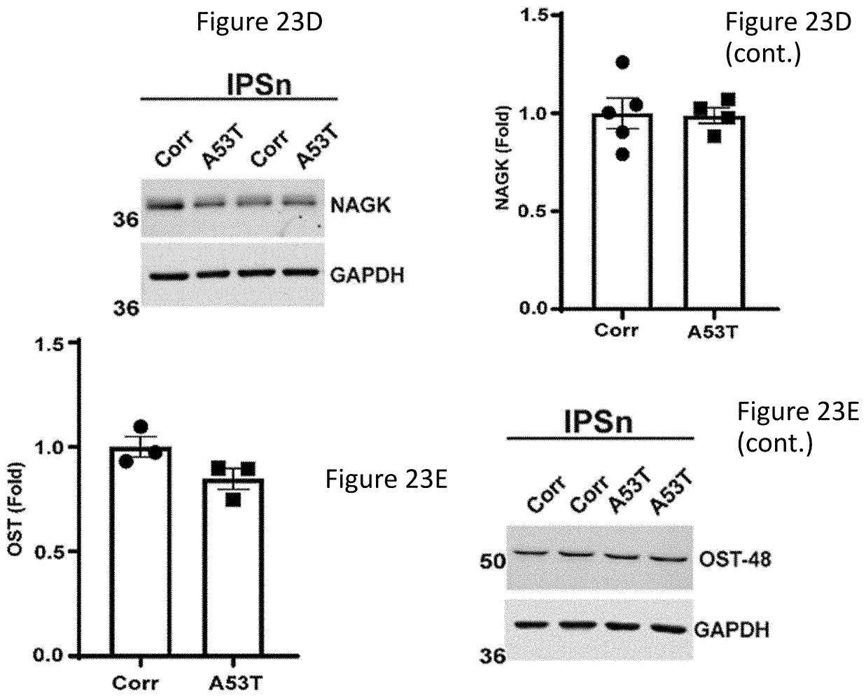

We next considered the possibility that reduced N-glycan synthesis was a result of low intracellular glucose or impaired turnover within the initial steps of glycolysis that provide essential HBP substrates (FIG. 18A). However, targeted metabolomic analysis of A53T iPSn instead indicated a ˜4 fold accumulation of intracellular glucose and glucose-6-phosphate (G-6-P), and no change was observed in hexokinase protein levels (FIG. 18B, C; FIG. 23A). Furthermore, uridine 5′-triphosphate (UTP), which is utilized downstream in the HBP by UDP-N-acetylhexosamine pyrophosphorylase (UAP1) to generate UPD-GlcNAc, was also elevated by ˜4 fold (FIG. 18D). No change was found in other HBP metabolites including glutamine or glutamate (FIG. 18E, F), which may be due to their involvement in multiple metabolic pathways beyond the HBP. Next, we measured the expression levels of the most critical enzymes in the HBP including the rate-limiting GFPT2, and DPAGT1. While the mRNA of DPAGT1 was not changed, GFPT2 mRNA was dramatically reduced in A53T iPSn compared to isogenic controls (FIG. 18G, H). We confirmed that GFPT2 protein was reduced by western blot analysis, a finding that was reproducible in all a-syn accumulation models examined, including SH-SY5Y cells, A53T iPSn, and A53T transgenic mice (FIG. 18I-K). GFPT2 reduction was also observed in a separate PD patient line that was previously characterized and accumulates pathological a-syn through a triplication in the SNCA genomic region (SNCA-3x) (Stojkovska et al., 2022) (FIG. 23B). The relevance of these findings to human disease was further confirmed by the analysis of DLB patient brain, which showed a ˜70% reduction in GFPT2 protein (FIG. 18L). Measurement of other enzymes in the HBP, including glucosamine 6-phosphate N-acetyltransferase (GNA1) and N-acetyl-D-glucosamine kinase (NAGK), showed no change by western blot analysis in iPSn (FIG. 23C, D). Finally, we measured the levels of a critical component of oligosaccharyltransferase complex, OST48, which catalyzes the en-bloc transfer of the dolichol-linked oligosaccharide onto proteins in the ER (Kelleher et al., 1992). Western blot analysis indicated no change in OST48 in iPSn (FIG. 23E) suggesting that N-glycosylation downstream of DPAGT1 is not perturbed in PD iPSn, and is consistent with a lack of tunicamycin response in A53T iPSn (FIG. 17A). Together, these data suggest that impaired N-glycan synthesis in PD iPSn occurs specifically through reduced GFPT2.

GFPT2 Expression is Upregulated in Response to Tunicamycin and Disrupted in PD Patient Neurons

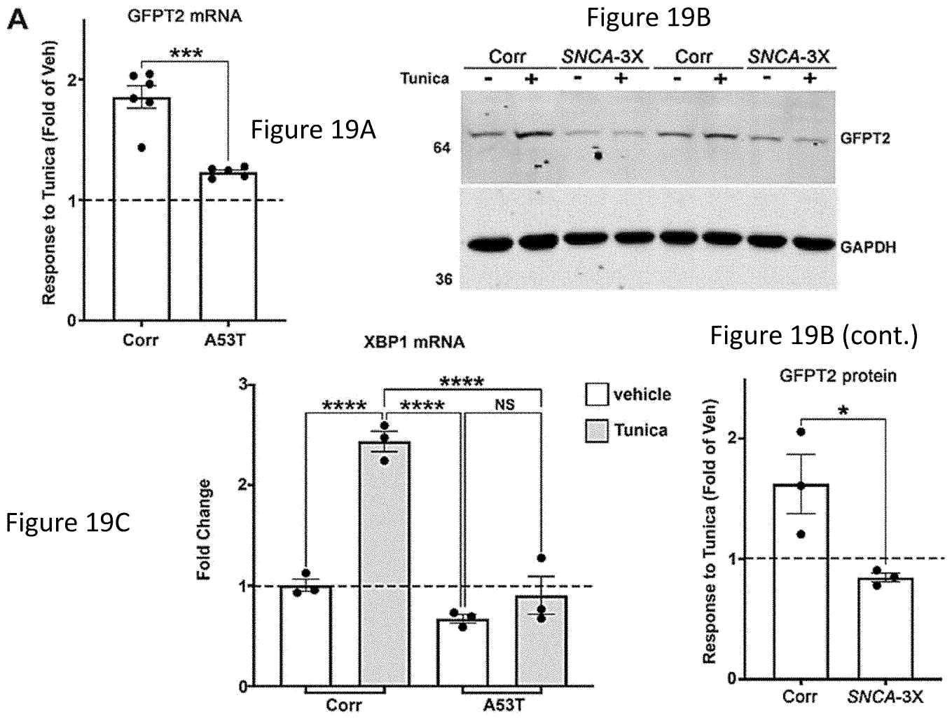

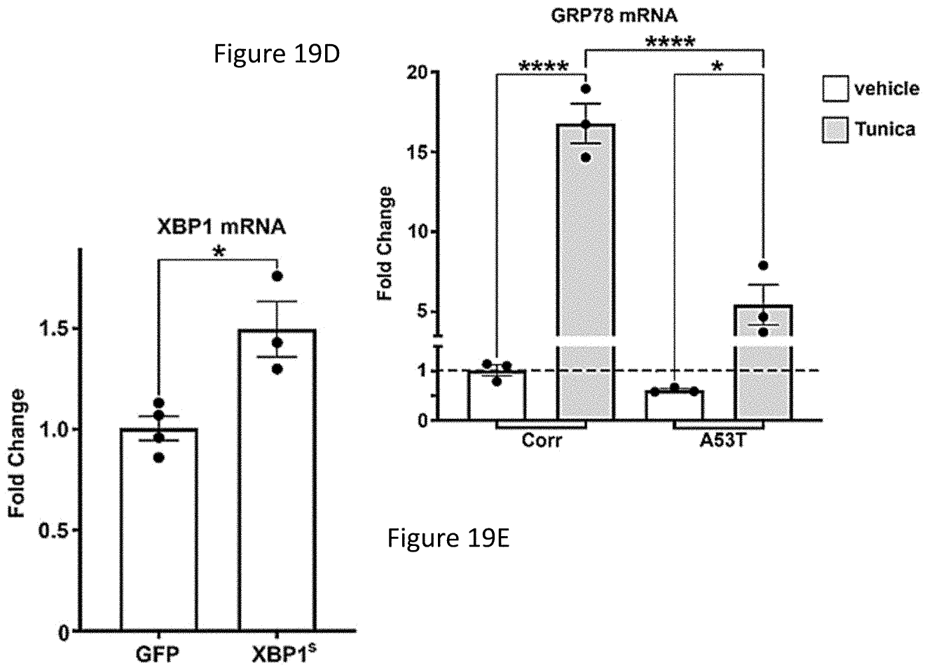

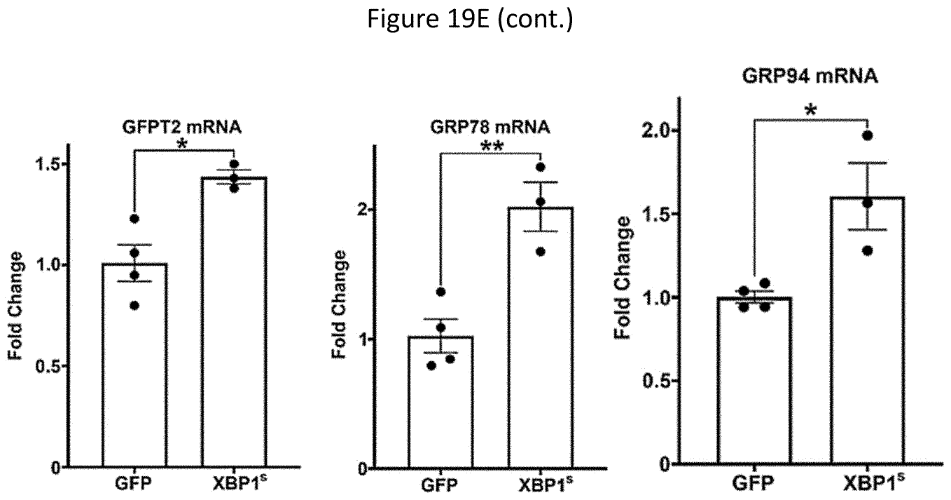

We next sought to determine the mechanism of GFPT2 reduction. Since GFPT2 mRNA was reduced in A53T iPSn, we hypothesized that transcriptional regulation could be perturbed. Previous studies showed that GFAT1, the non-neuronal isozyme of GFPT2 that is expressed on a separate gene, is transcriptionally controlled by the unfolded protein response (UPR) (Wang et al., 2014). Additionally, our previous work showed that the UPR is dysfunctional in PD iPSn (Stojkovska et al., 2022), providing a potential explanation for reduced GFPT2 levels. To examine the relationship between the UPR and GFPT2 in PD models, we first tested if triggering the UPR could induce the expression of GFPT2 in wild-type (wt) midbrain neurons similar to that of non-neuronal GFAT1. Treatment of wt iPSn with tunicamycin induced a ˜2-fold upregulation of GFPT2 mRNA, suggesting that the neuronal HBP is also under regulatory control by the UPR (FIG. 19A). Conversely, A53T iPSn showed a significantly reduced response to tunicamycin of only ˜1.2 fold compared to controls (FIG. 19A). These findings were confirmed by GFPT2 western blot of PD patient iPSn that express SNCA-3x and isogenic controls. Tunicamycin induced a 60% elevation of GFPT2 protein in isogenic controls but had no effect in SNCA-3x iPSn (FIG. 19B). Examination of other UPR-responsive transcripts, including XBP1 and its downstream transcriptional target GRP78 also revealed that A53T iPSn were less responsive to tunicamycin (FIG. 19C, D). These data indicate that GFPT2 reduction may occur from dysfunctional signal transduction in the UPR. To test this, we attempted to rescue GFPT2 levels in A53T iPSn by expressing the active, spliced form of XBP1 (XBP1^{S}), the transcription factor that induces expression of UPR-associated chaperones including GRP78 and GRP94. Overexpression of XBP1^{S} in A53T iPSn induced a 1.5-2 fold elevation of GFPT2 mRNA, but also other downstream UPR targets including GRP78 and GRP94 (FIG. 19E). Collectively, this suggests that GFPT2 reduction in PD occurs through a failure to trigger upstream UPR signaling pathways upon tunicamycin induced stress, and is consistent with previous findings (Stojkovska et al., 2022).

Genetic Enhancement of the HBP Rescues Lysosomal Dysfunction and Reduces Pathogenic a-Syn

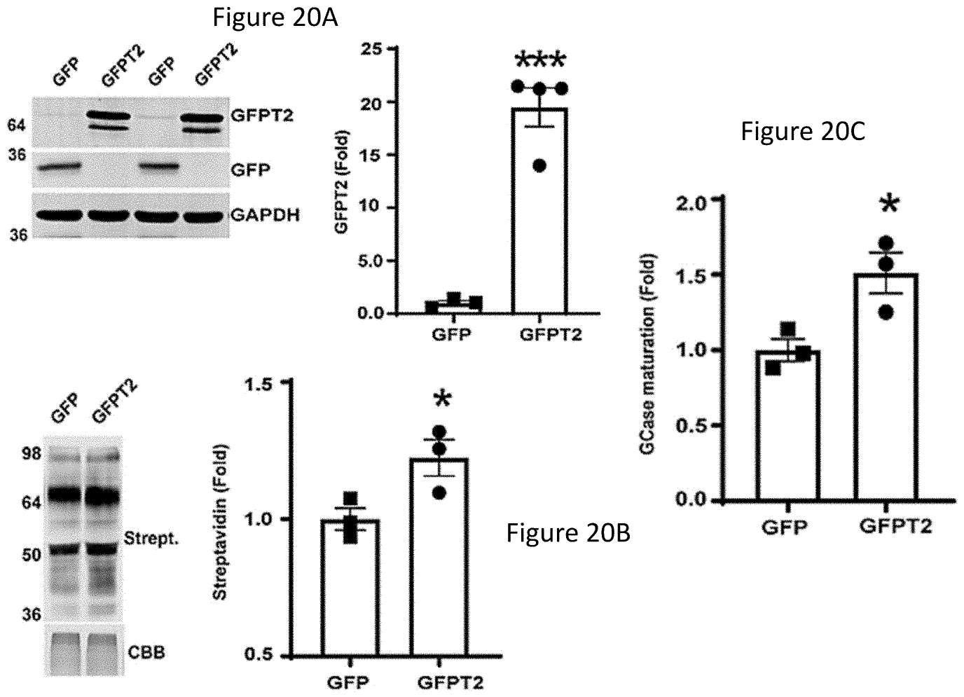

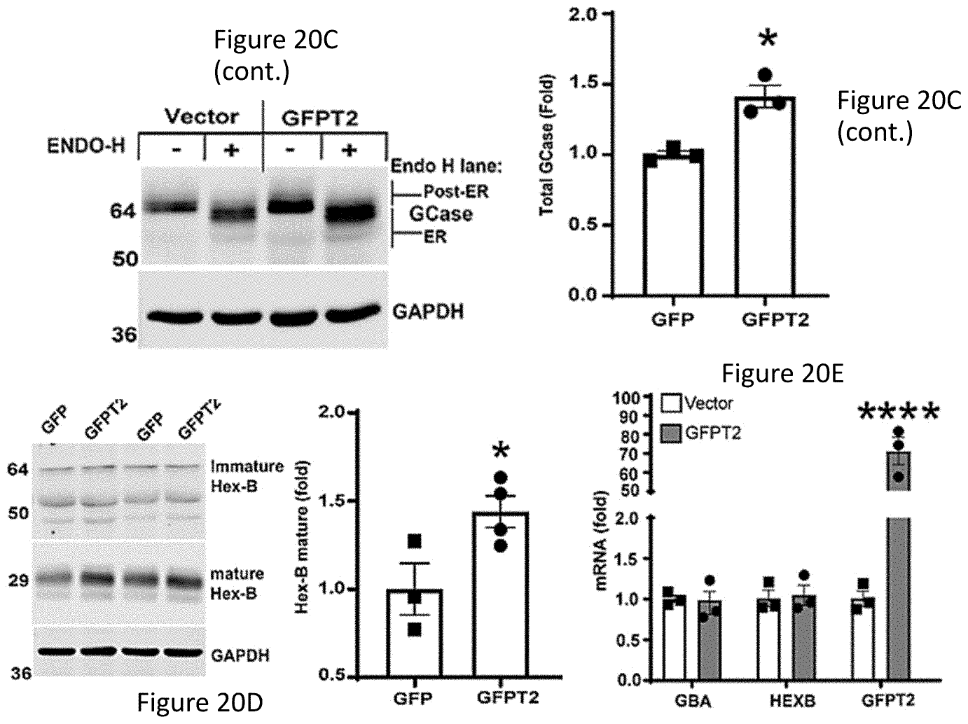

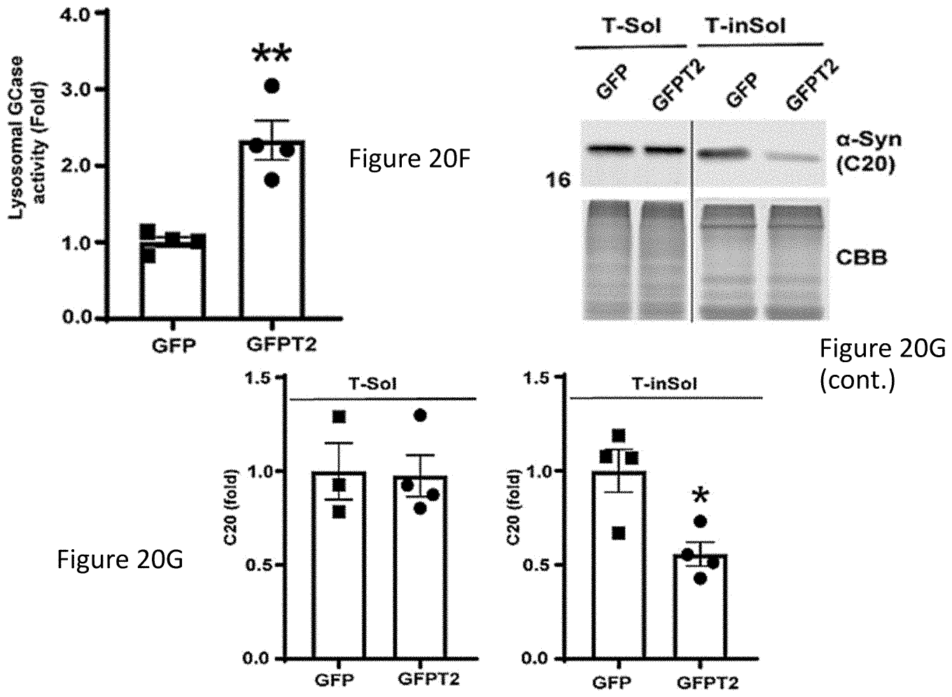

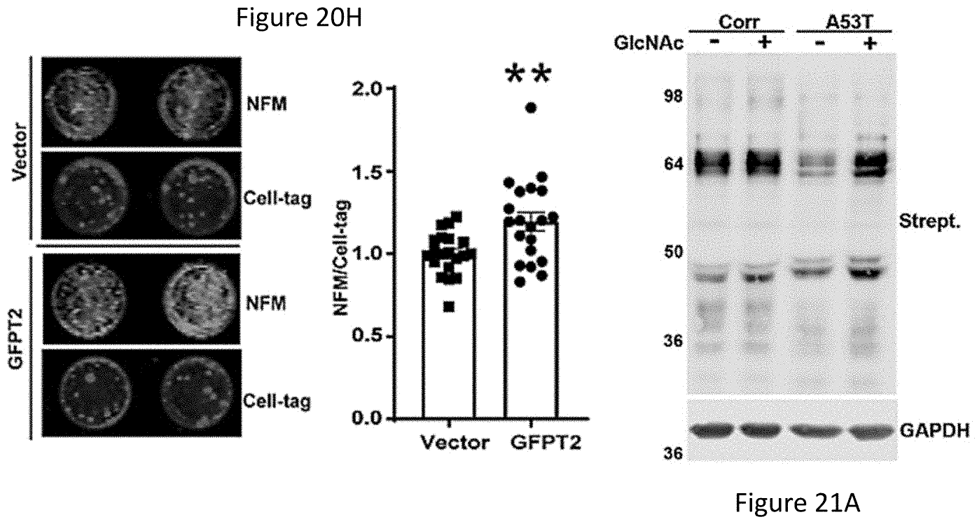

To directly examine the relationship between the HBP and lysosomal function in PD, we next determined if directly restoring GFPT2 protein could rescue N-glycosylation and downstream lysosomal phenotypes. To increase GFPT2, PD patient iPSn were transduced with lentivirus expressing GFPT2 under a phospho-glycerate kinase (PGK) promoter. Western blot confirmed that GFPT2 protein levels were increased in A53T iPSn compared to those infected with a control GFP-expressing lentivirus (FIG. 20A). Measurement of total N-glycosylated proteins by Con A revealed a significant elevation by GFPT2 overexpression (FIG. 20B). To determine if this elevation was sufficient to improve the maturation of lysosomal hydrolases, GCase was analyzed by western blot. We found a significant increase in the levels of glycosylated GCase migrating at 62 kDa, and an elevation in the mature (post-ER) forms migrating above 62 kDa (FIG. 20C). The effect on maturation was confirmed by digesting lysates with endoglycosidase H (Endo H), an enzyme that selectively cleaves protein-linked glycans in the ER, but not glycans that are modified in the Golgi. This revealed an increase in the levels of Endo H-resistant GCase upon GFPT2 overexpression (FIG. 20C). We next assessed the effect of GFPT2 on Hex B maturation by western blot. Similar to GCase, we found that GFPT2 overexpression could increase mature Hex B (29 kDa) by nearly 50% (FIG. 19D). mRNA analysis of GCase and Hex B showed no change by GFPT2 overexpression, suggesting that improved maturity occurred post-transcriptionally (FIG. 19E). To determine whether GFPT2 could elevate lysosomal function, we performed a live-cell GCase activity assay that can discriminate between activity that occurs in lysosomal and non-lysosomal compartments of neurons (Mazzulli et al., 2016). We found that lysosomal GCase activity was significantly elevated by GFPT2, indicating that GCase targeting to lysosomal compartments was improved (FIG. 19F). Importantly, this degree of lysosomal enhancement was sufficient to reduce insoluble, pathological forms of α-syn in A53T iPSn shown by sequential extraction/western blot analysis (FIG. 19G). Finally, we tested if GFPT2 overexpression could improve neuron viability, using an established neurofilament immunostaining assay that reflects total neurite content in cultures. This revealed a significant improvement in neurite content within the GFPT2-treated A53T iPSn compared to vector controls (FIG. 19H).

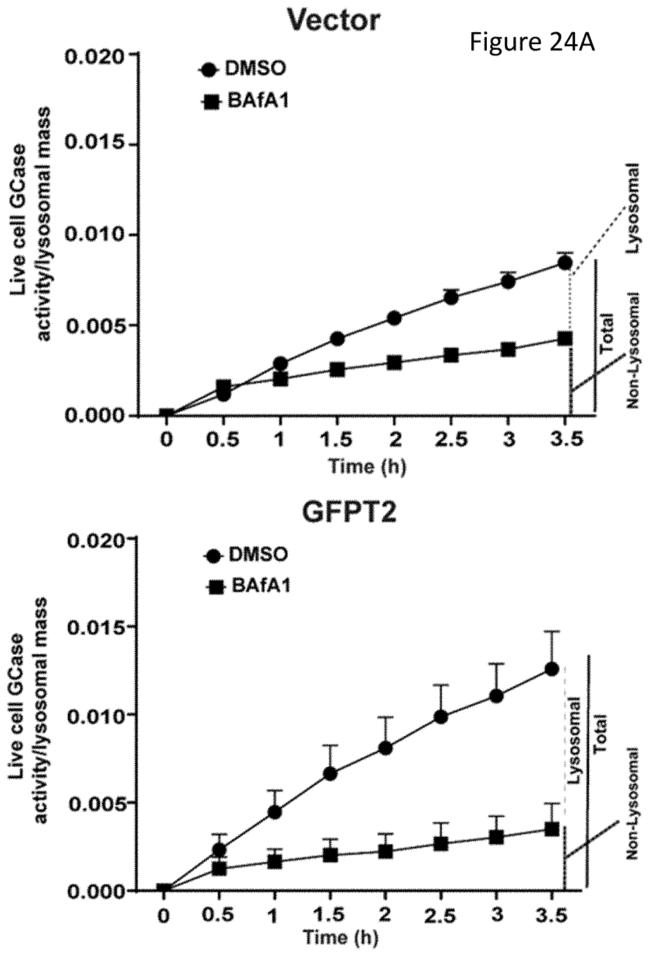

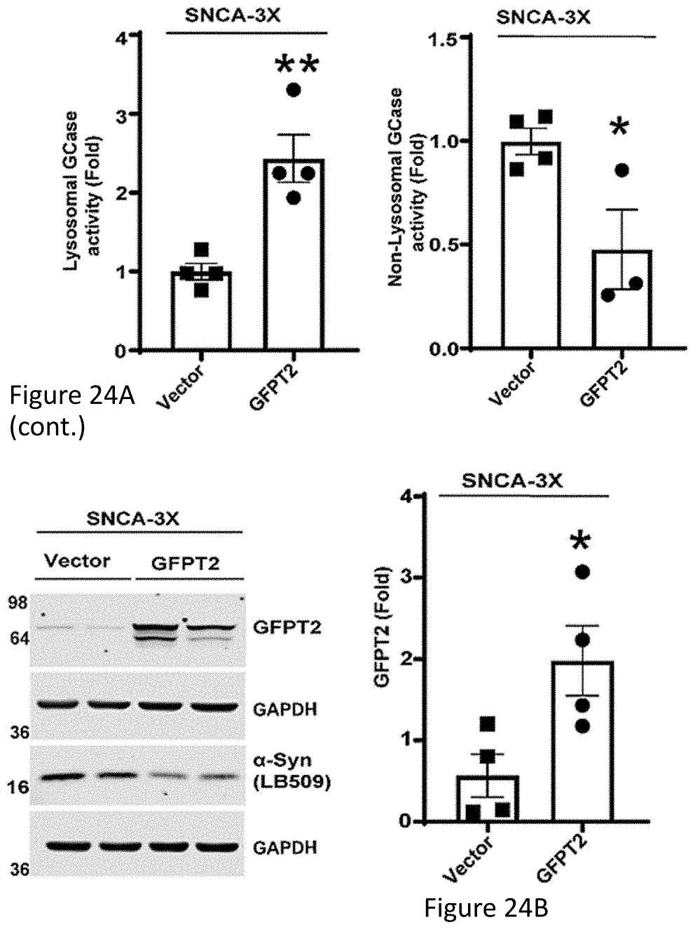

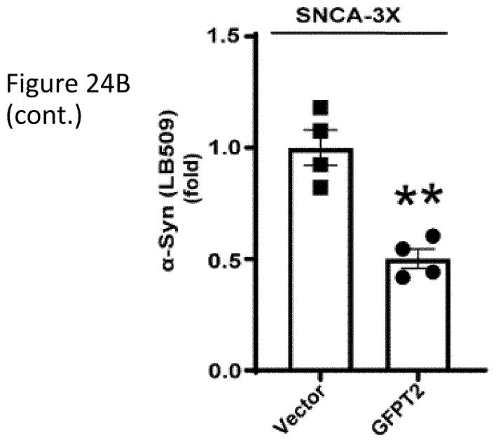

To determine if GFPT2 overexpression could also improve lysosomal function in a distinct iPSn patient line that accumulates wild-type a-syn, we treated SNCA-3x lines with lenti-GFPT2. Similar to A53T iPSn, GFPT2 elevated lysosomal GCase activity while decreasing non-lysosomal activity (FIG. 24A). This effect was sufficient to reduce pathological a-syn by 50% (FIG. 24B). Taken together, these data uncover a previously unrecognized role for the HBP in mediating lysosomal function and proteostasis in human midbrain neurons.

Pharmacological Enhancement of the HBP Restores Proteostasis in PD Patient iPSn.

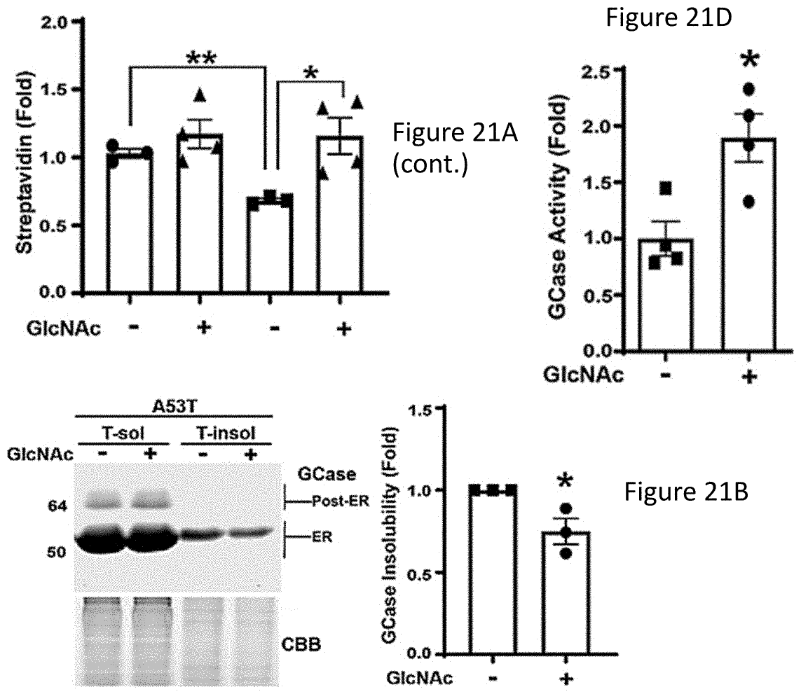

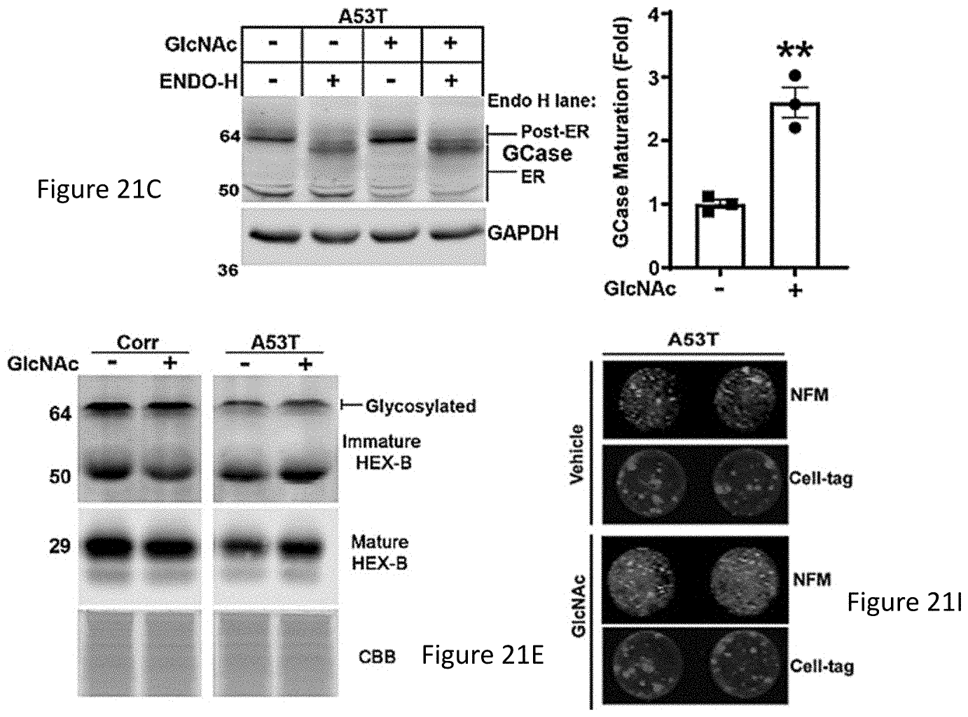

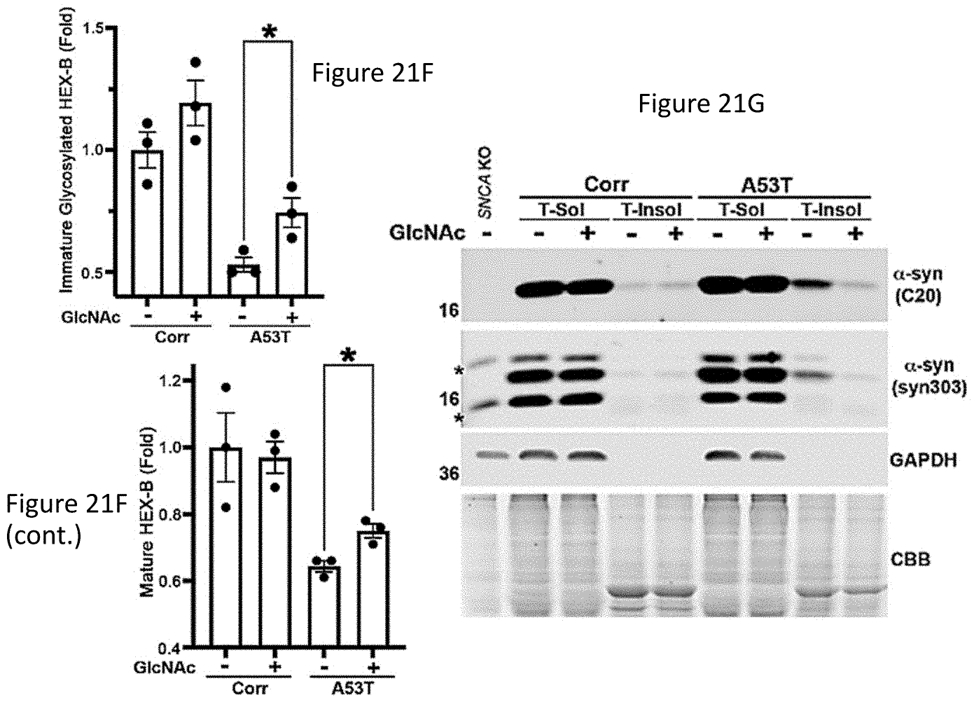

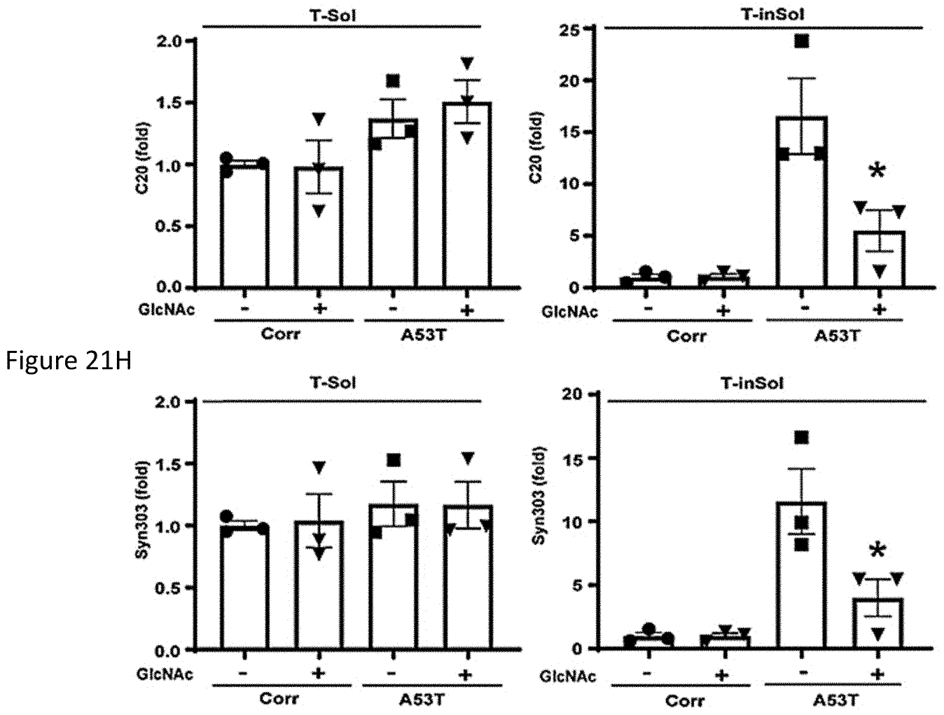

Having demonstrated that GFPT2 can rescue lysosomal function in PD patient neurons, we next determined if N-glycan synthesis could be rescued by the addition of HBP intermediates that act downstream of GFPT2. N-glycosylation can also be elevated through supplementation of N-acetylglucosamine (GlcNAc) by NAGK-mediated conversion into GlcNAc-6-P, which enters the HBP downstream of GFPT2 and ultimately forms UPD-GlcNAc (FIG. 18A). Previous studies have shown that exogenous addition of GlcNAc is utilized by the HBP and can improve N-glycosylation in cancer models (Abdel Rahman et al., 2013; Johswich et al., 2014; Wellen et al., 2010). It also can extend the lifespan of C. elegans (Denzel et al., 2014). Since we found that NAGK levels were not altered in PD iPSn (FIG. 23D), we treated cultures with GlcNAc and measured the levels of N-glycosylated proteins with Con A. We found that GlcNAc treatment completely restored the levels of N-glycosylation to control levels (FIG. 21A). Since we previously found that PD neurons accumulated misfolded, aggregated GCase in the ER (Stojkovska et al., 2022), we next determined if GlcNAc could improve its folding and solubility. Sequential extraction/western blot analysis of GCase showed reduction in the proportion of insoluble GCase by GlcNAc (FIG. 21B), and a 2-fold increase in the levels of mature, soluble GCase (FIG. 21C). GlcNAc improved the amount of properly folded GCase since activity levels increased by nearly 2-fold (FIG. 21D). GlcNAc supplementation also enhanced the glycosylated and mature forms of Hex B (FIG. 21E, F). GlcNAc treatment resulted in a dramatic ˜75% reduction in pathological α-syn and improved neuron viability (FIG. 21G, I). Together, these results show that HBP stimulation by GlcNAc supplementation can enhance N-glycosylation, lysosomal hydrolase trafficking, and activity, which in turn can reverse deleterious effects of α-syn in A53T iPSn.

### DISCUSSION

Although changes in glucose metabolism and protein aggregation are two well-established features of age-related neurodegenerative disorders, their mechanistic connection was not fully understood. Reduced glucose utilization in the brain is associated with a decline in neural function and disease severity in PD patients, but it was previously unclear whether these changes are a cause or consequence of disease. We find that a decline in the HBP leads to proteostasis failure, resulting in lysosomal dysfunction and protein aggregation. Improving the HBP through GFPT2 or GlcNAc can restore lysosomal function, demonstrating a direct relationship between glucose flux and proteostasis in PD. Impaired glucose utilization is evident in PD iPSn by the accumulation of intracellular glucose, G-6-P, and UTP that is utilized by the HBP to generate precursors required for N-glycosylation. The HBP is an off-shoot of glycolysis that normally utilizes a portion of the total intracellular glucose. However, a chronic reduction of GFPT2 activity over time could result in dramatic increase of glucose, as we observe in our long-term midbrain cultures aged for 90 days. Furthermore, the HBP can increase glucose flux under periods of stress to maintain proteostasis, including activation of the unfolded protein response in the ER (Wang et al., 2014). Under proteomic stress, it is likely that chronic GFPT2 reduction in PD neurons causes dramatic increase in glucose levels due to the lack of conversion into N-glycan precursors.

Our data indicates that under physiological conditions, the HBP integrates metabolic information with the UPR and lysosomes through altering intracellular glucose flux. Alterations in glucose flux through the HBP directly influence protein folding, trafficking, and lysosomal clearance pathways to maintain proteostasis. In PD, this balance is severely perturbed by reduced glucose flux through the HBP. This was demonstrated by rescue experiments, where stimulating the HBP improved N-glycosylation, lysosomal enzyme trafficking and activity, and reduced toxic a-syn aggregation (FIGS. 4 and 5). Perturbed protein trafficking is thought to be a key pathological process in PD, and our data suggest that enhancing the HBP alone can rescue this phenotype. Of critical importance is that HBP stimulation was able to improve the lysosomal activity of GCase, given its link to PD and DLB as an established genetic risk factor. Furthermore, reduced activity of wild-type GCase appears to be a common feature of synucleinopathies (Mazzulli et al., 2011). Reduced GCase folding in the ER occurs as a result of GCase mutations as in the case of Gaucher disease, but also by α-syn accumulation. Our previous study indicated that wild-type GCase accumulates and forms aggregates in the ER compartment, thereby reducing lysosomal function (Stojkovska et al., 2022). Importantly, GlcNAc treatment improved GCase solubility and function in PD iPSn. Our findings offer a novel method to enhance the folding and stability of lysosomal hydrolases, which could be used as future therapies to enhance lysosomal function and reduce toxic protein aggregates.

We found that reduced N-glycan synthesis occurred through a reduction in the rate limiting enzyme, GFPT2. The reduction of both mRNA and protein indicates that the effect is likely due to transcriptional reduction or reduced RNA stability, although more studies are required to delineate the mechanism. Previous studies showed that the HBP can be activated by the UPR through transcriptional upregulation of GFAT1. Similarly, we found that triggering the UPR through tunicamycin treatment stimulated GFPT2 expression in controls, but not in PD neurons (FIG. 20). This is consistent with previous work demonstrating that misfolded proteins fail to elicit a UPR response in PD (Stojkovska et al., 2022). Furthermore, overexpression of XBP1s could improve GFPT2 expression, as well as other UPR targets including GRP78 and GRP94 (FIG. 20E). Collectively our studies suggest that GFPT2 reduction occurs through failure of a transcriptional response when exposed to stress induced by protein misfolding, resulting in lysosomal dysfunction and augmented α-syn accumulation.

Taken together, these findings highlight the fundamental biological role of HBP in protein N-glycosylation and proteostasis, and indicate that HBP failure plays an important role in neurodegeneration. Future work aimed at increasing the activity of GFPT2 or enhancing flux downstream in the pathway with GlcNAc or potent derivatives may provide a novel class of lysosomal enhancers to combat protein aggregation. Since altered glucose metabolism and protein aggregation are features of many neurodegenerative diseases, HBP enhancers may provide benefit in several neurodegenerative disorders. They may also be useful in slowing physiological aging by promoting proteostasis.

### Example 4—Supplemental Material for Example 3

Methods

### I. EXPERIMENTAL MODEL AND SUBJECT DETAILS

H4, Human Neuroglioma Cell Culture

Human H4 neuroglioma cells were maintained in Optimem media containing 5% fetal bovine serum (FBS), 200 μg/ml Geneticin and Hygromycin, and 1% penicillin/streptomycin as described previously (Mazzulli et al., 2011). These cells express α-syn under the control of a tetracycline-responsive promoter, which can be turned off by the addition of 1 μg/ml doxycycline (DOX) in the media.

SH-SY5Y Cell Culture

Vector and α-syn expressing SH-SY5Y cells were generated as described previously (Cuddy et al., 2019). These cells were cultured in DMEM supplemented with 10% FBS, 1% penicillin/streptomycin and 200 M G418. Differentiation was induced with all trans-retinoic acid (10 M) for 5 days.

iPS Cell Culture and Neuronal Differentiation

iPS cell culture and differentiation into midbrain dopaminergic was done as previously published protocol (Mazzulli et al., 2016). Briefly, Human iPSCs were cultured on matrigel coated 6 well plates in mTeSR1 media. Human iPS lines harbouring A53T α-syn and matching isogenic corrected lines were generously gifted by Dr. R. Jaenisch (Whitehead Institute of MIT) and extensively characterized previously (Soldner et al., 2011). iPS lines having SNCA gene triplication were previously described and extensively characterized (Mazzulli et al., 2016; Stojkovska et al., 2022). iPSC differentiation into midbrain DA neurons was achieved using a mixture of growth factors for 40 days (Kriks et al., 2011). Neurons were maintained in neurobasal media (Thermofisher Scientific, #21103-049) containing NeuroCult SM1 supplement (Stem Cell Technologies, Inc. #05711) and 1% penicillin/streptomycin until used for experiments. Each batch of neurons was subjected to stringent quality control and were analyzed for maturation using the location of α-synuclein into synapse, by colocalization with synapsin by immunohistochemistry. The ratio of biii-Tubulin/GAPDH was used to assess the efficiency of differentiation between batches.

Control and Transgenic Alpha-Synuclein Mouse Lines

Synucleinopathy mouse model that expresses human A53T α-syn driven by the prion promoter (PrP) were previously described (Giasson et al., 2002). Brain samples were harvested from 10-14 old symptomatic mice as described in the figure legends from pathological regions including cerebellum and spinal cord.

### II. Method Details

### IIA: Biochemistry and Molecular Biology

Removal of N-Linked Oligosaccharides from Glycoproteins by PNGase F

PNGase F was purchased from New England Biolabs (NEB-P0704s). Triton extracted cell lysates were subjected to PNGase digestion following the manufacturer's protocol. Briefly, 20 μg of lysate was mixed with 1 μl of Glycoprotein Denaturing Buffer (10×) and milliQ water to make a 10 μl total reaction volume. The proteins were denatured by boiling the samples at 100° C. for 10 minutes. Samples were then chilled on ice for 10 secs, followed by addition of 2 μl GlycoBuffer 2 (10×), 2 μl 10% NP-40 and 6 μl H2O. PNGase F (1 μl) was added and mixed gently. The reaction mix was incubated at 37° C. for 1 hour followed by western analysis.

Metabolic Labeling for Measurement of N-Glycan Incorporation into N-Glycosylated Proteins.

Neurons were labeled with Ac4 ManNAz (Sigma-900917-100 μm). This was followed by harvesting of neurons at 18, 36, 54, and 72 h. The cells were then extracted in triton lysis buffer and protein assay was performed using DC™ Protein Assay Kit (Bio-Rad). The lysates were subjected to Biotin-phosphine reaction. In a reaction volume of 50 μl cell lysate, biotin phosphine (Cayman chemical-13581-50 M) was added and were incubated at room temperature overnight in a thermomixer. The reaction mix was subjected to western blot analysis using streptavidin-IRDye 800 conjugated detection reagent to detect biotinylated (N-glycosylated proteins).

Immunoprecipitation of N-Glycosylated Proteins

To a 500 μl neuronal lysate (1 μg/μl), 20 g/ml biotinylated Concanavalin (Con-A) was added, and the reaction mixture was incubated overnight at 4° C. rotating end over end. NeutrAvidin agarose beads (29204, ThermoFisher, Scientific-25 l) were used to recover Con-A bound proteins at 4° C. for 1 h. The mixture was spun at 2500×g for 2 min to collect the beads. The beads were washed with PBS 3 times followed by elution of N-glycosylated proteins in 2×SDS buffer by boiling the samples at 95° C. for 10 min.

Measurement of Protein Synthesis with SUrface SEnsing of Translation (Sunset) Assay.

Midbrain neurons were treated with puromycin (5 μg/mL) for 1 hr and harvested in cold PBS by centrifugation at 400×g for 5 minutes. The cell pellets were subjected to homogenization in RIPA buffer (10 mM Tris/Cl pH 7.5, 150 mM NaCl, 5 mM EDTA, 0.1% SDS, 1% Triton-100 and 1% deoxycholate) and subjected to centrifugation at 21,000×g for 20 min at 4° C. The RIPA extracted lysates were incubated in ice-water slurry on a rocker for 30 minutes and then freeze thawed three times followed by ultracentrifugation at 100,000×g for 30 minutes at 4° C. The protein concentrations was measured by DC™ Protein Assay Kit (Bio-Rad). Lysates were subjected to SDS-PAGE and western blot analysis using anti-puromycin antibody.

Targeted Metabolomics

Midbrain neurons cultured in a 6 well format were placed on ice and quickly washed with ice-cold 0.9% NaCl. The plates were then placed on dry ice, followed by addition of 1 ml/well of 80% methanol extraction mix, containing 17-isotopically labeled internal standards (Cambridge Isotope Laboratories). Cells were thoroughly scrapped and transferred to pre-chilled Eppendorf tubes. The tubes were vortexed for 10 min at 4° C. The samples were spun down at 21,000×g for 10 min at 4° C., and the supernatant was speedvac dried and shipped on dry ice to MIT metabolomics core. The pellet was resolubilized in 2% SDS lysis buffer and subjected protein assay by DC™ Protein Assay Kit (#500, Bio-Rad). The data was normalized to total protein.

Measurement of RNA. Total RNA was extracted and isolated using the PureLink RNA Mini Kit (Thermofisher Scientific). cDNA was synthesized using the RevertAid First Strand cDNA Synthesis Kit (Thermofisher Scientific). Real-time PCR was performed on an Applied Biosystems 7500 Fast system using pre-designed Taqman-primer probe sets: DPAGT1 (Hs00609752_m1), GFPT2 (Hs01049570_m1), XBP1U (Hs02856596_m1), XBP1s (Hs03929085_g1), GRP78 (Hs99999174_m1), HSP90B1/GRP94 (Hs00427665_g1), GBA1 (Hs00164683_m1), HexB (Hs01077594_m1) and ACTB (Hs01060665_g1). The quantifications represent the fold change of target mRNA expression normalized to beta-actin (ACTB) levels by delta-Ct method. The values are mean and s.e.m of biological replicates (n=3-6) with two technical replicates for each.

Sequential Protein Extraction and Western Blotting Analysis

Protein extraction was done as described in detail (Stojkovska and Mazzulli, 2021). Briefly, midbrain neurons or mouse brain tissue were harvested in cold PBS by centrifugation at 400×g for 5 minutes. Supernatant was discarded and the cell pellets were subjected to homogenization in 1% Triton X-100 buffer (1% Triton X-100, 20 mM HEPES pH 7.4, 150 mM NaCl, 10% glycerol, 1 mM EDTA, 1.5 mM MgCl2, 1 mM phenylmethanesulfonyl fluoride (PMSF), 50 mM NaF, 2 mM Na orthovanadate, and a protease inhibitor cocktail (Roche diagnostics, https://www.roche.com, #11-836-170-001)) by homogenization. The Triton extracted lysates were incubated in ice-water slurry on a rocker for 30 minutes and then freeze thawed three times followed by ultracentrifugation at 100,000×g for 30 minutes at 4° C. The Supernatant (triton soluble) s collected, and Triton-insoluble pellets were further extracted in 2% SDS lysis buffer by boiling for 10 minutes, followed by sonication and then ultracentrifugation at 100,000×g for 30 minutes at 22° C. The protein concentrations of the Triton and SDS fractions was measured DC™ Protein Assay Kit (Bio-Rad). Extracted protein lysates were subjected to SDS-PAGE followed by transfer of proteins onto a PVDF membrane (0.45 um pore size; Millipore), and post-fixed in 0.4% paraformaldehyde. Membranes were blocked in a 1:1 mixture of 1×TBS and Odyssey blocking buffer (Li-Cor Biosciences) for 1 h at RT, followed by overnight incubation with primary antibodies at 4° C. diluted in a 1:1 mixture of 1×TBS-Tween and Odyssey blocking buffer. The following day, secondary antibodies were added for 1 hour, and the membranes were scanned using a Li-Cor Biosciences infrared imaging system. Protein band intensities was quantified by the ImageStudio software ver 3.1.

Frozen cerebellum dissected from control and A53T mice were homogenized in 1% Triton X-100 buffer in 1:5 weight/volume ratio. The lysates were incubated in ice-water slurry for 30 minutes, subjected to two freeze/thaw cycles and ultracentrifuged at 100,000×g, 4° C. for 30 minutes. Supernatant (Triton-soluble fraction) was subjected to SDS-PAGE followed western blot analysis.

Control and DLB human post-mortem frontal cortical tissues were obtained from the Northwestern University Alzheimer's disease pathology core and sequentially extracted by a 5-step extraction protocol using high salt buffer, 1% Triton X-100, 1% Triton+30% sucrose (Sigma) and 1% sarkosyl (Sigma). Briefly, brain tissues were homogenized in high-salt buffer (HSB) (50 mM Tris-HCl pH 7.4, 750 mM NaCl, 10 mM NaF, 5 mM EDTA) with protease and protein phosphatase inhibitors, followed by incubation on ice for 20 minutes and centrifugation at 100,000×g for 30 minutes at 4° C. The pellets were then re-extracted with HSB, followed by sequential extractions with 1% Triton X-100-containing HSB and 1% Triton X-100-containing HSB with 30% sucrose. The pellets were then resuspended and homogenized in 1% sarkosyl-containing HSB, rotated at 4° C. overnight and centrifuged at 100,000×g for 30 min. The resulting sarkosyl-insoluble pellets were washed once with PBS and resuspended in PBS by brief sonication. The 1% triton fraction was subjected to western blot analysis for the detection of GFPT2 protein.

Analysis of post-ER GCase: This assay was performed as described in detail (Cuddy and Mazzulli, 2021). Briefly, 40 ug of lysate were subjected to Endoglycosidase H (Endo H) digestion as per the manufacturer's protocol (New England Biolabs, https://www.neb.com) at 37° C. for 2 h. Duplicate samples without enzyme were incubated under the same conditions serving as control (undigested samples). The digestion was stopped by the addition of 5× Laemmeli sample buffer and digested and undigested samples were run on 10% SDS-PAGE gels for 3-4 h at 120V. This was followed by western blot analysis using anti-GCase (#G4171, Sigma, 1:500) antibody. Fluorescent secondary antibodies were used for detection (IRDye800 conjugated anti-rabbit, Licor Biosciences). Endo H resistant bands migrating 62-64 kDa were measured as post-ER forms and maturation was calculated by quantifying the post-ER/ER ratios. Quantification was performed on Image Studio software (Licor). GAPDH was used as a loading control. The measurements were done in triplicate for 2-to-3 distinct primary culture batches. Standard error of the mean values were graphed, analyzed by student's t-test, and p values <0.05 were considered significant.

Live-cell lysosomal GCase activity assay. The procedure and analysis method for the activity assay has been previously described in detail (Cuddy and Mazzulli, 2021). Briefly, one day prior to the assay, cells were treated with 1 mg/ml cascade dextran blue (Life Technologies) in a 96-well plate for 24 hours. This was followed by treatment of cells with DMSO or 200 nM bafilomycin A1 for 1 hour at 37° C. The cells were subjected to 1 hour pulse chase with an artificial fluorescent GCase substrate, 100 ug/ml 5-(pentafluoro-benzoylamino) fluorescein di-ß-D-glucopyranoside (PFB-FDGluc; Life Technologies), at 37° C. The fluorescence signal was measured every 30 minutes for the span of 3-4 hours on a plate reader (Ex=485 nm, Em=530 nm, for the GCase substrates; Ex=400 nm, Em=430 nm for cascade dextran blue). For the analysis, the GCase fluorescence signal was normalized to either lysosomal mass by using cascade dextran blue signal or total cell volume by quantifying CellTag 700 staining signal.

### IIB: Imaging Analysis

Neurofilament Toxicity Assay

iPSC-neurons 96 well format were fixed in 4% paraformaldehyde in PBS for 20 minutes, followed by incubation in 0.3% Triton X-100 (PBS) for 20 min. The cells were then blocked with Odyssey blocking buffer (Li-Cor) for 1 h. Anti-neurofilament antibody (1:1000, mouse IgG 2H3, Developmental Studies Hybridoma Bank, University of Iowa, Iowa City, Iowa) was incubated overnight in blocking buffer at 4° C., followed by washing in PBS with 0.1% Tween for 20 min. IRdye 800-conjugated anti-mouse IgG antibodies (1:1000 dilution, Li-Cor) was incubated in blocking buffer for 1 hour and CellTag™ 700 (Li-Cor) was also added for normalization. The Cells were washed four times in PBS-0.1% Tween and scanned on an Odyssey infrared imaging system (Li-Cor). Neurofilament intensity was determined by Image Studio software (version 2.1 Li-Cor) and normalized to cell volume.

### IIC. Lentivirus Generation and Transduction

Generation of the GFPT2 Plasmid and Transduction in iPSC-Neurons

pENTR223-GFPT2 (ccsbBroadEn_07515) containing cDNA plasmid obtained from DNASU plasmid repository (http://dnasu.org/DNASU/GetCloneDetail.do?cloneid=514451 #sequence). A stop codon was inserted by site-directed mutagenesis (SDM) using following mutagenesis primers (5′-CAAGTCTGTAACTGTGGAATGACCAACTTTCTTGTACAAAGT-3′ SEQ ID NO” 13 and 5′-CAACTTTGTACAAGAAAGTTGGTCATTCCACAGTTACAGACTTG-3′ SEQ ID NO: 14). SDM was performed using the materials and procedures from the QuikChange XL Site-Directed Mutagenesis Kit (Agilent). The stop codon insertion was confirmed by sequencing. pENTR223-GFPT2 with stop codon was subcloned into pER4 for lentivirus production.

XBP1^{s} Lentiviral Construct

Lentiviral construct Overexpressing XBp1s (VB900007-1013-https://en.vectorbuilder.com/vector/VB900007-1013mfa.html) was obtained from Vector Builder.

Lentivirus Preparation for Transduction iPSC Neurons

Lentivirus production was achieved by transfecting HEK293FT (Invitrogen, R70007) cells with the lentiviral plasmid expressing the target along with a packaging vector (psPAX2) and an envelope vector (VSV-G). The transfection was performed with X-tremeGENE transfection reagent (Roche #6366236001) as described previously (Cuddy et al., 2019). Lentiviral particles were collected from the media and concentrated using Lenti-X concentrator (Clontech, #631232). The number of viral particles was determined by HIV1-p24 antigen ELISA kit (ZeptoMetrix, #801111) as per the manufacturer's protocol. The viral particle number and the number of plated cells was used to calculate the multiplicity of infection (MOI). For GFPT2 Overexpression the A53T neurons were infected at MOI-3 and harvested 2 weeks post-infection. A53T neurons were infected with XBP1^{s} lentivirus at MOI-1 and harvested 16 days post-infection. Neurons infected with lentivirus expressing GFP was used as control.

### IID. Pharmacology

Tunicamycin Treatment for Inhibition of N-Glycosylation

Neurons were treated with or without 5 ug/ml tunicamycin (EMD Millipore/Calbiochem) for 24 hrs. Cells were extracted in 1% Triton X-100 buffer and analyzed by western blot for Hex B, Con-A, or GFPT2 as described in “Sequential protein extraction and western blotting analysis”. For mRNA measurement, total RNA was extracted and isolated as described in “Measurement of mRNA.” cDNA was synthesized and subjected to real-time PCR using Taqman-primer probe sets for GFPT2, XBP1U, and GRP78.

N-Acetylglucosamine (GlcNAc) Treatment

iPSC neurons were supplemented with or without GlcNAc (A3286-sigma) at 10 mM for 7 days in PBS. Media was changed every other day for the duration of the experiment.

Quantification and Statistical Analysis

Quantification methods of western blots and images have been described above. In each quantification, a single plot point indicates a separate biological replicate (individual culture well). The quantitative data are taken from at least two distinct iPSC passages/differentiation batches. The value of n and what n represents is indicated in each figure legend. Analyzed data was plotted and tested for statistical significance using the GraphPad Prism software. Statistical significance between two samples was determined using a paired or unpaired t-test. For more than two conditions, significance was determined using a one-way ANOVA with Tukey's multiple comparison test. A p-value of <0.05 was considered significant (*p<0.05, **p<0.01, ***p<0.001, ****p<0.0001). For each quantification, the type of error bar used and statistical test is specified in the figure legends.

Key Resources Table

| | | |
|---|---|---|
| REAGENT or RESOURCE | SOURCE | IDENTIFIER |

Antibodies

| | | |
|---|---|---|
| Rabbit polyclonal anti-alpha synuclein | Santa Cruz | Cat #sc-7011-R |
| (C-20) | | RRID: AB_2192953 |
| Concanavalin-A, biotinylated | Vector Laboratories | Cat #B-1005-5 |
| Mouse monoclonal anti-alpha synuclein | Abcam | Cat #ab27766 |
| (LB509) | | RRID: AB_727020 |
| Rabbit polyclonal anti-GFP | Sigma Aldrich | Cat #G1544 |
| | | RRID: AB_439690 |
| Mouse monoclonal anti-alpha synuclein | Biolegend | Cat #824301 |
| (303) | | RRID: AB_2564879 |
| Mouse monoclonal anti-β3-tubulin | Biolegend | Cat #802001 |
| | | RRID: AB_2564645 |
| Rabbit polyclonal anti-GFPT2 | Abcam | Cat # ab190966 |
| | | RRID: AB_2868470 |
| Mouse monoclonal anti-GAPDH | Millipore | Cat #CB1001 |
| | | RRID: AB_2107426 |
| Rabbit polyclonal anti-glucocerebrosidase | Sigma | Cat #G4171 |
| (GCase) | | RRID: AB_1078958 |
| Mouse Monoclonal anti-puromycin | millipore sigma | Cat # MABE343 |
| | | RRID: AB_2566826 |
| Mouse monoclonal anti-Hexosaminidase B | Santa Cruz | Cat #sc-376781 |
| (HexB) | | RRID: AB_2909474 |
| Neurofilament | Biolegend | Cat #SMI-312R |
| | | RRID: AB_2314906 |
| Mouse monoclonal anti-oligosaccharyltransferase | Santa Cruz | Cat # sc-74408 |
| (OST48) antibody | | RRID: AB_1125745 |
| Mouse monoclonal anti- Hexokinase-1 | Santa Cruz | Cat #sc-46695 |
| (HXK I) | | |
| Mouse monoclonal anti - | Santa Cruz | Cat #sc- sc-374519 |
| phosphoglucosamine acetylase (GNA1) | | RRID: AB_10986418 |
| antibody | | |
| Mouse monoclonal anti - GlcNAc kinase | Santa Cruz | Cat #sc-390499 |
| (NAGK) antibody | | |
| N-Azidoacetylmannosamine-tetraacylated | Sigma | Cat # 900917 |
| (Ac4ManNAz) | | |
| Secondary antibody: Alexa Fluor 488 Goat | Invitrogen | Cat #A11034 |
| anti-rabbit IgG secondary (H + L) | | RRID: AB_2576217 |
| Secondary antibody: Alexa Fluor 488 Goat | Invitrogen | Cat #A11029 |
| anti-mouse IgG secondary (H + L) | | RRID: AB_138404 |
| Secondary antibody: Alexa Fluor 568 Goat | Invitrogen | Cat #A11036 |
| anti-rabbit IgG secondary (H + L) | | RRID: AB_10563566 |
| Secondary antibody: Alexa Fluor 568 Goat | Invitrogen | Cat #A11031 |
| anti-mouse IgG secondary (H + L) | | RRID: AB_144696 |
| Secondary antibody: Alexa Fluor 680 Goat | Invitrogen | Cat #A21058 |
| anti-mouse IgG secondary (H + L) | | RRID: AB_2535724 |
| Secondary antibody: IRdye 800CW goat | Li-Cor Biosciences | Cat #926-32210 |
| anti-mouse IgG secondary (H + L) | | RRID: AB_621842 |
| Secondary antibody: IRdye 800CW goat | Li-Cor Biosciences | Cat #926-32211 |
| anti-rabbit IgG secondary (H + L) | | RRID: AB_621843 |
| Secondary antibody: IRDye800 CW | Li-Cor Biosciences | Cat #92632230 |
| anti-streptavidin | | |

Bacterial and virus strains

| | | |
|---|---|---|
| lenti-pER4 (HIV, replication incompetent) | Mazzulli et al., | N/A |
| | 2011 | |
| pER4 GFPT2 lentivirus | This paper | N/A |
| pLV[Exp]-EGFP: T2A: Puro- | This paper | N/A |
| EF1A > hXBP1[NM_001079539.1] | | |

Biological samples

| | | |
|---|---|---|
| Human brain tissue of control, DLB, DLB + | Northwestern | N/A |
| AD patients | University | |
| | Alzheimer's disease | |
| | pathology core | |
| | (CNADC). | |
| | Stojkovska et al., | |
| | 2022 | |
| Mouse Brain tissue from Control and A53T | Giasson et al., 2002 | N/A |
| mice | | |

Chemicals, peptides, and recombinant proteins

| | | |
|---|---|---|
| Bafilomycin A1 | Santa Cruz | Cat #SC-201550 |
| Cascade Dextran Blue | Life Technologies | Cat #D1976 |
| Conduritol β epoxide (CBE) | Millipore | Cat #234599 |
| Doxycycline (DOX) | Sigma | Cat #D3447 |
| Fetal bovine serum (FBS), heat-inactivated | Thermo Fisher | Cat #10438026 |
| | Scientific | |
| Geneticin (G418) | Thermo Fisher | Cat #10131027 |
| | Scientific | |
| L-glutamine | Gibco | Cat #25030081 |
| Hygromycin B | Thermo Fisher | Cat #10687010 |
| | Scientific | |
| Paraformaldehyde (10%, methanol-free) | Polysciences, Inc. | Cat #40181 |
| Penicillin/Streptomycin | Thermo Fisher | Cat #10378016 |
| | Scientific | |
| Phenylmethyl sulfonyl fluoride (PMSF) | Sigma | Cat #78830 |
| Protease Inhibitor Cocktail (PIC) | Roche | Cat #11836170001 |
| N-Lauroylsarcosine sodium salt (sarkosyl) | Sigma | Cat #L9150 |
| Sodium dodecyl sulfate (SDS) | Sigma | Cat #L4509 |
| Sodium orthovanadate (Na₃VO₄) | Sigma | Cat #450243 |
| Sodium fluoride (NaF) | Sigma | Cat #201154 |
| Sucrose | Sigma | Cat #S1888 |
| Triton X-100 | Sigma | Cat #T8787 |
| Tunicamycin (Tunic) | EMD Millipore/ | Cat #654380 |
| | Calbiochem | |
| 5-(pentafluoro-benzoylamino) fluorescein | Life Technologies | Cat #P11947 |
| di-β-D-glucopyranoside (PFB-FDGluc) | | |

Critical commercial assays

| | | |
|---|---|---|
| CellTag 700 | Li-Cor Biosciences | Cat #926-41090 |
| Endoglycosidase H | New England | Cat #P0702L |
| | Biolabs | |
| HIV1-p24 Antigen ELISA Kit | Zeptometrix | Cat #0801111 |
| DC ™ Protein Assay Kit | Bio-Rad | #500 |
| RevertAid First Strand cDNA Synthesis Kit | Thermo Fisher | Cat #K1621 |
| | Scientific | |
| RNeasy Mini Prep Kit | QIAGEN | Cat #74104 |
| Quantitative RT-PCR: DPAGT1 | Thermo Fisher | Cat #4331182 |
| (Hs00609752_m1) | Scientific | |
| Quantitative RT-PCR: GRP78 | Thermo Fisher | Cat #4331182 |
| (ID: Hs99999174_m1) | Scientific | |
| Quantitative RT-PCR: GFPT2 | Thermo Fisher | Cat #4331182 |
| (Hs01049570_m1) | Scientific | |
| Quantitative RT-PCR: | Thermo Fisher | Cat #4331182 |
| XBP1U(Hs02856596_m1) | Scientific | |
| Quantitative RT-PCR: XBP1-S | Thermo Fisher | Cat #4331182 |
| (ID: Hs03929085_g1) | Scientific | |
| Quantitative RT-PCR: HSP90B1/GRP94 | Thermo Fisher | Cat #4331182 |
| (Hs00427665_g1) | Scientific | |
| Quantitative RT-PCR: GBA1 | Thermo Fisher | Cat #4331182 |
| (ID: Hs00164683_m1) | Scientific | |
| Quantitative RT-PCR: HexB | Thermo Fisher | Cat #4331182 |
| (Hs01077594_m1) and | Scientific | |
| Quantitative RT-PCR: ACTB | Thermo Fisher | Cat #4331182 |
| (Hs01060665_g1) | Scientific | |
| QuikChange XL Site-Directed Mutagenesis | Agilent | Cat #200517 |
| Kit | | |

Deposited data
Experimental models: Cell lines

| | | |
|---|---|---|
| H4 neuroglioma cells | Mazzulli et al., | N/A |
| | 2011; From: | |
| | Pamela McLean | |
| | (Mayo Clinic, | |
| | Jacksonville, | |
| | Florida, USA) | |
| GM15010 (SNCA Triplication, 3x-1) | Stojkovska et al., | N/A |
| | 2022; clinical and | |
| | other information | |
| | can be obtained | |
| | from the Coriell | |
| | Cell Repository | |
| ND00196 (SNCA Triplication, 3x-2) | Stojkovska et al., | N/A |
| | 2022; clinical and | |
| | other information | |
| | can be obtained | |
| | from the Coriell | |
| | Cell Repository | |
| ND34391 (SNCA Triplication, Est. 3X). | Stojkovska et al., | N/A |
| | 2022; Mazzulli et | |
| | al., 2016a; Zunke et | |
| | al., 2018; Cuddy et | |
| | al., 2019; Coriell | |
| | Cell Repository | |
| SNCA Triplication, line 1A | Mazzulli et al, | N/A |
| | PNAS 2016 | |
| A53T alpha-synuclein and isogenic control | Soldner et al., | N/A |
| | Cell, 2011 | |
| SH-SY5Y cells, female origin | Cuddy et al., 2019 | ATCC Cat #CRL22-66 |

Experimental models: Organisms/strains
Oligonucleotides- See Table S4
Recombinant DNA

| | | |
|---|---|---|
| pENTR223-GFPT2 (ccsbBroadEn_07515) | Addgene | Cat # |
| | | HsCD0000313027077 |
| lenti-pER4 | Mazzulli et al., | N/A |
| | 2011 | |
| pER4 GFPT2 lentivirus | This paper | N/A |
| pLV[Exp]-EGFP: T2A: Puro- | This paper | VB900007-1013mfa |
| EF1A > hXBP1[NM_001079539.1] | | |

Software and algorithms

| | | |
|---|---|---|
| GraphPad Prism V6.0 software | GraphPad | https://www.graphpad.com/ |
| | | scientific-software/prism/ |
| Odyssey software (Image Studio V3.1.4) | Li-Cor Biosciences | https://www.licor.com/bio/ |
| | | image-studio/ |
| Snap gene V5.3 software | SnapGene | https://www.snapgene.com |

Other

| | | |
|---|---|---|
| Intercept blocking buffer | Li-Cor Biosciences | Cat #927-70001 |
| Lenti-X concentrator | Clontech | Cat #631232 |
| Matrigel | Fisher | Cat #CB-40234 |
| mTeSR1 media | StemCell | Cat #85850 |
| | Technologies | |
| Neurobasal SM1 media | Thermo Fisher | Cat #21103-049 |
| | Scientific | |
| NeuroCult SM1 supplement | StemCell | Cat #05711 |
| | Technologies | |
| NeutrAvidin agarose beads | Thermo Fisher | Cat #29204 |
| | Scientific | |
| PVDF transfer membrane, | Millipore | Cat #IPFL00010 |
| 0.45 □m pore size | | |
| X-tremeGENE HP DNA Transfection | Roche | Cat #6366236001 |
| Reagent | | |

### REFERENCES

Abdel Rahman, A. M., Ryczko, M., Pawling, J., and Dennis, J. W. (2013). Probing the hexosamine biosynthetic pathway in human tumor cells by multitargeted tandem mass spectrometry. ACS Chem Biol 8, 2053-2062. 10.1021/cb4004173.
Cai, R., Zhang, Y., Simmering, J. E., Schultz, J. L., Li, Y., Fernandez-Carasa, I., Consiglio, A., Raya, A., Polgreen, P. M., Narayanan, N. S., et al. (2019). Enhancing glycolysis attenuates Parkinson's disease progression in models and clinical databases. J Clin Invest 129, 4539-4549. 10.1172/JC1129987.
Carrera, I. A., Matthijs, G., Perez, B., and Cerda, C. P. (2012). DPAGT1-CDG: report of a patient with fetal hypokinesia phenotype. Am J Med Genet A 158A, 2027-2030. 10.1002/ajmg.a.35472.
Chang, D., Nalls, M. A., Hallgrimsdottir, I. B., Hunkapiller, J., van der Brug, M., Cai, F., Kerchner, G. A., Ayalon, G., Bingol, B., Sheng, M., et al. (2017). A meta-analysis of genome-wide association studies identifies 17 new Parkinson's disease risk loci. Nature genetics 49, 1511-1516. 10.1038/ng.3955.
Chung, C. Y., Khurana, V., Auluck, P. K., Tardiff, D. F., Mazzulli, J. R., Soldner, F., Baru, V., Lou, Y., Freyzon, Y., Cho, S., et al. (2013). Identification and rescue of alpha-synuclein toxicity in Parkinson patient-derived neurons. Science 342, 983-987. 10.1126/science.1245296.
Cooper, A. A., Gitler, A. D., Cashikar, A., Haynes, C. M., Hill, K. J., Bhullar, B., Liu, K., Xu, K., Strathearn, K. E., Liu, F., et al. (2006). Alpha-synuclein blocks ER-Golgi traffic and Rab1 rescues neuron loss in Parkinson's models. Science (New York, N.Y.) 313, 324-328. 10.1126/science.1129462.
Cuddy, L. K., and Mazzulli, J. R. (2021). Analysis of lysosomal hydrolase trafficking and activity in human iPSC-derived neuronal models. STAR Protoc 2, 100340. 10.1016/j.xpro.2021.100340.
Cuddy, L. K., Wani, W. Y., Morella, M. L., Pitcairn, C., Tsutsumi, K., Fredriksen, K., Justman, C. J., Grammatopoulos, T. N., Belur, N. R., Zunke, F., et al. (2019). Stress-Induced Cellular Clearance Is Mediated by the SNARE Protein ykt6 and Disrupted by alpha-Synuclein. Neuron 104, 869-884 e811. 10.1016/j.neuron.2019.09.001.
Denzel, M. S., and Antebi, A. (2015). Hexosamine pathway and (ER) protein quality control. Curr Opin Cell Biol 33, 14-18. 10.1016/j.ceb.2014.10.001.
Denzel, M. S., Storm, N. J., Gutschmidt, A., Baddi, R., Hinze, Y., Jarosch, E., Sommer, T., Hoppe, T., and Antebi, A. (2014). Hexosamine pathway metabolites enhance protein quality control and prolong life. Cell 156, 1167-1178. 10.1016/j.cell.2014.01.061.
Dong, Y. Y., Wang, H., Pike, A. C. W., Cochrane, S. A., Hamedzadeh, S., Wyszyński, F. J., Bushell, S. R., Royer, S. F., Widdick, D. A., Sajid, A., et al. (2018). Structures of DPAGT1 Explain Glycosylation Disease Mechanisms and Advance TB Antibiotic Design. Cell 175, 1045-1058.e1016. 10.1016/j.cell.2018.10.037.
Firbank, M. J., Yarnall, A. J., Lawson, R. A., Duncan, G. W., Khoo, T. K., Petrides, G. S., O'Brien, J. T., Barker, R. A., Maxwell, R. J., Brooks, D. J., and Burn, D. J. (2017). Cerebral glucose metabolism and cognition in newly diagnosed Parkinson's disease: ICICLE-PD study. J Neurol Neurosurg Psychiatry 88, 310-316. 10.1136/jnnp-2016-313918.
Fredriksen, K., Aivazidis, S., Sharma, K., Burbidge, K. J., Pitcairn, C., Zunke, F., Gelyana, E., and Mazzulli, J. R. (2021). Pathological alpha-syn aggregation is mediated by glycosphingolipid chain length and the physiological state of alpha-syn in vivo. Proc Natl Acad Sci USA 118. 10.1073/pnas.2108489118.
Freeze, H. H., Eklund, E. A., Ng, B. G., and Patterson, M. C. (2015). Neurological aspects of human glycosylation disorders. Annual review of neuroscience 38, 105-125. 10.1146/annurev-neuro-071714-034019.
Giasson, B. I., Duda, J. E., Quinn, S. M., Zhang, B., Trojanowski, J. Q., and Lee, V. M. (2002). Neuronal alpha-synucleinopathy with severe movement disorder in mice expressing A53T human alpha-synuclein. Neuron 34, 521-533. 10.1016/s0896-6273(02)00682-7.
Gitler, A. D., Bevis, B. J., Shorter, J., Strathearn, K. E., Hamamichi, S., Su, L. J., Caldwell, K. A., Caldwell, G. A., Rochet, J. C., McCaffery, J. M., et al. (2008). The Parkinson's disease protein alpha-synuclein disrupts cellular Rab homeostasis. Proc Natl Acad Sci USA 105, 145-150. 10.1073/pnas.0710685105.
Goyal, M. S., Vlassenko, A. G., Blazey, T. M., Su, Y., Couture, L. E., Durbin, T. J., Bateman, R. J., Benzinger, T. L., Morris, J. C., and Raichle, M. E. (2017). Loss of Brain Aerobic Glycolysis in Normal Human Aging. Cell Metab 26, 353-360 e353. 10.1016/j.cmet.2017.07.010.
Hasilik, A., and Neufeld, E. F. (1980). Biosynthesis of lysosomal enzymes in fibroblasts. Synthesis as precursors of higher molecular weight. J Biol Chem 255, 4937-4945.
Hebert, D. N., Lamriben, L., Powers, E. T., and Kelly, J. W. (2014). The intrinsic and extrinsic effects of N-linked glycans on glycoproteostasis. Nat Chem Biol 10, 902-910. 10.1038/nchembio.1651.
Heifetz, A., Keenan, R. W., and Elbein, A. D. (1979). Mechanism of action of tunicamycin on the UDP-GlcNAc:dolichyl-phosphate Glc-NAc-1-phosphate transferase. Biochemistry 18, 2186-2192. 10.1021/bi00578a008.
Hipp, M. S., Kasturi, P., and Hartl, F. U. (2019). The proteostasis network and its decline in ageing. Nat Rev Mol Cell Biol 20, 421-435. 10.1038/s41580-019-0101-y.
Holtbernd, F., Gagnon, J. F., Postuma, R. B., Ma, Y., Tang, C. C., Feigin, A., Dhawan, V., Vendette, M., Soucy, J. P., Eidelberg, D., and Montplaisir, J. (2014). Abnormal metabolic network activity in REM sleep behavior disorder. Neurology 82, 620-627. 10.1212/WNL.0000000000000130.
Hu, M. T., Taylor-Robinson, S. D., Chaudhuri, K. R., Bell, J. D., Labbe, C., Cunningham, V. J., Koepp, M. J., Hammers, A., Morris, R. G., Turjanski, N., and Brooks, D. J. (2000). Cortical dysfunction in non-demented Parkinson's disease patients: a combined (31)P-MRS and (18)FDG-PET study. Brain 123 (Pt 2), 340-352. 10.1093/brain/123.2.340.
Johswich, A., Longuet, C., Pawling, J., Abdel Rahman, A., Ryczko, M., Drucker, D. J., and Dennis, J. W. (2014). N-glycan remodeling on glucagon receptor is an effector of nutrient sensing by the hexosamine biosynthesis pathway. J Biol Chem 289, 15927-15941. 10.1074/jbc.M114.563734.
Kelleher, D. J., Kreibich, G., and Gilmore, R. (1992). Oligosaccharyltransferase activity is associated with a protein complex composed of ribophorins I and II and a 48 kd protein. Cell 69, 55-65. 10.1016/0092-8674(92)90118-v.
Klein, A. D., and Mazzulli, J. R. (2018). Is Parkinson's disease a lysosomal disorder?Brain 141, 2255-2262. 10.1093/brain/awy147.
Kriks, S., Shim, J. W., Piao, J., Ganat, Y. M., Wakeman, D. R., Xie, Z., Carrillo-Reid, L., Auyeung, G., Antonacci, C., Buch, A., et al. (2011). Dopamine neurons derived from human ES cells efficiently engraft in animal models of Parkinson's disease. Nature 480, 547-551. 10.1038/nature10648.
Labbadia, J., and Morimoto, R. I. (2015). The biology of proteostasis in aging and disease. Annual review of biochemistry 84, 435-464. 10.1146/annurev-biochem-060614-033955.
Mazzulli, J. R., Xu, Y. H., Sun, Y., Knight, A. L., McLean, P. J., Caldwell, G. A., Sidransky, E., Grabowski, G. A., and Krainc, D. (2011). Gaucher disease glucocerebrosidase and alpha-synuclein form a bidirectional pathogenic loop in synucleinopathies. Cell 146, 37-52. 10.1016/j.cell.2011.06.001.
Mazzulli, J. R., Zunke, F., Isacson, O., Studer, L., and Krainc, D. (2016). alpha-Synuclein-induced lysosomal dysfunction occurs through disruptions in protein trafficking in human midbrain synucleinopathy models. Proc Natl Acad Sci USA 113, 1931-1936. 10.1073/pnas.1520335113.
Niethammer, M., and Eidelberg, D. (2012). Metabolic brain networks in translational neurology: concepts and applications. Ann Neurol 72, 635-647. 10.1002/ana.23631.
Oaks, A. W., Marsh-Armstrong, N., Jones, J. M., Credle, J. J., and Sidhu, A. (2013). Synucleins antagonize endoplasmic reticulum function to modulate dopamine transporter trafficking. PLoS One 8, e70872. 10.1371/journal.pone.0070872.
Oki, T., Yamazaki, K., Kuromitsu, J., Okada, M., and Tanaka, I. (1999). cDNA cloning and mapping of a novel subtype of glutamine:fructose-6-phosphate amidotransferase (GFAT2) in human and mouse. Genomics 57, 227-234. 10.1006/geno.1999.5785.
Pappata, S., Santangelo, G., Aarsland, D., Vicidomini, C., Longo, K., Bronnick, K., Amboni, M., Erro, R., Vitale, C., Caprio, M. G., et al. (2011). Mild cognitive impairment in drug-naive patients with PD is associated with cerebral hypometabolism. Neurology 77, 1357-1362. 10.1212/WNL.0b013e3182315259.
Peppard, R. F., Martin, W. R., Carr, G. D., Grochowski, E., Schulzer, M., Guttman, M., McGeer, P. L., Phillips, A. G., Tsui, J. K., and Calne, D. B. (1992). Cerebral glucose metabolism in Parkinson's disease with and without dementia. Arch Neurol 49, 1262-1268. 10.1001/archneur.1992.00530360060019.
Robak, L. A., Jansen, I. E., van Rooij, J., Uitterlinden, A. G., Kraaij, R., Jankovic, J., International Parkinson's Disease Genomics, C., Heutink, P., and Shulman, J. M. (2017). Excessive burden of lysosomal storage disorder gene variants in Parkinson's disease. Brain 140, 3191-3203. 10.1093/brain/awx285.
Saxon, E., and Bertozzi, C. R. (2000). Cell surface engineering by a modified Staudinger reaction. Science 287, 2007-2010. 10.1126/science.287.5460.2007.
Sidransky, E., and Lopez, G. (2012). The link between the GBA gene and parkinsonism. The Lancet. Neurology 11, 986-998. 10.1016/s1474-4422(12)70190-4.
Soldner, F., Laganiere, J., Cheng, A. W., Hockemeyer, D., Gao, Q., Alagappan, R., Khurana, V., Golbe, L. I., Myers, R. H., Lindquist, S., et al. (2011). Generation of isogenic pluripotent stem cells differing exclusively at two early onset Parkinson point mutations. Cell 146, 318-331. 10.1016/j.cell.2011.06.019.
Spillantini, M. G., Schmidt, M. L., Lee, V. M., Trojanowski, J. Q., Jakes, R., and Goedert, M. (1997). Alpha-synuclein in Lewy bodies. Nature 388, 839-840. 10.1038/42166.
Stojkovska, I., Krainc, D., and Mazzulli, J. R. (2017). Molecular mechanisms of alpha-synuclein and GBA1 in Parkinson's disease. Cell and tissue research. 10.1007/s00441-017-2704-y.
Stojkovska, I., and Mazzulli, J. R. (2021). Detection of pathological alpha-synuclein aggregates in human iPSC-derived neurons and tissue. STAR Protoc 2, 100372. 10.1016/j.xpro.2021.100372.
Stojkovska, I., Wani, W. Y., Zunke, F., Belur, N. R., Pavlenko, E. A., Mwenda, N., Sharma, K., Francelle, L., and Mazzulli, J. R. (2022). Rescue of alpha-synuclein aggregation in Parkinson's patient neurons by synergistic enhancement of ER proteostasis and protein trafficking. Neuron 110, 436-451 e411. 10.1016/j.neuron.2021.10.032.
Tang, C. C., Poston, K. L., Dhawan, V., and Eidelberg, D. (2010). Abnormalities in metabolic network activity precede the onset of motor symptoms in Parkinson's disease. J Neurosci 30, 1049-1056. 10.1523/JNEUROSCI.4188-09.2010.
Thayanidhi, N., Helm, J. R., Nycz, D. C., Bentley, M., Liang, Y., and Hay, J. C. (2010). Alpha-synuclein delays endoplasmic reticulum (ER)-to-Golgi transport in mammalian cells by antagonizing ER/Golgi SNAREs. Mol Biol Cell 21, 1850-1863. 10.1091/mbc.E09-09-0801.
Tsika, E., Moysidou, M., Guo, J., Cushman, M., Gannon, P., Sandaltzopoulos, R., Giasson, B. I., Krainc, D., Ischiropoulos, H., and Mazzulli, J. R. (2010). Distinct region-specific alpha-synuclein oligomers in A53T transgenic mice: implications for neurodegeneration. J Neurosci 30, 3409-3418. 10.1523/JNEUROSCI.4977-09.2010.
Wang, Z. V., Deng, Y., Gao, N., Pedrozo, Z., Li, D. L., Morales, C. R., Criollo, A., Luo, X., Tan, W., Jiang, N., et al. (2014). Spliced X-box binding protein 1 couples the unfolded protein response to hexosamine biosynthetic pathway. Cell 156, 1179-1192. 10.1016/j.cell. 2014.01.014.
Wellen, K. E., Lu, C., Mancuso, A., Lemons, J. M., Ryczko, M., Dennis, J. W., Rabinowitz, J. D., Coller, H. A., and Thompson, C. B. (2010). The hexosamine biosynthetic pathway couples growth factor-induced glutamine uptake to glucose metabolism. Genes Dev 24, 2784-2799. 10.1101/gad.1985910.
Wu, X., Rush, J. S., Karaoglu, D., Krasnewich, D., Lubinsky, M. S., Waechter, C. J., Gilmore, R., and Freeze, H. H. (2003). Deficiency of UDP-GlcNAc:Dolichol Phosphate N-Acetylglucosamine-1 Phosphate Transferase (DPAGT1) causes a novel congenital disorder of Glycosylation Type Ij. Human mutation 22, 144-150. 10.1002/humu.10239.
Zheng, B., Liao, Z., Locascio, J. J., Lesniak, K. A., Roderick, S. S., Watt, M. L., Eklund, A. C., Zhang-James, Y., Kim, P. D., Hauser, M. A., et al. (2010). PGC-1Alpha, a potential therapeutic target for early intervention in Parkinson's disease. Sci Transl Med 2, 52ra73. 10.1126/scitranslmed.3001059.
Zunke, F., Moise, A. C., Belur, N. R., Gelyana, E., Stojkovska, I., Dzaferbegovic, H., Toker, N. J., Jeon, S., Fredriksen, K., and Mazzulli, J. R. (2018). Reversible Conformational Conversion of alpha-Synuclein into Toxic Assemblies by Glucosylceramide. Neuron 97, 92-107 e110. 10.1016/j.neuron.2017.12.012.

In accordance with this disclosure, various other compounds, varied structurally, stereochemically and/or configurationally, are available through such incorporated synthetic procedures and techniques or straight-forward modifications thereof, such modifications as would also be known and understood by those skilled in the art and made aware of this invention, such procedures, techniques and modifications limited only by the commercial or synthetic availability of any corresponding reagent or starting material.

We claim:

1. A method for treating and/or preventing a disease, disorder, or condition that is associated with accumulation of α-synuclein in a subject in need thereof, the method comprising administering to the subject a therapeutically effective amount of (a) at least one ER-Golgi trafficking enhancer in combination with at least one ER protein folding enhancer, or (b) at least one N-glycosylation enhancer, or a pharmaceutical composition comprising a therapeutically effective amount of (a) at least one ER-Golgi trafficking enhancer and at least one ER protein folding enhancer, or (b) at least one N-glycosylation enhancer.

2. The method of claim 1, wherein the disease, disorder, or condition is a neurodegenerative disease, disorder, or condition selected from the group consisting of Parkinson's disease, Lewy body dementia, Alzheimer's disease, amyotrophic lateral sclerosis (ALS), multiple system atrophy, Huntington's disease, Prion disease, frontotemporal dementia, Picks disease, progressive supranuclear palsy, progeria, and any combinations thereof.

3. The method of claim 1, wherein the disorder is a rapid eye movement (REM) behavior sleep disorder linked to Parkinson's disease and/or GBA1 mutation carriers.

4. The method of claim 1, wherein the disorder is a pediatric lysosomal storage disorder selected from the group consisting of glycogen storage disorder, neuronal ceroid lipofuscinosis disorder, sphingolipid storage disorder, cholesterol storage disorder, fatty acid storage disorder, and any combinations thereof.

5. The method of claim 1, wherein the disease, disorder, or condition is a protein misfolding and/or amyloidosis disease, disorder, or condition selected from the group consisting of cataract caused by α-crystallin aggregation, systemic amyloidosis, type 2 diabetes characterized by amylin aggregation, alpha-1-antitrypsin deficiency liver disease, and any combinations thereof.

6. The method of claim 1, wherein the subject is administered the at least one ER-Golgi trafficking enhancer in combination with the at least one ER protein folding enhancer.

7. The method of claim 6, wherein the ER-Golgi trafficking enhancer is selected from the group consisting of farnesyltransferase inhibitor (FTI), ykt6 activator, Rab1A activator, and any combinations thereof.

8. The method of claim 7, wherein the FTI is selected from the group consisting of LNK-754, lonafarnib, gliotoxin, gingerol, tipifarnib, α-hydroxy farnesyl phosphonic acid, manumycin A, L-744832 dihydrochloride, FTI-277 trifluoroacetate salt, FTI-276 trifluoroacetate salt, FTase inhibitor II, and FTase inhibitor I.

9. The method of claim 7, wherein the FTI is selected from the following compounds or a pharmaceutically acceptable salt thereof:

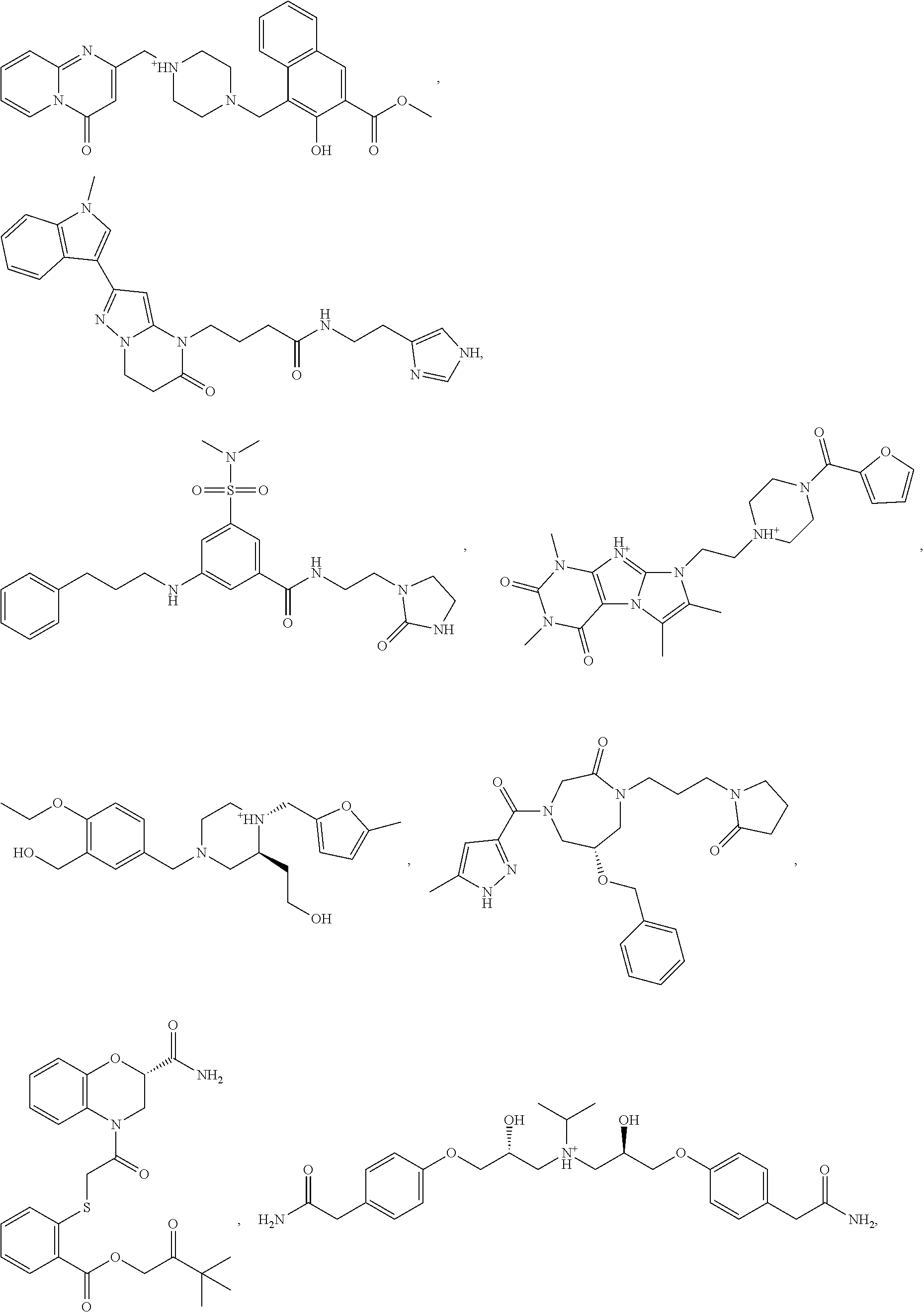

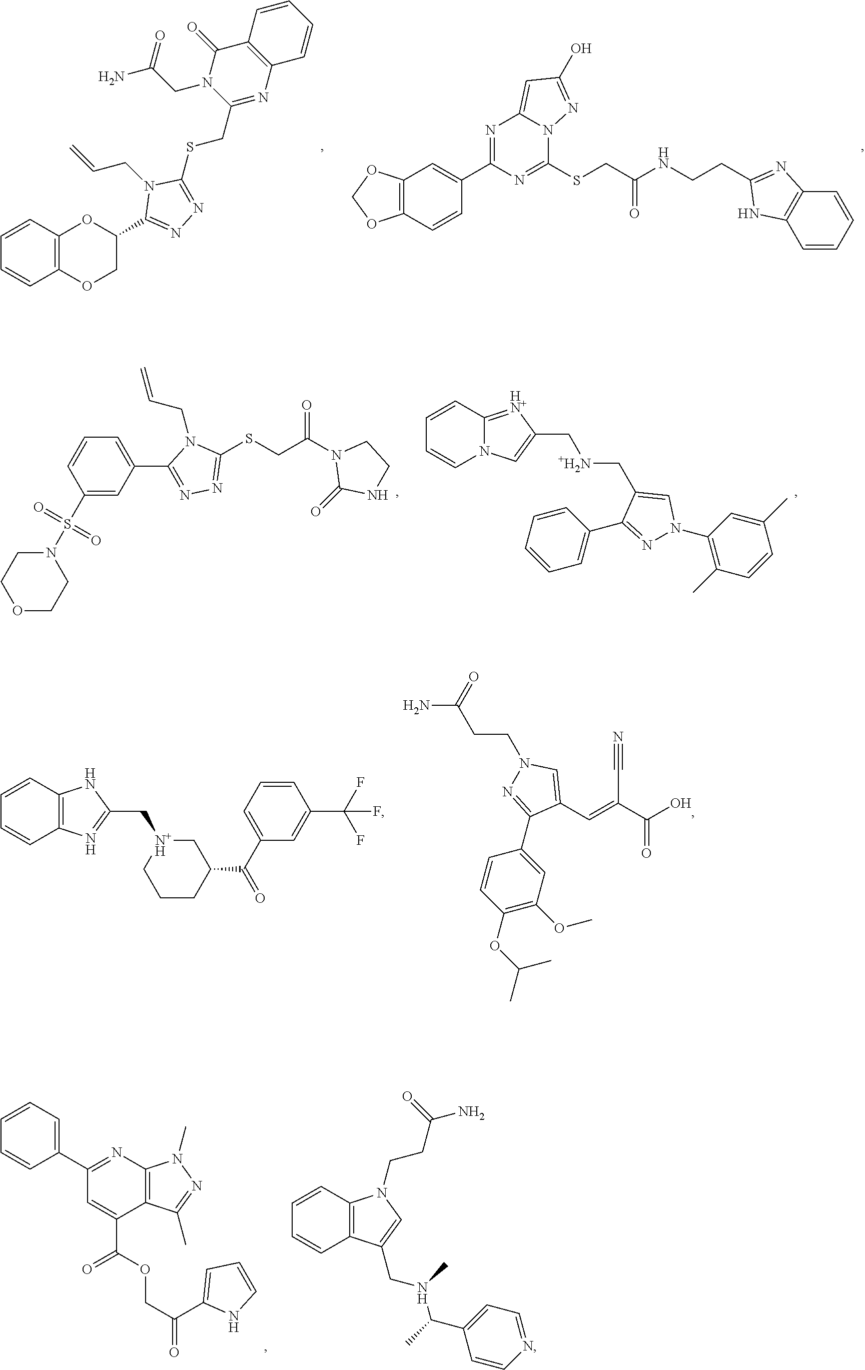

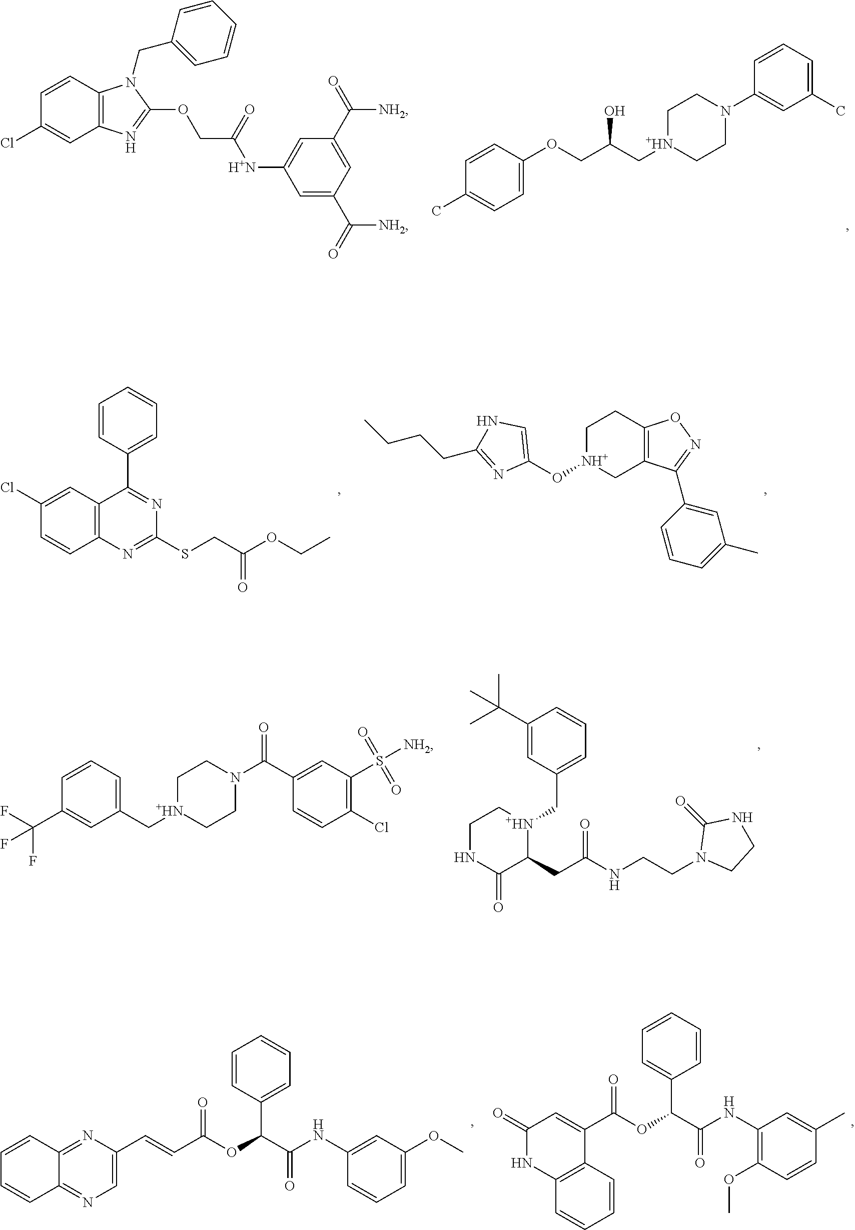

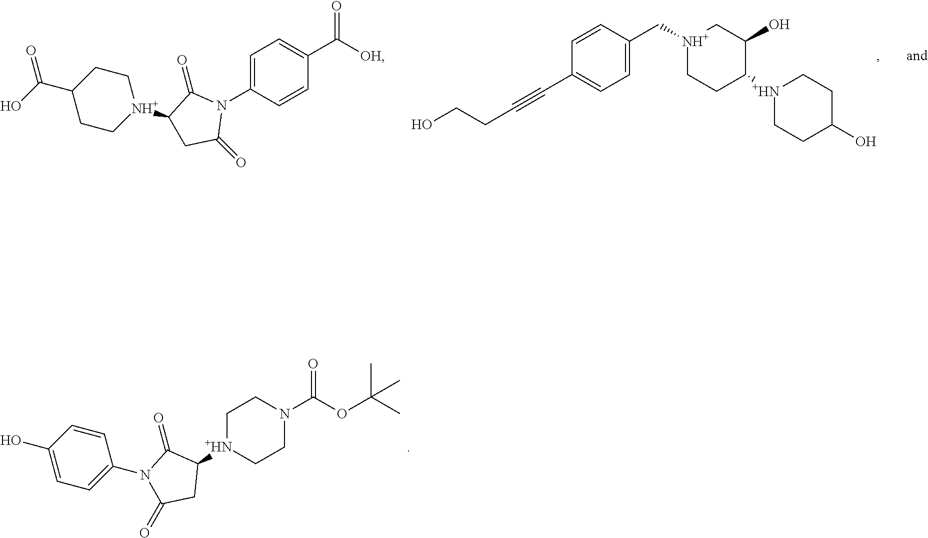 [SMILES: O=C(O)c1ccc(N2C(=O)C[C@@H]([NH+]3CCC(C(=O)O)CC3)C2=O)cc1 ; OCCC#Cc1ccc(C[N@H+]2CC[C@@H]([NH+]3CCC(O)CC3)[C@H](O)C2)cc1 ; CC(C)(C)OC(=O)N1CC[NH+]([C@H]2CC(=O)N(c3ccc(O)cc3)C2=O)CC1]

10. The method of claim 6, wherein the ykt6 activator is selected from the following compounds or a pharmaceutically acceptable salt thereof:

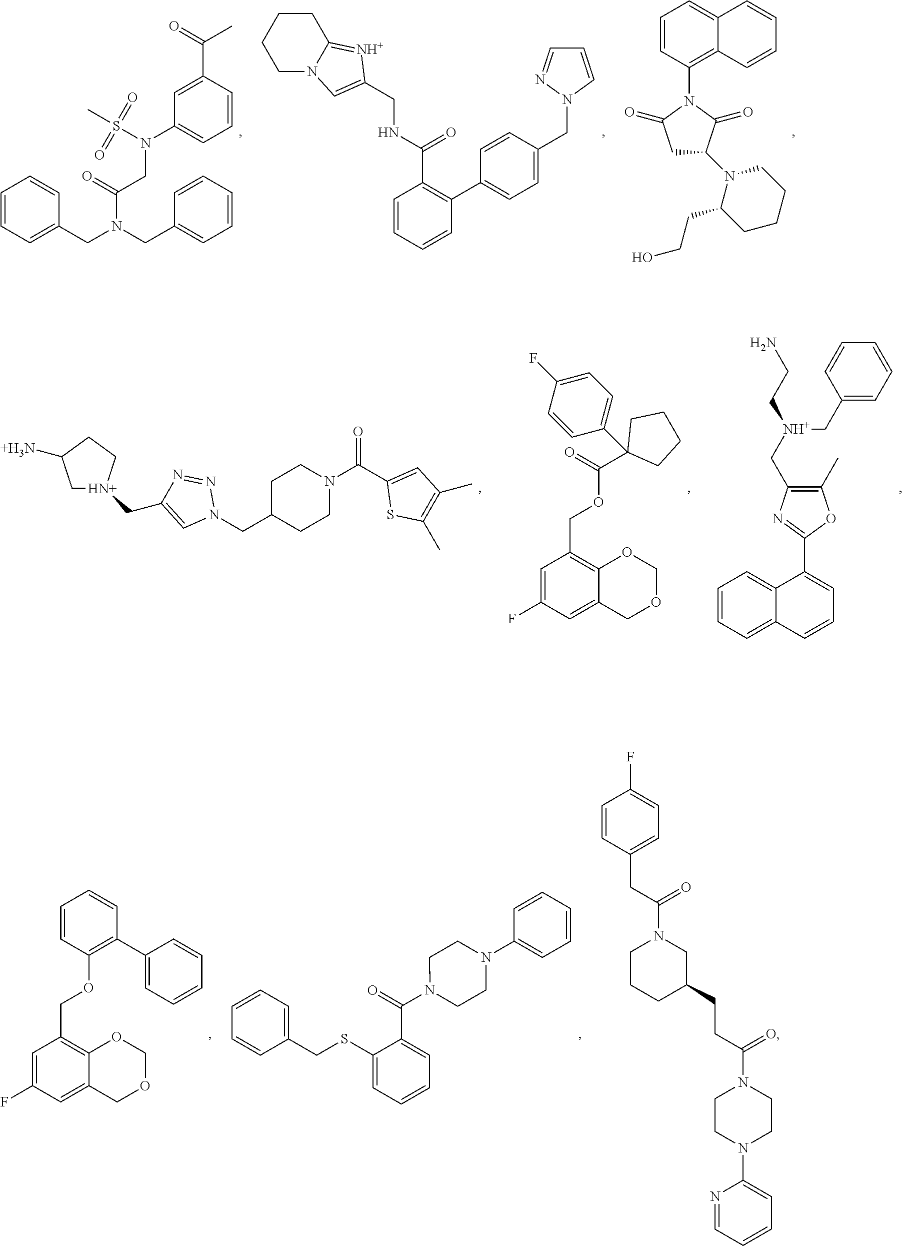

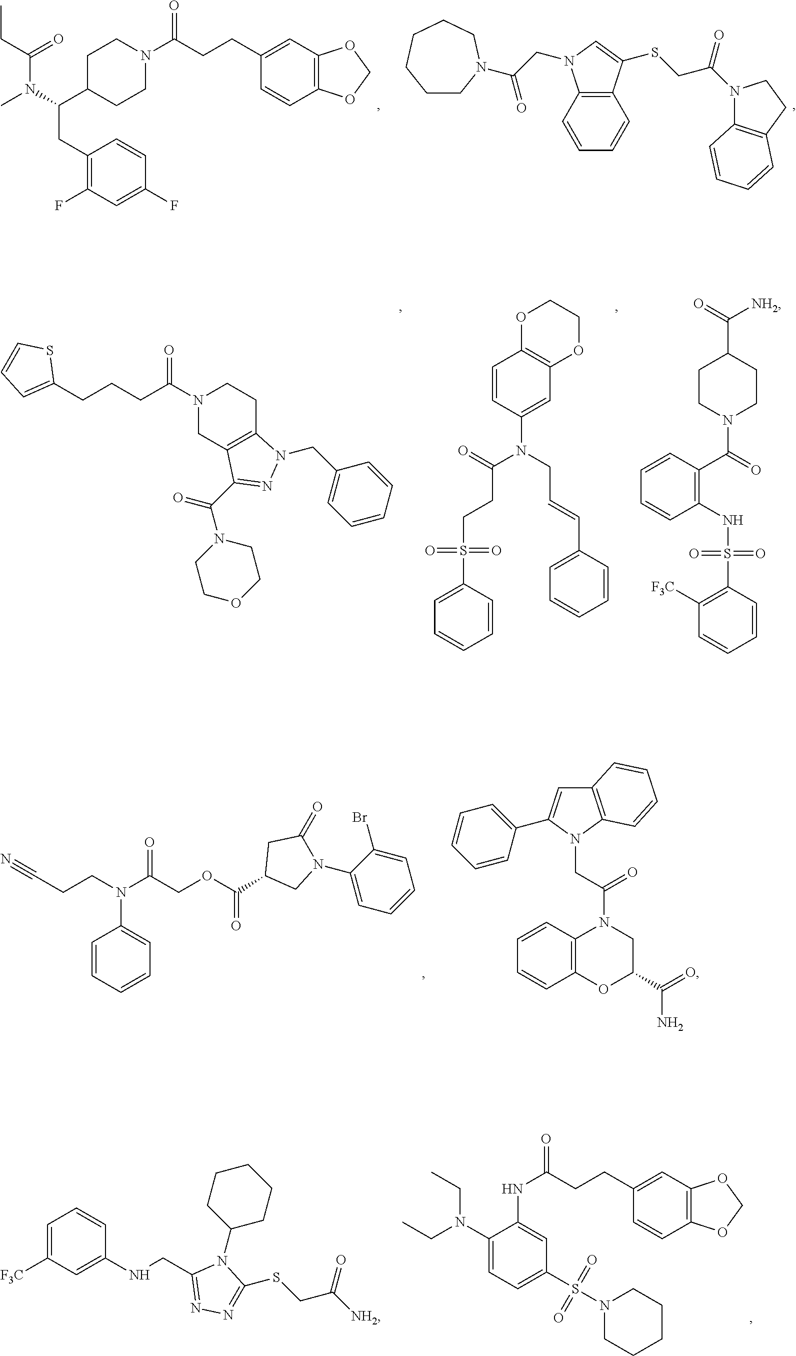

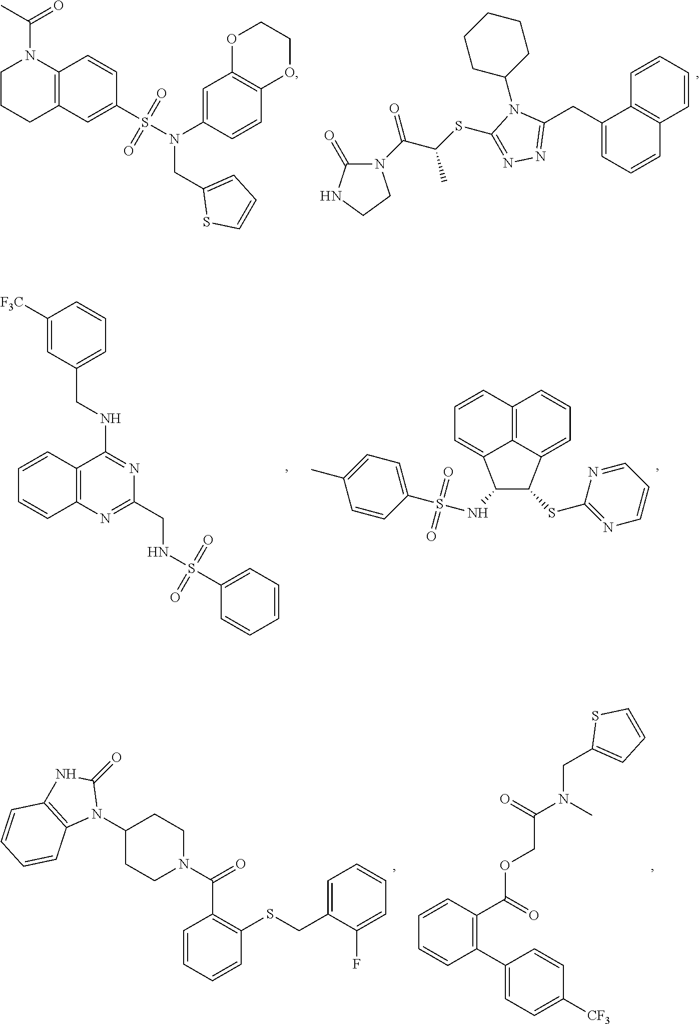

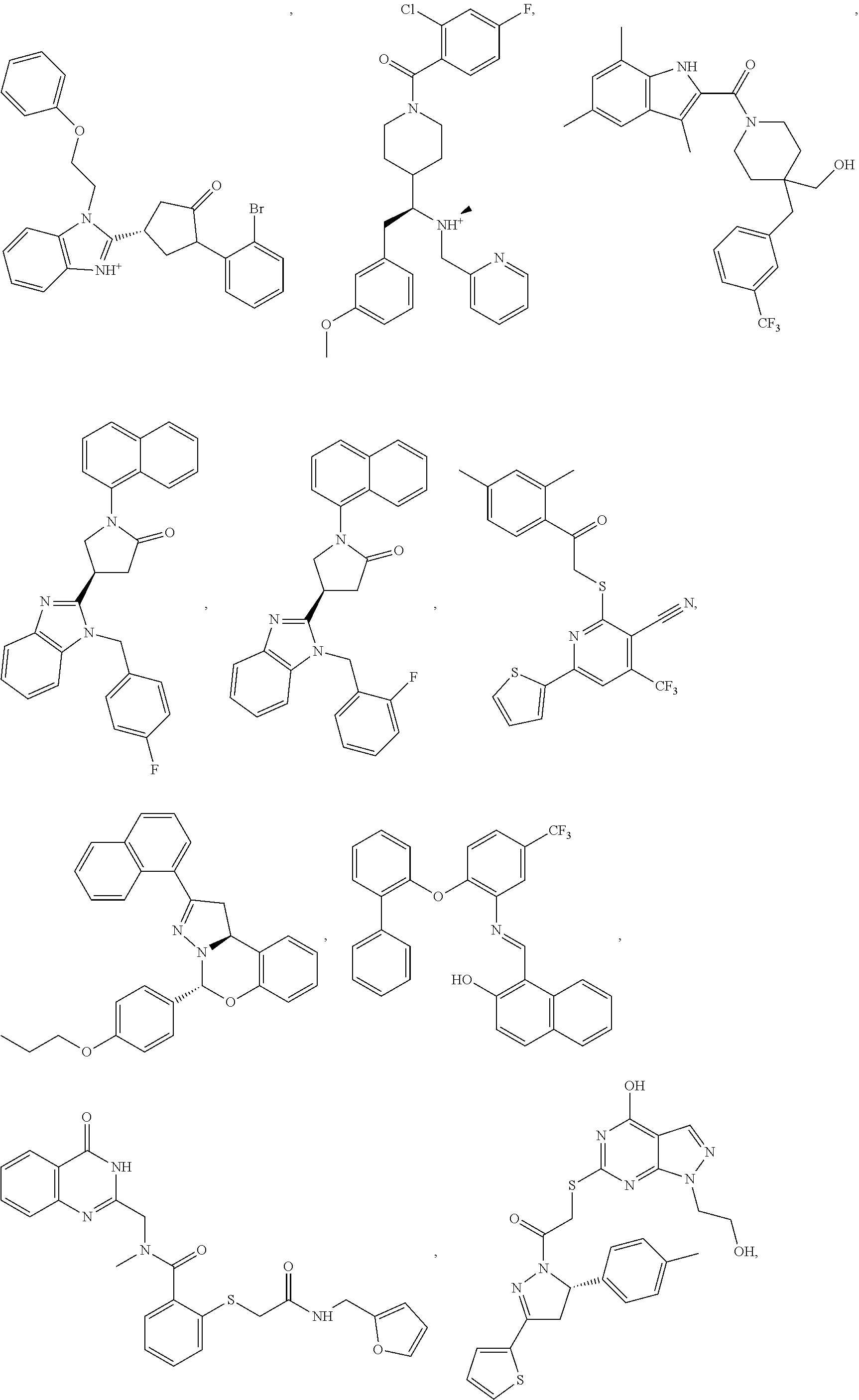

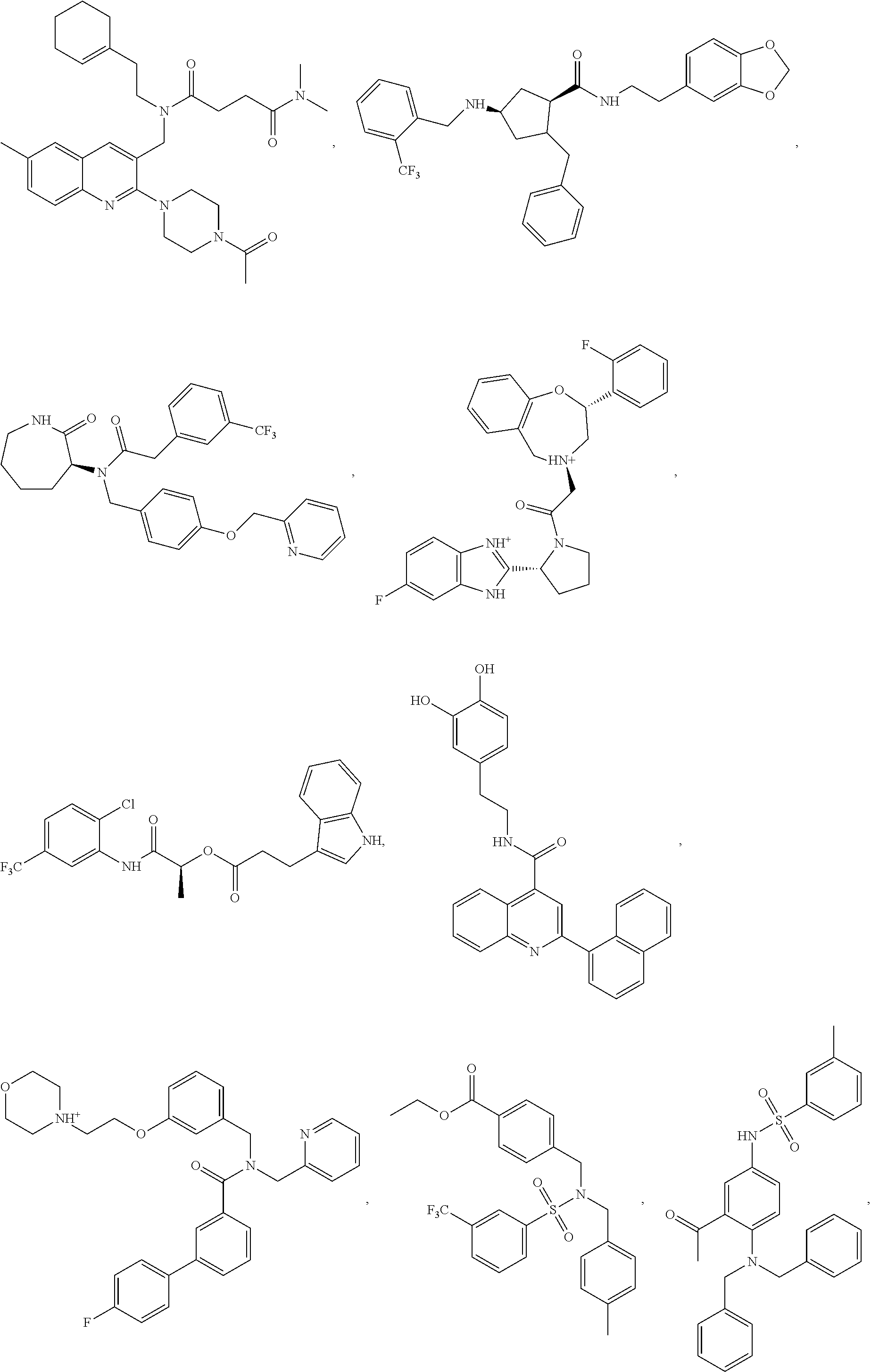

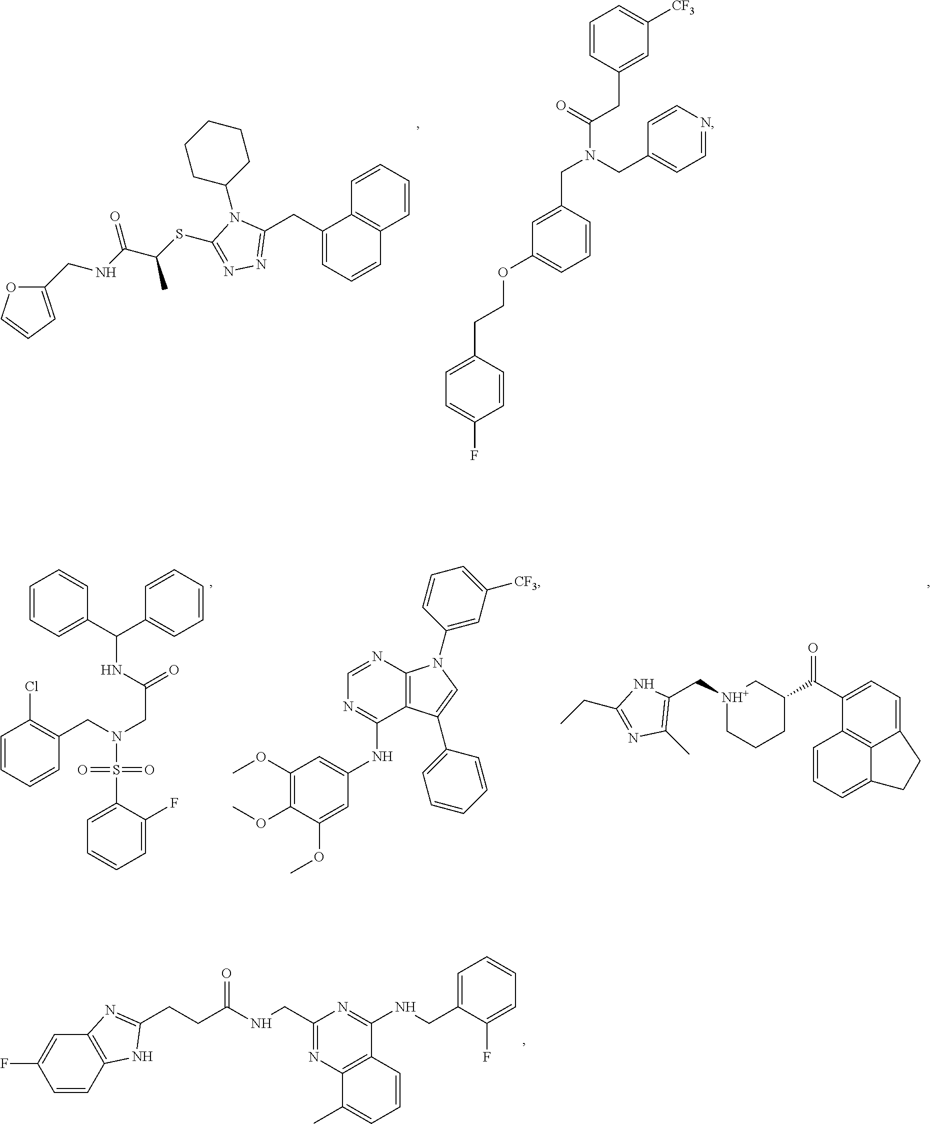 [SMILES: C[C@H](Sc1nnc(Cc2cccc3ccccc23)n1C1CCCCC1)C(=O)NCc1ccco1 ; O=C(Cc1cccc(C(F)(F)F)c1)N(Cc1ccncc1)Cc1cccc(OCCc2ccc(F)cc2)c1 ; O=C(CN(Cc1ccccc1Cl)S(=O)(=O)c1ccccc1F)NC(c1ccccc1)c1ccccc1 ; COc1cc(Nc2ncnc3c2c(-c2ccccc2)cn3-c2cccc(C(F)(F)F)c2)cc(OC)c1OC ; CCc1nc(C)c(C[N@@H+]2CCC[C@@H](C(=O)c3ccc4c5c(cccc35)CC4)C2)[nH]1 ; Cc1cccc2c(NCc3ccccc3F)nc(CNC(=O)CCc3nc4cc(F)ccc4[nH]3)nc12]

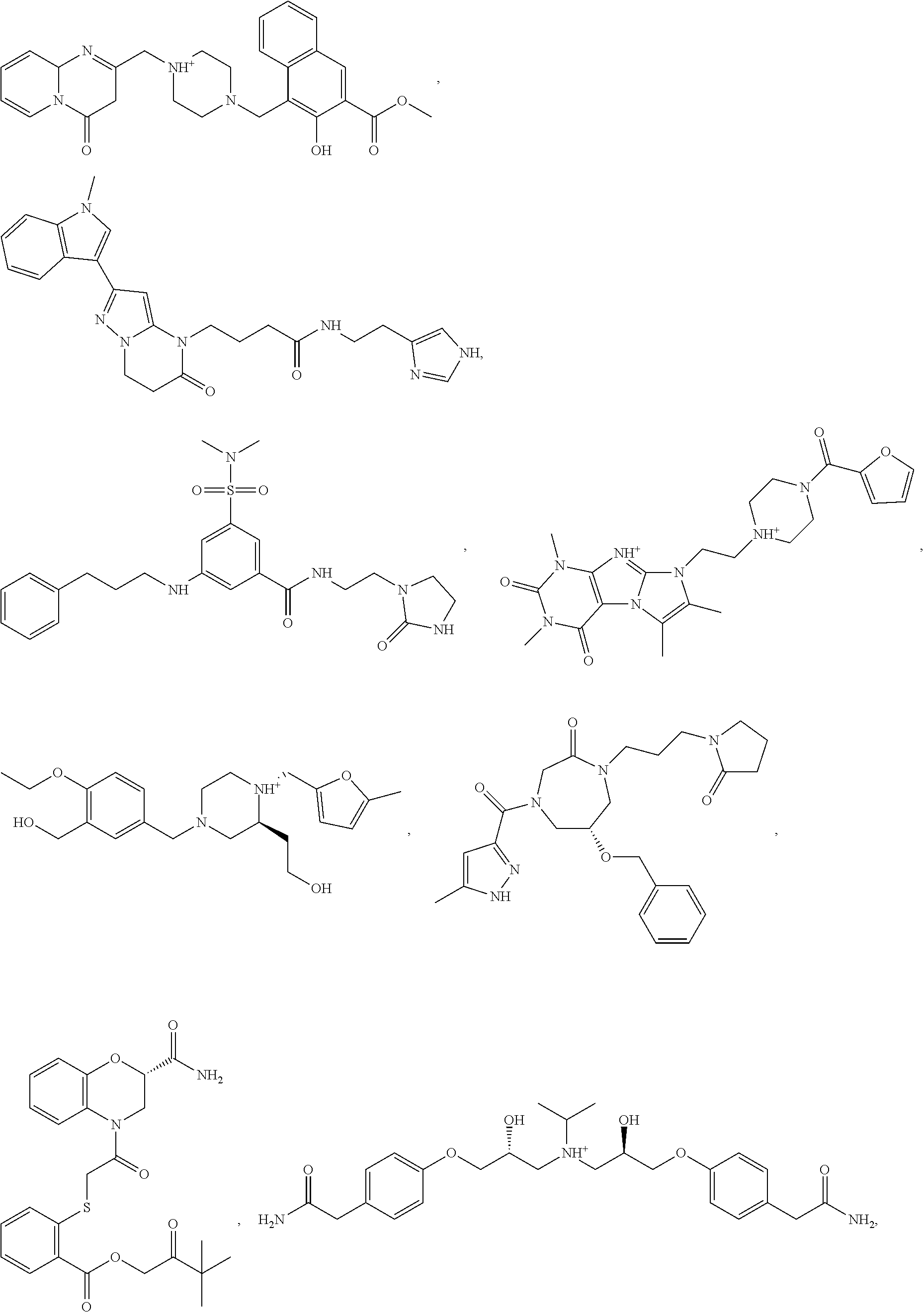

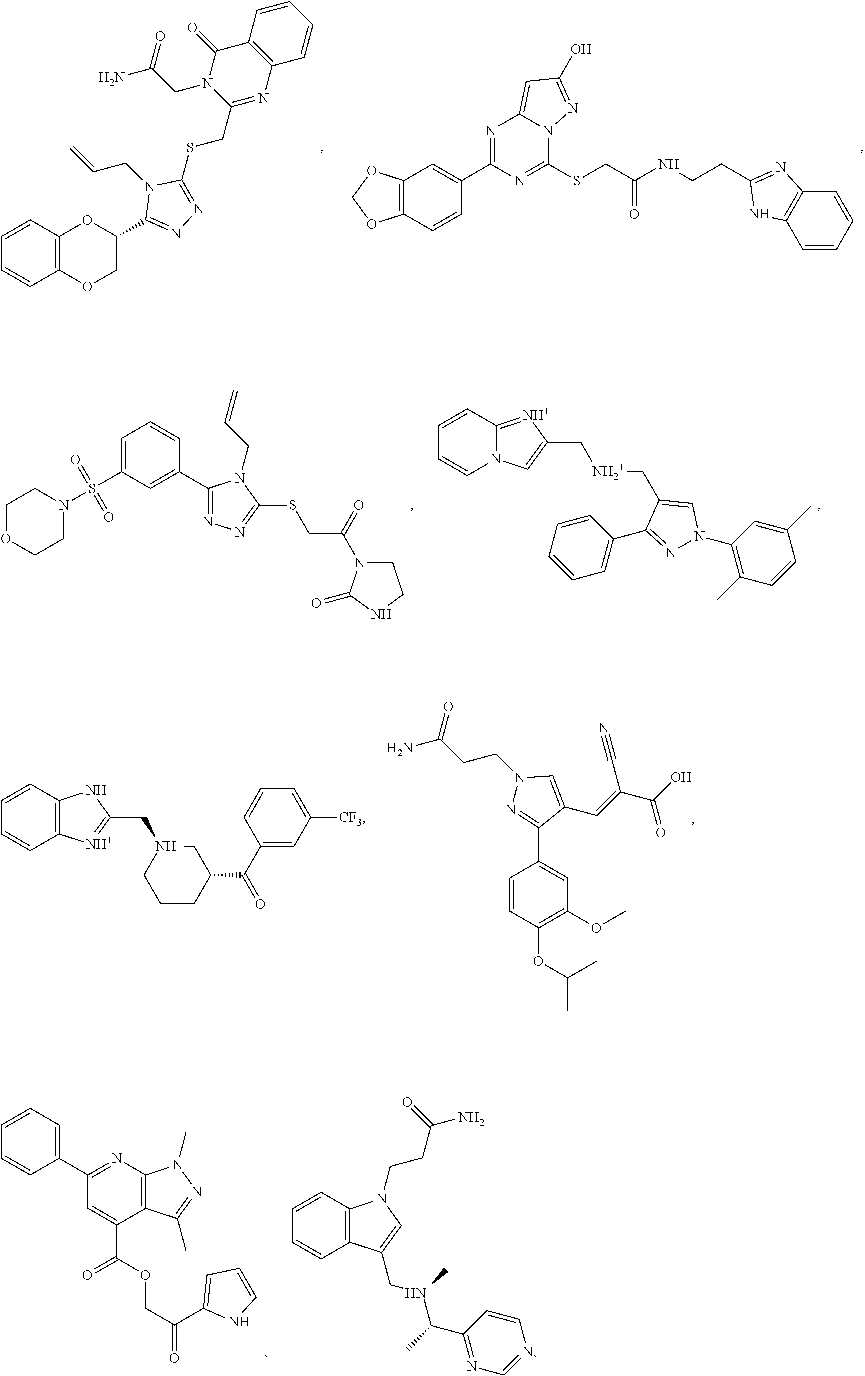

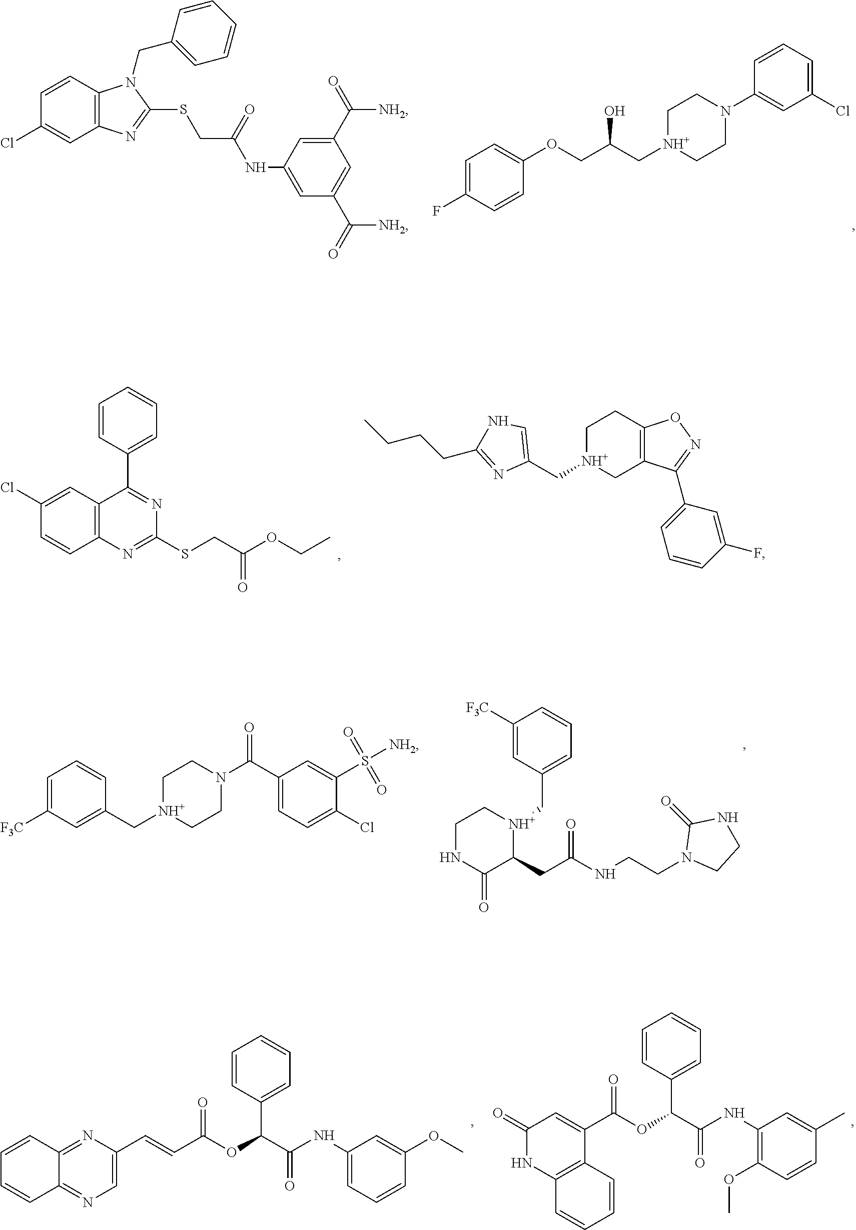

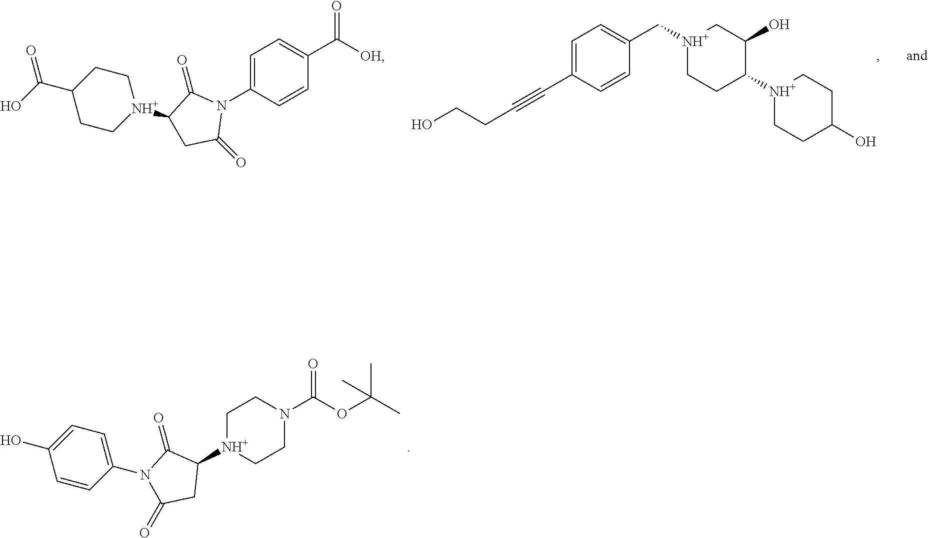

11. The method of claim 7, wherein the FTI is LNK-754.

12. The method of claim 6, wherein the ER protein folding enhancer is selected from the group consisting of Ryanodine receptor (RyR) inhibitor, inositol triphosphate (IP3) receptor inhibitor, activator of sarco/endoplasmic reticulum Ca²⁺-ATPase (SERCA), and any combinations thereof.

13. The method of claim 12, wherein the RyR inhibitor is selected from the group consisting of diltiazem (DILT), dantrolene (DANT), 1,1′-diheptyl-4,4′-bipyridinium (DHBP), JTV 519 fumarate, ruthenium red, and ryanodine.

14. The method of claim 13, wherein the RyR inhibitor is selected from the group consisting of DILT, DANT, and DHBP. The method of claim 6, wherein the subject is administered LNK-754 and DILT.

15. The method of claim 6, wherein the administration of the at least one ER-Golgi trafficking enhancer and the at least one ER protein folding enhancer produces a synergistic effect.

16. The method of claim 1, wherein the subject is administered the N-glycosylation enhancer.

17. The method of claim 16, wherein the N-glycosylation enhancer is selected from the group consisting of N-acetylglucosamine, N-acetylglucosamine-6-acetate, L-glutamine, fructose-6-phosphate, an allosteric activator of glutamine:F-6-P transaminase-1, and an allosteric activator of glutamine:F-6-P transaminase-2.

18. The method of claim 1, wherein the subject is administered the at least one ER-Golgi trafficking enhancer, the at least one ER protein folding enhancer, and the at least one N-glycosylation enhancer.

19. The method of claim 18, wherein the administration of the at least one ER-Golgi trafficking enhancer, the at least one ER protein folding enhancer, and the at least one N-glycosylation enhancer produces a synergistic effect.

20. The method of claim 1, wherein β-glucocerebrosidase (GCase) maturation level is increased in the subject after administration; wherein GCase solubility is increased in the subject after administration; wherein the amount of α-synuclein is reduced in the subject after administration; wherein endoplasmic reticulum (ER) morphology is improved in the subject after administration; or any combination thereof.
